(19) United States
(12) Patent Application Publication
(10) Pub. No.: US 2025/0011298 A1
(43) Pub. Date: Jan. 9, 2025

(54) CRYSTALLINE FORMS OF A CDK9 INHIBITOR AND USES THEREOF

(71) Applicant: PRELUDE THERAPEUTICS INCORPORATED, Wilmington, DE (US)

(72) Inventors: Ganfeng CAO, Chadds Ford, PA (US); Liang LU, Wilmington, DE (US); Andrew COMBS, Kennett Square, PA (US); Qun LI, Newark, DE (US); Huaping ZHANG, Newark, DE (US)

(21) Appl. No.: 18/700,928

(22) PCT Filed: Oct. 14, 2022
(86) PCT No.: PCT/US2022/078143

§ 371 Date: Apr. 12, 2024

Related U.S. Application Data
(60) Provisional application No. 63/255,562, filed on Oct. 14, 2021.

(51) Int. Cl. C07D 401/04; A61K 31/4439; A61K 31/635

(52) U.S. Cl. CPC C07D 401/04; A61K 31/4439; A61K 31/635

(57) ABSTRACT

The present disclosure provides novel crystalline forms of a compound that acts as a CDK9 modulator, processes for preparing the novel crystalline forms of a compound that acts as a CDK9 modulator, and uses thereof.

### CROSS-REFERENCE TO RELATED APPLICATION

This application claims the benefit of U.S. Provisional Application No. 63/255,562, filed Oct. 14, 2021, the entirety of which is incorporated by reference herein.

### FIELD

The present disclosure provides novel crystalline forms of a compound that acts as a CDK9 modulator, processes for preparing the novel crystalline forms of a compound that acts as a CDK9 modulator, and uses thereof.

### BACKGROUND

U.S. patent Provisional application Ser. No. 17/018,005, filed on Sep. 11, 2020 and published as US20210070761 on Mar. 11, 2021, discloses compounds that act as modulators of cyclin-dependent kinases (CDK) including CDK9, a family of serine/threonine kinases whose activities are dependent on association and activation by cyclins, play critical roles in regulating cell cycle and gene transcription (Malumbres, M. (2014). “Cyclin-dependent kinases.” Genome Biol 15(6): 122). “CDK9: A key player in cancer and other diseases.” J Cell Biochem 119(2): 1273-1284; Soutourina, J. (2018). As the master regulator controlling releasing of paused Pol II from the promoter, CDK9 plays pivotal roles in promoting gene expression. Consistently, inhibition of CDK9 triggers global down-regulation of gene expression (Olson, C. M., et al. (2018). “Pharmacological perturbation of CDK9 using selective CDK9 inhibition or degradation.” Nat Chem Biol 14(2): 163-170), among which are short-lived transcripts, such as the oncogene, c-Myc, and Mcl-1, a member of pro-survival Bcl-2 family of proteins that promote cancer cell survival (Chen, R., et al. (2005). “Transcription inhibition by flavopiridol: mechanism of chronic lymphocytic leukemia cell death.” Blood 106(7): 2513-2519; Youle, R. J. and A. Strasser (2008). “The BCL-2 protein family: opposing activities that mediate cell death.” Nat Rev Mol Cell Biol 9(1): 47-59), suggesting an indirect approach to target Mcl-1 to treat cancer (Krystof, V., et al. (2012). “Perspective of cyclin-dependent kinase 9 (CDK9) as a drug target.” Curr Pharm Des 18(20): 2883-2890). Indeed, several CDK9 inhibitors have been developed and showed promising anti-cancer activities in preclinical models and have been advanced into the clinic (Boffo, S., et al. (2018). “CDK9 inhibitors in acute myeloid leukemia.” J Exp Clin Cancer Res 37(1): 36). Interestingly, a recent study found that CDK9 inhibition also reactivates epigenetically silenced tumor suppressor genes, adding another line of evidence that supports targeting CDK9 for cancer therapy (Zhang, H., et al., (2018). “Targeting CDK9 Reactivates Epigenetically Silenced Genes in Cancer.” Cell 175(5): 1244-1258.e1226). For example, the compounds modulating the activity of CDK9, such as CDK9 inhibition, may be beneficial in the treatment of a disease or disorder associated with aberrant CDK activity, such as a cancer and a tumor cell. One particular compound is compound has a formula of

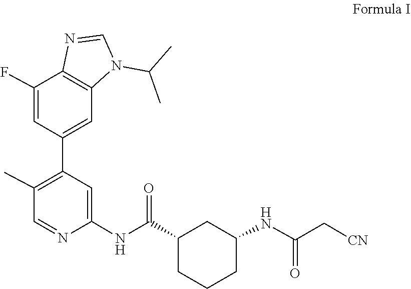 [SMILES: Cc1cnc(NC(=O)[C@H]2CCC[C@@H](NC(=O)CC#N)C2)cc1-c1cc(F)c2ncn(C(C)C)c2c1]

The compound of Formula I, as described or provided for herein, the ability of the compound to affect CDK9 activity, or the absence of such activity, methods for preparation of the compound of Formula I, as described or provided for herein, and other related compounds are disclosed in U.S. patent Provisional application Ser. No. 17/018,005, published as US20210070761 on Mar. 11, 2021, the contents of which are incorporated herein by reference in their entirety.

There remains a need in the art for improved forms of the compound of Formula I, as described or provided for herein, with improved properties. There also remains a need in the art for improved processes for preparing the compound of Formula I. The present embodiments described herein fulfill these needs and others.

### SUMMARY

The present disclosure provides novel crystalline forms of a compound of Formula I, as provided herein, processes for preparing the crystalline forms of the compound, and optionally isolating such crystalline forms.

The compound of Formula I can be crystallized with or without a coformer and is superior in properties. In some embodiments, crystalline forms of the compound of Formula I without a coformer are distinguished from the prior art by improved stability, processability and can also be used in pharmaceutical formulations. In some embodiments, crystalline forms of the compound of Formula I with coformers are distinguished from the prior art by improved stability, processability and can also be used in pharmaceutical formulations.

In some embodiments, crystalline forms of the compound of Formula I, as described herein, are provided.

In some embodiments, the compound of Formula I, as provided herein, can be crystallized without a coformer. In some embodiments, crystalline forms of the compound of Formula I without a coformer are provided. In some embodiments, crystalline Form I and Form II of the compound of Formula I without a coformer are provided.

In some embodiments, the crystalline Form I of the compound of Formula I is provided. In some embodiments, the crystalline Form I is characterized by an X-ray powder diffraction pattern comprising one or more peaks at about 7.2±0.5 degrees 2θ, at about 8.0±0.5 degrees 2θ, at about 10.1±0.5 degrees 2θ, at about 11.3±0.5 degrees 2θ, at about 13.0±0.5 degrees 2θ, at about 14.4±0.5 degrees 2θ, at about 15.3±0.5 degrees 2θ, at about 16.8±0.5 degrees 2θ, at about 18.2±0.5 degrees 2θ, at about 20.9±0.5 degrees 2θ, at about 21.6±0.5 degrees 2θ, at about 22.2±0.5 degrees 2θ, and at about 23.1±0.5 degrees 2θ. In some embodiments, the crystalline Form I is characterized by an X-ray powder diffraction pattern comprising one or more d-spacing values at about 12.3±0.5 degrees angstroms, at about 11.0±0.5 degrees angstroms, at about 8.7±0.5 degrees angstroms, at about 7.8±0.5 degrees angstroms, at about 6.8±0.5 degrees angstroms, at about 6.2±0.5 degrees angstroms, at about 5.8±0.5 degrees angstroms, at about 5.3±0.5 degrees angstroms, at about 4.9±0.5 degrees angstroms, at about 4.3±0.5 degrees angstroms, at about 4.1±0.5 degrees angstroms, at about 4.0±0.5 degrees angstroms, and at about 3.8±0.5 degrees angstroms.

In some embodiments, the crystalline Form II of the compound of Formula I is provided. In some embodiments, the crystalline Form I I is characterized by an X-ray powder diffraction pattern comprising one or more peaks at about 7.3±0.5 degrees 2θ, at about 8.1±0.5 degrees 2θ, at about 10.3±0.5 degrees 2θ, at about 11.5±0.5 degrees 2θ, at about 13.1±0.5 degrees 2θ, at about 15.4±0.5 degrees 2θ, at about 16.1±0.5 degrees 2θ, at about 17.0±0.5 degrees 2θ, at about 18.3±0.5 degrees 2θ, at about 19.2±0.5 degrees 2θ, at about 21.0±0.5 degrees 2θ, at about 21.7±0.5 degrees 2θ, at about 22.3±0.5 degrees 2θ, and at about 23.1±0.5 degrees 2θ. In some embodiments, the crystalline Form II is characterized by an X-ray powder diffraction pattern comprising one or more d-spacing values at about 12.1±0.5 degrees angstroms, at about 10.9±0.5 degrees angstroms, at about 8.6±0.5 degrees angstroms, at about 7.7±0.5 degrees angstroms, at about 6.8±0.5 degrees angstroms, at about 5.7±0.5 degrees angstroms, at about 5.5±0.5 degrees angstroms, at about 5.2±0.5 degrees angstroms, at about 4.8±0.5 degrees angstroms, at about 4.6±0.5 degrees angstroms, at about 4.2±0.5 degrees angstroms, at about 4.1±0.5 degrees angstroms, at about 4.0±0.5 degrees angstroms, and at about 3.8±0.5 degrees angstroms.

In some embodiments, the compound of Formula I, as provided herein, can be crystallized with a coformer. In some embodiments, crystalline forms of the compound of Formula I with a coformer are provided. In some embodiments, crystalline Form III, Form IV, Form V, Form VI, Form VII, Form VIII, Form IX, Form X, Form XI, Form XII, and Form XIII of the compound of Formula I with a coformer are provided. In some embodiments, the coformer is a coformer provided and described herein. In some embodiments, the coformer is succinic acid, adipic acid, fumaric acid, glutaric acid, gentisic acid, hydrochloric acid, 1-hydroxy-2-naphthoic acid, salicylic acid, oxalic acid, or D-(−)-tartaric acid.

In some embodiments, the crystalline Form III comprising the compound of Formula I and succinic acid in the molar ratio of about 1:1 is provided. In some embodiments, the crystalline Form III is characterized by an X-ray powder diffraction pattern comprising one or more peaks at about 5.8±0.5 degrees 2θ, at about 8.8±0.5 degrees 2θ, at about 10.5±0.5 degrees 2θ, at about 12.4±0.5 degrees 2θ, at about 14.4±0.5 degrees 2θ, at about 17.5±0.5 degrees 2θ, at about 17.9±0.5 degrees 2θ, at about 18.5±0.5 degrees 2θ, at about 19.5±0.5 degrees 2θ, at about 20.0±0.5 degrees 2θ, at about 20.7±0.5 degrees 2θ, at about 21.4±0.5 degrees 2θ, at about 22.4±0.5 degrees 2θ, at about 22.7±0.5 degrees 2θ, at about 23.6±0.5 degrees 2θ, at about 24.0±0.5 degrees 2θ, at about 24.7±0.5 degrees 2θ, at about 25.9±0.5 degrees 2θ, at about 26.4±0.5 degrees 2θ, at about 27.6±0.5 degrees 2θ, at about 29.0±0.5 degrees 2θ, at about 31.6±0.5 degrees 2θ, and at about 39.5±0.5 degrees 2θ. In some embodiments, the crystalline Form III is characterized by an X-ray powder diffraction pattern comprising one or more d-spacing values at about 15.1±0.5 degrees angstroms, at about 10.0±0.5 degrees angstroms, at about 8.4±0.5 degrees angstroms, at about 7.1±0.5 degrees angstroms, at about 6.2±0.5 degrees angstroms, at about 5.1±0.5 degrees angstroms, at about 4.9±0.5 degrees angstroms, at about 4.8±0.5 degrees angstroms, at about 4.6±0.5 degrees angstroms, at about 4.4±0.5 degrees angstroms, at about 4.3±0.5 degrees angstroms, at about 4.2±0.5 degrees angstroms, at about 4.0±0.5 degrees angstroms, at about 3.9±0.5 degrees angstroms, at about 3.8±0.5 degrees angstroms, at about 3.7±0.5 degrees angstroms, at about 3.6±0.5 degrees angstroms, at about 3.4±0.5 degrees angstroms, at about 3.4±0.5 degrees angstroms, at about 3.2±0.5 degrees angstroms, at about 3.1±0.5 degrees angstroms, at about 2.8±0.5 degrees angstroms, and at about 2.3±0.5 degrees angstroms.

In some embodiments, the crystalline Form IV comprising the compound of Formula I and glutaric acid in the molar ratio of about 2:1 is provided. In some embodiments, the crystalline Form IV is characterized by an X-ray powder diffraction pattern comprising one or more peaks at about 4.5±0.5 degrees 2θ, at about 6.0±0.5 degrees 2θ, at about 8.9±0.5 degrees 2θ, at about 11.1±0.5 degrees 2θ, at about 11.7±0.5 degrees 2θ, at about 13.2±0.5 degrees 2θ, at about 16.3±0.5 degrees 2θ, at about 17.1±0.5 degrees 2θ, at about 17.6±0.5 degrees 2θ, at about 18.4±0.5 degrees 2θ, at about 19.7±0.5 degrees 2θ, at about 20.5±0.5 degrees 2θ, at about 21.0±0.5 degrees 2θ, at about 21.9±0.5 degrees 2θ, at about 24.0±0.5 degrees 2θ, at about 24.7±0.5 degrees 2θ, at about 25.0±0.5 degrees 2θ, at about 26.2±0.5 degrees 2θ, and at about 29.2±0.5 degrees 2θ. In some embodiments, the crystalline Form IV is characterized by an X-ray powder diffraction pattern comprising one or more d-spacing values at about 19.4±0.5 degrees angstroms, at about 14.7±0.5 degrees angstroms, at about 10.0±0.5 degrees angstroms, at about 7.9±0.5 degrees angstroms, at about 7.5±0.5 degrees angstroms, at about 6.7±0.5 degrees angstroms, at about 5.4±0.5 degrees angstroms, at about 5.2±0.5 degrees angstroms, at about 5.0±0.5 degrees angstroms, at about 4.8±0.5 degrees angstroms, at about 4.5±0.5 degrees angstroms, at about 4.3±0.5 degrees angstroms, at about 4.2±0.5 degrees angstroms, at about 4.1±0.5 degrees angstroms, at about 3.7±0.5 degrees angstroms, at about 3.6±0.5 degrees angstroms, at about 3.6±0.5 degrees angstroms, at about 3.4±0.5 degrees angstroms, and at about 3.1±0.5 degrees angstroms.

In some embodiments, the crystalline Form V comprising the compound of Formula I and adipic acid in the molar ratio of about 1:1 is provided. In some embodiments, the crystalline Form V is characterized by an X-ray powder diffraction pattern comprising one or more peaks at about 4.7±0.5 degrees 2θ, at about 7.4±0.5 degrees 2θ, at about 9.2±0.5 degrees 2θ, at about 11.2±0.5 degrees 2θ, at about 13.8±0.5 degrees 2θ, at about 17.2±0.5 degrees 2θ, at about 18.1±0.5 degrees 2θ, at about 18.9±0.5 degrees 2θ, at about 25.1±0.5 degrees 2θ, and at about 25.9±0.5 degrees 2θ. In some embodiments, the crystalline Form V is characterized by an X-ray powder diffraction pattern comprising one or more d-spacing values at about 18.8±0.5 degrees angstroms, at about 11.9±0.5 degrees angstroms, at about 9.6±0.5 degrees angstroms, at about 7.9±0.5 degrees angstroms, at about 6.4±0.5 degrees angstroms, at about 5.1±0.5 degrees angstroms, at about 4.9±0.5 degrees angstroms, at about 4.7±0.5 degrees angstroms, at about 3.6±0.5 degrees angstroms, and at about 3.4±0.5 degrees angstroms.

In some embodiments, the crystalline Form VI comprising the compound of Formula I and fumaric acid in the molar ratio of about 2:1 is provided. In some embodiments, the crystalline Form VI is characterized by an X-ray powder diffraction pattern comprising one or more peaks at about 7.4±0.5 degrees 2θ, at about 9.5±0.5 degrees 2θ, at about 13.6±0.5 degrees 2θ, at about 14.7±0.5 degrees 2θ, at about 15.5±0.5 degrees 2θ, at about 16.4±0.5 degrees 2θ, at about 17.2±0.5 degrees 2θ, at about 18.2±0.5 degrees 2θ, at about 19.4±0.5 degrees 2θ, at about 20.5±0.5 degrees 2θ, at about 21.5±0.5 degrees 2θ, at about 23.5±0.5 degrees 2θ, at about 24.8±0.5 degrees 2θ, at about 25.7±0.5 degrees 2θ, at about 26.9±0.5 degrees 2θ, at about 29.4±0.5 degrees 2θ, and at about 30.7±0.5 degrees 2θ. In some embodiments, the crystalline Form VI is characterized by an X-ray powder diffraction pattern comprising one or more d-spacing values at about 12.0±0.5 degrees angstroms, at about 9.3±0.5 degrees angstroms, at about 6.5±0.5 degrees angstroms, at about 6.0±0.5 degrees angstroms, at about 5.7±0.5 degrees angstroms, at about 5.4±0.5 degrees angstroms, at about 5.2±0.5 degrees angstroms, at about 4.9±0.5 degrees angstroms, at about 4.6±0.5 degrees angstroms, at about 4.3±0.5 degrees angstroms, at about 4.1±0.5 degrees angstroms, at about 3.8±0.5 degrees angstroms, at about 3.6±0.5 degrees angstroms, at about 3.5±0.5 degrees angstroms, at about 3.3±0.5 degrees angstroms, at about 3.0±0.5 degrees angstroms, and at about 2.9±0.5 degrees angstroms.

In some embodiments, the crystalline Form VII comprising the compound of Formula I and fumaric acid in the molar ratio of about 2:1 is provided. In some embodiments, the crystalline Form VII is characterized by an X-ray powder diffraction pattern comprising one or more peaks at about 4.7±0.5 degrees 2θ, at about 5.8±0.5 degrees 2θ, at about 10.6±0.5 degrees 2θ, at about 11.3±0.5 degrees 2θ, at about 11.8±0.5 degrees 2θ, at about 12.6±0.5 degrees 2θ, at about 13.1±0.5 degrees 2θ, at about 14.0±0.5 degrees 2θ, at about 16.0±0.5 degrees 2θ, at about 17.0±0.5 degrees 2θ, at about 17.5±0.5 degrees 2θ, at about 18.7±0.5 degrees 2θ, at about 19.3±0.5 degrees 2θ, at about 21.2±0.5 degrees 2θ, at about 22.1±0.5 degrees 2θ, at about 24.2±0.5 degrees 2θ, at about 24.7±0.5 degrees 2θ, at about 26.2±0.5 degrees 2θ, and at about 27.4±0.5 degrees 2θ. In some embodiments, the crystalline Form VII is characterized by an X-ray powder diffraction pattern comprising one or more d-spacing values at about 18.9±0.5 degrees angstroms, at about 15.2±0.5 degrees angstroms, at about 8.4±0.5 degrees angstroms, at about 7.8±0.5 degrees angstroms, at about 7.5±0.5 degrees angstroms, at about 7.0±0.5 degrees angstroms, at about 6.8±0.5 degrees angstroms, at about 6.3±0.5 degrees angstroms, at about 5.5±0.5 degrees angstroms, at about 5.2±0.5 degrees angstroms, at about 5.1±0.5 degrees angstroms, at about 4.8±0.5 degrees angstroms, at about 4.6±0.5 degrees angstroms, at about 4.2±0.5 degrees angstroms, at about 4.0±0.5 degrees angstroms, at about 3.7±0.5 degrees angstroms, at about 3.6±0.5 degrees angstroms, at about 3.4±0.5 degrees angstroms, and at about 3.2±0.5 degrees angstroms.

In some embodiments, the crystalline Form VIII comprising the compound of Formula I and fumaric acid in the molar ratio of about 1:1 is provided. In some embodiments, the crystalline Form VIII is characterized by an X-ray powder diffraction pattern comprising one or more peaks at about 3.9±0.5 degrees 2θ, at about 5.7±0.5 degrees 2θ, at about 7.1±0.5 degrees 2θ, at about 8.6±0.5 degrees 2θ, at about 10.3±0.5 degrees 2θ, at about 12.1±0.5 degrees 2θ, at about 14.1±0.5 degrees 2θ, at about 17.1±0.5 degrees 2θ, at about 19.1±0.5 degrees 2θ, at about 20.6±0.5 degrees 2θ, at about 22.2±0.5 degrees 2θ, at about 23.0±0.5 degrees 2θ, at about 24.3±0.5 degrees 2θ, at about 26.0±0.5 degrees 2θ, at about 26.5±0.5 degrees 2θ, at about 28.5±0.5 degrees 2θ, at about 34.6±0.5 degrees 2θ, at about 35.4±0.5 degrees 2θ, at about 36.8±0.5 degrees 2θ, and at about 39.5±0.5 degrees 2θ. In some embodiments, the crystalline Form VIII is characterized by an X-ray powder diffraction pattern comprising one or more d-spacing values at about 22.5±0.5 degrees angstroms, at about 15.5±0.5 degrees angstroms, at about 12.5±0.5 degrees angstroms, at about 10.3±0.5 degrees angstroms, at about 8.6±0.5 degrees angstroms, at about 7.3±0.5 degrees angstroms, at about 6.3±0.5 degrees angstroms, at about 5.2±0.5 degrees angstroms, at about 4.7±0.5 degrees angstroms, at about 4.3±0.5 degrees angstroms, at about 4.0±0.5 degrees angstroms, at about 3.9±0.5 degrees angstroms, at about 3.7±0.5 degrees angstroms, at about 3.4±0.5 degrees angstroms, at about 3.4±0.5 degrees angstroms, at about 3.1±0.5 degrees angstroms, at about 2.6±0.5 degrees angstroms, at about 2.5±0.5 degrees angstroms, at about 2.4±0.5 degrees angstroms, and at about 2.3±0.5 degrees angstroms.

In some embodiments, the crystalline Form IX comprising the compound of Formula I and D-(−)-tartaric acid is provided. In some embodiments, the crystalline Form INX is characterized by an X-ray powder diffraction pattern comprising one or more peaks at about 5.3±0.5 degrees 2θ, at about 6.8±0.5 degrees 2θ, at about 9.0±0.5 degrees 2θ, at about 10.0±0.5 degrees 2θ, at about 15.5±0.5 degrees 2θ, at about 17.3±0.5 degrees 2θ, at about 18.2±0.5 degrees 2θ, at about 18.8±0.5 degrees 2θ, at about 19.9±0.5 degrees 2θ, at about 20.9±0.5 degrees 2θ, at about 21.3±0.5 degrees 2θ, at about 22.7±0.5 degrees 2θ, at about 23.6±0.5 degrees 2θ, at about 24.3±0.5 degrees 2θ, at about 25.5±0.5 degrees 2θ, at about 26.0±0.5 degrees 2θ, at about 27.1±0.5 degrees 2θ, at about 28.0±0.5 degrees 2θ, at about 28.8±0.5 degrees 2θ, at about 29.8±0.5 degrees 2θ, at about 33.4±0.5 degrees 2θ, at about 34.2±0.5 degrees 2θ, at about 36.3±0.5 degrees 2θ, at about 38.6±0.5 degrees 2θ, and at about 39.1±0.5 degrees 2θ. In some embodiments, the crystalline Form IX is characterized by an X-ray powder diffraction pattern comprising one or more d-spacing values at about 16.7±0.5 degrees angstroms, at about 12.9±0.5 degrees angstroms, at about 9.8±0.5 degrees angstroms, at about 8.8±0.5 degrees angstroms, at about 5.7±0.5 degrees angstroms, at about 5.1±0.5 degrees angstroms, at about 4.9±0.5 degrees angstroms, at about 4.7±0.5 degrees angstroms, at about 4.5±0.5 degrees angstroms, at about 4.3±0.5 degrees angstroms, at about 4.2±0.5 degrees angstroms, at about 3.9±0.5 degrees angstroms, at about 3.8±0.5 degrees angstroms, at about 3.7±0.5 degrees angstroms, at about 3.5±0.5 degrees angstroms, at about 3.4±0.5 degrees angstroms, at about 3.3±0.5 degrees angstroms, at about 3.2±0.5 degrees angstroms, at about 3.1±0.5 degrees angstroms, at about 3.0±0.5 degrees angstroms, at about 2.7±0.5 degrees angstroms, at about 2.6±0.5 degrees angstroms, at about 2.5±0.5 degrees angstroms, at about 2.3±0.5 degrees angstroms, and at about 2.3±0.5 degrees angstroms.

In some embodiments, the crystalline Form X comprising the compound of Formula I and hydrochloric acid is provided. In some embodiments, the crystalline Form X is characterized by an X-ray powder diffraction pattern comprising one or more peaks as shown in FIG. 50.

In some embodiments, the crystalline Form XI comprising the compound of Formula I and salicylic acid in the molar ratio of about 2:1 is provided. In some embodiments, the crystalline Form XI is characterized by an X-ray powder diffraction pattern comprising one or more peaks as shown in FIG. 52.

In some embodiments, the crystalline Form XII comprising the compound of Formula I and oxalic acid in the molar ratio of about 1:1 is provided. In some embodiments, the crystalline Form XII is characterized by an X-ray powder diffraction pattern comprising one or more peaks as shown in FIG. 54.

In some embodiments, the crystalline Form XIII of the compound of Formula I and 1-hydroxy-2-naphthoic acid is provided. In some embodiments, the crystalline Form XIII is characterized by an X-ray powder diffraction pattern comprising one or more peaks as shown in FIG. 59.

In some embodiments, a pharmaceutical composition comprising a crystalline form of the compound of Formula I, as described or provided for herein, is provided. In some embodiments, a pharmaceutical composition comprising the crystalline Form I, Form II, Form III, Form IV, Form V, Form VI, Form VII, Form VIII, Form IX, Form X, Form XI, Form, XII or Form XIII, as described and provided herein, is provided. In some embodiments, a pharmaceutical composition comprising the crystalline Form I, as described and provided herein, is provided. In some embodiments, a pharmaceutical composition comprising the crystalline Form II, as described and provided herein, is provided. In some embodiments, a pharmaceutical composition comprising the crystalline Form III, as described and provided herein, is provided. In some embodiments, a pharmaceutical composition comprising the crystalline Form VI, as described and provided herein, is provided. In some embodiments, a pharmaceutical composition comprising the crystalline Form V, as described and provided herein, is provided. In some embodiments, a pharmaceutical composition comprising the crystalline Form VI, as described and provided herein, is provided. In some embodiments, a pharmaceutical composition comprising the crystalline Form VII, as described and provided herein, is provided. In some embodiments, a pharmaceutical composition comprising the crystalline Form VIII comprising the compound of Formula I is provided. In some embodiments, a pharmaceutical composition comprising the crystalline Form IX, as described and provided herein, is provided. In some embodiments, a pharmaceutical composition comprising the crystalline Form X, as described and provided herein, is provided. In some embodiments, a pharmaceutical composition comprising the crystalline Form XI, as described and provided herein, is provided. In some embodiments, a pharmaceutical composition comprising the crystalline Form XII, as described and provided herein, is provided. In some embodiments, a pharmaceutical composition comprising the crystalline Form XIII, as described and provided herein, is provided.

In some embodiments, the pharmaceutical composition comprising a crystalline form of Forms I-XIII, as described or provided herein, further comprising a pharmaceutically acceptable excipient. In some embodiments, the pharmaceutical composition comprises the crystalline Form I. In some embodiments, the pharmaceutical composition comprises the crystalline Form II. In some embodiments, the pharmaceutical composition comprises the crystalline Form III. In some embodiments, the pharmaceutical composition comprises the crystalline Form VI. In some embodiments, the pharmaceutical composition comprises the crystalline Form V. In some embodiments, the pharmaceutical composition comprises the crystalline Form VI. In some embodiments, the pharmaceutical composition comprises the crystalline Form VII. In some embodiments, the pharmaceutical composition comprises the crystalline Form VIII. In some embodiments, the pharmaceutical composition comprises the crystalline Form IX. In some embodiments, the pharmaceutical composition comprises the crystalline Form X. In some embodiments, the pharmaceutical composition comprises the crystalline Form XI. In some embodiments, the pharmaceutical composition comprises the crystalline Form XII. In some embodiments, the pharmaceutical composition comprises the crystalline Form XIII.

In some embodiments, processes for preparing crystalline forms of the compound of Formula I with or without a coformer, as described or provided for herein, comprising crystallizing the compound to form the crystalline form and optionally isolating the crystalline form is provided.

In some embodiments, methods of inhibiting a CDK enzyme are provided, the method comprising: contacting the CDK enzyme with an effective amount of a crystalline form as provided herein, or a pharmaceutically acceptable salt or solvate thereof, or a pharmaceutical composition comprising the same.

In some embodiments, methods of treating a disease or disorder associated with aberrant CDK activity in a subject or a subject in need thereof are provided, the method comprising administering to the subject, a crystalline form as provided herein, or a pharmaceutically acceptable salt or solvate thereof, or a pharmaceutical composition comprising the same.

In some embodiments, methods of treating cancer in a subject or a subject in need thereof are provided, the method comprising administering to the subject, a crystalline form as provided herein, or a pharmaceutically acceptable salt or solvate a pharmaceutical composition comprising the same

In some embodiments, methods of inducing apoptosis in a cancer or tumor cell in a subject or a subject in need thereof are provided, the method comprising contacting the cancer or tumor cell with, or administering to the subject, an effective amount of a crystalline form as provided herein, or a pharmaceutically acceptable salt or solvate thereof, or a pharmaceutical composition as provided herein.

In some embodiments, methods of inhibiting phosphorylation of Ser2RNAP2 in a cancer or tumor cell in a subject or a subject in need thereof are provided, the method comprising contacting the cancer or tumor cell with, or administering to the subject, an effective amount of a crystalline form as provided herein, or a pharmaceutically acceptable salt or solvate thereof, or a pharmaceutical composition as provided herein.

In some embodiments, methods of reducing the level of induced myeloid leukemia cell differentiation protein Mcl-1 (MCL1) in a cancer or tumor cell in a subject or a subject in need thereof are provided, the method comprising contacting the cancer or tumor cell with, or administering to the subject, an effective amount of a crystalline form as provided herein, or a pharmaceutically acceptable salt or solvate thereof, or a pharmaceutical composition as provided herein.

In some embodiments, methods of reducing the level of MYC protein in a cancer or tumor cell in a subject or a subject in need thereof are provided, the method comprising contacting the cancer or tumor cell with, or administering to the subject, an effective amount of a crystalline form as provided herein, or a pharmaceutically acceptable salt or solvate thereof, or a pharmaceutical composition as provided herein.

In some embodiments, methods of inhibiting proliferation of a cancer or tumor cell in a subject or a subject in need thereof are provided, the method comprising contacting the cancer or tumor cell with, or administering to the subject, an effective amount of a crystalline form as provided herein, or a pharmaceutically acceptable salt or solvate thereof, or a pharmaceutical composition as provided herein.

In some embodiments, pharmaceutical composition comprising one or more of the crystalline forms provided herein, or pharmaceutically acceptable salts or solvates thereof, are provided.

The details of one or more embodiments are set forth in the description below. Other features, objects, and advantages of the present teachings will be apparent from the description of examples and also from the appending claims.

### BRIEF DESCRIPTION OF THE DRAWINGS

FIG. 1 shows an X-ray powder diffraction pattern of the amorphous form of the compound of Formula I.

FIG. 2 shows an X-ray powder diffraction pattern of the crystalline Form I of the compound of Formula I.

FIG. 3 shows a Differential Scanning Calorimetry (DSC) thermogram of the crystalline Form I.

FIG. 4 shows a thermogravimetric analysis (TGA) thermogram of the crystalline Form.

FIG. 5 shows a Proton Nuclear Magnetic Resonance Spectroscopy (¹H NMR) analysis of the crystalline Form I.

FIG. 6 shows an X-ray powder diffraction pattern of the crystalline Form II of the compound of Formula I.

FIG. 7 shows a Differential Scanning Calorimetry (DSC) thermogram of the crystalline Form II.

FIG. 8 shows a thermogravimetric analysis (TGA) thermogram of the crystalline Form II.

FIG. 9 shows a Proton Nuclear Magnetic Resonance Spectroscopy (¹H NMR) analysis of the crystalline Form II.

FIG. 10 shows a Proton Nuclear Magnetic Resonance Spectroscopy (¹H NMR) analysis of the crystalline Form I on a large scale.

FIG. 11 shows an X-ray powder diffraction pattern of the crystalline Form I of the compound of Formula I on a large scale.

FIG. 12 shows an X-ray powder diffraction pattern of the crystalline Form III of the compound of Formula I and succinic acid.

FIG. 13 shows a Differential Scanning Calorimetry (DSC) thermogram of the crystalline Form I I.

FIG. 14 shows a thermogravimetric analysis (TGA) thermogram of the crystalline Form III.

FIG. 15 shows a high-performance liquid chromatography analysis (HPLC) of the crystalline Form III.

FIG. 16 shows a Proton Nuclear Magnetic Resonance Spectroscopy (¹H NMR) analysis of the crystalline Form III.

FIG. 17 shows a Proton Nuclear Magnetic Resonance Spectroscopy (¹H NMR) comparison of the compound of Formula I and the crystalline Form III.

FIG. 18 shows an X-ray powder diffraction pattern of the crystalline Form IV of the compound of Formula I and glutaric acid.

FIG. 19 shows a Differential Scanning Calorimetry (DSC) thermogram of the crystalline Form IV.

FIG. 20 shows a thermogravimetric analysis (TGA) thermogram of the crystalline Form IV.

FIG. 21 shows a high-performance liquid chromatography analysis (HPLC) of the crystalline Form IV.

FIG. 22 shows a Proton Nuclear Magnetic Resonance Spectroscopy (¹H NMR) analysis of the crystalline Form IV.

FIG. 23 shows a Proton Nuclear Magnetic Resonance Spectroscopy (¹H NMR) comparison of the compound of Formula I and the crystalline Form IV.

FIG. 24 shows an X-ray powder diffraction pattern of the crystalline Form V of the compound of Formula I and adipic acid.

FIG. 25 shows a Differential Scanning Calorimetry (DSC) thermogram of the crystalline Form V.

FIG. 26 shows a thermogravimetric analysis (TGA) thermogram of the crystalline Form V.

FIG. 27 shows a high-performance liquid chromatography analysis (HPLC) of the crystalline Form V.

FIG. 28 shows a Proton Nuclear Magnetic Resonance Spectroscopy (¹H NMR) analysis of the crystalline Form V.

FIG. 29 shows a Proton Nuclear Magnetic Resonance Spectroscopy (¹H NMR) comparison of the compound of Formula I and the crystalline Form V.

FIG. 30 shows an X-ray powder diffraction pattern of the crystalline Form VI of the compound of Formula I and gentisic acid.

FIG. 31 shows a Differential Scanning Calorimetry (DSC) thermogram of the crystalline Form VI.

FIG. 32 shows a thermogravimetric analysis (TGA) thermogram of the crystalline Form VI.

FIG. 33 shows a high-performance liquid chromatography analysis (HPLC) of the crystalline Form VI.

FIG. 34 shows a Proton Nuclear Magnetic Resonance Spectroscopy (¹H NMR) analysis of the crystalline Form VI.

FIG. 35 shows a Proton Nuclear Magnetic Resonance Spectroscopy (¹H NMR) comparison of the compound of Formula I and the crystalline Form VI.

FIG. 36 shows an X-ray powder diffraction pattern of the crystalline Form VII of the compound of Formula I and fumaric acid.

FIG. 37 shows a Differential Scanning Calorimetry (DSC) thermogram of the crystalline Form VII.

FIG. 38 shows a thermogravimetric analysis (TGA) thermogram of the crystalline Form VII.

FIG. 39 shows a high-performance liquid chromatography analysis (HPLC) of the crystalline Form VII.

FIG. 40 shows a Proton Nuclear Magnetic Resonance Spectroscopy (¹H NMR) analysis of the crystalline Form VII.

FIG. 41 shows a Proton Nuclear Magnetic Resonance Spectroscopy (¹H NMR) comparison of the compound of Formula I and the crystalline Form VII.

FIG. 42 shows an X-ray powder diffraction pattern of the crystalline Form VIII of the compound of Formula I and fumaric acid.

FIG. 43 shows a Differential Scanning Calorimetry (DSC) thermogram of the crystalline Form VIII.

FIG. 44 shows a thermogravimetric analysis (TGA) thermogram of the crystalline Form VIII.

FIG. 45 shows a Proton Nuclear Magnetic Resonance Spectroscopy (¹H NMR) analysis of the crystalline Form VIII.

FIG. 46 shows an X-ray powder diffraction pattern of the crystalline Form IX of the compound of Formula I and D-(−)-tartaric acid.

FIG. 47 shows a Differential Scanning Calorimetry (DSC) thermogram of the crystalline Form IX.

FIG. 48 shows a thermogravimetric analysis (TGA) thermogram of the crystalline Form IX.

FIG. 49 shows a Proton Nuclear Magnetic Resonance Spectroscopy (¹H NMR) analysis of the crystalline Form IX.

FIG. 50 shows an X-ray powder diffraction pattern overlay of the crystalline Form X of the compound of Formula I and hydrochloride acid prepared from methylethylketone (MEK), n-butanol (n-BuOH), and n-propanol.

FIG. 51 shows a Differential Scanning Calorimetry (DSC) thermogram of the crystalline Form X.

FIG. 52 shows an X-ray powder diffraction pattern of the crystalline Form XI of the compound of Formula I and salicylic acid.

FIG. 53 shows a Proton Nuclear Magnetic Resonance Spectroscopy (¹HMR) analysis of the crystalline Form XI.

FIG. 54 shows an X-ray powder diffraction pattern of the crystalline Form XII of the compound of Formula I and oxalic acid.

FIG. 55 shows a Differential Scanning Calorimetry (DSC) thermogram of the crystalline Form XII.

FIG. 56 shows a high-performance liquid chromatography analysis (HPLC) of the crystalline Form XII.

FIG. 57 shows a Proton Nuclear Magnetic Resonance Spectroscopy (¹H NMR) analysis of the crystalline Form XII.

FIG. 58 shows a Carbon 13 Nuclear Magnetic Resonance Spectroscopy (¹³C NMR) analysis of the crystalline Form XII.

FIG. 59 shows an X-ray powder diffraction pattern of the crystalline Form XIII of the compound of Formula I and 1-hydroxy-2-naphthoic acid.

### DETAILED DESCRIPTION

The term “salt” or “salts” may refer to any acid addition salts, including addition salts of free acids or addition salts of free bases. All of these salts (or other similar salts) may be prepared by conventional means. All such salts are acceptable, provided that they are non-toxic and do not substantially interfere with the desired pharmacological activity.

The term “therapeutically effective amount” means the amount of a crystalline form that, when administered to a mammal for treating a state, disorder or condition, is sufficient to effect a treatment (as defined below). The “therapeutically effective amount” will vary depending on the compound, the disease and its severity, the age, weight, physical condition and responsiveness of the mammal to be treated.

The term “pharmaceutically acceptable” means biologically or pharmacologically compatible for in vivo use in animals or humans, and preferably means approved by a regulatory agency of the Federal or a State government or listed in the U.S. Pharmacopeia or other generally recognized pharmacopeia for use in animals, and more particularly in humans.

As used herein, the terms “treat,” “treated,” or “treating” means both therapeutic treatment and prophylactic measures wherein the object is to slow down (lessen) an undesired physiological condition, disorder, or disease or obtain beneficial or desired clinical results. For purposes of this invention, beneficial or desired clinical results include, but are not limited to, alleviation of symptoms; diminishment of the extent of the condition, disorder, or disease; stabilized (i.e., not worsening) state of condition, disorder, or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of the condition, disorder, or disease. Treatment includes eliciting a clinically significant response without excessive levels of side effects. Treatment also includes prolonging survival as compared to expected survival if not receiving treatment. Thus, “treatment of pain” or “treating pain” means an activity that alleviates or ameliorates any of the primary phenomena or secondary symptoms associated with the pain or other condition described herein.

The term “additive” is defined as the interaction of two or more agents so that their combined effect is the same as the sum of their individual effects. For example, if the effect of drug A alone in treating a disease is 25%, and the effect of drug B alone in treating a disease is 25%, but when the two drugs are combined, the effect in treating the disease is 50%, the effect of A and B is additive.

The term “pharmaceutically acceptable” or “therapeutically acceptable” refers to molecular entities and compositions that are physiologically tolerable and preferably do not typically produce an allergic or similar untoward reaction, such as gastric upset, dizziness, and the like, when administered to a human. Preferably, as used herein, the term “pharmaceutically acceptable” means approved by a regulatory agency of the Federal or a State government or listed in the U.S. Pharmacopeia or other generally recognized pharmacopeia (e.g., Remington's Pharmaceutical Sciences, Mack Publishing Co. (A. R. Gennaro edit. 1985)) for use in animals, and more particularly in humans.

The term “about”, “ca.”, or “approximately” means plus or minus 5%. In some embodiments, the term “about”, “ca.”, or “approximately” means plus or minus 10%.

The present embodiments provide methods to crystallize a compound Formula I with or without a coformer, as described or provided for herein.

In some embodiments, processes for preparing a crystalline form of the compound having a formula of Formula I with or without a coformer as described or provided herein. In some embodiments, processes for preparing a crystalline form of the compound of Formula I without a coformer as described or provided herein. In some embodiments, processes for preparing a crystalline form of the compound of Formula I with a coformer as described or provided herein. In some embodiments, the process comprises co-crystallizing the compound of Formula I and the coformer to form the crystalline form of the compound and the coformer and optionally isolating the crystalline form of the compound and the coformer. In some embodiments, the process comprises slurrying the compound and the coformer in an organic solvent to form the crystalline form therefrom. In some embodiments, the process further comprises washing the slurry with the organic solvent.

In some embodiments, crystalline forms of the compound of Formula I with a coformer are provided. In some embodiments, the coformer is an acid. In some embodiments, the acid is a pharmaceutically acceptable acid. In some embodiments, the pharmaceutically acceptable acid is selected from succinic acid, adipic acid, fumaric acid, glutaric acid, gentisic acid, hydrochloric acid, 1-hydroxy-2-naphthoic acid, salicylic acid, oxalic acid, and D-(−)-tartaric acid or. In some embodiments, the pharmaceutically acceptable acid is succinic acid, adipic acid, fumaric acid, glutaric acid, gentisic acid, hydrochloric acid, 1-hydroxy-2-naphthoic acid, salicylic acid, oxalic acid, or D-(−)-tartaric acid, or any combination thereof. In some embodiments, the pharmaceutically acceptable acid is succinic acid. In some embodiments, the pharmaceutically acceptable acid is adipic acid. In some embodiments, the pharmaceutically acceptable acid is fumaric acid. In some embodiments, the pharmaceutically acceptable acid is glutaric acid. In some embodiments, the pharmaceutically acceptable acid is gentisic acid. In some embodiments, the pharmaceutically acceptable acid is hydrochloric acid. In some embodiments, the pharmaceutically acceptable acid is 1-hydroxy-2-naphthoic acid. In some embodiments, the pharmaceutically acceptable acid is salicylic acid. In some embodiments, the pharmaceutically acceptable acid is oxalic acid. In some embodiments, the pharmaceutically acceptable acid is D-(−)-tartaric acid.

A compound of Formula I, as described or provided for herein, or the pharmaceutically acceptable salt thereof, can be prepared according to the synthesis described in U.S. patent Provisional application Ser. No. 17/018,005, published as US20210070761 on Mar. 11, 2021, which is hereby incorporated by reference in its entirety or according to the synthesis as described or provided for herein. For example, in some embodiments, a compound of Formula I, or the pharmaceutically acceptable salt thereof, can be prepared according to the synthesis described in U.S. patent Provisional application Ser. No. 17/018,005, which is hereby incorporated by reference in its entirety.

An amorphous form of the compound of Formula I, or the pharmaceutically acceptable salt thereof, can be prepared according to the synthesis described in U.S. Patent Provisional Application Ser. No. 17/018,005, published as US20210070761 on Mar. 11, 2021, which is hereby incorporated by reference in its entirety. The amorphous form of the compound of Formula I or the pharmaceutically acceptable salt thereof can then be isolated using silica gel chromatographs. Silica gel chromatographs may not be feasible for large-scale manufacturing of the compound for commercial production. Therefore, a crystalline form is needed that can be better used in the manufacturing and use of pharmaceutical compositions. Although in some instances, preparing crystal forms of compounds can be straightforward, this was not the case for a compound of Formula I. The present embodiments provide for the surprising and unexpected result of crystalline forms of Formula I. In some embodiments, the crystalline form is Form I, Form II, Form III, Form IV, Form V, Form VI, Form VII, Form VIII, Form IX, Form X, Form XI, Form XII, or Form XIII as provided for herein. In some embodiments, the crystalline form is Form I, as provided for herein. In some embodiments, the crystalline form is Form II, as provided for herein. In some embodiments, the crystalline form is Form III, as provided for herein. In some embodiments, the crystalline form is Form VI, as provided for herein. In some embodiments, the crystalline form is Form V, as provided for herein. In some embodiments, the crystalline form is Form VI, as provided for herein. In some embodiments, the crystalline form is Form VII, as provided for herein. In some embodiments, the crystalline form is Form VIII, as provided for herein. In some embodiments, the crystalline form is Form IX, as provided for herein. In some embodiments, the crystalline form is Form X, as provided for herein. In some embodiments, the crystalline form is Form XI, as provided for herein. In some embodiments, the crystalline form is Form XII, as provided for herein. In some embodiments, the crystalline form is or Form XIII as provided for herein.

In some embodiments, crystalline forms of the compound having a formula of Formula I without a coformer as described or provided herein. In some embodiments, the crystalline form is Form I or Form II. In some embodiments, the crystalline form is Form I. In some embodiments, the crystalline form is Form II.

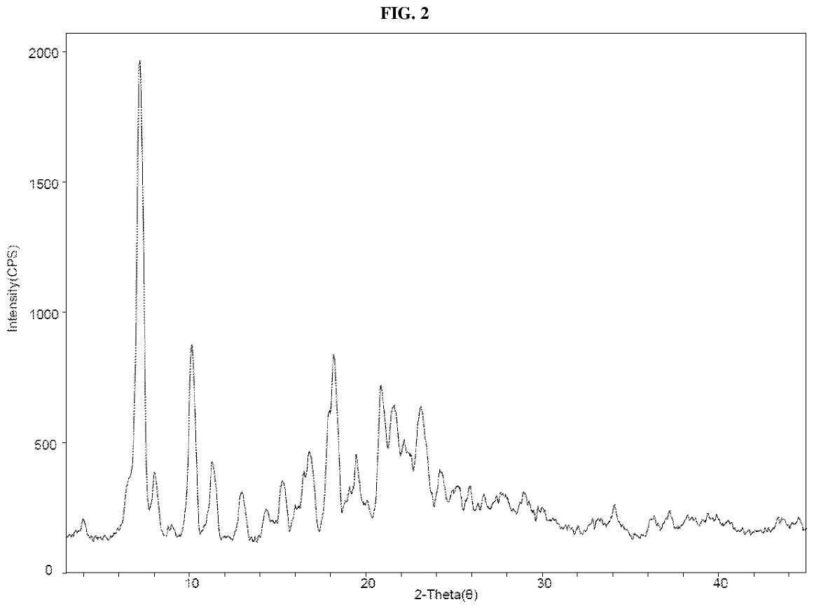

In some embodiments, the crystalline Form I of the compound of Formula I is provided. In some embodiments, the crystalline Form I is characterized by an X-ray powder diffraction pattern substantially, as shown in FIG. 2. In some embodiments, the crystalline Form I is characterized by an X-ray powder diffraction pattern comprising one or more peaks, as provided in Table 2. In some embodiments, the crystalline Form I is characterized by an X-ray powder diffraction pattern comprising substantially all of, or all of, the peaks as provided in Table 2.

In some embodiments, the crystalline Form I is characterized by an X-ray powder diffraction pattern comprising a peak at about 7.2±0.5 degrees 2θ.

In some embodiments, the crystalline Form I is characterized by an X-ray powder diffraction pattern comprising a peak at about 8.0±0.5 degrees 2θ.

In some embodiments, the crystalline Form I is characterized by an X-ray powder diffraction pattern comprising a peak at about 10.1±0.5 degrees 2θ.

In some embodiments, the crystalline Form I is characterized by an X-ray powder diffraction pattern comprising a peak at about 11.3±0.5 degrees 2θ.

In some embodiments, the crystalline Form I is characterized by an X-ray powder diffraction pattern comprising a peak at about 13.0±0.5 degrees 2θ.

In some embodiments, the crystalline Form I is characterized by an X-ray powder diffraction pattern comprising a peak at about 14.4±0.5 degrees 2θ.

In some embodiments, the crystalline Form I is characterized by an X-ray powder diffraction pattern comprising a peak at about 15.3±0.5 degrees 2θ.

In some embodiments, the crystalline Form I is characterized by an X-ray powder diffraction pattern comprising a peak at about 16.8±0.5 degrees 2θ.

In some embodiments, the crystalline Form I is characterized by an X-ray powder diffraction pattern comprising a peak at about 18.2±0.5 degrees 2θ.

In some embodiments, the crystalline Form I is characterized by an X-ray powder diffraction pattern comprising a peak at about 20.9±0.5 degrees 2θ.

In some embodiments, the crystalline Form I is characterized by an X-ray powder diffraction pattern comprising a peak at about 21.6±0.5 degrees 2θ.

In some embodiments, the crystalline Form I is characterized by an X-ray powder diffraction pattern comprising a peak at about 22.2±0.5 degrees 2θ.

In some embodiments, the crystalline Form I is characterized by an X-ray powder diffraction pattern comprising a peak at about 23.1±0.5 degrees 2θ.

In some embodiments, the crystalline Form I is characterized by an X-ray powder diffraction pattern comprising peaks at about 7.2±0.5 degrees 2θ, and at about 10.1±0.5 degrees 2θ.

In some embodiments, the crystalline Form I is characterized by an X-ray powder diffraction pattern comprising peaks at about 7.2±0.5 degrees 2θ, and at about 18.2±0.5 degrees 2θ.

In some embodiments, the crystalline Form I is characterized by an X-ray powder diffraction pattern comprising peaks at about 7.2±0.5 degrees 2θ, and at about 20.9±0.5 degrees 2θ.

In some embodiments, the crystalline Form I is characterized by an X-ray powder diffraction pattern comprising peaks at about 7.2±0.5 degrees 2θ, and at about 21.6±0.5 degrees 2θ.

In some embodiments, the crystalline Form I is characterized by an X-ray powder diffraction pattern comprising peaks at about 7.2±0.5 degrees 2θ, and at about 23.1±0.5 degrees 2θ.

In some embodiments, the crystalline Form I is characterized by an X-ray powder diffraction pattern comprising peaks at about 7.2±0.5 degrees 2θ, at about 10.1±0.5 degrees 2θ, and at about 18.2±0.5 degrees 2θ.

In some embodiments, the crystalline Form I is characterized by an X-ray powder diffraction pattern comprising peaks at about 7.2±0.5 degrees 2θ, at about 10.1±0.5 degrees 2θ, at about 18.2±0.5 degrees 2θ, at about 20.9±0.5 degrees 2θ, at about 21.6±0.5 degrees 2θ, at about 23.1±0.5 degrees 2θ.

In some embodiments, the crystalline Form I is characterized by an X-ray powder diffraction pattern comprising one or more peaks at about 7.2±0.5 degrees 2θ, at about 10.1±0.5 degrees 2θ, at about 18.2±0.5 degrees 2θ, at about 20.9±0.5 degrees 2θ, at about 21.6±0.5 degrees 2θ, at about 23.1±0.5 degrees 2θ.

In some embodiments, the crystalline Form I is characterized by an X-ray powder diffraction pattern comprising a peak at about 7.2±0.5 degrees 2θ, at about 8.0±0.5 degrees 2θ, at about 10.1±0.5 degrees 2θ, at about 11.3±0.5 degrees 2θ, at about 13.0±0.5 degrees 2θ, at about 14.4±0.5 degrees 2θ, at about 15.3±0.5 degrees 2θ, at about 16.8±0.5 degrees 2θ, at about 18.2±0.5 degrees 2θ, at about 20.9±0.5 degrees 2θ, at about 21.6±0.5 degrees 2θ, at about 22.2±0.5 degrees 2θ, and at about 23.1±0.5 degrees 2θ.

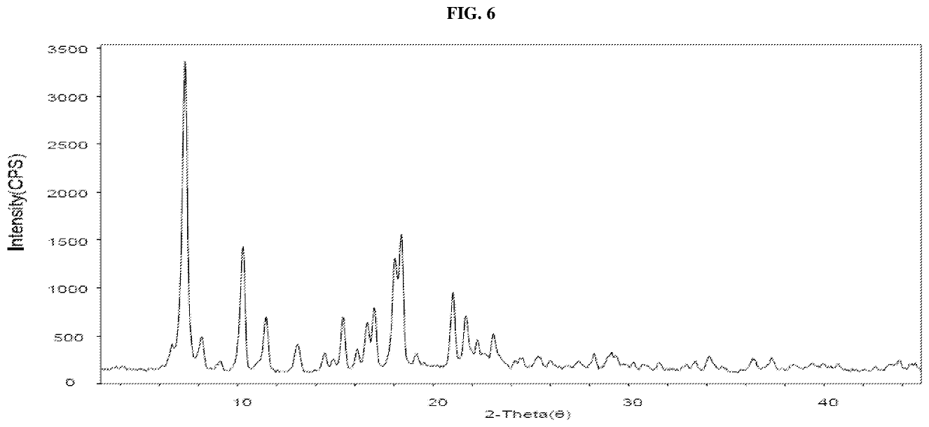

In some embodiments, the crystalline Form II is characterized by an X-ray powder diffraction pattern substantially, as shown in FIG. 6. In some embodiments, the crystalline Form II is characterized by an X-ray powder diffraction pattern comprising one or more peaks, as provided in Table 2. In some embodiments, the crystalline Form II is characterized by an X-ray powder diffraction pattern comprising substantially all of, or all of, the peaks as provided in Table 2.

In some embodiments, the crystalline Form II is characterized by an X-ray powder diffraction pattern comprising a peak at about 7.3±0.5 degrees 2θ.

In some embodiments, the crystalline Form II is characterized by an X-ray powder diffraction pattern comprising a peak at about 8.1±0.5 degrees 2θ.

In some embodiments, the crystalline Form II is characterized by an X-ray powder diffraction pattern comprising a peak at about 10.3±0.5 degrees 2θ.

In some embodiments, the crystalline Form II is characterized by an X-ray powder diffraction pattern comprising a peak at about 11.5±0.5 degrees 2θ.

In some embodiments, the crystalline Form II is characterized by an X-ray powder diffraction pattern comprising a peak at about 13.1±0.5 degrees 2θ.

In some embodiments, the crystalline Form II is characterized by an X-ray powder diffraction pattern comprising a peak at about 15.4±0.5 degrees 2θ.

In some embodiments, the crystalline Form II is characterized by an X-ray powder diffraction pattern comprising a peak at about 16.1±0.5 degrees 2θ.

In some embodiments, the crystalline Form II is characterized by an X-ray powder diffraction pattern comprising a peak at about 17.0±0.5 degrees 2θ.

In some embodiments, the crystalline Form II is characterized by an X-ray powder diffraction pattern comprising a peak at about 18.3±0.5 degrees 2θ.

In some embodiments, the crystalline Form II is characterized by an X-ray powder diffraction pattern comprising a peak at about 19.2±0.5 degrees 2θ.

In some embodiments, the crystalline Form II is characterized by an X-ray powder diffraction pattern comprising a peak at about 21.0±0.5 degrees 2θ.

In some embodiments, the crystalline Form II is characterized by an X-ray powder diffraction pattern comprising a peak at about 21.7±0.5 degrees 2θ.

In some embodiments, the crystalline Form II is characterized by an X-ray powder diffraction pattern comprising a peak at about 22.3±0.5 degrees 2θ.

In some embodiments, the crystalline Form II is characterized by an X-ray powder diffraction pattern comprising a peak at about and at about 23.1±0.5 degrees 2θ.

In some embodiments, the crystalline Form II is characterized by an X-ray powder diffraction pattern comprising peaks at about 7.3±0.5 degrees 2θ and at about 10.3±0.5 degrees 2θ.

In some embodiments, the crystalline Form II is characterized by an X-ray powder diffraction pattern comprising peaks at about 7.3±0.5 degrees 2θ and at about 11.5±0.5 degrees 2θ.

In some embodiments, the crystalline Form II is characterized by an X-ray powder diffraction pattern comprising peaks at about 7.3±0.5 degrees 2θ and at about 15.4±0.5 degrees 2θ.

In some embodiments, the crystalline Form II is characterized by an X-ray powder diffraction pattern comprising peaks at about 7.3±0.5 degrees 2θ and at about 17.0±0.5 degrees 2θ.

In some embodiments, the crystalline Form II is characterized by an X-ray powder diffraction pattern comprising peaks at about 7.3±0.5 degrees 2θ and at about 18.3±0.5 degrees 2θ.

In some embodiments, the crystalline Form II is characterized by an X-ray powder diffraction pattern comprising peaks at about 7.3±0.5 degrees 2θ and at about 21.0±0.5 degrees 2θ.

In some embodiments, the crystalline Form II is characterized by an X-ray powder diffraction pattern comprising peaks at about 7.3±0.5 degrees 2θ, at about 10.3±0.5 degrees 2θ, at about 11.5±0.5 degrees 2θ, at about 15.4±0.5 degrees 2θ, at about 17.0±0.5 degrees 2θ, at about 18.3±0.5 degrees 2θ, and at about 21.0±0.5 degrees 2θ.

In some embodiments, the crystalline Form II is characterized by an X-ray powder diffraction pattern comprising one or more peaks at about 7.3±0.5 degrees 2θ, at about 10.3±0.5 degrees 2θ, at about 11.5±0.5 degrees 2θ, at about 15.4±0.5 degrees 2θ, at about 17.0±0.5 degrees 2θ, at about 18.3±0.5 degrees 2θ, and at about 21.0±0.5 degrees 2θ.

In some embodiments, the crystalline Form II is characterized by an X-ray powder diffraction pattern comprising one or more peaks at about 7.3±0.5 degrees 2θ, at about 8.1±0.5 degrees 2θ, at about 10.3±0.5 degrees 2θ, at about 11.5±0.5 degrees 2θ, at about 13.1±0.5 degrees 2θ, at about 15.4±0.5 degrees 2θ, at about 16.1±0.5 degrees 2θ, at about 17.0±0.5 degrees 2θ, at about 18.3±0.5 degrees 2θ, at about 19.2±0.5 degrees 2θ, at about 21.0±0.5 degrees 2θ, at about 21.7±0.5 degrees 2θ, at about 22.3±0.5 degrees 2θ, and at about 23.1±0.5 degrees 2θ.

In some embodiments, crystalline forms of the compound having a formula of Formula I with a coformer as described or provided herein. In some embodiments, the coformer is succinic acid, adipic acid, fumaric acid, glutaric acid, gentisic acid, hydrochloric acid, 1-hydroxy-2-naphthoic acid, salicylic acid, oxalic acid, or D-(−)-tartaric acid, or any combination thereof. In some embodiments, the coformer is succinic acid. In some embodiments, the coformer is adipic acid. In some embodiments, the coformer is fumaric acid. In some embodiments, the coformer is glutaric acid. In some embodiments, the coformer is gentisic acid. In some embodiments, the coformer is hydrochloric acid. In some embodiments, the coformer is 1-hydroxy-2-naphthoic acid. In some embodiments, the coformer is salicylic acid. In some embodiments, the coformer is oxalic acid. In some embodiments, the coformer is D-(−)-tartaric acid. In some embodiments, the crystalline form is any of Forms III-XIII. In some embodiments, the crystalline form is Form III. In some embodiments, the crystalline form is Form IV. In some embodiments, the crystalline form is Form V. In some embodiments, the crystalline form is Form VI. In some embodiments, the crystalline form is Form VII. In some embodiments, the crystalline form is Form VIII. In some embodiments, the crystalline form is Form IX. In some embodiments, the crystalline form is Form X. In some embodiments, the crystalline form is Form XI. In some embodiments, the crystalline form is Form XII. In some embodiments, the crystalline form is Form XIII.

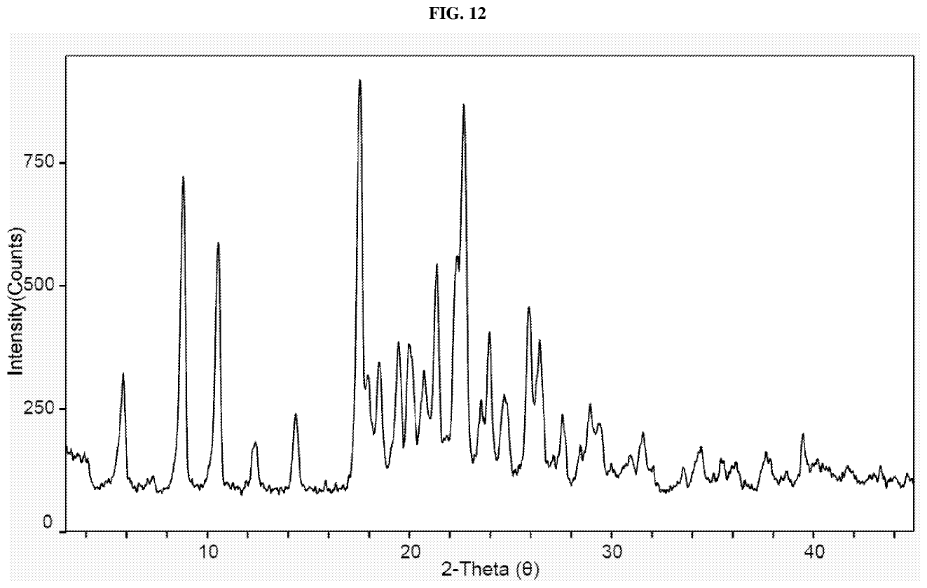

In some embodiments, the crystalline Form III is characterized by an X-ray powder diffraction pattern substantially, as shown in FIG. 12. In some embodiments, the crystalline Form III is characterized by an X-ray powder diffraction pattern comprising one or more peaks, as provided in Table 5. In some embodiments, the crystalline Form III is characterized by an X-ray powder diffraction pattern comprising substantially all of, or all of, the peaks as provided in Table 5.

In some embodiments, the crystalline Form III is characterized by an X-ray powder diffraction pattern comprising a peak at about 5.8±0.5 degrees 2θ.

In some embodiments, the crystalline Form III is characterized by an X-ray powder diffraction pattern comprising a peak at about 8.8±0.5 degrees 2θ.

In some embodiments, the crystalline Form III is characterized by an X-ray powder diffraction pattern comprising a peak at about 10.5±0.5 degrees 2θ.

In some embodiments, the crystalline Form III is characterized by an X-ray powder diffraction pattern comprising a peak at about 12.4±0.5 degrees 2θ.

In some embodiments, the crystalline Form III is characterized by an X-ray powder diffraction pattern comprising a peak at about 14.4±0.5 degrees 2θ.

In some embodiments, the crystalline Form III is characterized by an X-ray powder diffraction pattern comprising a peak at about 17.5±0.5 degrees 2θ.

In some embodiments, the crystalline Form III is characterized by an X-ray powder diffraction pattern comprising a peak at about 17.9±0.5 degrees 2θ.

In some embodiments, the crystalline Form III is characterized by an X-ray powder diffraction pattern comprising a peak at about 18.5±0.5 degrees 2θ.

In some embodiments, the crystalline Form III is characterized by an X-ray powder diffraction pattern comprising a peak at about 19.5±0.5 degrees 2θ.

In some embodiments, the crystalline Form III is characterized by an X-ray powder diffraction pattern comprising a peak at about 20.0±0.5 degrees 2θ.

In some embodiments, the crystalline Form III is characterized by an X-ray powder diffraction pattern comprising a peak at about 20.7±0.5 degrees 2θ.

In some embodiments, the crystalline Form III is characterized by an X-ray powder diffraction pattern comprising a peak at about 21.4±0.5 degrees 2θ.

In some embodiments, the crystalline Form III is characterized by an X-ray powder diffraction pattern comprising a peak at about 22.4±0.5 degrees 2θ.

In some embodiments, the crystalline Form III is characterized by an X-ray powder diffraction pattern comprising a peak at about 22.7±0.5 degrees 2θ.

In some embodiments, the crystalline Form III is characterized by an X-ray powder diffraction pattern comprising a peak at about 23.6±0.5 degrees 2θ.

In some embodiments, the crystalline Form III is characterized by an X-ray powder diffraction pattern comprising a peak at about 24.0±0.5 degrees 2θ.

In some embodiments, the crystalline Form III is characterized by an X-ray powder diffraction pattern comprising a peak at about 24.7±0.5 degrees 2θ.

In some embodiments, the crystalline Form III is characterized by an X-ray powder diffraction pattern comprising a peak at about 25.9±0.5 degrees 2θ.

In some embodiments, the crystalline Form III is characterized by an X-ray powder diffraction pattern comprising a peak at about 26.4±0.5 degrees 2θ.

In some embodiments, the crystalline Form III is characterized by an X-ray powder diffraction pattern comprising a peak at about 27.6±0.5 degrees 2θ.

In some embodiments, the crystalline Form III is characterized by an X-ray powder diffraction pattern comprising a peak at about 29.0±0.5 degrees 2θ.

In some embodiments, the crystalline Form III is characterized by an X-ray powder diffraction pattern comprising a peak at about 31.6±0.5 degrees 2θ.

In some embodiments, the crystalline Form III is characterized by an X-ray powder diffraction pattern comprising a peak at about and at about 39.5±0.5 degrees 2θ.

In some embodiments, the crystalline Form III is characterized by an X-ray powder diffraction pattern comprising peaks at about 8.8±0.5 degrees 2θ and at about 10.5±0.5 degrees 2θ.

In some embodiments, the crystalline Form III is characterized by an X-ray powder diffraction pattern comprising peaks at about 8.8±0.5 degrees 2θ and at about 17.5±0.5 degrees 2θ.

In some embodiments, the crystalline Form III is characterized by an X-ray powder diffraction pattern comprising peaks at about 8.8±0.5 degrees 2θ and at about 21.4±0.5 degrees 2θ.

In some embodiments, the crystalline Form III is characterized by an X-ray powder diffraction pattern comprising peaks at about 8.8±0.5 degrees 2θ and at about 22.7±0.5 degrees 2θ.

In some embodiments, the crystalline Form III is characterized by an X-ray powder diffraction pattern comprising peaks at about 8.8±0.5 degrees 2θ and at about 25.9±0.5 degrees 2θ.

In some embodiments, the crystalline Form III is characterized by an X-ray powder diffraction pattern comprising peaks at about 8.8±0.5 degrees 2θ, at about 10.5±0.5 degrees 2θ, at about 17.5±0.5 degrees 2θ, at about 21.4±0.5 degrees 2θ, at about 22.7±0.5 degrees 2θ, and at about 25.9±0.5 degrees 2θ.

In some embodiments, the crystalline Form III is characterized by an X-ray powder diffraction pattern comprising one or more peaks at about 8.8±0.5 degrees 2θ, at about 10.5±0.5 degrees 2θ, at about 17.5±0.5 degrees 2θ, at about 21.4±0.5 degrees 2θ, at about 22.7±0.5 degrees 2θ, and at about 25.9±0.5 degrees 2θ.

In some embodiments, the crystalline Form III is characterized by an X-ray powder diffraction pattern comprising one or more peaks at about 5.8±0.5 degrees 2θ, at about 8.8±0.5 degrees 2θ, at about 10.5±0.5 degrees 2θ, at about 12.4±0.5 degrees 2θ, at about 14.4±0.5 degrees 2θ, at about 17.5±0.5 degrees 2θ, at about 17.9±0.5 degrees 2θ, at about 18.5±0.5 degrees 2θ, at about 19.5±0.5 degrees 2θ, at about 20.0±0.5 degrees 2θ, at about 20.7±0.5 degrees 2θ, at about 21.4±0.5 degrees 2θ, at about 22.4±0.5 degrees 2θ, at about 22.7±0.5 degrees 2θ, at about 23.6±0.5 degrees 2θ, at about 24.0±0.5 degrees 2θ, at about 24.7±0.5 degrees 2θ, at about 25.9±0.5 degrees 2θ, at about 26.4±0.5 degrees 2θ, at about 27.6±0.5 degrees 2θ, at about 29.0±0.5 degrees 2θ, at about 31.6±0.5 degrees 2θ, and at about 39.5±0.5 degrees 2θ.

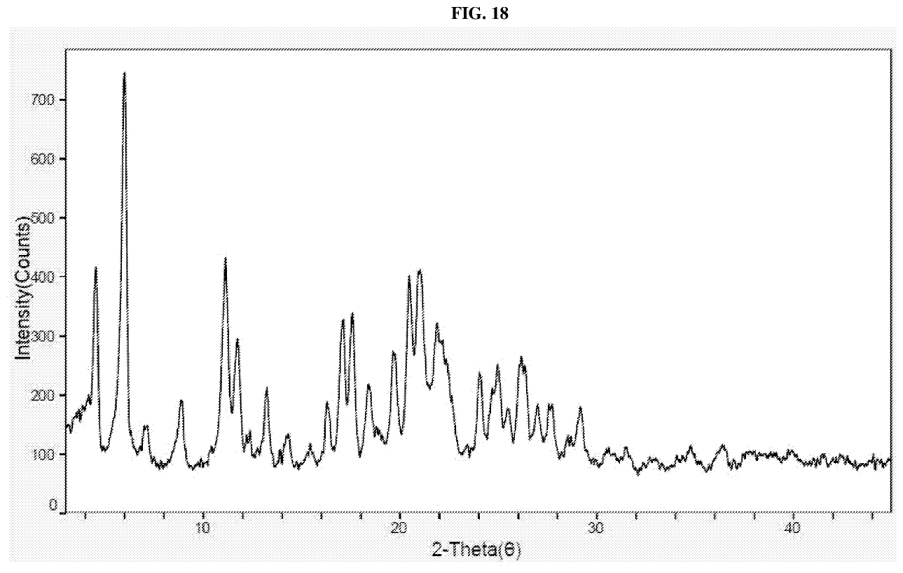

In some embodiments, the crystalline Form IV is characterized by an X-ray powder diffraction pattern substantially, as shown in FIG. 18. In some embodiments, the crystalline Form IV is characterized by an X-ray powder diffraction pattern comprising one or more peaks, as provided in Table 6. In some embodiments, the crystalline Form IV is characterized by an X-ray powder diffraction pattern comprising substantially all of or all of the peaks as provided in Table 6.

In some embodiments, the crystalline Form IV is characterized by an X-ray powder diffraction pattern comprising a peak at about 4.5±0.5 degrees 2θ.

In some embodiments, the crystalline Form IV is characterized by an X-ray powder diffraction pattern comprising a peak at about 6.0±0.5 degrees 2θ.

In some embodiments, the crystalline Form IV is characterized by an X-ray powder diffraction pattern comprising a peak at about 8.9±0.5 degrees 2θ.

In some embodiments, the crystalline Form IV is characterized by an X-ray powder diffraction pattern comprising a peak at about 11.1±0.5 degrees 2θ.

In some embodiments, the crystalline Form IV is characterized by an X-ray powder diffraction pattern comprising a peak at about 11.7±0.5 degrees 2θ.

In some embodiments, the crystalline Form IV is characterized by an X-ray powder diffraction pattern comprising a peak at about 13.2±0.5 degrees 2θ.

In some embodiments, the crystalline Form IV is characterized by an X-ray powder diffraction pattern comprising a peak at about 16.3±0.5 degrees 2θ.

In some embodiments, the crystalline Form IV is characterized by an X-ray powder diffraction pattern comprising a peak at about 17.1±0.5 degrees 2θ.

In some embodiments, the crystalline Form IV is characterized by an X-ray powder diffraction pattern comprising a peak at about 17.6±0.5 degrees 2θ.

In some embodiments, the crystalline Form IV is characterized by an X-ray powder diffraction pattern comprising a peak at about 18.4±0.5 degrees 2θ.

In some embodiments, the crystalline Form IV is characterized by an X-ray powder diffraction pattern comprising a peak at about 19.7±0.5 degrees 2θ.

In some embodiments, the crystalline Form IV is characterized by an X-ray powder diffraction pattern comprising a peak at about 20.5±0.5 degrees 2θ.

In some embodiments, the crystalline Form IV is characterized by an X-ray powder diffraction pattern comprising a peak at about 21.0±0.5 degrees 2θ.

In some embodiments, the crystalline Form IV is characterized by an X-ray powder diffraction pattern comprising a peak at about 21.9±0.5 degrees 2θ.

In some embodiments, the crystalline Form IV is characterized by an X-ray powder diffraction pattern comprising a peak at about 24.0±0.5 degrees 2θ.

In some embodiments, the crystalline Form IV is characterized by an X-ray powder diffraction pattern comprising a peak at about 24.7±0.5 degrees 2θ.

In some embodiments, the crystalline Form IV is characterized by an X-ray powder diffraction pattern comprising a peak at about 25.0±0.5 degrees 2θ.

In some embodiments, the crystalline Form IV is characterized by an X-ray powder diffraction pattern comprising a peak at about 26.2±0.5 degrees 2θ.

In some embodiments, the crystalline Form IV is characterized by an X-ray powder diffraction pattern comprising a peak and at about 29.2±0.5 degrees 2θ.

In some embodiments, the crystalline Form IV is characterized by an X-ray powder diffraction pattern comprising peaks at about 4.5±0.5 degrees 2θ and at about 6.0±0.5 degrees 2θ.

In some embodiments, the crystalline Form IV is characterized by an X-ray powder diffraction pattern comprising peaks at about 4.5±0.5 degrees 2θ and at about 8.9±0.5 degrees 2θ.

In some embodiments, the crystalline Form IV is characterized by an X-ray powder diffraction pattern comprising peaks at about 4.5±0.5 degrees 2θ and at about 11.1±0.5 degrees 2θ.

In some embodiments, the crystalline Form IV is characterized by an X-ray powder diffraction pattern comprising peaks at about 4.5±0.5 degrees 2θ and at about 11.7±0.5 degrees 2θ.

In some embodiments, the crystalline Form IV is characterized by an X-ray powder diffraction pattern comprising peaks at about 4.5±0.5 degrees 2θ and at about 17.1±0.5 degrees 2θ.

In some embodiments, the crystalline Form IV is characterized by an X-ray powder diffraction pattern comprising peaks at about 4.5±0.5 degrees 2θ and at about 17.6±0.5 degrees 2θ.

In some embodiments, the crystalline Form IV is characterized by an X-ray powder diffraction pattern comprising peaks at about 4.5±0.5 degrees 2θ and at about 20.5±0.5 degrees 2θ.

In some embodiments, the crystalline Form IV is characterized by an X-ray powder diffraction pattern comprising peaks at about 4.5±0.5 degrees 2θ and at about 21.0±0.5 degrees 2θ.

In some embodiments, the crystalline Form IV is characterized by an X-ray powder diffraction pattern comprising peaks at about 4.5±0.5 degrees 2θ, at about 6.0±0.5 degrees 2θ, at about 8.9±0.5 degrees 2θ, at about 11.1±0.5 degrees 2θ, at about 11.7±0.5 degrees 2θ, at about 17.1±0.5 degrees 2θ, at about 17.6±0.5 degrees 2θ, at about 20.5±0.5 degrees 2θ, and at about 21.0±0.5 degrees 2θ.

In some embodiments, the crystalline Form IV is characterized by an X-ray powder diffraction pattern comprising one or more peaks at about 4.5±0.5 degrees 2θ, at about 6.0±0.5 degrees 2θ, at about 8.9±0.5 degrees 2θ, at about 11.1±0.5 degrees 2θ, at about 11.7±0.5 degrees 2θ, at about 17.1±0.5 degrees 2θ, at about 17.6±0.5 degrees 2θ, at about 20.5±0.5 degrees 2θ, and at about 21.0±0.5 degrees 2θ.

In some embodiments, the crystalline Form IV is characterized by an X-ray powder diffraction pattern comprising one or more peaks at about 4.5±0.5 degrees 2θ, at about 6.0±0.5 degrees 2θ, at about 8.9±0.5 degrees 2θ, at about 11.1±0.5 degrees 2θ, at about 11.7±0.5 degrees 2θ, at about 13.2±0.5 degrees 2θ, at about 16.3±0.5 degrees 2θ, at about 17.1±0.5 degrees 2θ, at about 17.6±0.5 degrees 2θ, at about 18.4±0.5 degrees 2θ, at about 19.7±0.5 degrees 2θ, at about 20.5±0.5 degrees 2θ, at about 21.0±0.5 degrees 2θ, at about 21.9±0.5 degrees 2θ, at about 24.0±0.5 degrees 2θ, at about 24.7±0.5 degrees 2θ, at about 25.0±0.5 degrees 2θ, at about 26.2±0.5 degrees 2θ, and at about 29.2±0.5 degrees 2θ.

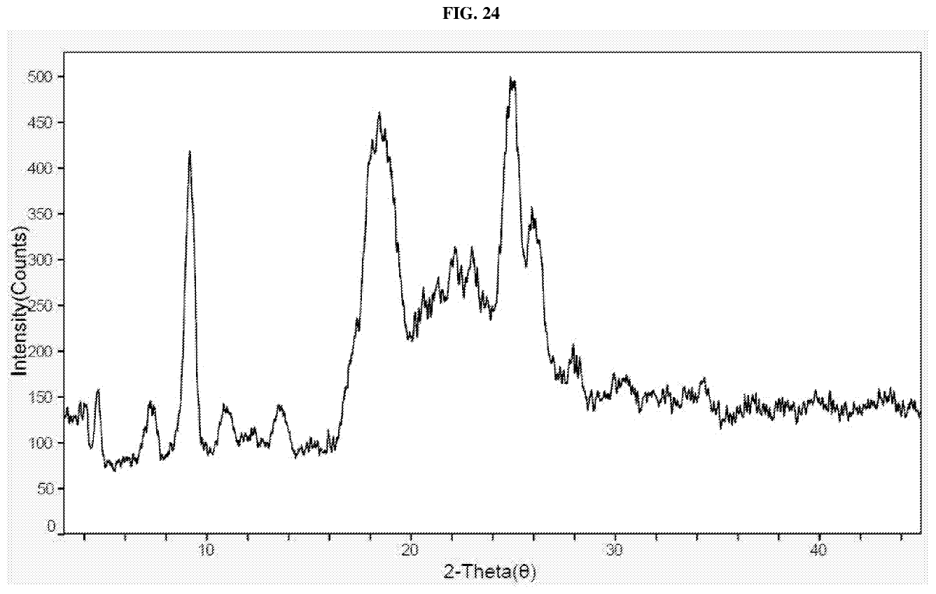

In some embodiments, the crystalline Form V is characterized by an X-ray powder diffraction pattern substantially, as shown in FIG. 24. In some embodiments, the crystalline Form V is characterized by an X-ray powder diffraction pattern comprising one or more peaks, as provided in Table 7. In some embodiments, the crystalline Form V is characterized by an X-ray powder diffraction pattern comprising substantially all of, or all of, the peaks as provided in Table 7.

In some embodiments, the crystalline Form V is characterized by an X-ray powder diffraction pattern comprising a peak at about 4.7±0.5 degrees 2θ.

In some embodiments, the crystalline Form V is characterized by an X-ray powder diffraction pattern comprising a peak at about 7.4±0.5 degrees 2θ.

In some embodiments, the crystalline Form V is characterized by an X-ray powder diffraction pattern comprising a peak at about 9.2±0.5 degrees 2θ.

In some embodiments, the crystalline Form V is characterized by an X-ray powder diffraction pattern comprising a peak at about 11.2±0.5 degrees 2θ.

In some embodiments, the crystalline Form V is characterized by an X-ray powder diffraction pattern comprising a peak at about 13.8±0.5 degrees 2θ.

In some embodiments, the crystalline Form V is characterized by an X-ray powder diffraction pattern comprising a peak at about 17.2±0.5 degrees 2θ.

In some embodiments, the crystalline Form V is characterized by an X-ray powder diffraction pattern comprising a peak at about 18.1±0.5 degrees 2θ.

In some embodiments, the crystalline Form V is characterized by an X-ray powder diffraction pattern comprising a peak at about 18.9±0.5 degrees 2θ.

In some embodiments, the crystalline Form V is characterized by an X-ray powder diffraction pattern comprising a peak at about 25.1±0.5 degrees 2θ.

In some embodiments, the crystalline Form V is characterized by an X-ray powder diffraction pattern comprising a peak and at about 25.9±0.5 degrees 2θ.

In some embodiments, the crystalline Form V is characterized by an X-ray powder diffraction pattern comprising peaks at about 9.2±0.5 degrees 2θ and at about 18.1±0.5 degrees 2θ.

In some embodiments, the crystalline Form V is characterized by an X-ray powder diffraction pattern comprising peaks at about 9.2±0.5 degrees 2θ and at about 25.1±0.5 degrees 2θ.

In some embodiments, the crystalline Form V is characterized by an X-ray powder diffraction pattern comprising peaks at about 9.2±0.5 degrees 2θ and at about 25.9±0.5 degrees 2θ.

In some embodiments, the crystalline Form V is characterized by an X-ray powder diffraction pattern comprising one or more peaks at about 9.2±0.5 degrees 2θ, at about 18.1±0.5 degrees 2θ, at about 25.1±0.5 degrees 2θ, and at about 25.9±0.5 degrees 2θ.

In some embodiments, the crystalline Form V is characterized by an X-ray powder diffraction pattern comprising one or more peaks at about 4.7±0.5 degrees 2θ, at about 7.4±0.5 degrees 2θ, at about 9.2±0.5 degrees 2θ, at about 11.2±0.5 degrees 2θ, at about 13.8±0.5 degrees 2θ, at about 17.2±0.5 degrees 2θ, at about 18.1±0.5 degrees 2θ, at about 18.9±0.5 degrees 2θ, at about 25.1±0.5 degrees 2θ, and at about 25.9±0.5 degrees 2θ.

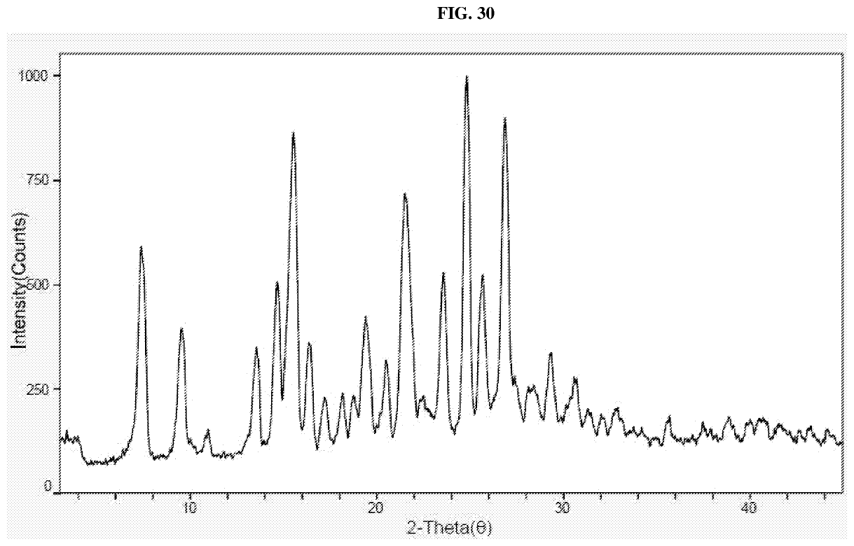

In some embodiments, the crystalline Form VI is characterized by an X-ray powder diffraction pattern substantially, as shown in FIG. 30. In some embodiments, the crystalline Form VI is characterized by an X-ray powder diffraction pattern comprising one or more peaks, as provided in Table 8. In some embodiments, the crystalline Form VI is characterized by an X-ray powder diffraction pattern comprising substantially all of, or all of, the peaks as provided in Table 8.

In some embodiments, the crystalline Form VI is characterized by an X-ray powder diffraction pattern comprising a peak at about 7.4±0.5 degrees 2θ.

In some embodiments, the crystalline Form VI is characterized by an X-ray powder diffraction pattern comprising a peak at about 9.5±0.5 degrees 2θ.

In some embodiments, the crystalline Form VI is characterized by an X-ray powder diffraction pattern comprising a peak at about 13.6±0.5 degrees 2θ.

In some embodiments, the crystalline Form VI is characterized by an X-ray powder diffraction pattern comprising a peak at about 14.7±0.5 degrees 2θ.

In some embodiments, the crystalline Form VI is characterized by an X-ray powder diffraction pattern comprising a peak at about 15.5±0.5 degrees 2θ.

In some embodiments, the crystalline Form VI is characterized by an X-ray powder diffraction pattern comprising a peak at about 16.4±0.5 degrees 2θ.

In some embodiments, the crystalline Form VI is characterized by an X-ray powder diffraction pattern comprising a peak at about 17.2±0.5 degrees 2θ.

In some embodiments, the crystalline Form VI is characterized by an X-ray powder diffraction pattern comprising a peak at about 18.2±0.5 degrees 2θ.

In some embodiments, the crystalline Form VI is characterized by an X-ray powder diffraction pattern comprising a peak at about 19.4±0.5 degrees 2θ.

In some embodiments, the crystalline Form VI is characterized by an X-ray powder diffraction pattern comprising a peak at about 20.5±0.5 degrees 2θ.

In some embodiments, the crystalline Form VI is characterized by an X-ray powder diffraction pattern comprising a peak at about 21.5±0.5 degrees 2θ.

In some embodiments, the crystalline Form VI is characterized by an X-ray powder diffraction pattern comprising a peak at about 23.5±0.5 degrees 2θ.

In some embodiments, the crystalline Form VI is characterized by an X-ray powder diffraction pattern comprising a peak at about 24.8±0.5 degrees 2θ.

In some embodiments, the crystalline Form VI is characterized by an X-ray powder diffraction pattern comprising a peak at about 25.7±0.5 degrees 2θ.

In some embodiments, the crystalline Form VI is characterized by an X-ray powder diffraction pattern comprising a peak at about 26.9±0.5 degrees 2θ.

In some embodiments, the crystalline Form VI is characterized by an X-ray powder diffraction pattern comprising a peak at about 29.4±0.5 degrees 2θ.

In some embodiments, the crystalline Form VI is characterized by an X-ray powder diffraction pattern comprising a peak and at about 30.7±0.5 degrees 2θ.

In some embodiments, the crystalline Form VI is characterized by an X-ray powder diffraction pattern comprising peaks at about 7.4±0.5 degrees 2θ and at about 9.5±0.5 degrees 2θ.

In some embodiments, the crystalline Form VI is characterized by an X-ray powder diffraction pattern comprising peaks at about 7.4±0.5 degrees 2θ and at about 14.7±0.5 degrees 2θ.

In some embodiments, the crystalline Form VI is characterized by an X-ray powder diffraction pattern comprising peaks at about 7.4±0.5 degrees 2θ and at about 15.5±0.5 degrees 2θ.

In some embodiments, the crystalline Form VI is characterized by an X-ray powder diffraction pattern comprising peaks at about 7.4±0.5 degrees 2θ and at about 21.5±0.5 degrees 2θ.

In some embodiments, the crystalline Form VI is characterized by an X-ray powder diffraction pattern comprising peaks at about 7.4±0.5 degrees 2θ and at about 23.5±0.5 degrees 2θ.

In some embodiments, the crystalline Form VI is characterized by an X-ray powder diffraction pattern comprising peaks at about 7.4±0.5 degrees 2θ and at about 24.8±0.5 degrees 2θ.

In some embodiments, the crystalline Form VI is characterized by an X-ray powder diffraction pattern comprising peaks at about 7.4±0.5 degrees 2θ and at about 25.7±0.5 degrees 2θ.

In some embodiments, the crystalline Form VI is characterized by an X-ray powder diffraction pattern comprising peaks at about 7.4±0.5 degrees 2θ and at about 26.9±0.5 degrees 2θ.

In some embodiments, the crystalline Form VI is characterized by an X-ray powder diffraction pattern comprising peaks at about 7.4±0.5 degrees 2θ, at about 9.5±0.5 degrees 2θ, at about 14.7±0.5 degrees 2θ, at about 15.5±0.5 degrees 2θ, at about 21.5±0.5 degrees 2θ, at about 23.5±0.5 degrees 2θ, at about 24.8±0.5 degrees 2θ, at about 25.7±0.5 degrees 2θ, and at about 26.9±0.5 degrees 2θ.

In some embodiments, the crystalline Form VI is characterized by an X-ray powder diffraction pattern comprising one or more peaks at about 7.4±0.5 degrees 2θ, at about 9.5±0.5 degrees 2θ, at about 14.7±0.5 degrees 2θ, at about 15.5±0.5 degrees 2θ, at about 21.5±0.5 degrees 2θ, at about 23.5±0.5 degrees 2θ, at about 24.8±0.5 degrees 2θ, at about 25.7±0.5 degrees 2θ, and at about 26.9±0.5 degrees 2θ.

In some embodiments, the crystalline Form VI is characterized by an X-ray powder diffraction pattern comprising one or more peaks at about 7.4±0.5 degrees 2θ, at about 9.5±0.5 degrees 2θ, at about 13.6±0.5 degrees 2θ, at about 14.7±0.5 degrees 2θ, at about 15.5±0.5 degrees 2θ, at about 16.4±0.5 degrees 2θ, at about 17.2±0.5 degrees 2θ, at about 18.2±0.5 degrees 2θ, at about 19.4±0.5 degrees 2θ, at about 20.5±0.5 degrees 2θ, at about 21.5±0.5 degrees 2θ, at about 23.5±0.5 degrees 2θ, at about 24.8±0.5 degrees 2θ, at about 25.7±0.5 degrees 2θ, at about 26.9±0.5 degrees 2θ, at about 29.4±0.5 degrees 2θ, and at about 30.7±0.5 degrees 2θ.

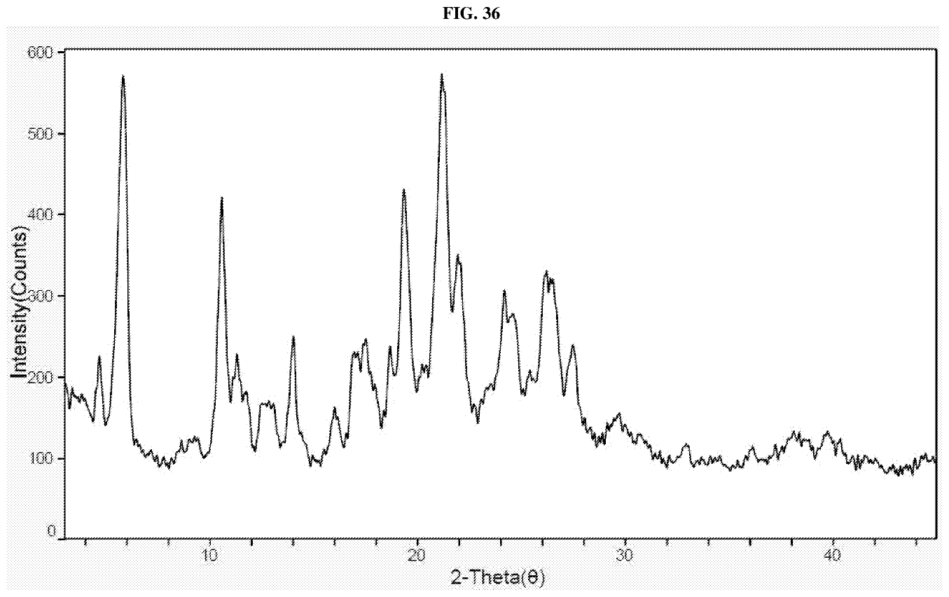

In some embodiments, the crystalline Form VII is characterized by an X-ray powder diffraction pattern substantially, as shown in FIG. 36. In some embodiments, the crystalline FoRm VII is characterized by an X-ray powder diffraction pattern comprising one or more peaks, as provided in Table 9. In some embodiments, the crystalline Form VII is characterized by an X-ray powder diffraction pattern comprising substantially all of, or all of, the peaks as provided in Table 9.

In some embodiments, the crystalline Form VII is characterized by an X-ray powder diffraction pattern comprising a peak at about 4.7±0.5 degrees 2θ.

In some embodiments, the crystalline Form VII is characterized by an X-ray powder diffraction pattern comprising a peak at about 5.8±0.5 degrees 2θ.

In some embodiments, the crystalline Form VII is characterized by an X-ray powder diffraction pattern comprising a peak at about 10.6±0.5 degrees 2θ.

In some embodiments, the crystalline Form VII is characterized by an X-ray powder diffraction pattern comprising a peak at about 11.3±0.5 degrees 2θ.

In some embodiments, the crystalline Form VII is characterized by an X-ray powder diffraction pattern comprising a peak at about 11.8±0.5 degrees 2θ.

In some embodiments, the crystalline Form VII is characterized by an X-ray powder diffraction pattern comprising a peak at about 12.6±0.5 degrees 2θ.

In some embodiments, the crystalline Form VII is characterized by an X-ray powder diffraction pattern comprising a peak at about 13.1±0.5 degrees 2θ.

In some embodiments, the crystalline Form VII is characterized by an X-ray powder diffraction pattern comprising a peak at about 14.0±0.5 degrees 2θ.

In some embodiments, the crystalline Form VII is characterized by an X-ray powder diffraction pattern comprising a peak at about 16.0±0.5 degrees 2θ.

In some embodiments, the crystalline Form VII is characterized by an X-ray powder diffraction pattern comprising a peak at about 17.0±0.5 degrees 2θ.

In some embodiments, the crystalline Form VII is characterized by an X-ray powder diffraction pattern comprising a peak at about 17.5±0.5 degrees 2θ.

In some embodiments, the crystalline Form VII is characterized by an X-ray powder diffraction pattern comprising a peak at about 18.7±0.5 degrees 2θ.

In some embodiments, the crystalline Form VII is characterized by an X-ray powder diffraction pattern comprising a peak at about 19.3±0.5 degrees 2θ.

In some embodiments, the crystalline Form VII is characterized by an X-ray powder diffraction pattern comprising a peak at about 21.2±0.5 degrees 2θ.

In some embodiments, the crystalline Form VII is characterized by an X-ray powder diffraction pattern comprising a peak at about 22.1±0.5 degrees 2θ.

In some embodiments, the crystalline Form VII is characterized by an X-ray powder diffraction pattern comprising a peak at about 24.2±0.5 degrees 2θ.

In some embodiments, the crystalline Form VII is characterized by an X-ray powder diffraction pattern comprising a peak at about 24.7±0.5 degrees 2θ.

In some embodiments, the crystalline Form VII is characterized by an X-ray powder diffraction pattern comprising a peak at about 26.2±0.5 degrees 2θ.

In some embodiments, the crystalline Form VII is characterized by an X-ray powder diffraction pattern comprising a peak and at about 27.4±0.5 degrees 2θ.

In some embodiments, the crystalline Form VII is characterized by an X-ray powder diffraction pattern comprising peaks at about 5.8±0.5 degrees 2θ and at about 10.6±0.5 degrees 2θ.

In some embodiments, the crystalline Form VII is characterized by an X-ray powder diffraction pattern comprising peaks at about 5.8±0.5 degrees 2θ and at about 19.3±0.5 degrees 2θ.

In some embodiments, the crystalline Form VII is characterized by an X-ray powder diffraction pattern comprising peaks at about 5.8±0.5 degrees 2θ and at about 21.2±0.5 degrees 2θ.

In some embodiments, the crystalline Form VII is characterized by an X-ray powder diffraction pattern comprising peaks at about 5.8±0.5 degrees 2θ and at about 22.1±0.5 degrees 2θ.

In some embodiments, the crystalline Form VII is characterized by an X-ray powder diffraction pattern comprising peaks at about 5.8±0.5 degrees 2θ and at about 24.2±0.5 degrees 2θ.

In some embodiments, the crystalline Form VII is characterized by an X-ray powder diffraction pattern comprising peaks at about 5.8±0.5 degrees 2θ and at about 26.2±0.5 degrees 2θ.

In some embodiments, the crystalline Form VII is characterized by an X-ray powder diffraction pattern comprising peaks at about 5.8±0.5 degrees 2θ, at about 10.6±0.5 degrees 2θ, at about 19.3±0.5 degrees 2θ, at about 21.2±0.5 degrees 2θ, at about 22.1±0.5 degrees 2θ, at about 24.2±0.5 degrees 2θ, and at about 26.2±0.5 degrees 2θ.

In some embodiments, the crystalline Form VII is characterized by an X-ray powder diffraction pattern comprising one or more peaks at about 5.8±0.5 degrees 2θ, at about 10.6±0.5 degrees 2θ, at about 19.3±0.5 degrees 2θ, at about 21.2±0.5 degrees 2θ, at about 22.1±0.5 degrees 2θ, at about 24.2±0.5 degrees 2θ, and at about 26.2±0.5 degrees 2θ.

In some embodiments, the crystalline Form VII is characterized by an X-ray powder diffraction pattern comprising one or more peaks at about 4.7±0.5 degrees 2θ, at about 5.8±0.5 degrees 2θ, at about 10.6±0.5 degrees 2θ, at about 11.3±0.5 degrees 2θ, at about 11.8±0.5 degrees 2θ, at about 12.6±0.5 degrees 2θ, at about 13.1±0.5 degrees 2θ, at about 14.0±0.5 degrees 2θ, at about 16.0±0.5 degrees 2θ, at about 17.0±0.5 degrees 2θ, at about 17.5±0.5 degrees 2θ, at about 18.7±0.5 degrees 2θ, at about 19.3±0.5 degrees 2θ, at about 21.2±0.5 degrees 2θ, at about 22.1±0.5 degrees 2θ, at about 24.2±0.5 degrees 2θ, at about 24.7±0.5 degrees 2θ, at about 26.2±0.5 degrees 2θ, and at about 27.4±0.5 degrees 2θ.

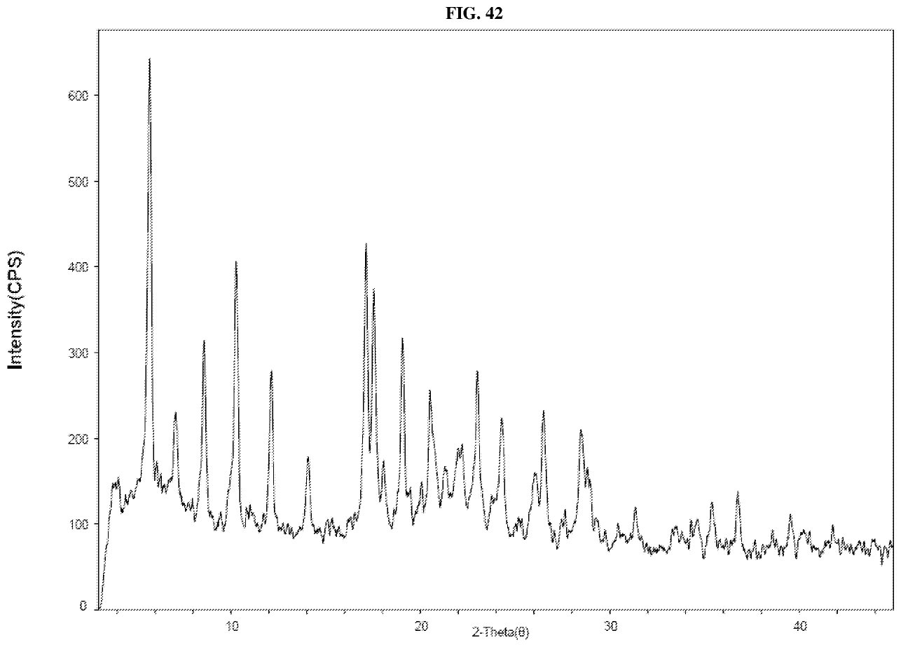

In some embodiments, the crystalline Form VIII is characterized by an X-ray powder diffraction pattern substantially, as shown in FIG. 42. In some embodiments, the crystalline Form VIII is characterized by an X-ray powder diffraction pattern comprising one or more peaks, as provided in Table 10. In some embodiments, the crystalline Form VIII is characterized by an X-ray powder diffraction pattern comprising substantially all of, or all of, the peaks as provided in Table 10.

In some embodiments, the crystalline Form VIII is characterized by an X-ray powder diffraction pattern comprising a peak at about 3.9±0.5 degrees 2θ.

In some embodiments, the crystalline Form VIII is characterized by an X-ray powder diffraction pattern comprising a peak at about 5.7±0.5 degrees 2θ.

In some embodiments, the crystalline Form VIII is characterized by an X-ray powder diffraction pattern comprising a peak at about 7.1±0.5 degrees 2θ.

In some embodiments, the crystalline Form VIII is characterized by an X-ray powder diffraction pattern comprising a peak at about 8.6±0.5 degrees 2θ.

In some embodiments, the crystalline Form VIII is characterized by an X-ray powder diffraction pattern comprising a peak at about 10.3±0.5 degrees 2θ.

In some embodiments, the crystalline Form VIII is characterized by an X-ray powder diffraction pattern comprising a peak at about 12.1±0.5 degrees 2θ.

In some embodiments, the crystalline Form VIII is characterized by an X-ray powder diffraction pattern comprising a peak at about 14.1±0.5 degrees 2θ.

In some embodiments, the crystalline Form VIII is characterized by an X-ray powder diffraction pattern comprising a peak at about 17.1±0.5 degrees 2θ.

In some embodiments, the crystalline Form VIII is characterized by an X-ray powder diffraction pattern comprising a peak at about 19.1±0.5 degrees 2θ.

In some embodiments, the crystalline Form VIII is characterized by an X-ray powder diffraction pattern comprising a peak at about 20.6±0.5 degrees 2θ.

In some embodiments, the crystalline Form VIII is characterized by an X-ray powder diffraction pattern comprising a peak at about 22.2±0.5 degrees 2θ.

In some embodiments, the crystalline Form VIII is characterized by an X-ray powder diffraction pattern comprising a peak at about 23.0±0.5 degrees 2θ.

In some embodiments, the crystalline Form VIII is characterized by an X-ray powder diffraction pattern comprising a peak at about 24.3±0.5 degrees 2θ.

In some embodiments, the crystalline Form VIII is characterized by an X-ray powder diffraction pattern comprising a peak at about 26.0±0.5 degrees 2θ.

In some embodiments, the crystalline Form VIII is characterized by an X-ray powder diffraction pattern comprising a peak at about 26.5±0.5 degrees 2θ.

In some embodiments, the crystalline Form VIII is characterized by an X-ray powder diffraction pattern comprising a peak at about 28.5±0.5 degrees 2θ.

In some embodiments, the crystalline Form VIII is characterized by an X-ray powder diffraction pattern comprising a peak at about 34.6±0.5 degrees 2θ.

In some embodiments, the crystalline Form VIII is characterized by an X-ray powder diffraction pattern comprising a peak at about 35.4±0.5 degrees 2θ.

In some embodiments, the crystalline Form VIII is characterized by an X-ray powder diffraction pattern comprising a peak at about 36.8±0.5 degrees 2θ.

In some embodiments, the crystalline Form VIII is characterized by an X-ray powder diffraction pattern comprising a peak and at about 39.5±0.5 degrees 2θ.

In some embodiments, the crystalline Form VIII is characterized by an X-ray powder diffraction pattern comprising peaks at about 3.9±0.5 degrees 2θ and at about 5.7±0.5 degrees 2θ.

In some embodiments, the crystalline Form VIII is characterized by an X-ray powder diffraction pattern comprising peaks at about 3.9±0.5 degrees 2θ and at about 8.6±0.5 degrees 2θ.

In some embodiments, the crystalline Form VIII is characterized by an X-ray powder diffraction pattern comprising peaks at about 3.9±0.5 degrees 2θ and at about 10.3±0.5 degrees 2θ.

In some embodiments, the crystalline Form VIII is characterized by an X-ray powder diffraction pattern comprising peaks at about 3.9±0.5 degrees 2θ and at about 12.1±0.5 degrees 2θ.

In some embodiments, the crystalline Form VIII is characterized by an X-ray powder diffraction pattern comprising peaks at about 3.9±0.5 degrees 2θ and at about 14.1±0.5 degrees 2θ.

In some embodiments, the crystalline Form VIII is characterized by an X-ray powder diffraction pattern comprising peaks at about 3.9±0.5 degrees 2θ and at about 17.1±0.5 degrees 2θ.

In some embodiments, the crystalline Form VIII is characterized by an X-ray powder diffraction pattern comprising peaks at about 3.9±0.5 degrees 2θ and at about 19.1±0.5 degrees 2θ.

In some embodiments, the crystalline Form VIII is characterized by an X-ray powder diffraction pattern comprising peaks at about 3.9±0.5 degrees 2θ and at about 20.6±0.5 degrees 2θ.

In some embodiments, the crystalline Form VIII is characterized by an X-ray powder diffraction pattern comprising peaks at about 3.9±0.5 degrees 2θ and at about 23.0±0.5 degrees 2θ.

In some embodiments, the crystalline Form VIII is characterized by an X-ray powder diffraction pattern comprising peaks at about 3.9±0.5 degrees 2θ and at about 24.3±0.5 degrees 2θ.

In some embodiments, the crystalline Form VIII is characterized by an X-ray powder diffraction pattern comprising peaks at about 3.9±0.5 degrees 2θ and at about 26.5±0.5 degrees 2θ.

In some embodiments, the crystalline Form VIII is characterized by an X-ray powder diffraction pattern comprising peaks at about 3.9±0.5 degrees 2θ and at about 28.5±0.5 degrees 2θ.

In some embodiments, the crystalline Form VIII is characterized by an X-ray powder diffraction pattern comprising peaks at about 3.9±0.5 degrees 2θ, at about 5.7±0.5 degrees 2θ, at about 8.6±0.5 degrees 2θ, at about 10.3±0.5 degrees 2θ, at about 12.1±0.5 degrees 2θ, at about 14.1±0.5 degrees 2θ, at about 17.1±0.5 degrees 2θ, at about 19.1±0.5 degrees 2θ, at about 20.6±0.5 degrees 2θ, at about 23.0±0.5 degrees 2θ, at about 24.3±0.5 degrees 2θ, at about 26.5±0.5 degrees 2θ, and at about 28.5±0.5 degrees 2θ.

In some embodiments, the crystalline Form VIII is characterized by an X-ray powder diffraction pattern comprising one or more peaks at about 3.9±0.5 degrees 2θ, at about 5.7±0.5 degrees 2θ, at about 8.6±0.5 degrees 2θ, at about 10.3±0.5 degrees 2θ, at about 12.1±0.5 degrees 2θ, at about 14.1±0.5 degrees 2θ, at about 17.1±0.5 degrees 2θ, at about 19.1±0.5 degrees 2θ, at about 20.6±0.5 degrees 2θ, at about 23.0±0.5 degrees 2θ, at about 24.3±0.5 degrees 2θ, at about 26.5±0.5 degrees 2θ, and at about 28.5±0.5 degrees 2θ.

In some embodiments, the crystalline Form VIII is characterized by an X-ray powder diffraction pattern comprising one or more peaks at about 3.9±0.5 degrees 2θ, at about 5.7±0.5 degrees 2θ, at about 7.1±0.5 degrees 2θ, at about 8.6±0.5 degrees 2θ, at about 10.3±0.5 degrees 2θ, at about 12.1±0.5 degrees 2θ, at about 14.1±0.5 degrees 2θ, at about 17.1±0.5 degrees 2θ, at about 19.1±0.5 degrees 2θ, at about 20.6±0.5 degrees 2θ, at about 22.2±0.5 degrees 2θ, at about 23.0±0.5 degrees 2θ, at about 24.3±0.5 degrees 2θ, at about 26.0±0.5 degrees 2θ, at about 26.5±0.5 degrees 2θ, at about 28.5±0.5 degrees 2θ, at about 34.6±0.5 degrees 2θ, at about 35.4±0.5 degrees 2θ, at about 36.8±0.5 degrees 2θ, and at about 39.5±0.5 degrees 2θ.

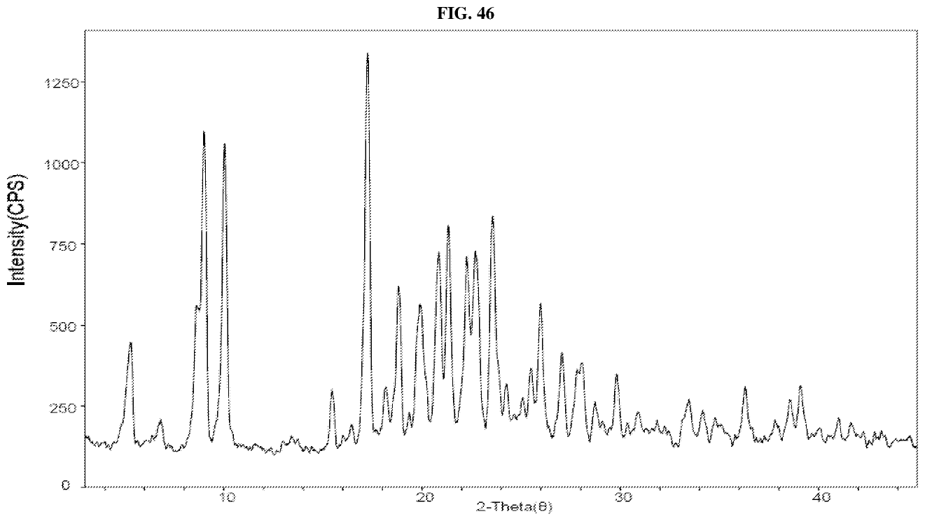

In some embodiments, the crystalline Form IX is characterized by an X-ray powder diffraction pattern substantially, as shown in FIG. 46. In some embodiments, the crystalline Form IX is characterized by an X-ray powder diffraction pattern comprising one or more peaks, as provided in Table 11. In some embodiments, the crystalline Form IX is characterized by an X-ray powder diffraction pattern comprising substantially all of, or all of, the peaks as provided in Table 11.

In some embodiments, the crystalline Form IX is characterized by an X-ray powder diffraction pattern comprising a peak at about 5.3±0.5 degrees 2θ.

In some embodiments, the crystalline Form IX is characterized by an X-ray powder diffraction pattern comprising a peak at about 6.8±0.5 degrees 2θ.

In some embodiments, the crystalline Form IX is characterized by an X-ray powder diffraction pattern comprising a peak at about 9.0±0.5 degrees 2θ.

In some embodiments, the crystalline Form IX is characterized by an X-ray powder diffraction pattern comprising a peak at about 10.0±0.5 degrees 2θ.

In some embodiments, the crystalline Form IX is characterized by an X-ray powder diffraction pattern comprising a peak at about 15.5±0.5 degrees 2θ.

In some embodiments, the crystalline Form IX is characterized by an X-ray powder diffraction pattern comprising a peak at about 17.3±0.5 degrees 2θ.

In some embodiments, the crystalline Form IX is characterized by an X-ray powder diffraction pattern comprising a peak at about 18.2±0.5 degrees 2θ.

In some embodiments, the crystalline Form IX is characterized by an X-ray powder diffraction pattern comprising a peak at about 18.8±0.5 degrees 2θ.

In some embodiments, the crystalline Form IX is characterized by an X-ray powder diffraction pattern comprising a peak at about 19.9±0.5 degrees 2θ.

In some embodiments, the crystalline Form IX is characterized by an X-ray powder diffraction pattern comprising a peak at about 20.9±0.5 degrees 2θ.

In some embodiments, the crystalline Form IX is characterized by an X-ray powder diffraction pattern comprising a peak at about 21.3±0.5 degrees 2θ.

In some embodiments, the crystalline Form IX is characterized by an X-ray powder diffraction pattern comprising a peak at about 22.7±0.5 degrees 2θ.

In some embodiments, the crystalline Form IX is characterized by an X-ray powder diffraction pattern comprising a peak at about 23.6±0.5 degrees 2θ.

In some embodiments, the crystalline Form IX is characterized by an X-ray powder diffraction pattern comprising a peak at about 24.3±0.5 degrees 2θ.

In some embodiments, the crystalline Form IX is characterized by an X-ray powder diffraction pattern comprising a peak at about 25.5±0.5 degrees 2θ.

In some embodiments, the crystalline Form IX is characterized by an X-ray powder diffraction pattern comprising a peak at about 26.0±0.5 degrees 2θ.

In some embodiments, the crystalline Form IX is characterized by an X-ray powder diffraction pattern comprising a peak at about 27.1±0.5 degrees 2θ.

In some embodiments, the crystalline Form IX is characterized by an X-ray powder diffraction pattern comprising a peak at about 28.0±0.5 degrees 2θ.

In some embodiments, the crystalline Form IX is characterized by an X-ray powder diffraction pattern comprising a peak at about 28.8±0.5 degrees 2θ.

In some embodiments, the crystalline Form IX is characterized by an X-ray powder diffraction pattern comprising a peak at about 29.8±0.5 degrees 2θ.

In some embodiments, the crystalline Form IX is characterized by an X-ray powder diffraction pattern comprising a peak at about 33.4±0.5 degrees 2θ.

In some embodiments, the crystalline Form IX is characterized by an X-ray powder diffraction pattern comprising a peak at about 34.2±0.5 degrees 2θ.

In some embodiments, the crystalline Form IX is characterized by an X-ray powder diffraction pattern comprising a peak at about 36.3±0.5 degrees 2θ.

In some embodiments, the crystalline Form IX is characterized by an X-ray powder diffraction pattern comprising a peak at about 38.6±0.5 degrees 2θ.

In some embodiments, the crystalline Form IX is characterized by an X-ray powder diffraction pattern comprising a peak and at about 39.1±0.5 degrees 2θ.

In some embodiments, the crystalline Form IX is characterized by an X-ray powder diffraction pattern comprising peaks at about 5.3±0.5 degrees 2θ and at about 9.0±0.5 degrees 2θ.

In some embodiments, the crystalline Form IX is characterized by an X-ray powder diffraction pattern comprising peaks at about 5.3±0.5 degrees 2θ and at about 10.0±0.5 degrees 2θ.

In some embodiments, the crystalline Form IX is characterized by an X-ray powder diffraction pattern comprising peaks at about 5.3±0.5 degrees 2θ and at about 17.3±0.5 degrees 2θ.

In some embodiments, the crystalline Form IX is characterized by an X-ray powder diffraction pattern comprising peaks at about 5.3±0.5 degrees 2θ and at about 18.8±0.5 degrees 2θ.

In some embodiments, the crystalline Form IX is characterized by an X-ray powder diffraction pattern comprising peaks at about 5.3±0.5 degrees 2θ and at about 19.9±0.5 degrees 2θ.

In some embodiments, the crystalline Form IX is characterized by an X-ray powder diffraction pattern comprising peaks at about 5.3±0.5 degrees 2θ and at about 20.9±0.5 degrees 2θ.

In some embodiments, the crystalline Form IX is characterized by an X-ray powder diffraction pattern comprising peaks at about 5.3±0.5 degrees 2θ and at about 21.3±0.5 degrees 2θ.

In some embodiments, the crystalline Form IX is characterized by an X-ray powder diffraction pattern comprising peaks at about 5.3±0.5 degrees 2θ and at about 22.7±0.5 degrees 2θ.

In some embodiments, the crystalline Form IX is characterized by an X-ray powder diffraction pattern comprising peaks at about 5.3±0.5 degrees 2θ and at about 23.6±0.5 degrees 2θ.

In some embodiments, the crystalline Form IX is characterized by an X-ray powder diffraction pattern comprising peaks at about 5.3±0.5 degrees 2θ and at about 26.0±0.5 degrees 2θ.

In some embodiments, the crystalline Form IX is characterized by an X-ray powder diffraction pattern comprising peaks at about 5.3±0.5 degrees 2θ, at about 9.0±0.5 degrees 2θ, at about 10.0±0.5 degrees 2θ, at about 17.3±0.5 degrees 2θ, at about 18.8±0.5 degrees 2θ, at about 19.9±0.5 degrees 2θ, at about 20.9±0.5 degrees 2θ, at about 21.3±0.5 degrees 2θ, at about 22.7±0.5 degrees 2θ, at about 23.6±0.5 degrees 2θ, and at about 26.0±0.5 degrees 2θ.

In some embodiments, the crystalline Form IX is characterized by an X-ray powder diffraction pattern comprising one or more peaks at about 5.3±0.5 degrees 2θ, at about 9.0±0.5 degrees 2θ, at about 10.0±0.5 degrees 2θ, at about 17.3±0.5 degrees 2θ, at about 18.8±0.5 degrees 2θ, at about 19.9±0.5 degrees 2θ, at about 20.9±0.5 degrees 2θ, at about 21.3±0.5 degrees 2θ, at about 22.7±0.5 degrees 2θ, at about 23.6±0.5 degrees 2θ, and at about 26.0±0.5 degrees 2θ.

In some embodiments, the crystalline Form IX is characterized by an X-ray powder diffraction pattern comprising one or more peaks at about 5.3±0.5 degrees 2θ, at about 6.8±0.5 degrees 2θ, at about 9.0±0.5 degrees 2θ, at about 10.0±0.5 degrees 2θ, at about 15.5±0.5 degrees 2θ, at about 17.3±0.5 degrees 2θ, at about 18.2±0.5 degrees 2θ, at about 18.8±0.5 degrees 2θ, at about 19.9±0.5 degrees 2θ, at about 20.9±0.5 degrees 2θ, at about 21.3±0.5 degrees 2θ, at about 22.7±0.5 degrees 2θ, at about 23.6±0.5 degrees 2θ, at about 24.3±0.5 degrees 2θ, at about 25.5±0.5 degrees 2θ, at about 26.0±0.5 degrees 2θ, at about 27.1±0.5 degrees 2θ, at about 28.0±0.5 degrees 2θ, at about 28.8±0.5 degrees 2θ, at about 29.8±0.5 degrees 2θ, at about 33.4±0.5 degrees 2θ, at about 34.2±0.5 degrees 2θ, at about 36.3±0.5 degrees 2θ, at about 38.6±0.5 degrees 2θ, and at about 39.1±0.5 degrees 2θ.

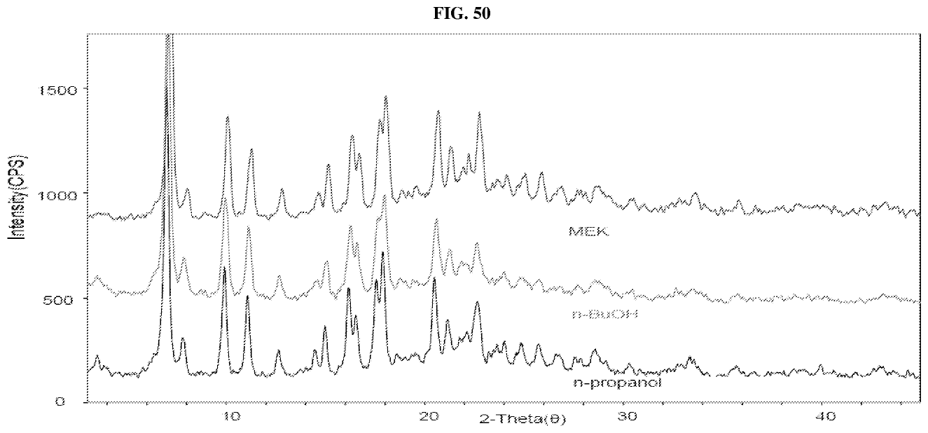

In some embodiments, the crystalline Form X is characterized by an X-ray powder diffraction pattern substantially, as shown in FIG. 50.

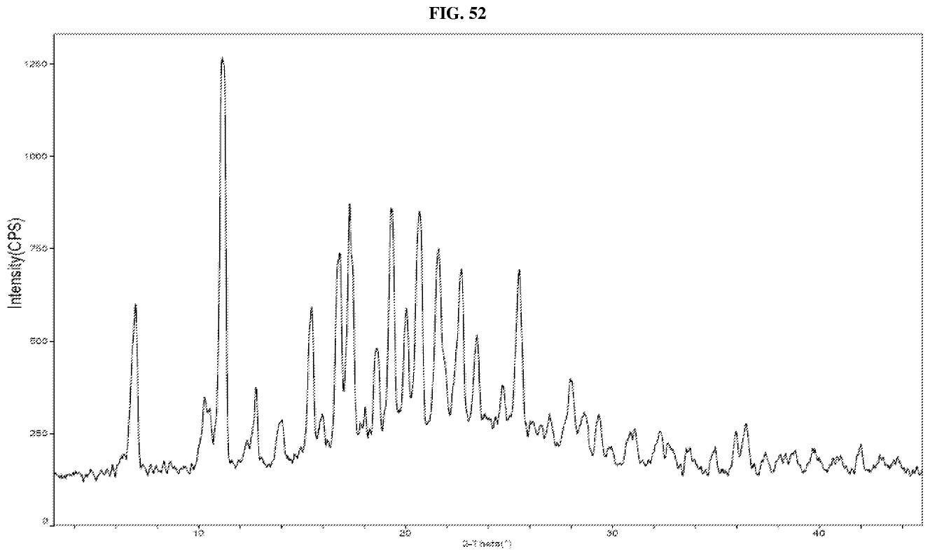

In some embodiments, the crystalline Form XI is characterized by an X-ray powder diffraction pattern substantially, as shown in FIG. 52.

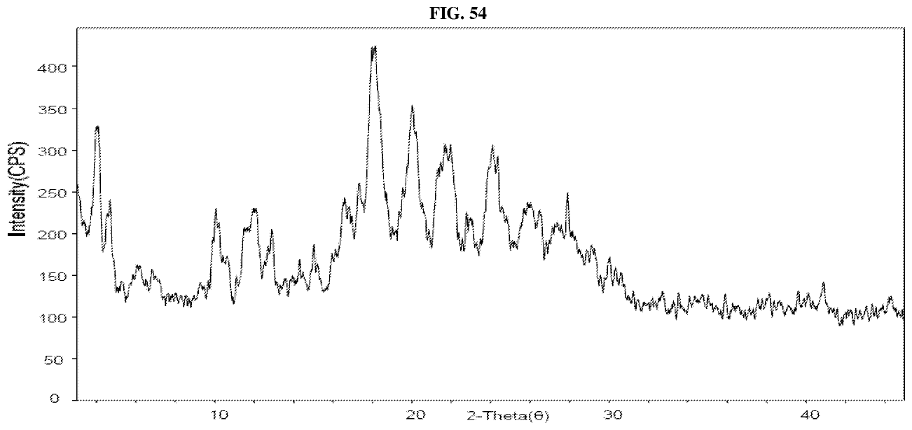

In some embodiments, the crystalline Form XII is characterized by an X-ray powder diffraction pattern substantially, as shown in FIG. 54.

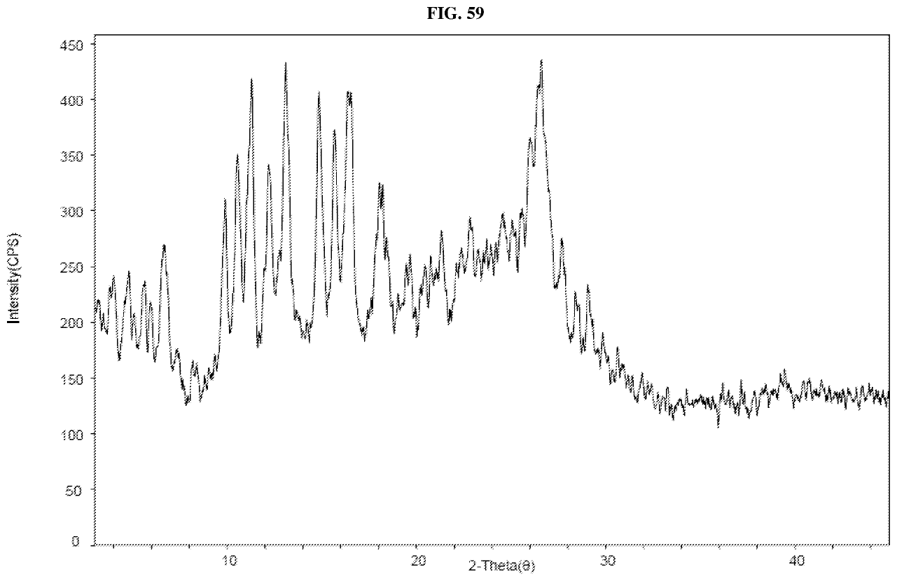

In some embodiments, the crystalline Form XIII is characterized by an X-ray powder diffraction pattern substantially, as shown in FIG. 59.

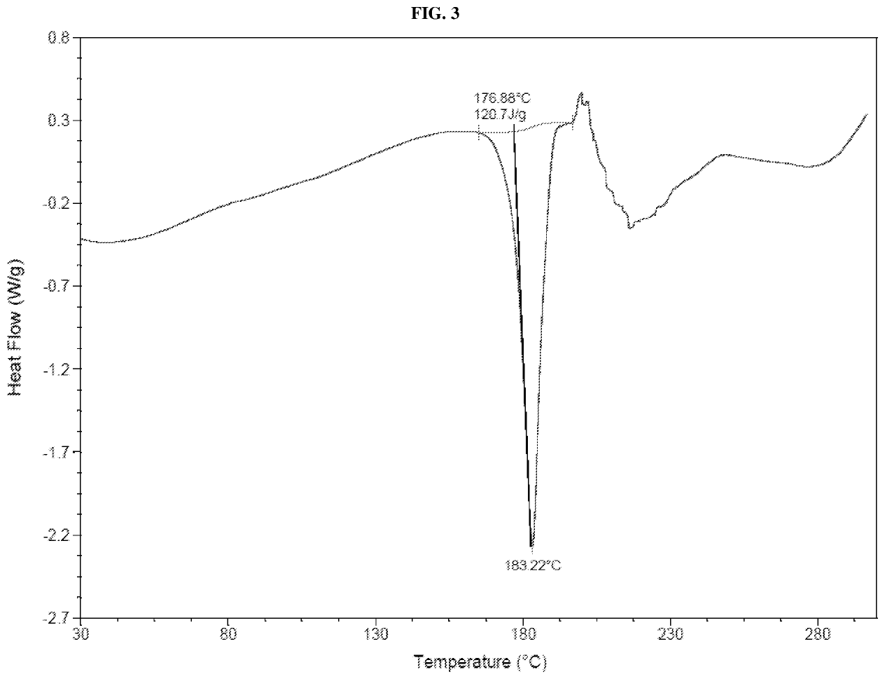

In some embodiments, the crystalline Forms I-XIII are characterized by a DSC thermogram. For example, the crystalline Form I to Form X are characterized by a DSC thermogram as shown in FIGS. 3, 7, 13, 19, 25, 31, 37, 43, 47, and 51 respectively.

In some embodiments, the crystalline Forms I-XIII are characterized by any combination of the above data.

In some embodiments, the X-ray powder diffraction peaks recited herein for particular embodiments can vary by ±0.4 degrees 2θ, by ±0.3 degrees 2θ, by ±0.2 degrees 2θ, or by ±0.1 degrees 2θ.

In some embodiments, the crystalline Form I is characterized by an X-ray powder diffraction pattern comprising a d-spacing value substantially, as shown in Table 1.

In some embodiments, the crystalline Form I is characterized by an X-ray powder diffraction pattern comprising one or more d-spacing values at about 12.3±0.5 degrees angstroms, at about 11.0±0.5 degrees angstroms, at about 8.7±0.5 degrees angstroms, at about 7.8±0.5 degrees angstroms, at about 6.8±0.5 degrees angstroms, at about 6.2±0.5 degrees angstroms, at about 5.8±0.5 degrees angstroms, at about 5.3±0.5 degrees angstroms, at about 4.9±0.5 degrees angstroms, at about 4.3±0.5 degrees angstroms, at about 4.1±0.5 degrees angstroms, at about 4.0±0.5 degrees angstroms, and at about 3.8±0.5 degrees angstroms.

In some embodiments, the crystalline Form II is characterized by an X-ray powder diffraction pattern comprising a d-spacing value substantially, as shown in Table 2.

In some embodiments, the crystalline Form II is characterized by an X-ray powder diffraction pattern comprising one or more d-spacing values at about 12.1±0.5 degrees angstroms, at about 10.9±0.5 degrees angstroms, at about 8.6±0.5 degrees angstroms, at about 7.7±0.5 degrees angstroms, at about 6.8±0.5 degrees angstroms, at about 5.7±0.5 degrees angstroms, at about 5.5±0.5 degrees angstroms, at about 5.2±0.5 degrees angstroms, at about 4.8±0.5 degrees angstroms, at about 4.6±0.5 degrees angstroms, at about 4.2±0.5 degrees angstroms, at about 4.1±0.5 degrees angstroms, at about 4.0±0.5 degrees angstroms, and at about 3.8±0.5 degrees angstroms.

In some embodiments, the crystalline Form III is characterized by an X-ray powder diffraction pattern comprising a d-spacing value substantially, as shown in Table 5.

In some embodiments, the crystalline Form III is characterized by an X-ray powder diffraction pattern comprising one or more d-spacing values at about 15.1±0.5 degrees angstroms, at about 10.0±0.5 degrees angstroms, at about 8.4±0.5 degrees angstroms, at about 7.1±0.5 degrees angstroms, at about 6.2±0.5 degrees angstroms, at about 5.1±0.5 degrees angstroms, at about 4.9±0.5 degrees angstroms, at about 4.8±0.5 degrees angstroms, at about 4.6±0.5 degrees angstroms, at about 4.4±0.5 degrees angstroms, at about 4.3±0.5 degrees angstroms, at about 4.2±0.5 degrees angstroms, at about 4.0±0.5 degrees angstroms, at about 3.9±0.5 degrees angstroms, at about 3.8±0.5 degrees angstroms, at about 3.7±0.5 degrees angstroms, at about 3.6±0.5 degrees angstroms, at about 3.4±0.5 degrees angstroms, at about 3.4±0.5 degrees angstroms, at about 3.2±0.5 degrees angstroms, at about 3.1±0.5 degrees angstroms, at about 2.8±0.5 degrees angstroms, and at about 2.3±0.5 degrees angstroms.

In some embodiments, the crystalline Form IV is characterized by an X-ray powder diffraction pattern comprising a d-spacing value substantially, as shown in Table 6.

In some embodiments, the crystalline Form IV is characterized by an X-ray powder diffraction pattern comprising one or more d-spacing values at about 19.4±0.5 degrees angstroms, at about 14.7±0.5 degrees angstroms, at about 10.0±0.5 degrees angstroms, at about 7.9±0.5 degrees angstroms, at about 7.5±0.5 degrees angstroms, at about 6.7±0.5 degrees angstroms, at about 5.4±0.5 degrees angstroms, at about 5.2±0.5 degrees angstroms, at about 5.0±0.5 degrees angstroms, at about 4.8±0.5 degrees angstroms, at about 4.5±0.5 degrees angstroms, at about 4.3±0.5 degrees angstroms, at about 4.2±0.5 degrees angstroms, at about 4.1±0.5 degrees angstroms, at about 3.7±0.5 degrees angstroms, at about 3.6±0.5 degrees angstroms, at about 3.6±0.5 degrees angstroms, at about 3.4±0.5 degrees angstroms, and at about 3.1±0.5 degrees angstroms.

In some embodiments, the crystalline Form V is characterized by an X-ray powder diffraction pattern comprising a d-spacing value substantially, as shown in Table 7.

In some embodiments, the crystalline Form V is characterized by an X-ray powder diffraction pattern comprising one or more d-spacing values at about 18.8±0.5 degrees angstroms, at about 11.9±0.5 degrees angstroms, at about 9.6±0.5 degrees angstroms, at about 7.9±0.5 degrees angstroms, at about 6.4±0.5 degrees angstroms, at about 5.1±0.5 degrees angstroms, at about 4.9±0.5 degrees angstroms, at about 4.7±0.5 degrees angstroms, at about 3.6±0.5 degrees angstroms, and at about 3.4±0.5 degrees angstroms.

In some embodiments, the crystalline Form VI is characterized by an X-ray powder diffraction pattern comprising a d-spacing value substantially, as shown in Table 8.

In some embodiments, the crystalline Form VI is characterized by an X-ray powder diffraction pattern comprising one or more d-spacing values at about 12.0±0.5 degrees angstroms, at about 9.3±0.5 degrees angstroms, at about 6.5±0.5 degrees angstroms, at about 6.0±0.5 degrees angstroms, at about 5.7±0.5 degrees angstroms, at about 5.4±0.5 degrees angstroms, at about 5.2±0.5 degrees angstroms, at about 4.9±0.5 degrees angstroms, at about 4.6±0.5 degrees angstroms, at about 4.3±0.5 degrees angstroms, at about 4.1±0.5 degrees angstroms, at about 3.8±0.5 degrees angstroms, at about 3.6±0.5 degrees angstroms, at about 3.5±0.5 degrees angstroms, at about 3.3±0.5 degrees angstroms, at about 3.0±0.5 degrees angstroms, and at about 2.9±0.5 degrees angstroms.

In some embodiments, the crystalline Form VII is characterized by an X-ray powder diffraction pattern comprising a d-spacing value substantially, as shown in Table 9.

In some embodiments, the crystalline Form VII is characterized by an X-ray powder diffraction pattern comprising one or more d-spacing values at about 18.9±0.5 degrees angstroms, at about 15.2±0.5 degrees angstroms, at about 8.4±0.5 degrees angstroms, at about 7.8±0.5 degrees angstroms, at about 7.5±0.5 degrees angstroms, at about 7.0±0.5 degrees angstroms, at about 6.8±0.5 degrees angstroms, at about 6.3±0.5 degrees angstroms, at about 5.5±0.5 degrees angstroms, at about 5.2±0.5 degrees angstroms, at about 5.1±0.5 degrees angstroms, at about 4.8±0.5 degrees angstroms, at about 4.6±0.5 degrees angstroms, at about 4.2±0.5 degrees angstroms, at about 4.0±0.5 degrees angstroms, at about 3.7±0.5 degrees angstroms, at about 3.6±0.5 degrees angstroms, at about 3.4±0.5 degrees angstroms, and at about 3.2±0.5 degrees angstroms.

In some embodiments, the crystalline Form VIII is characterized by an X-ray powder diffraction pattern comprising a d-spacing value substantially, as shown in Table 10.

In some embodiments, the crystalline Form VIII is characterized by an X-ray powder diffraction pattern comprising d-spacing values at about 22.5±0.5 degrees angstroms, at about 15.5±0.5 degrees angstroms, at about 12.5±0.5 degrees angstroms, at about 10.3±0.5 degrees angstroms, at about 8.6±0.5 degrees angstroms, at about 7.3±0.5 degrees angstroms, at about 6.3±0.5 degrees angstroms, at about 5.2±0.5 degrees angstroms, at about 4.7±0.5 degrees angstroms, at about 4.3±0.5 degrees angstroms, at about 4.0±0.5 degrees angstroms, at about 3.9±0.5 degrees angstroms, at about 3.7±0.5 degrees angstroms, at about 3.4±0.5 degrees angstroms, at about 3.4±0.5 degrees angstroms, at about 3.1±0.5 degrees angstroms, at about 2.6±0.5 degrees angstroms, at about 2.5±0.5 degrees angstroms, at about 2.4±0.5 degrees angstroms, and at about 2.3±0.5 degrees angstroms.

In some embodiments, the crystalline Form IX is characterized by an X-ray powder diffraction pattern comprising a d-spacing value substantially, as shown in Table 11.

In some embodiments, the crystalline Form IX is characterized by an X-ray powder diffraction pattern comprising d-spacing values at about 16.7±0.5 degrees angstroms, at about 12.9±0.5 degrees angstroms, at about 9.8±0.5 degrees angstroms, at about 8.8±0.5 degrees angstroms, at about 5.7±0.5 degrees angstroms, at about 5.1±0.5 degrees angstroms, at about 4.9±0.5 degrees angstroms, at about 4.7±0.5 degrees angstroms, at about 4.5±0.5 degrees angstroms, at about 4.3±0.5 degrees angstroms, at about 4.2±0.5 degrees angstroms, at about 3.9±0.5 degrees angstroms, at about 3.8±0.5 degrees angstroms, at about 3.7±0.5 degrees angstroms, at about 3.5±0.5 degrees angstroms, at about 3.4±0.5 degrees angstroms, at about 3.3±0.5 degrees angstroms, at about 3.2±0.5 degrees angstroms, at about 3.1±0.5 degrees angstroms, at about 3.0±0.5 degrees angstroms, at about 2.7±0.5 degrees angstroms, at about 2.6±0.5 degrees angstroms, at about 2.5±0.5 degrees angstroms, at about 2.3±0.5 degrees angstroms, and at about 2.3±0.5 degrees angstroms.

In some embodiments, the X-ray powder diffraction peaks recited herein for particular embodiments having d-spacing values can vary by ±4% nm, by ±3% nm, by ±2% nm, or by ±1% nm or by ±4% angstroms, by ±3% angstroms, by ±2% angstroms, or by ±1% angstroms.

One skilled in the art will understand that the relative intensities and positions of the peaks obtained by X-ray powder diffraction may vary depending upon, inter alia, the sample preparation technique, the sample mounting procedure, and the particular instrument employed. For example, in some embodiments, the listed X-ray powder diffraction pattern peaks for any of the crystalline Forms I-XIII are about ±0.2 degrees 2θ.

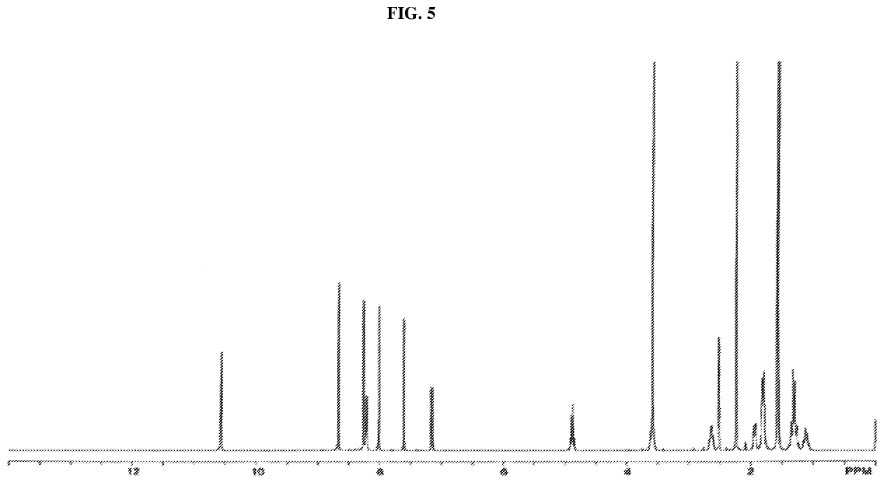

In some embodiments, the crystalline Forms I-XIII of the compound of Formula I are characterized using Proton Nuclear Magnetic Resonance Spectroscopy (¹H NMR). For example, FIGS. 5, 9, 16, 22, 28, 34, 40, 45, and 49 show Proton Nuclear Magnetic Resonance Spectroscopy (¹H NMR) analysis of the crystalline Forms I-IX, respectively. Other methods for characterizing the crystalline Forms I-XIII could also be used.

The crystalline Forms I-XIII can have any desired degree of purity relative to other substances or components in the preparation. In some embodiments, any form of the crystalline Forms I-XIII is provided such that it is substantially pure, such as, for example, having greater than 30%, greater than 40%, greater than 50%, greater than 60%, greater than 70%, greater than 80%, greater than 85%, greater than 90%, greater than 95%, greater than 96%, greater than 97%, greater than 98%, greater than 99%, greater than 99.2%, greater than 99.4%, greater than 99.5%, greater than 99.6%, greater than 99.7%, or greater than 99.9% purity, relative to other substances or components in the preparation.

In exemplary embodiments, any one of the crystalline forms as described or provided herein, such as the crystalline Forms I-XIII of the compound of Formula I, is about 45% to 95% pure, such as, for example, about 50% to 95% pure, about 55% to 90% pure, about 60% to 95% pure, or about 70% to 99% pure, relative to other substances or components in the preparation. In some embodiments, the crystalline any one of Forms I-XIII is about 95% to 99% pure. In some embodiments, the crystalline form is about 90% to 95% pure. In some embodiments, the crystalline form is about 85% to 90% pure. In some embodiments, the crystalline form is about 80% to 85% pure. In some embodiments, the crystalline form is about 75% to 80% pure. In some embodiments, the crystalline Form I is about 70% to 75% pure. In certain embodiments, the crystalline form is about 65% to 70% pure. In some embodiments, the crystalline form is about 60% to 65% pure. In other embodiments, the crystalline form is about 55% to 60% pure. In yet other embodiments, the crystalline Form I is about 50% to 55% pure. In some embodiments, the crystalline form is about 45% to 50% pure.

In some embodiments, any one of the crystalline forms as described or provided herein, such as the crystalline Forms I-XIII the compound of Formula I, may comprise one or more impurities and/or a degradation product, such as a hydrolysis product, acetylation product, a formylation product, an oxidation product, a water-mediated degradation product, and/or a deamidation product. In some embodiments, a composition comprising any one of the crystalline forms as described or provided herein, such as the crystalline Forms I-XIII of the compound of Formula I, may comprise one or more impurities and/or a degradation product, such as a hydrolysis product, acetylation product, a formylation product, an oxidation product, a water-mediated degradation product, and/or a deamidation product. In some embodiments, one or more impurities may be biologically active.

In some embodiments, any one of the crystalline forms as described or provided herein, such as the crystalline Forms I-XIII of the compound of Formula I, and/or the composition thereof comprising any one of the crystalline forms as described or provided herein, such as the crystalline Forms I-XIII of the compound of Formula I, can contain any desired purity relative to hydrolysis product(s). In some embodiments, the composition comprises less than about 10% by weight of hydrolysis product(s), relative to the total weight of any one of the crystalline forms as described or provided herein and/or the composition thereof, such as, for example, less than about 7.5 wt. %, less than about 5 wt. %, or less than about 2 wt. % of hydrolysis product(s). In some embodiments, any one of the crystalline forms as described or provided herein, such as the crystalline Forms I-XIII of the compound of Formula I, and/or the composition thereof comprises from about 0.05% to about 5% by weight of hydrolysis product(s). In some embodiments, any one of the crystalline forms as described or provided herein, such as the crystalline Forms I-XIII of the compound of Formula I, and/or the composition thereof comprises from about 0.05% to about 2% by weight of the hydrolysis product(s). In some embodiments, any one of the crystalline forms as described or provided herein, such as the crystalline Forms I-XIII of the compound of Formula I, and/or the composition thereof comprises from about 0.1% to about 2% by weight of the hydrolysis product(s). In some embodiments, any one of the crystalline forms as described or provided herein, such as the crystalline Forms I-XIII of the compound of Formula I, and/or the composition thereof comprises from about 0.01% to about 2% by weight of the hydrolysis product(s).

Alternatively, or in addition, any one of the crystalline forms as described or provided herein, such as the crystalline Forms I-XIII of the compound of Formula I, and/or the composition thereof comprising any one of the crystalline forms as described or provided herein, such as the crystalline Forms I-XIII of the compound of Formula I, can contain any desired purity relative to acetylation product(s). In some embodiments, the acetylation product may comprise less than 10% by weight of any one of the crystalline forms as described or provided herein, such as the crystalline Forms I-XIII of the compound of Formula I, and/or the compositions thereof. In some embodiments, the acetylation product may comprise less than 7.5% by weight of any one of the crystalline forms as described or provided herein, such as the crystalline Forms I-XIII of the compound of Formula I, and/or the composition thereof. In some embodiments, the acetylation product may comprise less than 5% by weight of any one of the crystalline forms as described or provided herein, such as the crystalline Forms I-XIII of the compound of Formula I, and/or the composition thereof. In some embodiments, the acetylation product may comprise less than 2% by weight of any one of the crystalline forms as described or provided herein, such as the crystalline Forms I-XIII of the compound of Formula I, and/or the composition thereof. In some embodiments, the acetylation product may comprise less than 1% by weight of any one of the crystalline forms as described or provided herein, such as the crystalline Forms I-XIII of the compound of Formula IL and/or the composition thereof. In some embodiments, the acetylation product may comprise less than 0.5% by weight of any one of the crystalline forms as described or provided herein and/or the composition thereof. In some embodiments, the acetylation product may comprise from about 0.05% to about 5% by weight of any one of the crystalline forms as described or provided herein and/or the composition thereof. In some embodiments, the acetylation product may comprise from about 0.05% to about 2% by weight of any one of the crystalline forms as described or provided herein and/or the composition thereof. In some embodiments, the acetylation product may comprise from about 0.1% to about 2% by weight of any one of the crystalline forms as described or provided herein and/or the composition thereof. In some embodiments, the acetylation product may comprise from about 0.01% to about 2% by weight of the composition.

Alternatively, or in addition, any one of the crystalline forms as described or provided herein, such as the crystalline Forms I-XIII of the compound of Formula I, and/or the composition thereof comprising any one of the crystalline forms as described or provided herein, such as the crystalline Forms I-XIII of the compound of Formula I, can contain any desired purity relative to formylation product(s). In some embodiments, the formylation product may comprise less than 10% by weight of any one of the crystalline forms as described or provided herein and/or the composition thereof. In some embodiments, the formylation product may comprise less than 7.5% by weight of any one of the crystalline forms as described or provided herein and/or the composition thereof. In some embodiments, the formylation product may comprise less than 5% by weight of any one of the crystalline forms as described or provided herein and/or the composition thereof. In some embodiments, the formylation product may comprise less than 2% by weight of any one of the crystalline forms as described or provided herein and/or the composition thereof. In some embodiments, the formylation product may comprise from about 0.05% to about 5% by weight of any one of the crystalline forms as described or provided herein and/or the composition thereof. In some embodiments, the formylation product may comprise from about 0.05% to about 2% by weight of any one of the crystalline forms as described or provided herein and/or the composition thereof. In some embodiments, the formylation product may comprise from about 0.1% to about 2% by weight of any one of the crystalline forms as described or provided herein and/or the composition thereof.

Alternatively, or in addition, any one of the crystalline forms as described or provided herein, such as the crystalline Forms I-XIII of the compound of Formula I, and/or the composition thereof comprising any one of the crystalline forms as described or provided herein, such as the crystalline Forms I-XIII of the compound of Formula I, can contain any desired purity relative to oxidation product(s). In some embodiments, the oxidation product may comprise less than 10% by weight of any one of the crystalline forms as described or provided herein and/or the composition thereof. In some embodiments, the oxidation product may comprise less than 7.5% by weight of any one of the crystalline forms as described or provided herein and/or the composition thereof. In some embodiments, the oxidation product may comprise less than 5% by weight of any one of the crystalline forms as described or provided herein and/or the composition thereof. In some embodiments, the oxidation product may comprise less than 2% by weight of any one of the crystalline forms as described or provided herein and/or the composition thereof. In some embodiments, the oxidation product may comprise from about 0.05% to about 5% by weight of any one of the crystalline forms as described or provided herein and/or the composition thereof. In some embodiments, the oxidation product may comprise from about 0.05% to about 2% by weight of any one of the crystalline forms as described or provided herein and/or the composition thereof. In some embodiments, the oxidation product may comprise from about 0.1% to about 2% by weight of any one of the crystalline forms as described or provided herein and/or the composition thereof. In some embodiments, the oxidation product may comprise from about 0.01% to about 2% by weight of any one of the crystalline forms as described or provided herein and/or the composition thereof.

Alternatively, or in addition, any one of the crystalline forms as described or provided herein, such as the crystalline Forms I-XIII of the compound of Formula I, and/or the composition thereof comprising any one of the crystalline forms as described or provided herein, such as the crystalline Forms I-XIII of the compound of Formula I, can contain any desired purity relative to water-mediated degradation product(s). In some embodiments, the water-mediated degradation product(s) may comprise less than 10% by weight of any one of the crystalline forms as described or provided herein and/or the composition thereof. In some embodiments, the water-mediated degradation product(s) may comprise less than 7.5% by weight of any one of the crystalline forms as described or provided herein and/or the composition thereof. In some embodiments, the water-mediated degradation product(s) may comprise less than 5% by weight of any one of the crystalline forms as described or provided herein and/or the composition thereof. In other embodiments, the water-mediated degradation product(s) may comprise less than 2% by weight of any one of the crystalline forms as described or provided herein and/or the composition thereof. In some embodiments, the water-mediated degradation product(s) may comprise from about 0.05% to about 5% by weight of any one of the crystalline forms as described or provided herein and/or the composition thereof. In exemplary embodiments, the water-mediated degradation product(s) may comprise from about 0.05% to about 2% by weight of any one of the crystalline forms as described or provided herein and/or the composition thereof. In some embodiments, the water-mediated degradation product(s) may comprise from about 0.1% to about 2% by weight of any one of the crystalline forms as described or provided herein and/or the composition thereof. In some embodiments, the water-mediated degradation product(s) may comprise from about 0.01% to about 2% by weight of any one of the crystalline forms as described or provided herein and/or the composition thereof

Alternatively, or in addition, any one of the crystalline forms as described or provided herein, such as the crystalline Forms I-XIII of the compound of Formula IL and/or the composition thereof comprising any one of the crystalline forms as described or provided herein, such as the crystalline Forms I-XIII of the compound of Formula I, can contain any desired purity relative to deamidation product(s). In some embodiments, the deamidation product may comprise less than 10% by weight of any one of the crystalline forms as described or provided herein and/or the composition thereof. In some embodiments, the deamidation product may comprise less than 7.5% by weight of any one of the crystalline forms as described or provided herein and/or the composition thereof. In some embodiments, the deamidation product may comprise less than 5% by weight of any one of the crystalline forms as described or provided herein and/or the composition thereof. In other embodiments, the deamidation product may comprise less than 2% by weight of any one of the crystalline forms as described or provided herein and/or the composition thereof. In some embodiments, the deamidation product may comprise from about 0.05% to about 5% by weight of any one of the crystalline forms as described or provided herein and/or the composition thereof. In some embodiments, the deamidation product may comprise from about 0.05% to about 2% by weight of any one of the crystalline forms as described or provided herein and/or the composition thereof. In some embodiments, the deamidation product may comprise from about 0.1% to about 2% by weight of any one of the crystalline forms as described or provided herein and/or the composition thereof. In some embodiments, the deamidation product may comprise from about 0.01% to about 2% by weight of any one of the crystalline forms as described or provided herein and/or the composition thereof.

In some embodiments, a composition is provided comprising any one of the crystalline forms as described or provided herein, such as the crystalline Forms I XLII of the compound of Formula I, and less than 10 wt. % such as less than 8 wt. %, less than 6 wt. %, less than 5 wt. %, less than 4 wt. %, less than 3 wt. %, less than 2 wt. %, less than 1 wt. %, less than 0.5 wt. %, or less than 0.25 wt. % of a combined total of a degradation product, such as a hydrolysis product, a formylation product, an oxidation product, a water-mediated degradation product, and/or a deamidation product.

In some embodiments, a composition is provided comprising any one of the crystalline forms as described or provided herein, such as the crystalline Forms I-XIII of the compound of Formula I, and less than 20 wt. % such as less than 18 wt. %, less than 16 wt. less than 14 wt. %, less than 12 wt. %, less than 10 wt. %, less than 8 wt. %, less than 6 wt %, less than 5 wt. %, less than 4 wt. %, less than 3 wt. %, less than 2 wt. %, less than 1 wt. %, less than 0.5 wt. %, or less than 0.25 wt. % of a combined total of a degradation product, such as a hydrolysis product, an acetylation product, a formylation product, an oxidation product, a water-mediated degradation product, and/or a deamidation product.

In some embodiments, a composition is provided comprising any one of the crystalline forms as described or provided herein, such as the crystalline Forms I-XIII of the compound of Formula I, and less than 10 wt. % such as less than 8 wt. %, less than 6 wt. %, less than 5 wt. %, less than 4 wt. %, less than 3 wt. %, less than 2 wt. %, less than 1 wt. %, less than 0.5 wt. %, or less than 0.25 wt. 3% of a combined total of one or more impurities and/or a degradation product, such as a hydrolysis product, a formylation product, an oxidation product, a water-mediated degradation product, and/or a deamidation product.

In some embodiments, a composition is provided comprising any one of the crystalline forms as described or provided herein, such as the crystalline Forms I-XIII of the compound of Formula I, and less than 20 wt. % such as less than 18 wt. %, less than 16 wt. %, less than 14 wt. %, less than 12 wt. 24, less than 10 wt. %, less than 8 wt. %, less than 6 wt. %, less than 5 wt. %, less than 4 wt. %, less than 3 wt. %, less than 2 wt. %, less than 1 wt, %, less than 0.5 wt. %, or less than 0.25 wt. % of a combined total of one or more impurities and/or a degradation product, such as a hydrolysis product, an acetylation product, a formylation product, an oxidation product, a water-mediated degradation product, and/or a deamidation product.

In some embodiments, a composition is provided comprising any one of the crystalline forms as described or provided herein, such as the crystalline Forms I-XIII of the compound of Formula I, and less than about 40 wt %, such as less than about 30 wt. %, less than about 20 wt. %, less than about 15 wt. %, less than about 10 wt. %, less than about 8 wt. %, less than about 6 wt. %, less than about 5 wt. %, less than about 4 wt. %, less than about 3 wt. %, less than about 2 wt. %, less than about 1 wt. %, less than about 0.5 wt. %, less than about 0.1 wt. %, or less than about 0.01 wt. % of amorphous form of the compound of Formula I.

In some embodiments, processes for preparing crystalline forms of the compound of Formula I are provided. In some embodiments, the crystalline any one of the crystalline forms as described or provided herein is produced by precipitating and crystallizing the compound of Formula I with or without a conformer and optionally isolating the crystalline any one of the crystalline forms as described or provided herein. In some embodiments, the crystalline any one of the crystalline forms as described or provided herein is prepared by slurrying the compound of Formula I with or without a conformer in an organic solvent and optionally isolating the crystalline any one of the crystalline forms as described or provided herein. In some embodiments, the crystalline any one of the crystalline forms as described or provided herein is prepared by slurring and crystallizing the compound of Formula I with or without a conformer in a super saturated organic solvent and optionally isolating the crystalline any one of the crystalline forms as described or provided herein.

Any suitable organic solvent can be used in this regard, such as, for example, acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, octane, and any combination thereof. Such solvents may include but are not limited to, acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, octane, and any combination thereof. In some embodiments, the organic solvent comprises acetonitrile. In some embodiments, the organic solvent comprises n-butanol. In some embodiments, the organic solvent comprises methyl ethyl ketone. In some embodiments, the organic solvent comprises methanol. In some embodiments, the organic solvent comprises ethyl acetate. In some embodiments, the organic solvent comprises acetone. In some embodiments, the organic solvent comprises tetrahydrofuran. In some embodiments, the organic solvent comprises 2-propanol. In some embodiments, the organic solvent comprises ethanol. In some embodiments, the organic solvent comprises isopropyl acetate. In some embodiments, the organic solvent comprises toluene. In some embodiments, the organic solvent comprises cyclohexane. In some embodiments, the organic solvent comprises dichloromethane. In some embodiments, the organic solvent comprises chloroform. In some embodiments, the organic solvent comprises H₂O. In some embodiments, the organic solvent comprises nitromethane. In some embodiments, the organic solvent comprises n-pentane. In some embodiments, the organic solvent comprises n-hexane. In some embodiments, the organic solvent comprises 1-propanol. In some embodiments, the organic solvent comprises methyl acetate. In some embodiments, the organic solvent comprises ethyl ether. In some embodiments, the organic solvent comprises octane.

The crystalline of any one of the crystalline forms as described or provided herein of the compound of Formula I may be identified, characterized, and distinguished from amorphous or oil form using any suitable manner. One skilled in the art will know many different methods of identification and characterization of the crystalline any one of the crystalline forms as described or provided herein. For example, the crystalline any one of the crystalline forms as described or provided herein of the compound of Formula I may be identified and characterized based on differences in diffraction, thermal, intensity, and/or spectroscopic properties of the amorphous and crystalline form. Suitable methods include, but are not limited to, X-ray diffractometry, Proton Nuclear Magnetic Resonance Spectroscopy (¹H NMR), Carbon Nuclear Magnetic Resonance Spectroscopy (¹³C NMR), thermogravimetric analysis (TGA), and differential scanning calorimetry (DSC).

In some embodiments, processes for preparing a crystalline form of a compound having a formula of

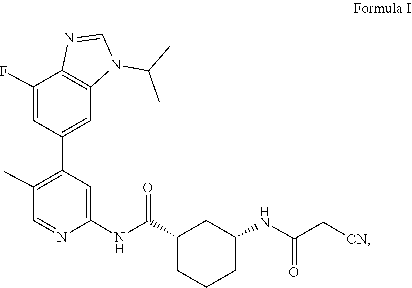 [SMILES: Cc1cnc(NC(=O)[C@H]2CCC[C@@H](NC(=O)CC#N)C2)cc1-c1cc(F)c2ncn(C(C)C)c2c1]

comprising crystallizing the compound of Formula I to form the crystalline Form I and optionally isolating the Form I. In some embodiments, the crystallizing comprises dissolving the compound in an organic solvent and crystallizing the compound to yield the Form I therefrom. In some embodiments, the organic solvent is selected from the group consisting of acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, octane.

In some embodiments, processes for preparing a crystalline form of a compound having a formula of

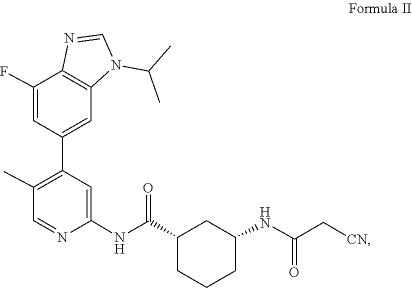 [SMILES: Cc1cnc(NC(=O)[C@H]2CCC[C@@H](NC(=O)CC#N)C2)cc1-c1cc(F)c2ncn(C(C)C)c2c1]

comprising crystallizing the compound of Formula I to form the crystalline form such as From I and Form II as described herein and optionally isolating the crystalline form. In some embodiments, the crystallizing comprises dissolving the compound in an organic solvent and crystallizing the compound to form the crystalline form therefrom. In some embodiments, the organic solvent is selected from the group consisting of acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, octane. In some embodiments, the organic solvent is selected from the group consisting of acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, octane, and any combination thereof. In some embodiments, the organic solvent is acetonitrile. In some embodiments, the organic solvent is n-butanol. In some embodiments, the organic solvent is methyl ethyl ketone. In some embodiments, the organic solvent is methanol. In some embodiments, the organic solvent is ethyl acetate. In some embodiments, the organic solvent is acetone. In some embodiments, the organic solvent is tetrahydrofuran. In some embodiments, the organic solvent is 2-propanol. In some embodiments, the organic solvent is ethanol. In some embodiments, the organic solvent is isopropyl acetate. In some embodiments, the organic solvent is toluene. In some embodiments, the organic solvent is cyclohexane. In some embodiments, the organic solvent is dichloromethane. In some embodiments, the organic solvent is chloroform. In some embodiments, the organic solvent is H₂O. In some embodiments, the organic solvent is nitromethane. In some embodiments, the organic solvent is n-pentane. In some embodiments, the organic solvent is n-hexane. In some embodiments, the organic solvent is 1-propanol. In some embodiments, the organic solvent is methyl acetate. In some embodiments, the organic solvent is ethyl ether. In some embodiments, the organic solvent is octane. In some embodiments, the organic solvent is any combination of acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, and octane

In some embodiments, processes for preparing a crystalline form of the compound of

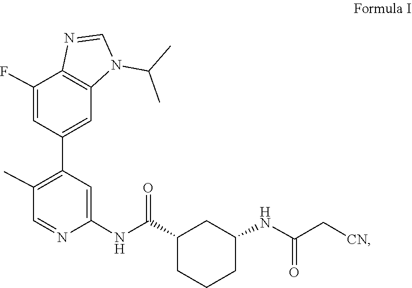 [SMILES: Cc1cnc(NC(=O)[C@H]2CCC[C@@H](NC(=O)CC#N)C2)cc1-c1cc(F)c2ncn(C(C)C)c2c1]

and a coformer as described or provided herein.

In some embodiments, the process comprises co-crystallizing the compound and the coformer to form the crystalline form of the compound of Formula I and the coformer such as the crystalline Forms III-XIII and optionally isolating the crystalline form of the compound and the coformer. In some embodiments, the process comprises slurrying the compound and the coformer in an organic solvent to form the crystalline form therefrom. In some embodiments, the process further comprises washing the slurry with the organic solvent. In some embodiments, the organic solvent is selected from the group consisting of acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, octane, and any combination thereof. In some embodiments, the organic solvent is acetonitrile. In some embodiments, the organic solvent is n-butanol. In some embodiments, the organic solvent is methyl ethyl ketone. In some embodiments, the organic solvent is methanol. In some embodiments, the organic solvent is ethyl acetate. In some embodiments, the organic solvent is acetone. In some embodiments, the organic solvent is tetrahydrofuran. In some embodiments, the organic solvent is 2-propanol. In some embodiments, the organic solvent is ethanol. In some embodiments, the organic solvent is isopropyl acetate. In some embodiments, the organic solvent is toluene. In some embodiments, the organic solvent is cyclohexane. In some embodiments, the organic solvent is dichloromethane. In some embodiments, the organic solvent is chloroform. In some embodiments, the organic solvent is H₂O. In some embodiments, the organic solvent is nitromethane. In some embodiments, the organic solvent is n-pentane. In some embodiments, the organic solvent is n-hexane. In some embodiments, the organic solvent is 1-propanol. In some embodiments, the organic solvent is methyl acetate. In some embodiments, the organic solvent is ethyl ether. In some embodiments, the organic solvent is octane. In some embodiments, the organic solvent is any combination of acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, and octane.

In some embodiments, the coformer is a coformer provided and described herein. In some embodiments, the coformer is succinic acid, adipic acid, fumaric acid, glutaric acid, gentisic acid, hydrochloric acid, 1-hydroxy-2-naphthoic acid, salicylic acid, oxalic acid, or D-(−)-tartaric acid. In some embodiments, the coformer is succinic acid, adipic acid, fumaric acid, glutaric acid, gentisic acid, hydrochloric acid, 1-hydroxy-2-naphthoic acid, salicylic acid, oxalic acid, or D-(−)-tartaric acid.

In some embodiments, the coformer is succinic acid. In some embodiments, the crystalline form of the compound of Formula I and succinic acid has a molar ratio of the compound to the succinic acid in any range from about 0.1:1 to about 2:1. In some embodiments, the ratio is in a range of about 0.1:1 to about 2:1. In some embodiments, the ratio is in a range of about 0.1:1 to about 1.9:1, about 0.2:1 to about 1.8:1, about 0.3:1 to about 1.7:1, about 0.4:1 to about 1.6:1, about 0.5:1 to about 1.5:1, about 0.6:1 to about 1.4:1, about 0.7:1 to about 1.3:1, about 0.8:1 to about 1.2:1, or about 0.9:1 to about 1.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the crystalline form is a crystalline Form III of the compound and succinic acid, wherein the molar ratio of the compound to the succinic acid is in any range from about 0.1:1 to about 2:1. In some embodiments, the crystalline form is a crystalline Form III of the compound and succinic acid, wherein the molar ratio of the compound to the succinic acid is in a range from about 0.1:1 to about 2:1. In some embodiments, the crystalline form is a crystalline Form III of the compound and succinic acid, wherein the molar ratio of the compound to the succinic acid is about 1:1.

In some embodiments, the coformer is glutaric acid. In some embodiments, the crystalline form of the compound of Formula I and glutaric acid has a molar ratio of the compound to the glutaric acid in any range from about 0.1:1 to about 4:1. In some embodiments, the ratio is in a range of about 0.1:1 to about 4:1. In some embodiments, the ratio is in a range of about 0.1:1 to about 3.9:1, about 0.2:1 to about 3.8:1, about 0.3:1 to about 3.7:1, about 0.4:1 to about 3.6:1, about 0.5:1 to about 3.5:1, about 0.6:1 to about 3.4:1, about 0.7:1 to about 3.3:1, about 0.8:1 to about 3.2:1, about 0.9:1 to about 3.1:1, about 1:1 to about 3:1, about 1.1:1 to about 2.9:1, about 1.2:1 to about 2.8:1, about 1.3:1 to about 2.7:1, about 1.4:1 to about 2.6:1, about 1.5:1 to about 2.5:1, about 1.6:1 to about 2.4:1, about 1.7:1 to about 1.3:1, about 1.8:1 to about 2.2:1, or about 1.9:1 to about 2.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the ratio is about 2.1:1. In some embodiments, the ratio is about 2.2:1. In some embodiments, the ratio is about 2.3:1. In some embodiments, the ratio is about 2.4:1. In some embodiments, the ratio is about 2.5:1. In some embodiments, the ratio is about 2.6:1. In some embodiments, the ratio is about 2.7:1. In some embodiments, the ratio is about 2.8:1. In some embodiments, the ratio is about 2.9:1. In some embodiments, the ratio is about 3:1. In some embodiments, the ratio is about 3.1:1. In some embodiments, the ratio is about 3.2:1. In some embodiments, the ratio is about 3.3:1. In some embodiments, the ratio is about 3.4:1. In some embodiments, the ratio is about 3.5:1. In some embodiments, the ratio is about 3.6:1. In some embodiments, the ratio is about 3.7:1. In some embodiments, the ratio is about 3.8:1. In some embodiments, the ratio is about 3.9:1. In some embodiments, the ratio is about 4:1. In some embodiments, the crystalline form is a crystalline Form IV of the compound and glutaric acid, wherein the molar ratio of the compound to the glutaric acid is in any range from about 0.1:1 to about 4:1. In some embodiments, the crystalline form is a crystalline Form IV of the compound and glutaric acid, wherein the molar ratio of the compound to the glutaric acid is in a range from about 0.1:1 to about 4:1. In some embodiments, the crystalline form is a crystalline Form IV of the compound and glutaric acid, wherein the molar ratio of the compound to the glutaric acid is about 2:1.

In some embodiments, the coformer is adipic acid. In some embodiments, the crystalline form of the compound of Formula I and adipic has a molar ratio of the compound to the adipic in any range from about 0.1:1 to about 2:1. In some embodiments, the ratio is in a range of about 0.1:1 to about 2:1. In some embodiments, the ratio is in a range of about 0.1:1 to about 1.9:1, about 0.2:1 to about 1.8:1, about 0.3:1 to about 1.7:1, about 0.4:1 to about 1.6:1, about 0.5:1 to about 1.5:1, about 0.6:1 to about 1.4:1, about 0.7:1 to about 1.3:1, about 0.8:1 to about 1.2:1, or about 0.9:1 to about 1.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the crystalline form is a crystalline Form V of the compound and adipic, wherein the molar ratio of the compound to the adipic is in any range from about 0.1:1 to about 2:1. In some embodiments, the crystalline form is a crystalline Form V of the compound and adipic, wherein the molar ratio of the compound to the adipic is in a range from about 0.1:1 to about 2:1. In some embodiments, the crystalline form is a crystalline Form V of the compound and adipic, wherein the molar ratio of the compound to the adipic is about 1:1.

In some embodiments, the coformer is gentisic acid. In some embodiments, the crystalline form of the compound of the Formula I and gentisic acid has a molar ratio of the compound to the gentisic acid in any range from about 0.1:1 to about 4:1. In some embodiments, the ratio is in a range of about 0.1:1 to about 4:1. In some embodiments, the ratio is in a range of about 0.1:1 to about 3.9:1, about 0.2:1 to about 3.8:1, about 0.3:1 to about 3.7:1, about 0.4:1 to about 3.6:1, about 0.5:1 to about 3.5:1, about 0.6:1 to about 3.4:1, about 0.7:1 to about 3.3:1, about 0.8:1 to about 3.2:1, about 0.9:1 to about 3.1:1, about 1:1 to about 3:1, about 1.1:1 to about 2.9:1, about 1.2:1 to about 2.8:1, about 1.3:1 to about 2.7:1, about 1.4:1 to about 2.6:1, about 1.5:1 to about 2.5:1, about 1.6:1 to about 2.4:1, about 1.7:1 to about 1.3:1, about 1.8:1 to about 2.2:1, or about 1.9:1 to about 2.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the ratio is about 2.1:1. In some embodiments, the ratio is about 2.2:1. In some embodiments, the ratio is about 2.3:1. In some embodiments, the ratio is about 2.4:1. In some embodiments, the ratio is about 2.5:1. In some embodiments, the ratio is about 2.6:1. In some embodiments, the ratio is about 2.7:1. In some embodiments, the ratio is about 2.8:1. In some embodiments, the ratio is about 2.9:1. In some embodiments, the ratio is about 3:1. In some embodiments, the ratio is about 3.1:1. In some embodiments, the ratio is about 3.2:1. In some embodiments, the ratio is about 3.3:1. In some embodiments, the ratio is about 3.4:1. In some embodiments, the ratio is about 3.5:1. In some embodiments, the ratio is about 3.6:1. In some embodiments, the ratio is about 3.7:1. In some embodiments, the ratio is about 3.8:1. In some embodiments, the ratio is about 3.9:1. In some embodiments, the ratio is about 4:1. In some embodiments, the crystalline form is a crystalline Form VI of the compound and gentisic acid, wherein the molar ratio of the compound to the gentisic acid is in any range from about 0.1:1 to about 4:1. In some embodiments, the crystalline form is a crystalline Form VI of the compound and gentisic acid, wherein the molar ratio of the compound to the gentisic acid is in a range from about 0.1:1 to about 4:1. In some embodiments, the crystalline form is a crystalline Form VI of the compound and gentisic acid, wherein the molar ratio of the compound to the gentisic acid is about 2:1.

In some embodiments, the coformer is fumaric acid. In some embodiments, the crystalline form of the compound of Formula I and fumaric has a molar ratio of the compound to the fumaric in any range from about 0.1:1 to about 4:1. In some embodiments, the ratio is in a range of about 0.1:1 to about 4:1. In some embodiments, the ratio is in a range of about 0.1:1 to about 3.9:1, about 0.2:1 to about 3.8:1, about 0.3:1 to about 3.7:1, about 0.4:1 to about 3.6:1, about 0.5:1 to about 3.5:1, about 0.6:1 to about 3.4:1, about 0.7:1 to about 3.3:1, about 0.8:1 to about 3.2:1, about 0.9:1 to about 3.1:1, about 1:1 to about 3:1, about 1.1:1 to about 2.9:1, about 1.2:1 to about 2.8:1, about 1.3:1 to about 2.7:1, about 1.4:1 to about 2.6:1, about 1.5:1 to about 2.5:1, about 1.6:1 to about 2.4:1, about 1.7:1 to about 1.3:1, about 1.8:1 to about 2.2:1, or about 1.9:1 to about 2.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the ratio is about 2.1:1. In some embodiments, the ratio is about 2.2:1. In some embodiments, the ratio is about 2.3:1. In some embodiments, the ratio is about 2.4:1. In some embodiments, the ratio is about 2.5:1. In some embodiments, the ratio is about 2.6:1. In some embodiments, the ratio is about 2.7:1. In some embodiments, the ratio is about 2.8:1. In some embodiments, the ratio is about 2.9:1. In some embodiments, the ratio is about 3:1. In some embodiments, the ratio is about 3.1:1. In some embodiments, the ratio is about 3.2:1. In some embodiments, the ratio is about 3.3:1. In some embodiments, the ratio is about 3.4:1. In some embodiments, the ratio is about 3.5:1. In some embodiments, the ratio is about 3.6:1. In some embodiments, the ratio is about 3.7:1. In some embodiments, the ratio is about 3.8:1. In some embodiments, the ratio is about 3.9:1. In some embodiments, the ratio is about 4:1. In some embodiments, the crystalline form is a crystalline Form VII of the compound and fumaric, wherein the molar ratio of the compound to the fumaric is in any range from about 0.1:1 to about 4:1. In some embodiments, the crystalline form is a crystalline Form VII of the compound and fumaric, wherein the molar ratio of the compound to the fumaric is in a range from about 0.1:1 to about 4:1. In some embodiments, the crystalline form is a crystalline Form VII of the compound and fumaric, wherein the molar ratio of the compound to the fumaric is about 2:1.

In some embodiments, the crystalline form of the compound of Formula I and fumaric has a molar ratio of the compound to the fumaric in any range from about 0.1:1 to about 2:1. In some embodiments, the ratio is in a range of about 0.1:1 to about 2:1. In some embodiments, the ratio is in a range of about 0.1:1 to about 1.9:1, about 0.2:1 to about 1.8:1, about 0.3:1 to about 1.7:1, about 0.4:1 to about 1.6:1, about 0.5:1 to about 1.5:1, about 0.6:1 to about 1.4:1, about 0.7:1 to about 1.3:1, about 0.8:1 to about 1.2:1, or about 0.9:1 to about 1.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the crystalline form is a crystalline Form VIII of the compound and fumaric, wherein the molar ratio of the compound to the fumaric is in any range from about 0.1:1 to about 2:1. In some embodiments, the crystalline form is a crystalline Form VIII of the compound and fumaric, wherein the molar ratio of the compound to the fumaric is in a range from about 0.1:1 to about 2:1. In some embodiments, the crystalline form is a crystalline Form VIII of the compound and fumaric, wherein the molar ratio of the compound to the fumaric is about 1:1.

In some embodiments, the coformer is D-(−)-tartaric acid. In some embodiments, the crystalline form of the compound of Formula I and D-(−)-tartaric acid has a molar ratio of the compound to the D-(−)-tartaric acid is in any range from about 0.1:1 to about 2:1. In some embodiments, the ratio is in a range of about 0.1:1 to about 2:1. In some embodiments, the ratio is in a range of about 0.1:1 to about 1.9:1, about 0.2:1 to about 1.8:1, about 0.3:1 to about 1.7:1, about 0.4:1 to about 1.6:1, about 0.5:1 to about 1.5:1, about 0.6:1 to about 1.4:1, about 0.7:1 to about 1.3:1, about 0.8:1 to about 1.2:1, or about 0.9:1 to about 1.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the crystalline form is a crystalline Form IX of the compound and D-(−)-tartaric acid, wherein the molar ratio of the compound to the D-(−)-tartaric acid is in any range from about 0.1:1 to about 2:1. In some embodiments, the ratio is in a range of about 0.1:1 to about 2:1. In some embodiments, the crystalline form is a crystalline Form IX of the compound and D-(−)-tartaric acid, wherein the molar ratio of the compound to the D-(−)-tartaric acid is in any range from about 0.1:1 to about 2:1. In some embodiments, the crystalline form is a crystalline Form IX of the compound and D-(−)-tartaric acid, wherein the molar ratio of the compound to the D-(−)-tartaric acid is in any range from about 0.1:1 to about 2:1. In some embodiments, the crystalline form is a crystalline Form IX of the compound and D-(−)-tartaric acid, wherein the molar ratio of the compound to the D-(−)-tartaric acid is about 1:1.

In some embodiments, the coformer is hydrochloric acid. In some embodiments, the crystalline form is a crystalline Form X of the compound and hydrochloric acid.

In some embodiments, the coformer is salicylic acid. In some embodiments, the crystalline form of the compound of Formula I and salicylic has a molar ratio of the compound to the salicylic in any range from about 0.1:1 to about 4:1. In some embodiments, the ratio is in a range of about 0.1:1 to about 4:1. In some embodiments, the ratio is in a range of about 0.1:1 to about 3.9:1, about 0.2:1 to about 3.8:1, about 0.3:1 to about 3.7:1, about 0.4:1 to about 3.6:1, about 0.5:1 to about 3.5:1, about 0.6:1 to about 3.4:1, about 0.7:1 to about 3.3:1, about 0.8:1 to about 3.2:1, about 0.9:1 to about 3.1:1, about 1:1 to about 3:1, about 1.1:1 to about 2.9:1, about 1.2:1 to about 2.8:1, about 1.3:1 to about 2.7:1, about 1.4:1 to about 2.6:1, about 1.5:1 to about 2.5:1, about 1.6:1 to about 2.4:1, about 1.7:1 to about 1.3:1, about 1.8:1 to about 2.2:1, or about 1.9:1 to about 2.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the ratio is about 2.1:1. In some embodiments, the ratio is about 2.2:1. In some embodiments, the ratio is about 2.3:1. In some embodiments, the ratio is about 2.4:1. In some embodiments, the ratio is about 2.5:1. In some embodiments, the ratio is about 2.6:1. In some embodiments, the ratio is about 2.7:1. In some embodiments, the ratio is about 2.8:1. In some embodiments, the ratio is about 2.9:1. In some embodiments, the ratio is about 3:1. In some embodiments, the ratio is about 3.1:1. In some embodiments, the ratio is about 3.2:1. In some embodiments, the ratio is about 3.3:1. In some embodiments, the ratio is about 3.4:1. In some embodiments, the ratio is about 3.5:1. In some embodiments, the ratio is about 3.6:1. In some embodiments, the ratio is about 3.7:1. In some embodiments, the ratio is about 3.8:1. In some embodiments, the ratio is about 3.9:1. In some embodiments, the ratio is about 4:1. In some embodiments, the crystalline form is a crystalline Form XI of the compound and salicylic, wherein the molar ratio of the compound to the salicylic is in any range from about 0.1:1 to about 4:1. In some embodiments, the crystalline form is a crystalline Form XI of the compound and salicylic, wherein the molar ratio of the compound to the salicylic is in a range from about 0.1:1 to about 4:1. In some embodiments, the crystalline form is a crystalline Form XI of the compound and salicylic, wherein the molar ratio of the compound to the salicylic is about 2:1.

In some embodiments, the coformer is oxalic acid. In some embodiments, the crystalline form of the compound of Formula I and oxalic acid has a molar ratio of the compound to the oxalic acid in any range from about 0.1:1 to about 2:1. In some embodiments, the ratio is in a range of about 0.1:1 to about 2:1. In some embodiments, the ratio is in a range of about 0.1:1 to about 1.9:1, about 0.2:1 to about 1.8:1, about 0.3:1 to about 1.7:1, about 0.4:1 to about 1.6:1, about 0.5:1 to about 1.5:1, about 0.6:1 to about 1.4:1, about 0.7:1 to about 1.3:1, about 0.8:1 to about 1.2:1, or about 0.9:1 to about 1.1:1. In some embodiments, the ratio is about 0.1:1. In some embodiments, the ratio is about 0.2:1. In some embodiments, the ratio is about 0.3:1. In some embodiments, the ratio is about 0.4:1. In some embodiments, the ratio is about 0.5:1. In some embodiments, the ratio is about 0.6:1. In some embodiments, the ratio is about 0.7:1. In some embodiments, the ratio is about 0.8:1. In some embodiments, the ratio is about 0.9:1. In some embodiments, the ratio is about 1:1. In some embodiments, the ratio is about 1.1:1. In some embodiments, the ratio is about 1.2:1. In some embodiments, the ratio is about 1.3:1. In some embodiments, the ratio is about 1.4:1. In some embodiments, the ratio is about 1.5:1. In some embodiments, the ratio is about 1.6:1. In some embodiments, the ratio is about 1.7:1. In some embodiments, the ratio is about 1.8:1. In some embodiments, the ratio is about 1.9:1. In some embodiments, the ratio is about 2:1. In some embodiments, the crystalline form is a crystalline Form XII of the compound and oxalic acid, wherein the molar ratio of the compound to the oxalic acid is in any range from about 0.1:1 to about 2:1. In some embodiments, the crystalline form is a crystalline Form XII of the compound and oxalic acid, wherein the molar ratio of the compound to the oxalic acid is in a range from about 0.1:1 to about 2:1. In some embodiments, the crystalline form is a crystalline Form XII of the compound and oxalic acid, wherein the molar ratio of the compound to the oxalic acid is about 1:1.

In some embodiments, the coformer is 1-hydroxy-2-naphthoic acid. In some embodiments, the crystalline form is a crystalline Form XIII of the compound and 1-hydroxy-2-naphthoic acid.

### Pharmaceutical Compositions/Formulations

Embodiments described herein can be used in pharmaceutical compositions and can be formulated by standard techniques using one or more physiologically acceptable carriers or excipients. In some embodiments, the formulations may contain a buffer and/or a preservative. Any crystalline form as described or provided herein, such as the crystalline Forms I-XIII of the compound of Formula I, and their physiologically acceptable salts, anhydrates, hydrates and/or solvates, can be formulated for administration by any suitable route, including via inhalation, topically, nasally, orally, parenterally (for example, intravenously, intraperitoneally, intravesically or intrathecally) or rectally in a vehicle comprising one or more pharmaceutically acceptable carriers, the proportion of which is determined by the route of administration and standard biological practice. Other routes of administration are also described herein and can be used as well.

In some embodiments, a pharmaceutical composition comprising a crystalline form of the compound of Formula I, as described or provided for herein, is provided. In some embodiments, a pharmaceutical composition comprising the crystalline Form I, Form II, Form III, Form IV, Form V, Form VI, Form VII, Form VIII, Form IX, Form X, Form XI, Form, XII or Form XIII, as described and provided herein, is provided. In some embodiments, a pharmaceutical composition comprising the crystalline Form I, as described and provided herein, is provided. In some embodiments, a pharmaceutical composition comprising the crystalline Form II, as described and provided herein, is provided. In some embodiments, a pharmaceutical composition comprising the crystalline Form III, as described and provided herein, is provided. In some embodiments, a pharmaceutical composition comprising the crystalline Form VI, as described and provided herein, is provided. In some embodiments, a pharmaceutical composition comprising the crystalline Form V, as described and provided herein, is provided. In some embodiments, a pharmaceutical composition comprising the crystalline Form VI, as described and provided herein, is provided. In some embodiments, a pharmaceutical composition comprising the crystalline Form VII, as described and provided herein, is provided. In some embodiments, a pharmaceutical composition comprising the crystalline Form VIII comprising the compound of Formula I is provided. In some embodiments, a pharmaceutical composition comprising the crystalline Form IX, as described and provided herein, is provided. In some embodiments, a pharmaceutical composition comprising the crystalline Form X, as described and provided herein, is provided. In some embodiments, a pharmaceutical composition comprising the crystalline Form XI, as described and provided herein, is provided. In some embodiments, a pharmaceutical composition comprising the crystalline Form XII, as described and provided herein, is provided. In some embodiments, a pharmaceutical composition comprising the crystalline Form XIII, as described and provided herein, is provided.

In some embodiments, the pharmaceutical composition comprising a crystalline form of Forms I-XIII, as described or provided herein, further comprising a pharmaceutically acceptable excipient. In some embodiments, the pharmaceutical composition comprises the crystalline Form I. In some embodiments, the pharmaceutical composition comprises the crystalline Form II. In some embodiments, the pharmaceutical composition comprises the crystalline Form III. In some embodiments, the pharmaceutical composition comprises the crystalline Form VI. In some embodiments, the pharmaceutical composition comprises the crystalline Form V. In some embodiments, the pharmaceutical composition comprises the crystalline Form VI. In some embodiments, the pharmaceutical composition comprises the crystalline Form VII. In some embodiments, the pharmaceutical composition comprises the crystalline Form VIII. In some embodiments, the pharmaceutical composition comprises the crystalline Form IX. In some embodiments, the pharmaceutical composition comprises the crystalline Form X. In some embodiments, the pharmaceutical composition comprises the crystalline Form XI. In some embodiments, the pharmaceutical composition comprises the crystalline Form XII. In some embodiments, the pharmaceutical composition comprises the crystalline Form XIII.

In some embodiments, pharmaceutical compositions are provided comprising effective amounts of any crystalline form as described or provided herein, such as the crystalline Forms I-XIII of the compound of Formula I, with, for example, pharmaceutically acceptable diluents, preservatives, solubilizers, emulsifiers, adjuvants and/or other carriers. Such compositions are known to one skilled in the art and the compositions can be formulated using standard techniques. For example, diluents of various buffer content such as, but not limited to, TRIS or other amines, carbonates, phosphates, amino acids, for example, glycinamide hydrochloride (especially in the physiological pH range), N-glycylglycine, sodium or potassium phosphate (dibasic, tribasic), etc. or TRIS-HCl or acetate), pH and ionic strength; additives such as detergents and solubilizing agents (e.g., surfactants such as Pluronics, Tween 20, Tween 80 (Polysorbate 80), Cremophor, polyols such as polyethylene glycol, propylene glycol, etc.), anti-oxidants (e.g., ascorbic acid, sodium metabisulfite), preservatives (e.g., Thimersol, benzyl alcohol, parabens, etc.) and bulking substances (e.g., sugars such as sucrose, lactose, mannitol, polymers such as polyvinylpyrrolidones or dextran, etc.); and/or incorporation of the material into particulate preparations of polymeric compounds such as polylactic acid, polyglycolic acid, etc. or into liposomes may be used. Hyaluronic acid may also be used. Such compositions can be employed to influence the physical state, stability, rate of in vivo release, and rate of in vivo clearance of a composition comprising the crystalline of any one of Forms I-XIII or the crystalline Form VIII as described herein. See, e.g., Remington's Pharmaceutical Sciences, 18th Ed. (1990, Mack Publishing Co., Easton, Pa. 18042) pages 1435-1712 which are herein incorporated by reference. Where a buffer is to be included in the formulations, the buffer can be, for example, but not limited to, sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminonethan, or mixtures thereof. Each buffer can be used independently or in combination with another buffer. In some embodiments, the buffer is glycylglycine, sodium dihydrogen phosphate, disodium hydrogen phosphate, sodium phosphate or mixtures thereof.

Where a pharmaceutically acceptable preservative is to be included in the formulations, the preservative can be, but is not limited to, phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof. In some embodiments, the preservative is phenol and/or m-cresol.

In some embodiments, the preservative is present in a concentration from about 0.1 mg/ml to about 100 mg/ml, more preferably in a concentration from about 0.1 mg/ml to about 50 mg/ml, about 0.1 mg/ml to about 25 mg/ml. In some embodiments, the preservative is present in a concentration from about 0.1 mg/ml to about 10 mg/ml.

The use of a preservative in pharmaceutical compositions is well known to the skilled person. For convenience, reference is made to Remington: The Science and Practice of Pharmacy, 19th edition, 1995.

In some embodiments, the formulation may further comprise a chelating agent where the chelating agent may be salts of ethlenediaminetetraacetic acid (EDTA), citric acid, and aspartic acid, and mixtures thereof.

In some embodiments, the chelating agent is present in a concentration from 0.1 mg/ml to 10 mg/ml, particularly in a concentration from 0.1 mg/ml to 5 mg/ml. In some embodiments, the chelating agent is present in a concentration from 0.1 mg/ml to 2 mg/ml. In some embodiments, the chelating agent is present in a concentration from 2 mg/ml to 5 mg/ml.

The use of a chelating agent in pharmaceutical compositions is well known to the skilled person. For convenience, reference is made to Remington: The Science and Practice of Pharmacy, 19th edition, 1995.

In some embodiments, the formulation may further comprise a stabilizer selected from the group of high molecular weight polymers or low molecular compounds where such stabilizers include, but are not limited to, polyethylene glycol (e.g., PEG 3350), polyvinylalcohol (PVA), polyvinylpyrrolidone, carboxymethylcellulose, different salts (e.g., sodium chloride), L-glycine, L-histidine, imidazole, arginine, lysine, isoleucine, aspartic acid, tryptophan, threonine and mixtures thereof. In some embodiments, the stabilizer is L-histidine, imidazole, arginine, or any combination thereof.

In some embodiments, the high molecular weight polymer is present in a concentration from 0.1 mg/ml to 100 mg/ml, in a concentration from 0.1 mg/ml to 50 mg/ml. In some embodiments, the high molecular weight polymer is present in a concentration from 0.1 mg/ml to 5 mg/ml. In some embodiments, the high molecular weight polymer is present in a concentration from 5 mg/ml to 10 mg/ml. In some embodiments, the high molecular weight polymer is present in a concentration from 10 mg/ml to 20 mg/ml. In some embodiments, the high molecular weight polymer is present in a concentration from 20 mg/ml to 30 mg/ml. In some embodiments, the high molecular weight polymer is present in a concentration from 30 mg/ml to 50 mg/ml.

In some embodiments, the low molecular weight polymer is present in a concentration from 0.1 mg/ml to 100 mg/ml. In some embodiments, the low molecular weight polymer is present in a concentration from 0.1 mg/mll to 50 mg/ml. In some embodiments, the low molecular weight polymer is present in a concentration from 0.1 mg/ml to 5 mg/ml. In some embodiments, the low molecular weight polymer compound is present in a concentration from 5 mg/ml to 10 mg/ml. In some embodiments, the low molecular weight polymer is present in a concentration from 10 mg/ml to 20 mg/ml. In some embodiments, the low molecular weight polymer is present in a concentration from 20 mg/ml to 30 mg/ml. In some embodiments, the low molecular weight polymer is present in a concentration from 30 mg/ml to 50 mg/ml. In some embodiments, the low molecular weight polymer is present in a concentration from 50 mg/ml to 60 mg/ml. In some embodiments, the low molecular weight polymer is present in a concentration from 60 mg/Ml to 80 mg/ml. In some embodiments, the low molecular weight polymer is present in a concentration from 80 mg/ml to 100 mg/ml.

The use of a stabilizer in pharmaceutical compositions is well known to the skilled person. For convenience, reference is made to Remington: The Science and Practice of Pharmacy, 19th edition, 1995.

In some embodiments, the formulation may comprise a surfactant where a surfactant can be a detergent, ethoxylated castor oil, polyglycolyzed glycerides, acetylated monoglycerides, sorbitan fatty acid esters, poloxamers, such as 188 and 407, polyoxyethylene sorbitan fatty acid esters, polyoxyethylene derivatives such as alkylated and alkoxylated derivatives (tweens, e.g., Tween-20, or Tween-80), monoglycerides or ethoxylated derivatives thereof, diglycerides or polyoxyethylene derivatives thereof, glycerol, cholic acid or derivatives thereof, lecithins, alcohols and phospholipids, glycerophospholipids (lecithins, kephalins, phosphatidyl serine), glyceroglycolipids (galactopyransoide), sphingophospholipids (sphingomyelin), and sphingoglycolipids (ceramides, gangliosides), DSS (docusate sodium, docusate calcium, docusate potassium, SDS (sodium dodecyl sulfate or sodium lauryl sulfate), dipalmitoyl phosphatidic acid, sodium caprylate, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g., 1-acyl-sn-glycero-3-phosphate esters of ethanolamine, choline, serine or threonine), alkyl, alkoxyl (alkyl ester), alkoxy (alkyl ether)-derivatives of lysophosphatidyl and phosphatidylcholines, e.g., lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, zwitterionic surfactants (e.g., N-alkyl-N,N-dimethylammonio-1-propanesulfonates, 3-cholamido-1-propyldimethylammonio-1-propanesulfonate, dodecylphosphocholine, myristoyl lysophosphatidylcholine, hen egg lysolecithin), cationic surfactants (quarternary ammonium bases) (e.g., cetyl-trimethylammonium bromide, cetylpyridinium chloride), non-ionic surfactants, polyethyleneoxide/polypropyleneoxide block copolymers (Pluronics/Tetronics, Triton X-100, Dodecyl β-D-glucopyranoside) or polymeric surfactants (Tween-40, Tween-80, Briji-35), fusidic acid derivatives—(e.g., sodium tauro-dihydrofusidate etc.), long-chain fatty acids and salts thereof C6-C12 (e.g., oleic acid and caprylic acid), acylcarnitines and derivatives, N_{α}-acylated derivatives of lysine, arginine or histidine, or side-chain acylated derivatives of lysine or arginine, N_{α}-acylated derivatives of dipeptide comprising any combination of lysine, arginine or histidine and a neutral or acidic amino acid, N_{α}-acylated derivative of a tripeptide comprising any combination of a neutral amino acid and two charged amino acids, imidazoline derivatives, or any mixture thereof.

The use of a surfactant in pharmaceutical compositions is well-known to the skilled person. For convenience, reference is made to Remington: The Science and Practice of Pharmacy, 19th edition, 1995.

The formulations may also comprise a pharmaceutically acceptable sweetener. In some embodiments, the sweetener comprises at least one intense sweetener such as, but not limited to, saccharin, sodium or calcium saccharin, aspartame, acesulfame potassium, sodium cyclamate, alitame, a dihydrochalcone sweetener, monellin, stevioside or sucralose (4,1′,6′-trichloro-4,1′,6′-trideoxygalactosucrose), preferably saccharin, sodium or calcium saccharin, and optionally a bulk sweetener such as sorbitol, mannitol, fructose, sucrose, maltose, isomalt, glucose, hydrogenated glucose syrup, xylitol, caramel or honey.

Intense sweeteners are conveniently employed in low concentrations. For example, in the case of sodium saccharin, the concentration may range from 0.04% to 0.1% (w/v) based on the total volume of the final formulation, or from about 0.06% in the low-dosage formulations and about 0.08% in the high-dosage ones. The bulk sweetener can effectively be used in larger quantities ranging from about 10% to about 35% or from about 10% to 15% (w/v).

The formulations may be prepared by conventional techniques, for example, as described in Remington's Pharmaceutical Sciences, 1985 or in Remington: The Science and Practice of Pharmacy, 19th edition, 1995, where such conventional techniques of the pharmaceutical industry involve dissolving and mixing the ingredients as appropriate to give the desired end product.

Administration of the compound or the formulations described herein may be carried out using any method known in the art. For example, the administration may be transdermal, parenteral, intravenous, intra-arterial, subcutaneous, intramuscular, intracranial, intraorbital, ophthalmic, intraventricular, intracapsular, intraspinal, intracisternal, intraperitoneal, intracerebroventricular, intrathecal, intranasal, aerosol, by suppositories, inhalation, or by oral administration. In some embodiments, the compound or formulation is administered intravenously or by injection.

For oral administration, the crystalline of any one of the crystalline Forms I-XIII or a therapeutically acceptable salt thereof can be formulated in unit dosage forms such as gel caps, caplets, granules, lozenges, bulk powders, capsules, or tablets. The tablets or capsules may be prepared by conventional means with pharmaceutically acceptable excipients, including binding agents, for example, pregelatinized maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulfate. Tablets can be coated by methods well known in the art.

Liquid preparations for oral administration can take the form of, for example, solutions, syrups, or suspensions, or they can be presented as a dry product for constitution with water or other suitable vehicles before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For topical administration, any of the crystalline or crystalline forms provided or described herein, such as the crystalline Forms I-XIII of the compound of Formula I, can be formulated in a pharmaceutically acceptable vehicle containing 0.1 to 10 percent, preferably 0.5 to 5 percent, of the active compound(s). Such formulations can be in the form of a cream, lotion, sublingual tablet, aerosols and/or emulsions and can be included in a transdermal or buccal patch of the matrix or reservoir type as are conventional in the art for this purpose.

For parenteral administration, any crystalline form as described or provided herein, such as the crystalline Forms I-XIII of the compound of Formula I, can be administered by either intravenous, subcutaneous, or intramuscular injection, in compositions with pharmaceutically acceptable vehicles or carriers. The crystalline form as described and provided herein can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles and can contain formulatory agents, for example, suspending, stabilizing, and/or dispersing agents. Additionally, the compound can be precipitated and stored in an ampule or other container and then dissolved in a solution prior to being administered to a subject.

For administration by injection, the compound can be used in solution and, for example, in a sterile aqueous vehicle which may also contain other solutes such as buffers or preservatives as well as sufficient quantities of pharmaceutically acceptable salts or of glucose to make the solution isotonic. In some embodiments, the pharmaceutical compositions may be formulated with a pharmaceutically acceptable carrier to provide sterile solutions or suspensions for injectable administration. In particular, injectables can be prepared in conventional forms, either as liquid solutions or suspensions, solid forms suitable for solution or suspensions in liquid prior to injection or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH-buffering agents, and the like. If desired, absorption enhancing preparations (e.g., liposomes) may be utilized. Suitable pharmaceutical carriers are described in “Remington's Pharmaceutical Sciences” by E. W. Martin.

For administration by inhalation, the compound may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch. For intranasal administration, the compound may be used, for example, as a liquid spray, as a powder or in the form of drops.

The compound can also be formulated in rectal compositions, for example, suppositories or retention enemas, for example, containing conventional suppository bases, for example, cocoa butter or other glycerides.

Furthermore, the compound can be formulated as a depot preparation. Such long-acting formulations can be administered by implantation (for example, subcutaneously or intramuscularly) or by intramuscular injection. Thus, for example, the compound can be formulated with suitable polymeric or hydrophobic materials (for example, as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack can also contain individual vials or other containers. The pack or dispenser device can be accompanied by instructions for administration.

### Dosages

Any crystalline form as described or provided herein, such as the crystalline Forms I-XIII of the compound of Formula I, may be administered to a patient at therapeutically effective doses to prevent, treat, or control diseases and disorders mediated, in whole or in part, by a GPCR-ligand interaction described herein. Pharmaceutical compositions comprising any crystalline form as described or provided herein, such as the crystalline Forms I-XIII of the compound of Formula I, may be administered to a patient in an amount sufficient to elicit an effective protective or therapeutic response in the patient. The dose will be determined by the efficacy of the particular compound employed and the condition of the subject, as well as the bodyweight or surface area of the area to be treated. The size of the dose also will be determined by the existence, nature, and extent of any adverse effects that accompany the administration of a particular compound or vector in a particular subject.

The amount and frequency of administration of the compound comprising any crystalline form as described or provided herein, such as the crystalline Forms I-XIII of the compound of Formula I, prepared according to a method described herein and/or the pharmaceutically acceptable salts thereof can be regulated according to the judgment of the attending clinician considering such factors as age, condition and size of the patient as well as the severity of the symptoms being treated. An ordinarily skilled physician or veterinarian can readily determine and prescribe the effective amount of the drug required to prevent, counter or arrest the progress of the condition. In general, it is contemplated that an effective amount would be from 0.001 mg/kg to 10 mg/kg body weight, and in particular from 0.01 mg/kg to 1 mg/kg body weight. More specifically, it is contemplated that an effective amount would be to continuously infuse by intravenous administration from 0.01 micrograms/kg body weight/min to 100 micrograms/kg body weight/min for a period of 12 hours to 14 days. It may be appropriate to administer the required dose as two, three, four or more sub-doses at appropriate intervals throughout the day. Sub-doses may be formulated as unit dosage forms, for example, containing 0.01 to 500 mg, and in particular 0.1 mg to 200 mg of active ingredient per unit dosage form.

In some embodiments, the pharmaceutical preparation is in a unit dosage form. In such form, the preparation is subdivided into suitably sized unit doses containing appropriate quantities of the active component, e.g., an effective amount to achieve the desired purpose. The quantity of active compound in a unit dose of preparation may be varied or adjusted from about 0.01 mg to about 1000 mg, from about 0.01 mg to about 750 mg, from about 0.01 mg to about 500 mg, or from about 0.01 mg to about 250 mug, according to the particular application. The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

### Medical Use

In some embodiments, the method comprises administering to a subject or a subject in need thereof an amount, such as a therapeutically effective amount of a crystalline form as provided herein such as the crystalline Forms I-XIII of the compound of Formula I, or a pharmaceutically acceptable salt or solvate thereof. Therapeutically effective amount of the crystalline form may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular crystalline form as provided herein chosen, the dosing regimen to be followed, whether it is administered in combination with other compounds, the timing of administration, the tissue to which it is administered, and the physical delivery system in which it is carried.

As used herein, the term “IC50” refers to the half-maximal inhibitory concentration of an inhibitor in inhibiting biological or biochemical function. This quantitative measure indicates how much of a particular inhibitor is needed to inhibit a given biological process (or component of a process, i.e., an enzyme, cell, cell receptor or microorganism) by half. In other words, it is the half-maximal (50%) inhibitory concentration (IC) of a substance (50% IC, or IC50). EC50 refers to the plasma concentration required for obtaining 50%> of a maximum effect in vivo.

In some embodiments, the subject methods utilize a CDK inhibitor with an IC50 value of about or less than a predetermined value, as ascertained in an in vitro assay. In some embodiments, the CDK inhibitor inhibits CDK with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above). In some embodiments, the CDK enzyme is CDK9.

In some embodiments, the subject method of inhibiting CDK enzyme comprises contacting the CDK enzyme with an effective amount of a crystalline form as provided herein or a pharmaceutically acceptable salt thereof as described herein. In some embodiments, the CDK enzyme is CDK9.

In some embodiments, the CDK inhibitor selectively inhibits CDK with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two, or three other CDKs. In some embodiments, the CDK inhibitor is a CDK9 inhibitor.

In some embodiments, the CDK inhibitor selectively inhibits CDK with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM (or in the range defined by and including any two numbers above), and said IC5 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other CDKs. In some embodiments, the CDK inhibitor is a CDK9 inhibitor.

In some embodiments, compounds described herein are in use for inhibiting a CDK enzyme in a subject, wherein the use comprises administering to the subject an effective amount of one or more crystalline forms as provided herein, such as the crystalline Forms I XIII of the crystalline form of Formula I, pharmaceutically acceptable salts, solvates, pharmaceutical compositions, or prodrugs thereof.

In some embodiments, provided herein are pharmaceutical compositions as described herein are in use for inhibiting a CDK enzyme in a subject, wherein the use comprises administering to the subject an effective amount of one or more pharmaceutical compositions as provided herein

In some embodiments, provided herein are uses of the crystalline Forms I-XIII of the compound of Formula I as provided herein in the manufacture of a formulation inhibiting a CDK enzyme in a subject, wherein the use comprises administering to the subject an effective amount of one or more crystalline forms as provided herein, such as the crystalline Forms I XIII of the compound of Formula I, pharmaceutically acceptable salts, solvates, pharmaceutical compositions, or prodrugs thereof.

In some embodiments, provided herein are uses of a pharmaceutical composition as described herein for inhibiting a CDK enzyme in a subject, wherein the use comprises administering to the subject an effective amount of one or more pharmaceutical compositions as described herein. In some embodiments, the CDK enzyme is CDK9.

The subject methods are useful for treating a disease or disorder condition associated with CDK. Any disease or disorder condition that results directly or indirectly from an abnormal activity or expression level of CDK can be an intended disease or disorder condition. In some embodiments, the said method for treating disease or disorder condition associated with CDK in a subject or a subject in need thereof comprises administering to the subject, a crystalline form as provided herein, such as the crystalline Forms I-XIII of the compound of Formula I, or a pharmaceutically acceptable salt thereof as described herein.

Different disease or disorder conditions associated with CDK have been reported. CDK has been implicated, for example, auto-immune diseases, neurodegeneration (such as Parkinson's disease, Alzheimer's disease and ischaemia), inflammatory diseases, viral infections and cancer such as, for example, colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinona, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, 1-lead and neck cancer, Heart cancer, Hemoglobinopathies such as b-thalassemia and sickle cell disease (SCD), Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene onChromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroecto dermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, said method is for treating a disease selected from the group consisting of tumor angiogenesis, chronic inflammatory diseases such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In other embodiments, said method is for treating a disease selected from breast cancer, lung cancer, pancreatic cancer, prostate cancer, colon cancer, ovarian cancer, uterine cancer, or cervical cancer. In some embodiments, the said method comprises administering to a subject or a subject in need thereof, a crystalline form as provided herein or a pharmaceutically acceptable salt thereof as described herein.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS) or epidermoid cancer.

In some embodiments, provided herein are crystalline forms as provided herein, such as the crystalline Forms I-XIII of the compound of Formula I, in use for treating a disease or disorder associated with aberrant CDK activity in a subject or a subject in need thereof, wherein the use comprises administering to the subject an effective amount of one or more crystalline forms as provided herein, such as the crystalline Forms I-XIII of the compound of Formula I, pharmaceutically acceptable salts, solvates, pharmaceutical compositions, or prodrugs thereof.

In some embodiments, provided herein are pharmaceutical compositions as described herein in use for treating a disease or disorder associated with aberrant CDK activity in a subject or a subject in need thereof, wherein the use comprises administering to the subject an effective amount of one or more pharmaceutical compositions as described herein.

In some embodiments, provided herein are uses of crystalline forms as provided herein, such as the crystalline Forms I-XIII of the compound of Formula I, in the manufacture of a formulation treating a disease or disorder associated with aberrant CDK activity in a subject or a subject in need thereof, wherein the use comprises administering to the subject an effective amount of one or more crystalline forms as provided herein, such as the crystalline Forms I-XIII of the compound of Formula I, pharmaceutically acceptable salts, solvates, pharmaceutical compositions, or prodrugs thereof.

In some embodiments, use of pharmaceutical compositions as described herein for treating a disease or disorder associated with aberrant CDK activity in a subject or a subject in need thereof, wherein the use comprises administering to the subject an effective amount of one or more pharmaceutical composition as described herein. In some embodiments, the disease or disorder associated with aberrant CDK activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

In some embodiments, provided herein are crystalline forms as provided herein, such as the crystalline Forms I-XIII of the compound of Formula I, in use for treating cancer in a subject or a subject in need thereof, wherein the use comprises administering to the subject an effective amount of one or more crystalline forms as provided herein, such as the crystalline Forms I-XIII of the compound of Formula I, pharmaceutically acceptable salts, solvates, pharmaceutical compositions, or prodrugs thereof.

In some embodiments, provided herein are pharmaceutical compositions as described herein in use for treating cancer in a subject or a subject in need thereof, wherein the use comprises administering to the subject an effective amount of one or more pharmaceutical compositions as described herein.

In some embodiments, provided herein are uses of crystalline forms as provided herein, such as the crystalline Forms I-XIII of the compound of Formula I, in the manufacture of a formulation treating cancer in a subject or a subject in need thereof, wherein the use comprises administering to the subject an effective amount of one or more crystalline forms as provided herein, such as the crystalline Forms I-XIII of the compound of Formula I, pharmaceutically acceptable salts, solvates, pharmaceutical compositions, or prodrugs thereof.

In some embodiments, provided herein are uses of pharmaceutical compositions as described herein for treating cancer in a subject or a subject in need thereof, wherein the use comprises administering to the subject an effective amount of one or more pharmaceutical compositions as described herein. In some embodiments, the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

In some embodiments, provided herein are methods of inducing apoptosis in a cancer or tumor cell in a subject or a subject in need thereof. In some embodiments, the methods comprise contacting the cancer or tumor cell or administering to the subject with an effective amount of a crystalline form as provided herein, such as the crystalline Forms I-XIII of the compound of Formula I, or a pharmaceutically acceptable salt or solvate thereof, or a pharmaceutical composition as described herein. In some embodiments, provided herein are methods of inducing apoptosis in a cancer or tumor cell in a subject or a subject in need thereof. In some embodiments, the methods comprise contacting the cancer or tumor cell with or administering to the subject an effective amount of a crystalline form as provided herein crystalline forms as provided herein, such as the crystalline Forms I-XIII of the compound of Formula I, or a pharmaceutically acceptable salt or solvate thereof. In some embodiments, provided herein are methods of inducing apoptosis in a cancer or tumor cell in a subject or a subject in need thereof, the methods comprising contacting the cancer or tumor cell with, or administering to the subject, an effective amount of a pharmaceutical composition as described herein. In some embodiments, the cancer or tumor has high levels of MYC amplification and overexpression. In some embodiments, the cancer cell or tumor is characterized as malignant. In some embodiments, the cancer cell or tumor is characterized as a hematological cancer cell or tumor. In some embodiments, the hematological cancer is a B-Cell Acute Lymphoblastic Leukemia (B-ALL), T-Cell Acute Lymphoblastic Leukemia (T-ALL), Acute Myeloid Leukemia (AML), non-Hodgkin's lymphoma, sarcoma, prostate, adenoid cystic carcinoma (ACC), or non-small cell lung cancer (NSCLC). In some embodiments, the tumor cell is from a solid tumor. In some embodiments, the solid tumor is pancreatic carcinoma, gastric and gastroesophageal carcinomas, NSCLC, or sarcoma. In some embodiments, methods described herein further comprise contacting the tumor cell with an additional therapeutic, such as a targeted therapy. In some embodiments, the targeted therapy is as described herein. In some embodiments, the targeted therapy is a BCL2 inhibitor. In some embodiments, the BCL2 inhibitor is venetoclax. In some embodiments, the method achieves a complete response, such as a complete tumor regression.

In some embodiments, provided herein are crystalline forms as provided herein, such as the crystalline Forms I-XIII of the compound of Formula I, in use for inducing apoptosis in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are uses of crystalline forms as provided herein, such as the crystalline Forms I-XIII of the compound of Formula I, for inducing apoptosis in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are uses of crystalline forms as provided herein, such as the crystalline Forms I-XIII of the compound of Formula I, in the manufacture of a formulation for inducing apoptosis in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are pharmaceutical compositions as described herein in use for inducing apoptosis in a cancer or tumor cell in a subject or a subject in need thereof comprising contacting the cancer or tumor cell with, or administering to the subject, an effective amount of the pharmaceutical composition as described herein.

In some embodiments, provided herein are uses of pharmaceutical compositions as described herein for inducing apoptosis in a cancer or tumor cell in a subject or a subject in need thereof comprising contacting the cancer or tumor cell with, or administering to the subject, an effective amount of the pharmaceutical composition as described herein.

In some embodiments, provided herein are uses of pharmaceutical compositions as described herein in the manufacture of a formulation for inducing apoptosis in a cancer or tumor cell in a subject or a subject in need thereof comprising contacting the cancer or tumor cell with, or administering to the subject, an effective amount of the pharmaceutical composition as described herein.

In some embodiments, provided herein are methods of inhibiting phosphorylation of Ser2RNAP2 in a cancer or tumor cell in a subject or a subject in need thereof comprising contacting the cancer or tumor cell with, or administering to the subject, an effective amount of a crystalline form as provided herein, such as the crystalline Forms I-XIII of the compound of Formula I, or a pharmaceutically acceptable salt or solvate thereof, or a pharmaceutical composition as described herein. In some embodiments, provided herein are methods of inhibiting phosphorylation of Ser2RNAP2 in a cancer or tumor cell in a subject or a subject in need thereof comprising contacting the cancer or tumor cell with, or administering to the subject, an effective amount of a crystalline form as provided herein, such as the crystalline Forms I-XIII of the compound of Formula I, or a pharmaceutically acceptable salt or solvate thereof. In some embodiments, provided herein are methods of inhibiting phosphorylation of Ser2RNAP2 in a cancer or tumor cell in a subject or a subject in need thereof comprising contacting the cancer or tumor cell with, or administering to the subject, an effective amount of a pharmaceutical composition as described herein. In some embodiments, the phosphorylation is inhibited by at least, or about, 10%, 20%, 30%, 40%, 50%, 60%, 70%, 80%, 90%, or 100%. In some embodiments, the cancer or tumor has high levels of MYC amplification and overexpression. In some embodiments, the cancer cell is malignant. In some embodiments, the cancer cell is a hematological cancer cell. In some embodiments, the hematological cancer is a B-Cell Acute Lymphoblastic Leukemia (B-ALL), T-Cell Acute Lymphoblastic Leukemia (T-ALL), Acute Myeloid Leukemia (AML), non-Hodgkin's lymphoma, sarcoma, prostate, adenoid cystic carcinoma (ACC), or non-small cell lung cancer (NSCLC). In some embodiments, the tumor cell is from a solid tumor. In some embodiments, the solid tumor is pancreatic carcinoma, gastric and gastroesophageal carcinomas, NSCLC, or sarcoma. In some embodiments, methods described herein further comprise contacting the tumor cell with a targeted therapy. In some embodiments, the targeted therapy is as described herein. In some embodiments, the targeted therapy is a BCL2 inhibitor. In some embodiments, the BCL2 inhibitor is venetoclax. In some embodiments, the method achieves a complete tumor regression.

In some embodiments, provided herein are crystalline forms as provided herein, such as the crystalline Forms I XIII of the compound of Formula I, in use for inhibiting phosphorylation of Ser2RNAP2 in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are uses of the crystalline forms as provided herein, such as the crystalline Forms I-XIII of the compound of Formula I, for inhibiting phosphorylation of Ser2RNAP2 in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are uses of crystalline forms as provided herein, such as the crystalline Forms I-XIII of the compound of Formula I, in the manufacture of a formulation for inhibiting phosphorylation of Ser2RNAP2 in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are pharmaceutical compositions in use for inhibiting phosphorylation of Ser2RNAP2 in a cancer or tumor cell in a subject or a subject in need thereof comprising contacting the cancer or tumor cell with, or administering to the subject, an effective amount of the pharmaceutical composition.

In some embodiments, provided herein are uses of pharmaceutical compositions for inhibiting phosphorylation of Ser2RNAP2 in a cancer or tumor cell in a subject or a subject in need thereof comprising contacting the cancer or tumor cell with, or administering to the subject, an effective amount of the pharmaceutical composition.

In some embodiments, provided herein are uses of pharmaceutical compositions in the manufacture of a formulation for inhibiting phosphorylation of Ser2RNAP2 in a cancer or tumor cell in a subject or a subject in need thereof comprising contacting the cancer or tumor cell with, or administering to the subject, an effective amount of the pharmaceutical composition.

In some embodiments, provided herein are methods of reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of a crystalline form as provided herein, such as the crystalline Forms I-XIII of the compound of Formula I, or a pharmaceutically acceptable salt or solvate thereof, or a pharmaceutical composition as described herein. In some embodiments, provided herein are methods of reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of a crystalline form as provided herein, or a pharmaceutically acceptable salt or solvate thereof. In some embodiments, provided herein are methods of reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of a pharmaceutical composition as described herein. In some embodiments, the cancer or tumor has high levels of MYC amplification and overexpression. In some embodiments, the cancer cell is malignant. In some embodiments, the cancer cell is a hematological cancer cell. In some embodiments, the hematological cancer is a B-Cell Acute Lymphoblastic Leukemia (B-ALL), T-Cell Acute Lymphoblastic Leukemia (T-ALL), Acute Myeloid Leukemia (AML), non-Hodgkin's lymphoma, sarcoma, prostate, adenoid cystic carcinoma (ACC), or non-small cell lung cancer (NSCLC) In some embodiments, the tumor cell is from a solid tumor. In some embodiments, the solid tumor is pancreatic carcinoma, gastric and gastroesophageal carcinomas, NSCLC, or sarcoma. In some embodiments, methods described herein further comprise contacting the tumor cell with a targeted therapy. In some embodiments, the targeted therapy is as described herein. In some embodiments, the targeted therapy is a BCL2 inhibitor. In some embodiments, the BCL2 inhibitor is venetoclax. In some embodiments, the method achieves a complete tumor regression.

In some embodiments, provided herein are crystalline forms as provided herein, such as the crystalline Forms I-XIII of the compound of Formula I, in use for reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of the crystalline form, or a pharmaceutically acceptable salt or solvate thereof. In some embodiments, the level of induced myeloid leukemia cell differentiation protein Mcl-1 (MCL1) is reduced by at least, or about, 10%, 20%, 30%, 40%, 50%, 60%, 70%, 80%, 90%, or 1000%.

In some embodiments, provided herein are uses of crystalline forms as provided herein, such as the crystalline Forms I-XIII of the compound of Formula I, for reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are uses of crystalline forms as provided herein, such as the crystalline Forms I-XIII of the compound of Formula I, in the manufacture of a formulation for reducing the level of induced myeloid leukemia cell differentiation protein Mcl-1 (MCL) in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are pharmaceutical compositions in use for reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of the pharmaceutical composition.

In some embodiments, provided herein are uses of pharmaceutical compositions for reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of the pharmaceutical composition.

In some embodiments, provided herein are uses of pharmaceutical compositions in the manufacture of a formulation for reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of the pharmaceutical composition.

In some embodiments, provided herein are methods of reducing the level of MYC protein in a cancer or tumor cell in a subject or a subject in need thereof comprising contacting the cancer or tumor cell with, or administering to the subject, an effective amount of a crystalline form as provided herein, such as the crystalline Forms I-XIII of the compound of Formula I, or a pharmaceutically acceptable salt or solvate thereof, or a pharmaceutical composition as described herein. In some embodiments, provided herein are methods of reducing the level of MYC protein in a cancer or tumor cell in a subject or a subject in need thereof comprising contacting the cancer or tumor cell with, or administering to the subject, an effective amount of a crystalline form as provided herein, or a pharmaceutically acceptable salt or solvate thereof. In some embodiments, provided herein are methods of reducing the level of MYC protein in a cancer or tumor cell in a subject or a subject in need thereof comprising contacting the cancer or tumor cell with, or administering to the subject, an effective amount of a pharmaceutical composition as described herein. In some embodiments, the cancer or tumor has high levels of MYC amplification and overexpression. In some embodiments, the cancer cell is malignant. In some embodiments, the cancer cell is a hematological cancer cell. In some embodiments, the hematological cancer is a B-Cell Acute Lymphoblastic Leukemia (B-ALL), T-Cell Acute Lymphoblastic Leukemia (T-ALL), Acute Myeloid Leukemia (AML), non-Hodgkin's lymphoma, sarcoma, prostate, adenoid cystic carcinoma (ACC), or non-small cell lung cancer (NSCLC). In some embodiments, the tumor cell is from a solid tumor. In some embodiments, the solid tumor is pancreatic carcinoma, gastric and gastroesophageal carcinomas, NSCLC, or sarcoma. In some embodiments, methods described herein further comprise contacting the tumor cell with a targeted therapy. In some embodiments, the targeted therapy is as described herein. In some embodiments, the targeted therapy is a BCL2 inhibitor. In some embodiments, the BCL2 inhibitor is venetoclax. In some embodiments, the method achieves a complete tumor regression. In some embodiments, the tumor is regressed by at least, or about, 10%, 20%, 30%, 40%, 50%, 60%, 70%, 80%, 90%, or 100%.

In some embodiments, provided herein are crystalline forms as provided herein, such as the crystalline Forms I-XIII of the compound of Formula I, in use for reducing the level of MYC protein in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are uses of crystalline forms as provided herein, such as the crystalline Forms I-XIII of the compound of Formula I, for reducing the level of MYC protein in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are uses of crystalline forms as provided herein, such as the crystalline Forms I-XIII of the compound of Formula I, in the manufacture of a formulation for reducing the level of MYC protein in a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are pharmaceutical compositions in use for reducing the level of MYC protein in a cancer or tumor cell in a subject or a subject in need thereof comprising contacting the cancer or tumor cell with, or administering to the subject, an effective amount of the pharmaceutical composition.

In some embodiments, provided herein are uses of pharmaceutical compositions for reducing the level of MYC protein in a cancer or tumor cell in a subject or a subject in need thereof comprising contacting the cancer or tumor cell with, or administering to the subject, an effective amount of the pharmaceutical composition.

In some embodiments, provided herein are uses of pharmaceutical compositions in the manufacture of a formulation for reducing the level of MYC protein in a cancer or tumor cell in a subject or a subject in need thereof comprising contacting the cancer or tumor cell with, or administering to the subject, an effective amount of the pharmaceutical composition.

In some embodiments, provided herein are methods of inhibiting proliferation of a cancer or tumor cell in a subject or a subject in need thereof comprising contacting the cancer or tumor cell with, or administering to the subject, an effective amount of a crystalline form as provided herein, or a pharmaceutically acceptable salt or solvate thereof, or a pharmaceutical composition as described herein. In some embodiments, provided herein are methods of inhibiting proliferation of a cancer or tumor cell in a subject or a subject in need thereof comprising contacting the cancer or tumor cell with, or administering to the subject, an effective amount of a crystalline form as provided herein as described herein, or a pharmaceutically acceptable salt or solvate thereof. In some embodiments, provided herein are methods of inhibiting proliferation of a cancer or tumor cell in a subject or a subject in need thereof comprising contacting the cancer or tumor cell with, or administering to the subject, an effective amount of a pharmaceutical composition as described herein. In some embodiments, the cancer or tumor has high levels of MYC amplification and overexpression. In some embodiments, the cancer cell is malignant. In some embodiments, the cancer cell is a hematological cancer cell. In some embodiments, the hematological cancer is a B-Cell Acute Lymphoblastic Leukemia (B-ALL), T-Cell Acute Lymphoblastic Leukemia (T-ALL), Acute Myeloid Leukemia (AML), non-Hodgkin's lymphoma, sarcoma, prostate, adenoid cystic carcinoma (ACC), or non-small cell lung cancer (NSCLC). In some embodiments, the tumor cell is from a solid tumor. In some embodiments, the solid tumor is pancreatic carcinoma, gastric and gastroesophageal carcinomas, NSCLC, or sarcoma. In some embodiments, methods described herein further comprise contacting the tumor cell with a targeted therapy. In some embodiments, the targeted therapy is as described herein. In some embodiments, the targeted therapy is a BCL2 inhibitor. In some embodiments, the BCL12 inhibitor is venetoclax. In some embodiments, the method achieves a complete tumor regression.

In some embodiments, provided herein are crystalline forms as provided herein, such as the crystalline Forms I-XIII of the compound of Formula I, in use for inhibiting proliferation of a cancer or tumor cell in a subject or a subject in need thereof comprising contacting the cancer or tumor cell with, or administering to the subject, an effective amount of the crystalline form, or a pharmaceutically acceptable salt or solvate thereof. In some embodiments, the proliferation is inhibited by at least, or about, 10%, 20%, 30%, 40%, 50%, 60%, 70%, 80%, 90%, or 100%.

In some embodiments, provided herein are uses of crystalline forms as provided herein, such as the crystalline Forms I-XIII of the compound of Formula I, for inhibiting proliferation of a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are uses of crystalline forms as provided herein, such as the crystalline Forms I-XIII of the compound of Formula I, in the manufacture of a formulation for inhibiting proliferation of a cancer or tumor cell in a subject or a subject in need thereof comprising contacting the cancer or tumor cell with, or administering to the subject, an effective amount of the crystalline form, or a pharmaceutically acceptable salt or solvate thereof.

In some embodiments, provided herein are pharmaceutical compositions in use for inhibiting proliferation of a cancer or tumor cell in a subject or a subject in need thereof comprising contacting the cancer or tumor cell with, or administering to the subject, an effective amount of the pharmaceutical composition.

In some embodiments, provided herein are uses of pharmaceutical compositions for inhibiting proliferation of a cancer or tumor cell in a subject or a subject in need thereof comprising contacting the cancer or tumor cell with, or administering to the subject, an effective amount of the pharmaceutical composition.

In some embodiments, provided herein are uses of pharmaceutical compositions in the manufacture of a formulation for inhibiting proliferation of a cancer or tumor cell in a subject or a subject in need thereof comprising contacting the cancer or tumor cell with, or administering to the subject, an effective amount of the pharmaceutical composition.

Crystalline forms as provided herein, such as the crystalline Forms I-XIII of the compound of Formula I, of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, crystalline forms as provided herein, such as the crystalline Forms I-XIII of the compound of Formula I, of the disclosure, as well as pharmaceutical compositions comprising thereof, can be administered to treat any of the described diseases, alone or in combination with one or more other agents.

In other methods, the crystalline forms as provided herein, such as the crystalline Forms I-XIII of the compound of Formula I, of the disclosure, as well as pharmaceutical compositions comprising thereof, can be administered in combination with agonists of nuclear receptors agents.

In other methods, the crystalline forms as provided herein, such as the crystalline Forms I-XIII of the compound of Formula I, of the disclosure, as well as pharmaceutical compositions comprising thereof, can be administered in combination with antagonists of nuclear receptors agents.

In other methods, the crystalline forms as provided herein, such as the crystalline Forms I-XIII of the compound of Formula I, of the disclosure, as well as pharmaceutical compositions comprising thereof, can be administered in combination with an anti-proliferative agent.

### Combination Therapies

For treating cancer and other proliferative diseases, the crystalline forms as provided herein, such as the crystalline Forms I-XIII of the compound of Formula I, can be used in combination with chemotherapeutic agents, agonists or antagonists of nuclear receptors, or other anti-proliferative agents. The compounds can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the crystalline forms as provided herein, such as the crystalline Forms I-XIII of the compound of Formula I, can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the crystalline forms as provided herein, such as the crystalline Forms I-XIII of the compound of Formula I, can be used in combination with targeted therapies, including JAK kinase inhibitors (e.g., Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g., Bortezomib, Carfilzomib), HDAC inhibitors (e.g., panobinostat, vorinostat), DNA methyl transferase inhibitors, dexamethasone, bromo and extra terminal family member (BET) inhibitors, BTK inhibitors (e.g., ibrutinib, acalabrutinib), BCL2 inhibitors (e.g., venetoclax), dual BCL2 family inhibitors (e.g., BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

For treating autoimmune or inflammatory conditions, the crystalline form as provided herein, such as the crystalline Forms I-XIII of the compound of Formula I, can be administered in combination with a corticosteroid such as triamcinolone, dexamethasone, fluocinolone, cortisone, prednisolone, or flumetholone.

For treating autoimmune or inflammatory conditions, the crystalline form as provided herein, such as the crystalline Forms I-XIII of the compound of Formula I, can be administered in combination with an immune suppressant such as fluocinolone acetonide (Retisert®), rimexolone (AL-2178, Vexol, Alcon), or cyclosporine (Restasis®).

In some embodiments, the crystalline forms as provided herein, such as the crystalline Forms I-XIII of the compound of Formula I, are used in methods of prevention (prevent or preventing) or prophylaxis of the diseases, disorders, or conditions provided herein. In some embodiments, the crystalline forms are used to prevent the recurrence of a condition or disease provided herein.

The disclosure is also directed to the following aspects:
Aspect 1. A crystalline form of a compound having a formula of

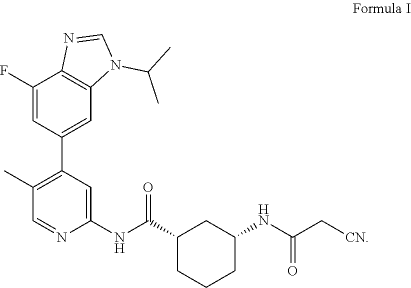 [SMILES: Cc1cnc(NC(=O)[C@H]2CCC[C@@H](NC(=O)CC#N)C2)cc1-c1cc(F)c2ncn(C(C)C)c2c1]

Aspect 2. A crystalline form of a compound having a formula of

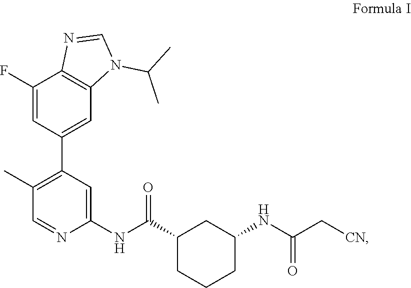 [SMILES: Cc1cnc(NC(=O)[C@H]2CCC[C@@H](NC(=O)CC#N)C2)cc1-c1cc(F)c2ncn(C(C)C)c2c1]

wherein the crystalline form is Form I.
Aspect 3. The crystalline form of aspect 2, wherein the crystalline Form I is characterized by an X-ray powder diffraction pattern comprising one or more peaks at about 7.2±0.5 degrees 2θ, at about 8.0±0.5 degrees 2θ, at about 10.1±0.5 degrees 2θ, at about 11.3±0.5 degrees 2θ, at about 13.0±0.5 degrees 2θ, at about 14.4±0.5 degrees 2θ, at about 15.3±0.5 degrees 2θ, at about 16.8±0.5 degrees 2θ, at about 18.2±0.5 degrees 2θ, at about 20.9±0.5 degrees 2θ, at about 21.6±0.5 degrees 2θ, at about 22.2±0.5 degrees 2θ, and at about 23.1±0.5 degrees 2θ.
Aspect 4. The crystalline form of aspect 2, wherein the crystalline Form I is characterized by an X-ray powder diffraction pattern comprising one or more peaks as shown in FIG. 2.
Aspect 5. The crystalline form of aspect 2, wherein the crystalline Form I is characterized by an X-ray powder diffraction pattern comprising one or more d-spacing values at about 12.3±0.5 degrees angstroms, at about 11.0±0.5 degrees angstroms, at about 8.7±0.5 degrees angstroms, at about 7.8±0.5 degrees angstroms, at about 6.8±0.5 degrees angstroms, at about 6.2±0.5 degrees angstroms, at about 5.8±0.5 degrees angstroms, at about 5.3±0.5 degrees angstroms, at about 4.9±0.5 degrees angstroms, at about 4.3±0.5 degrees angstroms, at about 4.1±0.5 degrees angstroms, at about 4.0±0.5 degrees angstroms, and at about 3.8±0.5 degrees angstroms.
Aspect 6. A crystalline form of a compound having a formula of

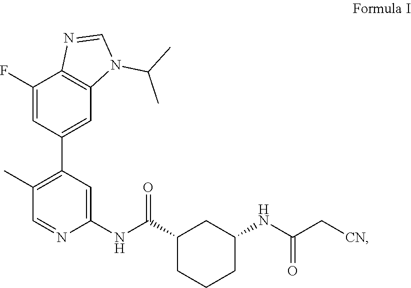 [SMILES: Cc1cnc(NC(=O)[C@H]2CCC[C@@H](NC(=O)CC#N)C2)cc1-c1cc(F)c2ncn(C(C)C)c2c1]

wherein the crystalline form is Form II.
Aspect 7. The crystalline form of aspect 6, wherein the crystalline Form II is characterized by an X-ray powder diffraction pattern comprising one or more peaks at about 7.3±0.5 degrees 2θ, at about 8.1±0.5 degrees 2θ, at about 10.3±0.5 degrees 2θ, at about 11.5±0.5 degrees 2θ, at about 13.1±0.5 degrees 2θ, at about 15.4±0.5 degrees 2θ, at about 16.1±0.5 degrees 2θ, at about 17.0±0.5 degrees 2θ, at about 18.3±0.5 degrees 2θ, at about 19.2±0.5 degrees 2θ, at about 21.0±0.5 degrees 2θ, at about 21.7±0.5 degrees 2θ, at about 22.3±0.5 degrees 2θ, and at about 23.1±0.5 degrees 2θ.
Aspect 8. The crystalline form of aspect 6, wherein the crystalline Form II is characterized by an X-ray powder diffraction pattern comprising one or more peaks as shown in FIG. 6.
Aspect 9. The crystalline form of aspect 6, wherein the crystalline Form II is characterized by an X-ray powder diffraction pattern comprising one or more d-spacing values at about 12.1±0.5 degrees angstroms, at about 10.9±0.5 degrees angstroms, at about 8.6±0.5 degrees angstroms, at about 7.7±0.5 degrees angstroms, at about 6.8±0.5 degrees angstroms, at about 5.7±0.5 degrees angstroms, at about 5.5±0.5 degrees angstroms, at about 5.2±0.5 degrees angstroms, at about 4.8±0.5 degrees angstroms, at about 4.6±0.5 degrees angstroms, at about 4.2±0.5 degrees angstroms, at about 4.1±0.5 degrees angstroms, at about 4.0±0.5 degrees angstroms, and at about 3.8±0.5 degrees angstroms.
Aspect 10. A crystalline form comprising a compound having a formula of

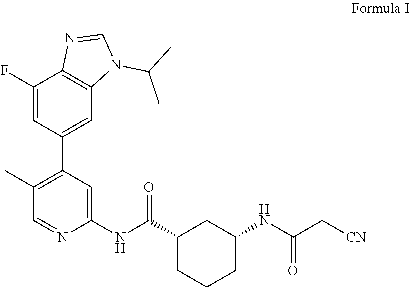 [SMILES: Cc1cnc(NC(=O)[C@H]2CCC[C@@H](NC(=O)CC#N)C2)cc1-c1cc(F)c2ncn(C(C)C)c2c1]

and a coformer.
Aspect 11. The crystalline form of aspect 10, wherein the coformer is an acid.
Aspect 12. The crystalline form of aspect 11, wherein the acid is a pharmaceutically acceptable acid.
Aspect 13. The crystalline form of aspect 12, wherein the pharmaceutically acceptable acid is selected from succinic acid, adipic acid, fumaric acid, glutaric acid, gentisic acid, hydrochloric acid, 1-hydroxy-2-naphthoic acid, salicylic acid, oxalic acid, and D-(−)-tartaric acid.
Aspect 14. A crystalline form comprising a compound having a formula of

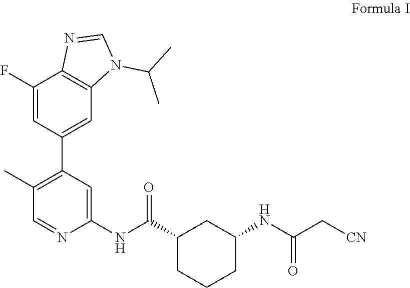 [SMILES: Cc1cnc(NC(=O)[C@H]2CCC[C@@H](NC(=O)CC#N)C2)cc1-c1cc(F)c2ncn(C(C)C)c2c1]

and succinic acid.
Aspect 15. The crystalline form of aspect 14, wherein the molar ratio of the compound of Formula I to succinic acid is about 1:1.
Aspect 16. The crystalline form of aspect 15, wherein the crystalline form is Form III.
Aspect 17. The crystalline form of aspect 16, wherein the crystalline Form III is characterized by an X-ray powder diffraction pattern comprising one or more peaks at about 5.8±0.5 degrees 2θ, at about 8.8±0.5 degrees 2θ, at about 10.5±0.5 degrees 2θ, at about 12.4±0.5 degrees 2θ, at about 14.4±0.5 degrees 2θ, at about 17.5±0.5 degrees 2θ, at about 17.9±0.5 degrees 2θ, at about 18.5±0.5 degrees 2θ, at about 19.5±0.5 degrees 2θ, at about 20.0±0.5 degrees 2θ, at about 20.7±0.5 degrees 2θ, at about 21.4±0.5 degrees 2θ, at about 22.4±0.5 degrees 2θ, at about 22.7±0.5 degrees 2θ, at about 23.6±0.5 degrees 2θ, at about 24.0±0.5 degrees 2θ, at about 24.7±0.5 degrees 2θ, at about 25.9±0.5 degrees 2θ, at about 26.4±0.5 degrees 2θ, at about 27.6±0.5 degrees 2θ, at about 29.0±0.5 degrees 2θ, at about 31.6±0.5 degrees 2θ, and at about 39.5±0.5 degrees 2θ.
Aspect 18. The crystalline form of aspect 16, wherein the crystalline Form III is characterized by an X-ray powder diffraction pattern comprising one or more peaks as shown in FIG. 12.
Aspect 19. The crystalline form of aspect 16, wherein the crystalline Form III is characterized by an X-ray powder diffraction pattern comprising one or more d-spacing values at about 15.1±0.5 degrees angstroms, at about 10.0±0.5 degrees angstroms, at about 8.4±0.5 degrees angstroms, at about 7.1±0.5 degrees angstroms, at about 6.2±0.5 degrees angstroms, at about 5.1±0.5 degrees angstroms, at about 4.9±0.5 degrees angstroms, at about 4.8±0.5 degrees angstroms, at about 4.6±0.5 degrees angstroms, at about 4.4±0.5 degrees angstroms, at about 4.3±0.5 degrees angstroms, at about 4.2±0.5 degrees angstroms, at about 4.0±0.5 degrees angstroms, at about 3.9±0.5 degrees angstroms, at about 3.8±0.5 degrees angstroms, at about 3.7±0.5 degrees angstroms, at about 3.6±0.5 degrees angstroms, at about 3.4±0.5 degrees angstroms, at about 3.4±0.5 degrees angstroms, at about 3.2±0.5 degrees angstroms, at about 3.1±0.5 degrees angstroms, at about 2.8±0.5 degrees angstroms, and at about 2.3±0.5 degrees angstroms.
Aspect 20. A crystalline form comprising a compound having a formula of

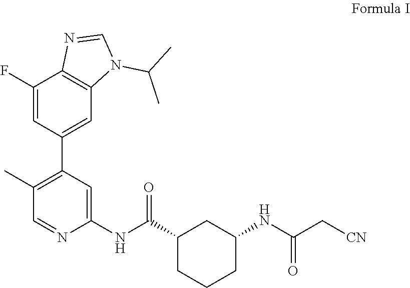 [SMILES: Cc1cnc(NC(=O)[C@H]2CCC[C@@H](NC(=O)CC#N)C2)cc1-c1cc(F)c2ncn(C(C)C)c2c1]

and glutaric acid.
Aspect 21. The crystalline form of aspect 20, wherein the molar ratio of the compound of Formula I to glutaric acid is about 2:1.
Aspect 22. The crystalline form of aspect 21, wherein the crystalline form is Form IV.
Aspect 23. The crystalline form of aspect 22, wherein the crystalline Form IV is characterized by an X-ray powder diffraction pattern comprising one or more peaks at about 4.5±0.5 degrees 2θ, at about 6.0±0.5 degrees 2θ, at about 8.9±0.5 degrees 2θ, at about 11.1±0.5 degrees 2θ, at about 11.7±0.5 degrees 2θ, at about 13.2±0.5 degrees 2θ, at about 16.3±0.5 degrees 2θ, at about 17.1±0.5 degrees 2θ, at about 17.6±0.5 degrees 2θ, at about 18.4±0.5 degrees 2θ, at about 19.7±0.5 degrees 2θ, at about 20.5±0.5 degrees 2θ, at about 21.0±0.5 degrees 2θ, at about 21.9±0.5 degrees 2θ, at about 24.0±0.5 degrees 2θ, at about 24.7±0.5 degrees 2θ, at about 25.0±0.5 degrees 2θ, at about 26.2±0.5 degrees 2θ, and at about 29.2±0.5 degrees 2θ.
Aspect 24. The crystalline form of aspect 22, wherein the crystalline Form IV is characterized by an X-ray powder diffraction pattern comprising one or more peaks as shown in FIG. 18.
Aspect 25. The crystalline form of aspect 22, wherein the crystalline Form IV is characterized by an X-ray powder diffraction pattern comprising one or more d-spacing values at about 19.4±0.5 degrees angstroms, at about 14.7±0.5 degrees angstroms, at about 10.0±0.5 degrees angstroms, at about 7.9±0.5 degrees angstroms, at about 7.5±0.5 degrees angstroms, at about 6.7±0.5 degrees angstroms, at about 5.4±0.5 degrees angstroms, at about 5.2±0.5 degrees angstroms, at about 5.0±0.5 degrees angstroms, at about 4.8±0.5 degrees angstroms, at about 4.5±0.5 degrees angstroms, at about 4.3±0.5 degrees angstroms, at about 4.2±0.5 degrees angstroms, at about 4.1±0.5 degrees angstroms, at about 3.7±0.5 degrees angstroms, at about 3.6±0.5 degrees angstroms, at about 3.6±0.5 degrees angstroms, at about 3.4±0.5 degrees angstroms, and at about 3.1±0.5 degrees angstroms.
Aspect 26. A crystalline form comprising a compound having a formula of

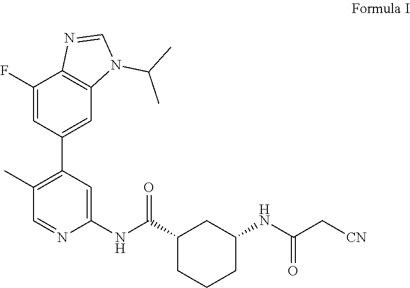 [SMILES: Cc1cnc(NC(=O)[C@H]2CCC[C@@H](NC(=O)CC#N)C2)cc1-c1cc(F)c2ncn(C(C)C)c2c1]

and adipic acid.
Aspect 27. The crystalline form of aspect 26, wherein the molar ratio of the compound of Formula I to adipic acid is about 1:1.
Aspect 28. The crystalline form of aspect 27, wherein the crystalline form is Form V.
Aspect 29. The crystalline form of aspect 28, wherein the crystalline Form V is characterized by an X-ray powder diffraction pattern comprising one or more peaks at about 4.7±0.5 degrees 2θ, at about 7.4±0.5 degrees 2θ, at about 9.2±0.5 degrees 2θ, at about 11.2±0.5 degrees 2θ, at about 13.8±0.5 degrees 2θ, at about 17.2±0.5 degrees 2θ, at about 18.1±0.5 degrees 2θ, at about 18.9±0.5 degrees 2θ, at about 25.1±0.5 degrees 2θ, and at about 25.9±0.5 degrees 2θ.
Aspect 30. The crystalline form of aspect 28, wherein the crystalline Form V is characterized by an X-ray powder diffraction pattern comprising one or more peaks as shown in FIG. 24.
Aspect 31. The crystalline form of aspect 28, wherein the crystalline Form V is characterized by an X-ray powder diffraction pattern comprising one or more d-spacing values at about 18.8±0.5 degrees angstroms, at about 11.9±0.5 degrees angstroms, at about 9.6±0.5 degrees angstroms, at about 7.9±0.5 degrees angstroms, at about 6.4±0.5 degrees angstroms, at about 5.1±0.5 degrees angstroms, at about 4.9±0.5 degrees angstroms, at about 4.7±0.5 degrees angstroms, at about 3.6±0.5 degrees angstroms, and at about 3.4±0.5 degrees angstroms.
Aspect 32. A crystalline form comprising a compound having a formula of

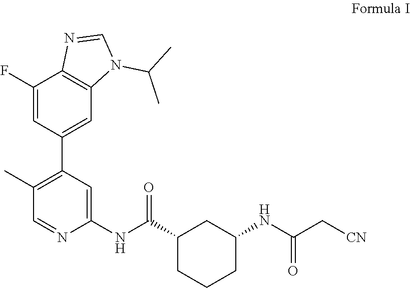 [SMILES: Cc1cnc(NC(=O)[C@H]2CCC[C@@H](NC(=O)CC#N)C2)cc1-c1cc(F)c2ncn(C(C)C)c2c1]

and gentisic acid.
Aspect 33. The crystalline form of aspect 32, wherein the molar ratio of the compound of Formula I to gentisic acid is about 2:1.
Aspect 34. The crystalline form of aspect 33, wherein the crystalline form is Form VI.
Aspect 35. The crystalline form of aspect 34, wherein the crystalline Form VI is characterized by an X-ray powder diffraction pattern comprising one or more peaks at about 7.4±0.5 degrees 2θ, at about 9.5±0.5 degrees 2θ, at about 13.6±0.5 degrees 2θ, at about 14.7±0.5 degrees 2θ, at about 15.5±0.5 degrees 2θ, at about 16.4±0.5 degrees 2θ, at about 17.2±0.5 degrees 2θ, at about 18.2±0.5 degrees 2θ, at about 19.4±0.5 degrees 2θ, at about 20.5±0.5 degrees 2θ, at about 21.5±0.5 degrees 2θ, at about 23.5±0.5 degrees 2θ, at about 24.8±0.5 degrees 2θ, at about 25.7±0.5 degrees 2θ, at about 26.9±0.5 degrees 2θ, at about 29.4±0.5 degrees 2θ, and at about 30.7±0.5 degrees 2θ.
Aspect 36. The crystalline form of aspect 34, wherein the crystalline Form VI is characterized by an X-ray powder diffraction pattern comprising one or more peaks as shown in FIG. 36.
Aspect 37. The crystalline form of aspect 34, wherein the crystalline Form VI is characterized by an X-ray powder diffraction pattern comprising one or more d-spacing values at about 12.0±0.5 degrees angstroms, at about 9.3±0.5 degrees angstroms, at about 6.5±0.5 degrees angstroms, at about 6.0±0.5 degrees angstroms, at about 5.7±0.5 degrees angstroms, at about 5.4±0.5 degrees angstroms, at about 5.2±0.5 degrees angstroms, at about 4.9±0.5 degrees angstroms, at about 4.6±0.5 degrees angstroms, at about 4.3±0.5 degrees angstroms, at about 4.1±0.5 degrees angstroms, at about 3.8±0.5 degrees angstroms, at about 3.6±0.5 degrees angstroms, at about 3.5±0.5 degrees angstroms, at about 3.3±0.5 degrees angstroms, at about 3.0±0.5 degrees angstroms, and at about 2.9±0.5 degrees angstroms.
Aspect 38. A crystalline form comprising a compound having a formula of

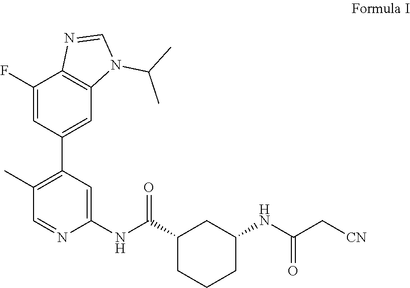 [SMILES: Cc1cnc(NC(=O)[C@H]2CCC[C@@H](NC(=O)CC#N)C2)cc1-c1cc(F)c2ncn(C(C)C)c2c1]

and fumaric acid.
Aspect 39. The crystalline form of aspect 38, wherein the molar ratio of the compound of Formula I to fumaric acid is about 2:1.
Aspect 40. The crystalline form of aspect 39, wherein the crystalline form is Formic.
Aspect 41. The crystalline form of aspect 40, wherein the crystalline Form VII is characterized by an X-ray powder diffraction pattern comprising one or more peaks at about 4.7±0.5 degrees 2θ, at about 5.8±0.5 degrees 2θ, at about 10.6±0.5 degrees 2θ, at about 11.3±0.5 degrees 2θ, at about 11.8±0.5 degrees 2θ, at about 12.6±0.5 degrees 2θ, at about 13.1±0.5 degrees 2θ, at about 14.0±0.5 degrees 2θ, at about 16.0±0.5 degrees 2θ, at about 17.0±0.5 degrees 2θ, at about 17.5±0.5 degrees 2θ, at about 18.7±0.5 degrees 2θ, at about 19.3±0.5 degrees 2θ, at about 21.2±0.5 degrees 2θ, at about 22.1±0.5 degrees 2θ, at about 24.2±0.5 degrees 2θ, at about 24.7±0.5 degrees 2θ, at about 26.2±0.5 degrees 2θ, and at about 27.4±0.5 degrees 2θ.
Aspect 42. The crystalline form of aspect 40, wherein the crystalline Form VII is characterized by an X-ray powder diffraction pattern comprising one or more peaks as shown in FIG. 30.
Aspect 43. The crystalline form of aspect 40, wherein the crystalline Form VII is characterized by an X-ray powder diffraction pattern comprising one or more d-spacing values at about 18.9±0.5 degrees angstroms, at about 15.2±0.5 degrees angstroms, at about 8.4±0.5 degrees angstroms, at about 7.8±0.5 degrees angstroms, at about 7.5±0.5 degrees angstroms, at about 7.0±0.5 degrees angstroms, at about 6.8±0.5 degrees angstroms, at about 6.3±0.5 degrees angstroms, at about 5.5±0.5 degrees angstroms, at about 5.2±0.5 degrees angstroms, at about 5.1±0.5 degrees angstroms, at about 4.8±0.5 degrees angstroms, at about 4.6±0.5 degrees angstroms, at about 4.2±0.5 degrees angstroms, at about 4.0±0.5 degrees angstroms, at about 3.7±0.5 degrees angstroms, at about 3.6±0.5 degrees angstroms, at about 3.4±0.5 degrees angstroms, and at about 3.2±0.5 degrees angstroms.
Aspect 44. The crystalline form of aspect 38, wherein the molar ratio of the compound of Formula I to fumaric acid is about 1:1.
Aspect 45. The crystalline form of aspect 39, wherein the crystalline form is Form VIII.
Aspect 46. The crystalline form of aspect 45, wherein the crystalline Form VIII is characterized by an X-ray powder diffraction pattern comprising one or more peaks at about 3.9±0.5 degrees 2θ, at about 5.7±0.5 degrees 2θ, at about 7.1±0.5 degrees 2θ, at about 8.6±0.5 degrees 2θ, at about 10.3±0.5 degrees 2θ, at about 12.1±0.5 degrees 2θ, at about 14.1±0.5 degrees 2θ, at about 17.1±0.5 degrees 2θ, at about 19.1±0.5 degrees 2θ, at about 20.6±0.5 degrees 2θ, at about 22.2±0.5 degrees 2θ, at about 23.0±0.5 degrees 2θ, at about 24.3±0.5 degrees 2θ, at about 26.0±0.5 degrees 2θ, at about 26.5±0.5 degrees 2θ, at about 28.5±0.5 degrees 2θ, at about 34.6±0.5 degrees 2θ, at about 35.4±0.5 degrees 2θ, at about 36.8±0.5 degrees 2θ, and at about 39.5±0.5 degrees 2θ.
Aspect 47. The crystalline form of aspect 45, wherein the crystalline Form VIII is characterized by an X-ray powder diffraction pattern comprising one or more peaks as shown in FIG. 42.
Aspect 48. The crystalline form of aspect 45, wherein the crystalline Form VIII is characterized by an X-ray powder diffraction pattern comprising one or more d-spacing values at about 22.5±0.5 degrees angstroms, at about 15.5±0.5 degrees angstroms, at about 12.5±0.5 degrees angstroms, at about 10.3±0.5 degrees angstroms, at about 8.6±0.5 degrees angstroms, at about 7.3±0.5 degrees angstroms, at about 6.3±0.5 degrees angstroms, at about 5.2±0.5 degrees angstroms, at about 4.7±0.5 degrees angstroms, at about 4.3±0.5 degrees angstroms, at about 4.0±0.5 degrees angstroms, at about 3.9±0.5 degrees angstroms, at about 3.7±0.5 degrees angstroms, at about 3.4±0.5 degrees angstroms, at about 3.4±0.5 degrees angstroms, at about 3.1±0.5 degrees angstroms, at about 2.6±0.5 degrees angstroms, at about 2.5±0.5 degrees angstroms, at about 2.4±0.5 degrees angstroms, and at about 2.3±0.5 degrees angstroms.
Aspect 49. A crystalline form comprising a compound having a formula of

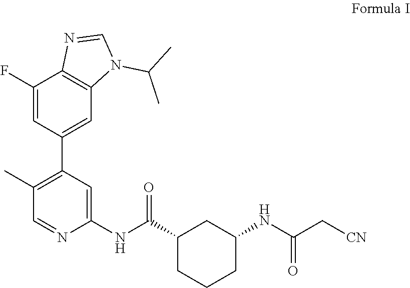 [SMILES: Cc1cnc(NC(=O)[C@H]2CCC[C@@H](NC(=O)CC#N)C2)cc1-c1cc(F)c2ncn(C(C)C)c2c1]

and D-(−)-tartaric acid.
Aspect 50. The crystalline form of aspect 49, wherein the molar ratio of the compound of Formula I to D-(−)-tartaric acid is about 1:1.
Aspect 51. The crystalline form of aspect 50, wherein the crystalline form is Form IX.
Aspect 52. The crystalline form of aspect 51, wherein the crystalline Form IX is characterized by an X-ray powder diffraction pattern comprising one or more peaks at about 5.3±0.5 degrees 2θ, at about 6.8±0.5 degrees 2θ, at about 9.0±0.5 degrees 2θ, at about 10.0±0.5 degrees 2θ, at about 15.5±0.5 degrees 2θ, at about 17.3±0.5 degrees 2θ, at about 18.2±0.5 degrees 2θ, at about 18.8±0.5 degrees 2θ, at about 19.9±0.5 degrees 2θ, at about 20.9±0.5 degrees 2θ, at about 21.3±0.5 degrees 2θ, at about 22.7±0.5 degrees 2θ, at about 23.6±0.5 degrees 2θ, at about 24.3±0.5 degrees 2θ, at about 25.5±0.5 degrees 2θ, at about 26.0±0.5 degrees 2θ, at about 27.1±0.5 degrees 2θ, at about 28.0±0.5 degrees 2θ, at about 28.8±0.5 degrees 2θ, at about 29.8±0.5 degrees 2θ, at about 33.4±0.5 degrees 2θ, at about 34.2±0.5 degrees 2θ, at about 36.3±0.5 degrees 2θ, at about 38.6±0.5 degrees 2θ, and at about 39.1±0.5 degrees 2θ.
Aspect 53. The crystalline form of aspect 51, wherein the crystalline Form IX is characterized by an X-ray powder diffraction pattern comprising one or more peaks as shown in FIG. 46.
Aspect 54. The crystalline form of aspect 51, wherein the crystalline Form IX is characterized by an X-ray powder diffraction pattern comprising one or more d-spacing values at about 16.7±0.5 degrees angstroms, at about 12.9±0.5 degrees angstroms, at about 9.8±0.5 degrees angstroms, at about 8.8±0.5 degrees angstroms, at about 5.7±0.5 degrees angstroms, at about 5.1±0.5 degrees angstroms, at about 4.9±0.5 degrees angstroms, at about 4.7±0.5 degrees angstroms, at about 4.5±0.5 degrees angstroms, at about 4.3±0.5 degrees angstroms, at about 4.2±0.5 degrees angstroms, at about 3.9±0.5 degrees angstroms, at about 3.8±0.5 degrees angstroms, at about 3.7±0.5 degrees angstroms, at about 3.5±0.5 degrees angstroms, at about 3.4±0.5 degrees angstroms, at about 3.3±0.5 degrees angstroms, at about 3.2±0.5 degrees angstroms, at about 3.1±0.5 degrees angstroms, at about 3.0±0.5 degrees angstroms, at about 2.7±0.5 degrees angstroms, at about 2.6±0.5 degrees angstroms, at about 2.5±0.5 degrees angstroms, at about 2.3±0.5 degrees angstroms, and at about 2.3±0.5 degrees angstroms.
Aspect 55. A crystalline form comprising a compound having a formula of

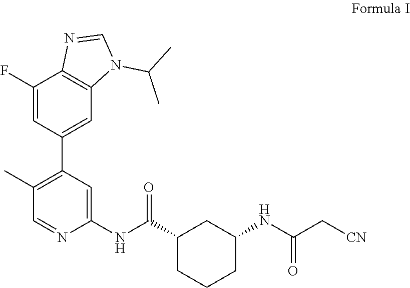 [SMILES: Cc1cnc(NC(=O)[C@H]2CCC[C@@H](NC(=O)CC#N)C2)cc1-c1cc(F)c2ncn(C(C)C)c2c1]

and hydrochloric acid.
Aspect 56. The crystalline form of aspect 55, wherein the crystalline form is Form X.
Aspect 57. The crystalline form of aspect 56, wherein the crystalline Form X is characterized by an X-ray powder diffraction pattern comprising one or more peaks as shown in FIG. 50.
Aspect 58. A crystalline form comprising a compound having a formula of

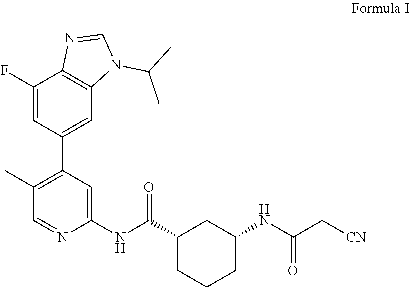 [SMILES: Cc1cnc(NC(=O)[C@H]2CCC[C@@H](NC(=O)CC#N)C2)cc1-c1cc(F)c2ncn(C(C)C)c2c1]

and salicylic acid.
Aspect 59. The crystalline form of aspect 58, wherein the molar ratio of the compound of Formula I to salicylic acid is about 2:1.
Aspect 60. The crystalline form of aspect 59, wherein the crystalline form is Form XI.
Aspect 61. The crystalline form of aspect 60, wherein the crystalline Form XI is characterized by an X-ray powder diffraction pattern comprising one or more peaks as shown in FIG. 52.
Aspect 62. A crystalline form comprising a compound having a formula of

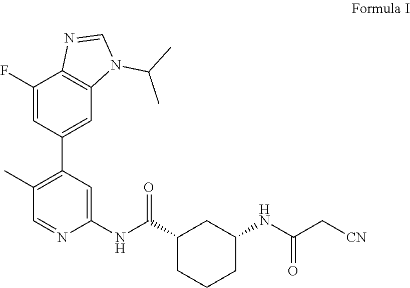 [SMILES: Cc1cnc(NC(=O)[C@H]2CCC[C@@H](NC(=O)CC#N)C2)cc1-c1cc(F)c2ncn(C(C)C)c2c1]

and oxalic acid.
Aspect 63. The crystalline form of aspect 62, wherein the molar ratio of the compound of Formula I to oxalic acid is about 1:1.
Aspect 64. The crystalline form of aspect 63, wherein the crystalline form is Form XII.
Aspect 65. The crystalline form of aspect 64, wherein the crystalline Form XII is characterized by an X-ray powder diffraction pattern comprising one or more peaks as shown in FIG. 54.
Aspect 66. A crystalline form comprising a compound having a formula of

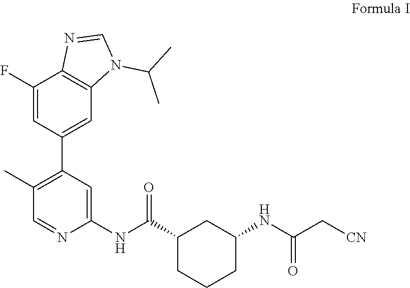 [SMILES: Cc1cnc(NC(=O)[C@H]2CCC[C@@H](NC(=O)CC#N)C2)cc1-c1cc(F)c2ncn(C(C)C)c2c1]

and 1-hydroxy-2-naphthoic acid.
Aspect 67. The crystalline form of aspect 66, wherein the crystalline form is Form XIII.
Aspect 68. The crystalline form of aspect 67, wherein the crystalline Form XIII is characterized by an X-ray powder diffraction pattern comprising one or more peaks as shown in FIG. 59.
Aspect 69. A pharmaceutical composition comprising a crystalline form of any one of aspects 1-68.
Aspect 70. A pharmaceutical composition comprising the crystalline Form I of any one of aspects 2-5.
Aspect 71. A pharmaceutical composition comprising the crystalline Form II of any one of aspects 6-9.
Aspect 72. A pharmaceutical composition comprising the crystalline Form III of any one of aspects 16-19.
Aspect 73. A pharmaceutical composition comprising the crystalline Form IV of any one of aspects 22-25.
Aspect 74. A pharmaceutical composition comprising the crystalline Form V of any one of aspects 28-31.
Aspect 75. A pharmaceutical composition comprising the crystalline Form VI of any one of aspects 34-37.
Aspect 76. A pharmaceutical composition comprising the crystalline Form VII of any one of aspects 40-43.
Aspect 77. A pharmaceutical composition comprising the crystalline Form VIII of any one of aspects 45-48.
Aspect 78. A pharmaceutical composition comprising the crystalline Form IX of any one of aspects 51-54.
Aspect 79. A pharmaceutical composition comprising the crystalline Form X of any one of aspects 56-57.
Aspect 80. A pharmaceutical composition comprising the crystalline Form XI of any one of aspects 60-61.
Aspect 81. A pharmaceutical composition comprising the crystalline Form XII of any one of aspects 64-65.
Aspect 82. A pharmaceutical composition comprising the crystalline Form XIII of any one of aspects 67-68.
Aspect 83. The pharmaceutical composition of any one of aspects 69-82, further comprising a pharmaceutically acceptable excipient.
Aspect 84. A process for preparing a crystalline form of any one of aspects 1-68, comprising crystallizing the compound of

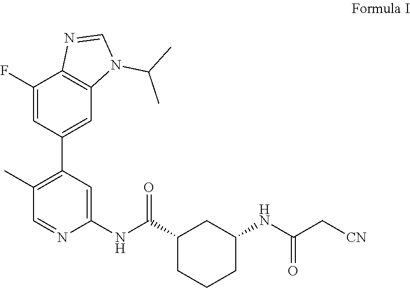 [SMILES: Cc1cnc(NC(=O)[C@H]2CCC[C@@H](NC(=O)CC#N)C2)cc1-c1cc(F)c2ncn(C(C)C)c2c1]

to form the crystalline form and optionally isolating the crystalline form.
Aspect 85. The process of aspect 84, wherein the crystallizing comprises dissolving the compound of Formula I in an organic solvent and crystallizing the compound of Formula I to form the crystalline Form I or Form II therefrom.
Aspect 86. The process of aspect 84, wherein the crystallizing comprises dissolving the compound of Formula I with a pharmaceutically acceptable acid in an organic solvent and crystallizing the compound of Formula I to form any one of the crystalline Form III to Form XIII therefrom.
Aspect 87. The process of aspect 86, wherein the pharmaceutically acceptable acid is selected from succinic acid, adipic acid, fumaric acid, glutaric acid, gentisic acid, hydrochloric acid, 1-hydroxy-2-naphthoic acid, salicylic acid, oxalic acid, and D-(−)-tartaric acid.
Aspect 88. The process of aspect 84, wherein the crystallizing comprises dissolving the compound of Formula I with succinic acid in an organic solvent and crystallizing the compound of Formula I to form the crystalline Form III therefrom.
Aspect 89. The process of any one of aspects 85-88, wherein the organic solvent is selected from the group consisting of acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, octane, and any combination thereof.
Aspect 90. The process of aspect 85, wherein the solvent is acetonitrile.
Aspect 91. The process of aspect 88, wherein the solvent is ethyl acetate.
Aspect 92. A method of inhibiting a CDK enzyme comprising: contacting the CDK enzyme with an effective amount of a crystalline form of any one of aspects 1-68, a pharmaceutical composition of any one of aspects 69-83, or a crystalline form prepared according to the process of any one of aspects 84-91.
Aspect 93. The method of aspect 92, wherein the CDK enzyme is CDK9.
Aspect 94. A method of treating a disease or disorder associated with aberrant CDK activity in a subject or a subject in need thereof comprising administering to the subject, a crystalline form of any one of aspects 1-68, a pharmaceutical composition of any one of aspects 69-83, or a crystalline form prepared according to the process of any one of aspects 84-91.
Aspect 95. The method of aspect 94, wherein the disease or disorder associated with aberrant CDK activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.
Aspect 96. A method of treating cancer in a subject or a subject in need thereof comprising administering to the subject, a crystalline form of any one of aspects 1-68, a pharmaceutical composition of any one of aspects 69-83, or a crystalline form prepared according to the process of any one of aspects 84-91.
Aspect 97. The method of aspect 96, wherein the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.
Aspect 98. A method of inducing apoptosis in a cancer or tumor cell in a subject or a subject in need thereof comprising contacting the cancer or tumor cell with, or administering to the subject, an effective amount of a crystalline form of any one of aspects 1-68, a pharmaceutical composition of any one of aspects 69-83, or a crystalline form prepared according to the process of any one of aspects 84-91.
Aspect 99. A method of inhibiting phosphorylation of Ser2RNAP2 in a cancer or tumor cell in a subject or a subject in need thereof comprising contacting the cancer or tumor cell with, or administering to the subject, an effective amount of a crystalline form of any one of aspects 1-68, a pharmaceutical composition of any one of aspects 69-83, or a crystalline form prepared according to the process of any one of aspects 84-91.
Aspect 100. A method of reducing the level of induced myeloid leukemia cell differentiation protein Mcl-1 (MCL1) in a cancer or tumor cell in a subject or a subject in need thereof comprising contacting the cancer or tumor cell with, or administering to the subject, an effective amount of a crystalline form of any one of aspects 1-68, a pharmaceutical composition of any one of aspects 69-83, or a crystalline form prepared according to the process of any one of aspects 84-91.
Aspect 101. A method of reducing the level of MYC protein in a cancer or tumor cell in a subject or a subject in need thereof comprising contacting the cancer or tumor cell with, or administering to the subject, an effective amount of a crystalline form of any one of aspects 1-68, a pharmaceutical composition of any one of aspects 69-83, or a crystalline form prepared according to the process of any one of aspects 84-91.
Aspect 102. A method of inhibiting proliferation of a cancer or tumor cell in a subject or a subject in need thereof comprising contacting the cancer or tumor cell with, or administering to the subject, an effective amount of a crystalline form of any one of aspects 1-68, a pharmaceutical composition of any one of aspects 69-83, or a crystalline form prepared according to the process of any one of aspects 84-91.
Aspect 103. The method of any one of aspects 98-102, wherein the cancer or tumor has high levels of MYC amplification and overexpression.
Aspect 104. The method of any one of aspects 98-103, wherein the cancer cell is malignant.
Aspect 105. The method of any one of aspects 98-103, wherein the cancer cell is a hematological cancer cell.
Aspect 106. The method of aspect 105, wherein the hematological cancer is a B-Cell Acute Lymphoblastic Leukemia (B-ALL), T-Cell Acute Lymphoblastic Leukemia (T-ALL), Acute Myeloid Leukemia (AML), non-Hodgkin's lymphoma, sarcoma, prostate, adenoid cystic carcinoma (ACC), or non-small cell lung cancer (NSCLC).
Aspect 107. The method of any one of aspects 98-102, wherein the tumor cell is from a solid tumor.
Aspect 108. The method of aspect 106, wherein the solid tumor is pancreatic carcinoma, gastric and gastroesophageal carcinomas, NSCLC, or sarcoma.
Aspect 109. The method according to aspects 107 or 108, further comprising contacting the tumor cell with a targeted therapy.
Aspect 110. The method of aspect 109, wherein the targeted therapy is a BCL2 inhibitor.
Aspect 111. The method of aspect 110, wherein the BCL2 inhibitor is venetoclax.
Aspect 112. The method of any one of aspects 109-111, wherein a complete tumor regression is achieved.

The following examples are illustrative, but not limiting, of the methods, crystalline forms, and pharmaceutical compositions described herein. Other suitable modifications and adaptations of the variety of conditions and parameters normally encountered in therapy, synthesis, and other embodiments disclosed herein are within the spirit and scope of the embodiments.

### EXAMPLES

### Example 1

### Preparations of Crystalline Form I of the Compound of Formula I

Crystalline Form I of the compound of Formula I was prepared following the procedure described herein. A single crystalline Form I of the compound of Formula I was obtained by the procedure as follows:

| Steps | Operation | Note |
|---|---|---|
| 1 | Add 1390 mg (2.917 mmol) of the amorphous compound of | |
| | Formula I to 20 mL of vial | |
| 2 | Added 5.5 mL of acetonitrile | |
| 3 | Stirred at 81° C. for 3 h, | slurry |
| 4 | Stirred at 65° C. for 5 h, | slurry |
| 5 | Cooled to room temperature and stirred overnight | slurry |
| 6 | Filtered and washed with 5 mL of acetonitrile | |
| 7 | | |
| 8 | Dried the cake at 48-50° C. under vacuum | |
| | for 48 h to get the product | |
| | Sample ID: Amorphous(0.730 g, 52.52%) | |
| 9 | Combined the mother liquid and washed liquid | |
| 10 | Removed some solvent until the total volume is about | |
| | 1.8 mL | |
| 11 | Stirred overnight to give a slurry | |
| 12 | Filtered and washed with 3 mL of acetonitrile/MTBE (1:1) | |
| | Dried the cake at 48-50° C. under vacuum for 8 h | |
| | to give 46 mg the crystalline Form I | |

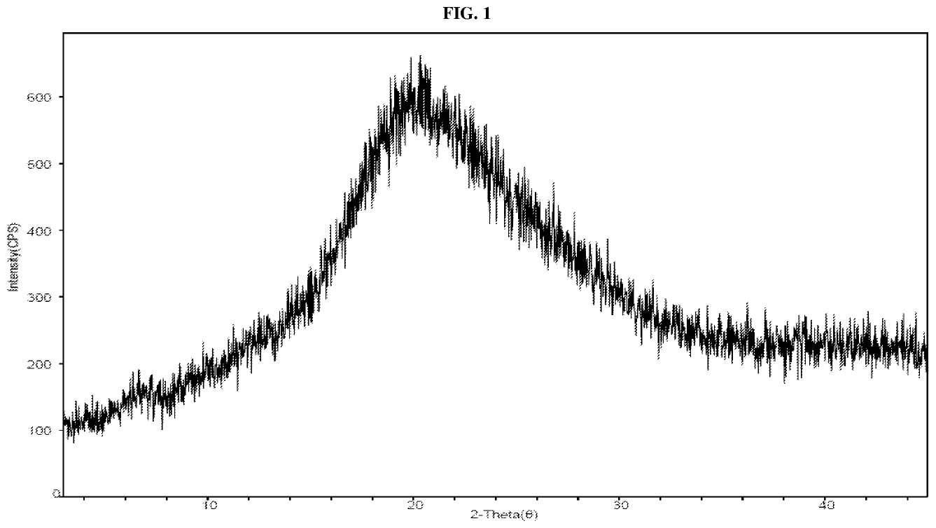

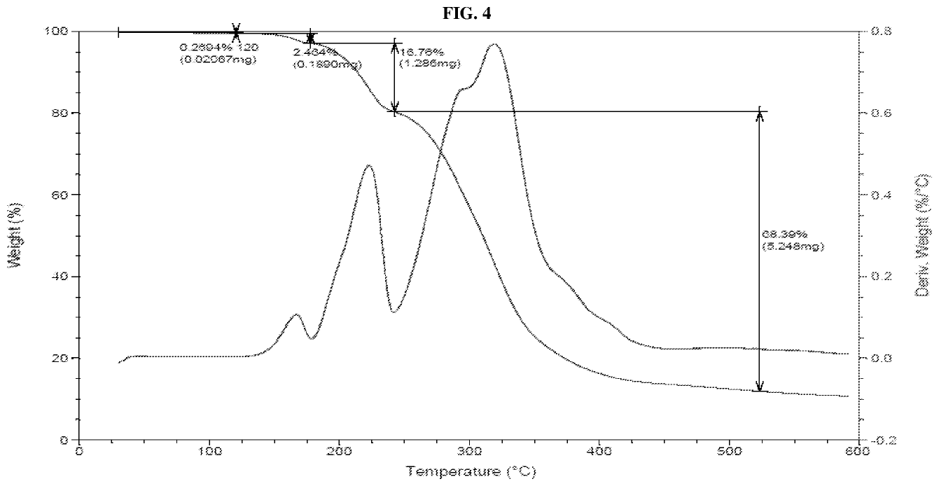

DSC analysis of this sample shows a melting onset at about 165° C. as shown in FIG. 3. TGA analysis of the crystalline Form I shows a degradation occurring after 176° C., as shown in FIG. 4. TGA analysis shows degradation to start between 100° C. and 125° C. FIG. 5 shows that ¹H NMR) analysis confirms the crystalline Form I only comprises the compound of Formula I and the crystalline Form I is more than 99% pure based on HPLC analysis. FIG. 1 shows the X-ray powder diffraction pattern for the amorphous compound of Formula I.

### Example 2

### Preparations of Crystalline Form I of the Compound of Formula I

Crystalline Form I of the compound of Formula I was also prepared following the procedure described herein. A single crystalline Form I of the compound of Formula I was obtained by the procedure as follows:

| Steps | Operation | Note |
|---|---|---|
| 1 | Assembled a clean and dry 50 mL RBF reactor | |
| | with magnetic stirring, thermocouple, and | |
| | condenser charged 7.1 g (0.015 mol) | |
| | of the amorphous compound of Formula I | |
| 2 | Charged 26 mL of acetonitrile and stirred | Clear solution |
| 3 | Stirred at 82° C. for 2 h | slurry |
| 4 | Stirred at 72° C. overnight | slurry |
| 5 | Cooled to 50° C. and stirred 60 min | slurry |
| 6 | Filtered and washed with 8 mL of cooled | |
| | acetonitrile | |
| 7 | Dried the cake at 48-50° C. under vacuum | |
| | for 30 h to get the crystalline Form I of | |
| | the compound of Formula I (3.80 g, 53.55%) | |

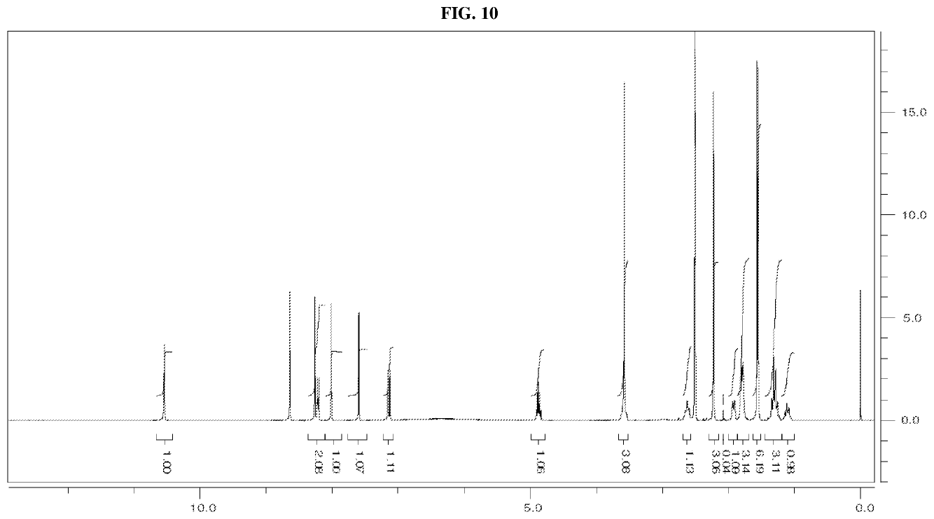

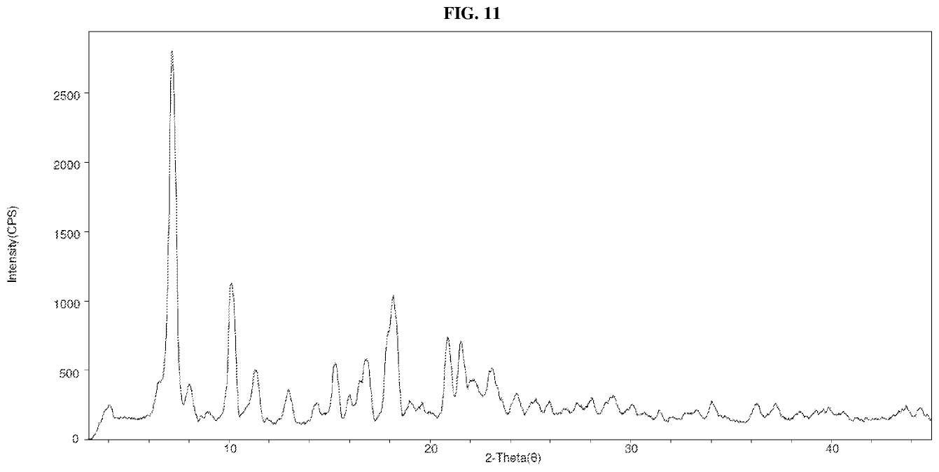

FIG. 10 shows that ¹H NMR analysis confirms the crystalline Form I only comprises the compound of Formula I and the crystalline Form I is more than 99% pure based on HPLC analysis. FIG. 11 shows the X-ray powder diffraction pattern for the crystalline Form I, which is consistent with the diffraction pattern as shown in FIG. 2. FIG. 11 shows the X-ray powder diffraction pattern for the crystalline Form I. Peak positions of the crystalline Form I are provided in Table 1.

TABLE 1

| Angle | d value |
|---|---|
| (2θ) | (Angstrom) |
| 4.04 | 21.8522 |
| 7.16 | 12.3359 |
| 8.002 | 11.0401 |
| 9.019 | 9.7966 |
| 10.12 | 8.7335 |
| 11.28 | 7.8377 |
| 12.96 | 6.8255 |
| 14.361 | 6.1626 |
| 15.3 | 5.7865 |
| 16 | 5.5346 |
| 16.78 | 5.279 |
| 18.16 | 4.8809 |
| 18.982 | 4.6715 |
| 19.619 | 4.5211 |
| 20.881 | 4.2508 |
| 21.559 | 4.1184 |
| 22.199 | 4.0011 |
| 23.12 | 3.8438 |
| 24.3 | 3.6597 |
| 25.261 | 3.5227 |
| 25.941 | 3.4319 |
| 27.378 | 3.2549 |
| 28.061 | 3.1772 |
| 29.141 | 3.0619 |
| 30.042 | 2.9721 |
| 31.421 | 2.8447 |
| 33.279 | 2.69 |
| 34.04 | 2.6316 |
| 36.3 | 2.4728 |
| 37.219 | 2.4138 |
| 38.44 | 2.3399 |
| 39.899 | 2.2576 |
| 43.72 | 2.0687 |

### Example 3

### Preparations of Crystalline Form II of the Compound of Formula I

Crystalline Form II of the compound of Formula I was prepared following the procedure described herein. A single crystalline Form II of the compound of Formula I was obtained by the procedure as follows:

| Steps | Operation | Note |
|---|---|---|
| 1 | Add 169 mg (0.355 mmol) of the amorphous compound | |
| | of Formula I | |
| 2 | Added 0.5 mL of acetonitrile and stirred | |
| 3 | Stirred at 83° C. for 2 h | slurry |
| 4 | Stirred at 60° C. for 5 h, | slurry |
| 5 | Cooled to room temperature and stirred overnight | slurry |
| 6 | Added 1.0 mL ethyl acetate and stirred | |
| 7 | Filtered and washed with 1.0 mL of ethyl acetate | |
| 8 | Dried the cake at 48-50° C. under vacuum for | |
| | 48 h to get the crystalline Form II of the | |
| | compound of Formula I (90 mg, 53.25%) | |

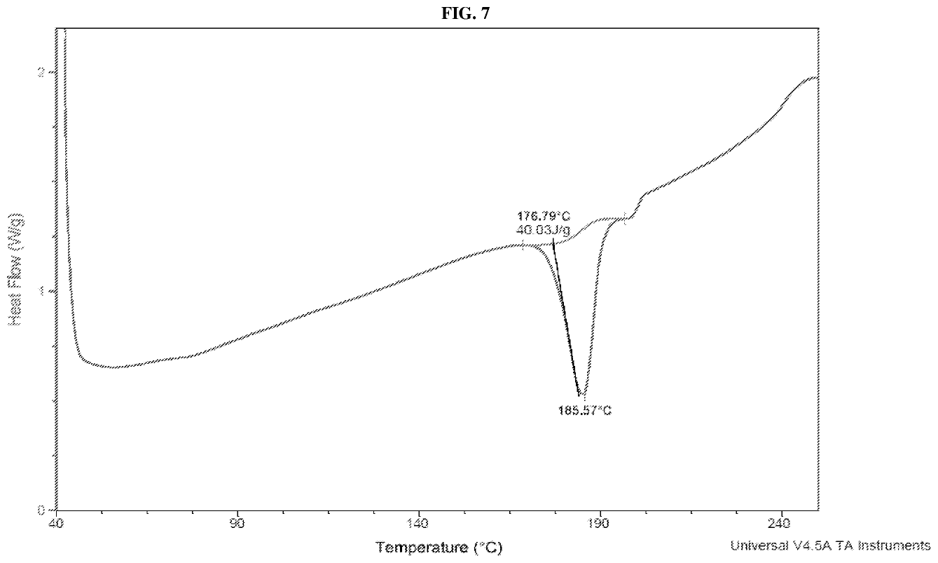

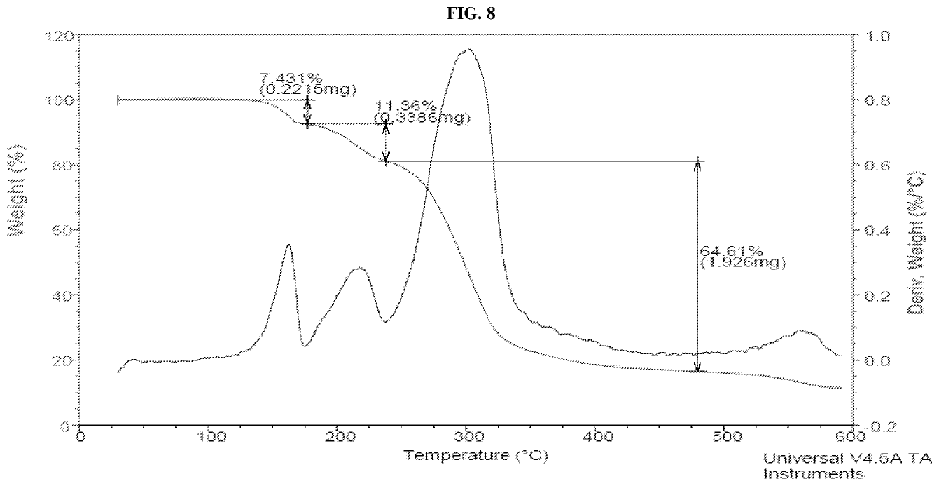

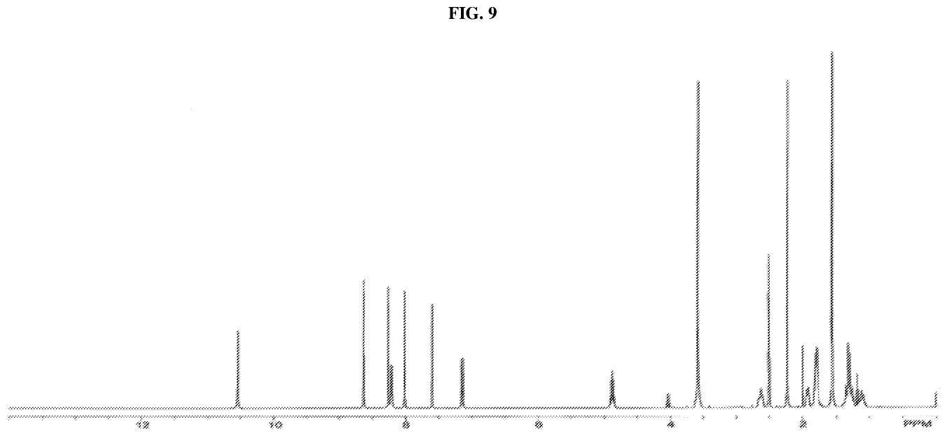

DSC analysis of this sample shows a melting onset at about 175° C. as shown in FIG. 7. TGA analysis of the crystalline Form II shows a degradation occurring after 176° C., as shown in FIG. 8. TGA analysis shows degradation to start between 120° C. and 140° C. FIG. 9 shows that ¹H NMR) analysis confirms the crystalline Form II only comprises the compound of Formula I and the crystal line Form II is more than 99% pure based on HPLC analysis. FIG. 6 shows the X-ray powder diffraction pattern for the crystalline Form II. Peak positions of the crystalline Form II are provided in Table 2.

TABLE 2

| Angle | d value |
|---|---|
| (2θ) | (Angstrom) |
| 7.3 | 12.0988 |
| 8.141 | 10.8517 |
| 9.101 | 9.709 |
| 10.279 | 8.5984 |
| 11.459 | 7.7155 |
| 13.079 | 6.7633 |
| 14.46 | 6.1205 |
| 15.4 | 5.7491 |
| 16.12 | 5.4939 |
| 16.98 | 5.2173 |
| 18.341 | 4.8331 |
| 19.159 | 4.6287 |
| 21.02 | 4.2229 |
| 21.679 | 4.0959 |
| 22.297 | 3.9838 |
| 23.099 | 3.8472 |
| 24.56 | 3.6216 |
| 25.341 | 3.5118 |
| 26.039 | 3.4191 |
| 27.459 | 3.2455 |
| 28.239 | 3.1576 |
| 29.139 | 3.0621 |
| 30.82 | 2.8988 |
| 31.597 | 2.8292 |
| 32.924 | 2.7182 |
| 33.419 | 2.6791 |
| 34.14 | 2.6241 |
| 36.34 | 2.4701 |
| 37.302 | 2.4086 |
| 38.479 | 2.3376 |
| 39.4 | 2.285 |
| 40.754 | 2.2122 |
| 43.841 | 2.0633 |

### Example 4

### Salt Screening of the Compound of Formula I

The salt screening of the compound of Formula I with coformer was carried out through the procedure as described herein. Pharmaceutically acceptable acids (including HCl, H₂SO₄, HBr, H₃PO₄, as shown in Table 3, were used for the salt screening. Crystalline Solids of the compound of Formula I with the respective succinate, adipic acid, fumaric acid, glutaric acid, gentisic acid, hydrochloric acid, 1-hydroxy-2-naphthoic acid, salicylic acid, oxalic acid and D-(−)-tartaric acid were obtained by mixing 0.05 mmol the compound of Formula I in free base (23.8 mg) with 1.05-1.10 equivalent of acids, respectively, as shown in Table 3.

TABLE 3

| Acid. | Results | Acid | Results |
|---|---|---|---|
| (+)-(1S)-CSA | Solution | L-(+)-Lactic Acid | Some solids |
| 1-hydroxy-2-Naphthoic | Crystalline Salts | L-(+)-Tartaric acid | Sticky solid |
| Acid, | | | |
| 1-Naphthalenesulphonic | Some solids | L-Pyroglutamic | Sticky solid |
| acid | | Acid | |
| Adipic acid | Crystalline Salts | L-Tartaric acid | Solution |
| Ascorbic acid | Sticky solid | Maleic acid | solution |
| Benzenesulfonic acid | Solution | Malic acid | solution |
| Benzoic acid | Solution | Malonic acid | Solution |
| Citric acid | Solution | Methanesulfonic | Sticky solid |
| | | acid | |
| D-Tartaric acid | Crystalline Salts | N-Acetyl-L- | Sticky solid |
| | | glutamic acid | |
| Ethane-1,2-disulfonic | Some solids | Nicotinic acid | Some solids |
| Acid | | | |
| Ethanesulfonic Acid | Solution | Oxalic acid | Crystalline Salts |
| Ethylsulfonic acid | Sticky solid | Phosphoric Acid | Solution |
| Fumaric acid | Crystalline Salts | R-(−)-Mandelic | Sticky solid |
| | | Acid | |
| Gentisic acid | Crystalline Salts | S-(+)-Mandelic | Sticky solid |
| | | Acid | |
| Gluconic acid | Solution | Saccharin | Sticky solid |
| Glutaric acid | Crystalline Salts | Salicylic acid | Crystalline Salts |
| Glycolic acid | Solution | Succinic acid | Crystalline Salts |
| HBr | Solution | Sulfuric acid | Solution |
| Hippuric acid | Solution | Toluenesulfonic | solution |
| | | acid hydrate | |
| Hydrochloric acid | Crystalline Salts | | |

### Example 5

Solvent Selection for Crystallization the Compound of Formula I with Pharmaceutically Acceptable Salts

Ten solvents were tested for the salt formation of the compound of Formula I with the pharmaceutically acceptable acids, as indicated in Table 4. The mixtures of the compound of Formula I and the respective acids as shown in Table 4 are individually screened against a list of solvents as listed in Table 4 and the results are shown in Table 4.

TABLE 4

| | Compound | Compound | Compound | Compound | Compound of |
|---|---|---|---|---|---|
| | of Formula I | of Formula I | of Formula I | of Formula I | Formula I |
| | and Succinic | and Fumaric | and Adipic | and Gentisic | and Glutaric |
| Solvent | acid | acid | acid | acid | acid |
| n-Butanol | Crystalline | Crystalline | Gel-like | Solution | Gel-like |
| (n-BuOH) | solids | solids | solids | | solids |
| Methyl ethyl | Crystalline | Gel-like | Gel-like | Solution | Gel-like |
| ketone | solids | solids | solids | | solids |
| (MEK) | | | | | |
| Methanol | Solution | Solution | Solution | Solution | Solution |
| Ethyl acetate | Crystalline | Gel-like | Gel-like | Solution | Gel-like |
| (EtOAc) | solids | solids | solids | | solids |
| Acetone | Crystalline | Gel-like | Gel-like | Solution | Gel-like |
| | solids | solids | solids | | solids |
| Tetrahydrofuran | Solution | Solution | Solution | Solution | Solution |
| (THF) | | | | | |
| isopropyl acetate | Crystalline | Gel-like | Gel-like | Some solids | Gel-like |
| (IPAc) | solids | solids | solids | | solids |
| Ethanol | Crystalline | Crystalline | Crystalline | Solution | Crystalline |
| (EtOH) | solids | solids | solids | | solids |
| Acetonitrile | Crystalline | Gel-like | Crystalline | Crystalline | Crystalline |
| (MeCN) | solids | solids | solids | solids | solids |
| 2-Propanol | Crystalline | Crystalline | Gel-like | Solution | Gel-like |
| (IPA) | solids | solids | solids | | solids |

### Example 6

### Preparation of the Crystalline Form III of the Compound of Formula I and Succinic Acid

Crystalline Form III of the compound of Formula I and succinic acid was obtained according to the procedure as follows:

| Steps | Operation |
|---|---|
| 1 | Added 119.5 mg of the compound of Formula I in free base |
| | (0.25 mmol, 1.0 eq.) |
| 2 | Added 4.0 mL of EtOAc and stirred for 2 minutes. |
| 3 | Added 32.5 mg (0.275 mmol, 1.1 eq.) of succinic acid. |
| 4 | Stirred continuously overnight |
| 5 | Filtered the slurry to give a solid |
| 6 | Dried the cake (40° C. under vacuum overnight) to get the |
| | crystalline Form III (130.3 mg, yielding 87.4%) |

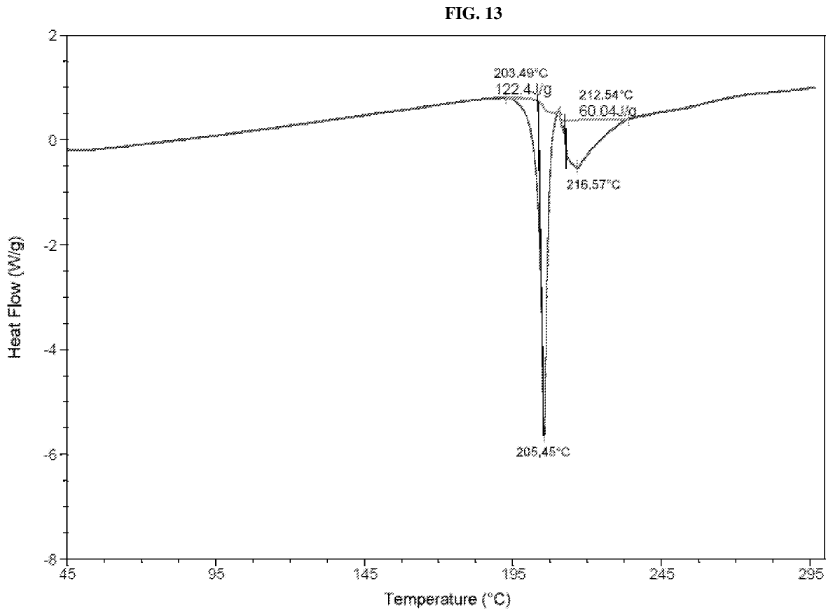

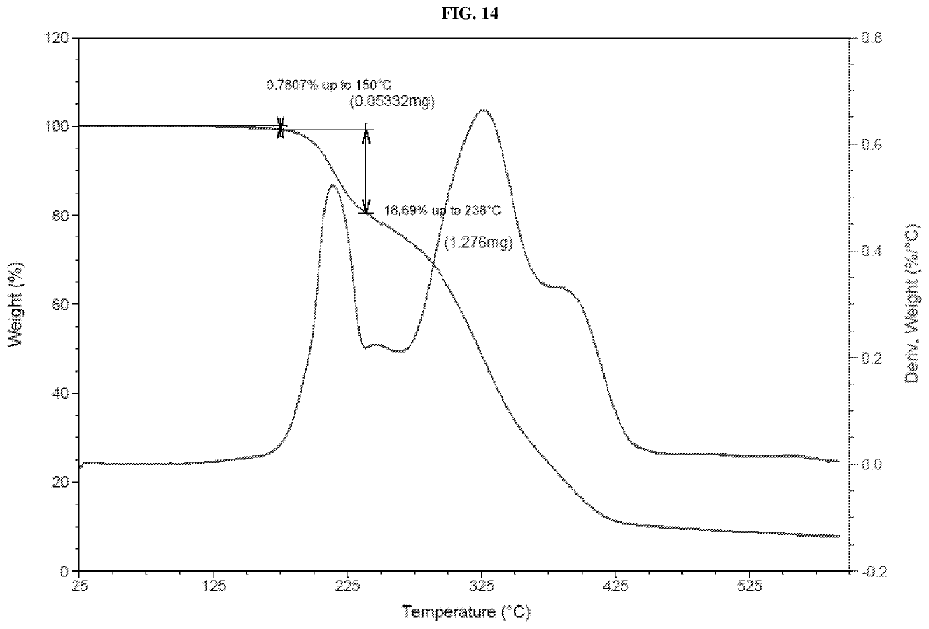

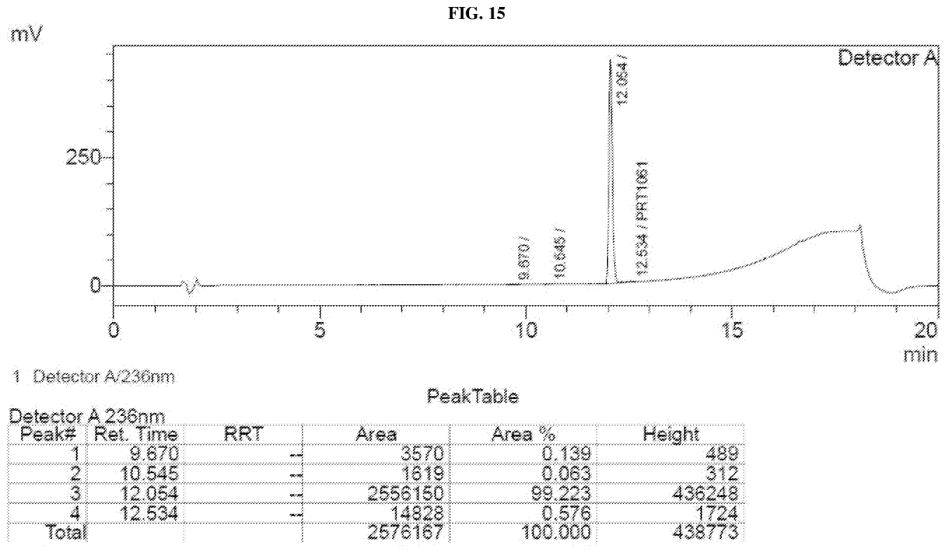

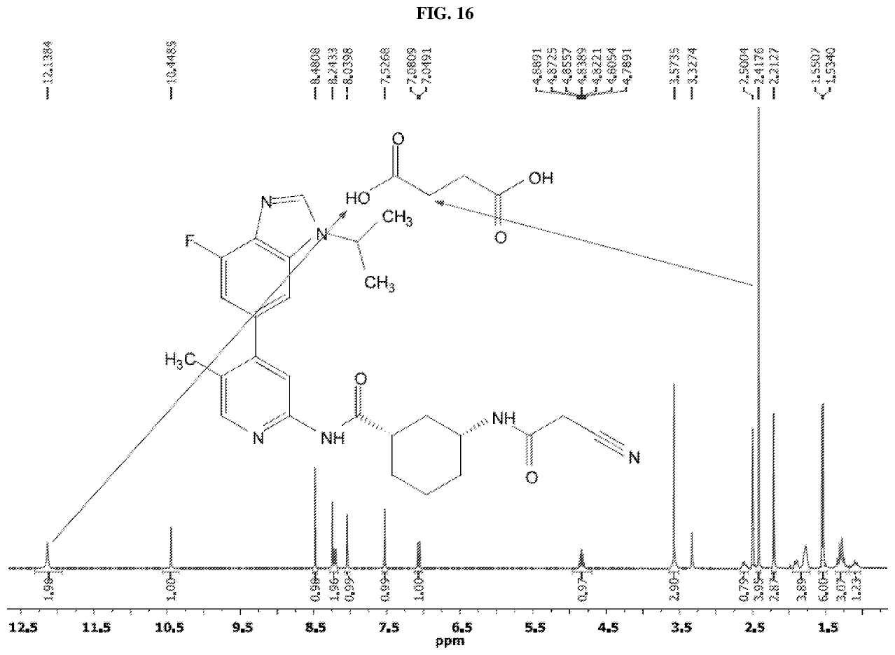

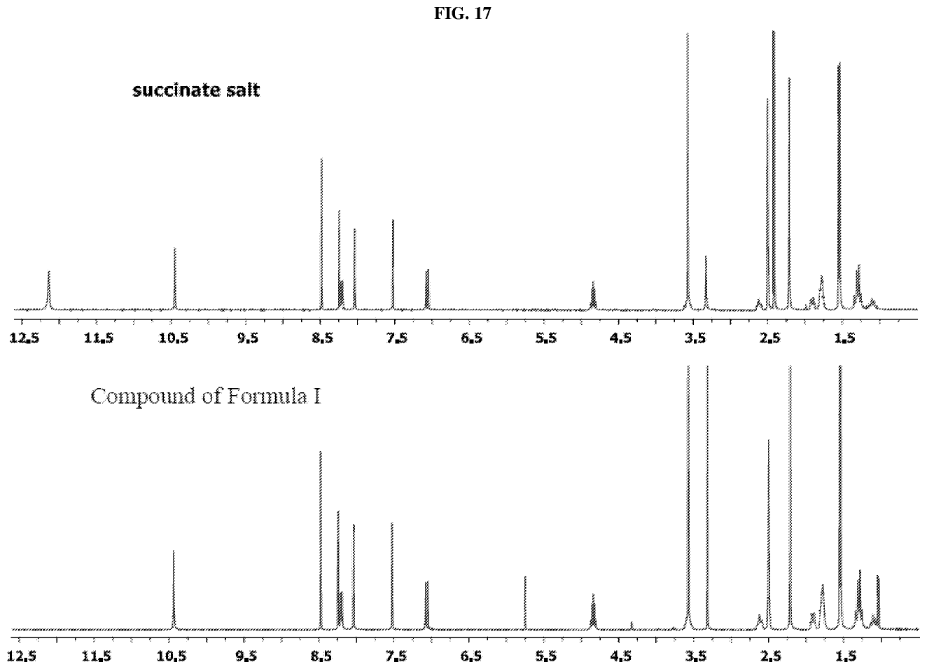

The crystallinity of the crystalline Form III was confirmed by XPRD as shown in FIG. 12. Its DSC and TGA were shown in FIGS. 13 and 14, respectively. The HPLC purity of the salt was 99.2% (FIG. 15), supported by its ¹H NMR spectrum (FIG. 16) and ¹H NMR comparison to that of the compound of Formula I (FIG. 17). The NMR of the compound of Formula I (FIG. 17) shows a distinct peak of NH connected to the pyridine ring at about 10.5 ppm. To determine the stoichiometric ratio of succinic acid to the compound of Formula I, the NMR signals at about 2.4 ppm (CH₂ of succinic acid), at about 12.1 ppm (COOH of succinic acid)), and at about 10.5 ppm (NH connected to pyridine ring of the compound of Formula I) are used. The quantitative ¹H NMR spectrum (FIG. 16) of the salt exhibited the stoichiometric ratio of the compound of Formula I and succinic acid was 1:1. FIG. 12 shows the X-ray powder diffraction pattern for the crystalline Form III. Peak positions of the crystalline Form III are provided in Table 5.

TABLE 5

| Angle | d value |
|---|---|
| (2θ) | (Angstrom) |
| 5.84 | 15.1206 |
| 8.8 | 10.0397 |
| 10.54 | 8.3864 |
| 12.38 | 7.1437 |
| 14.38 | 6.1542 |
| 17.54 | 5.0519 |
| 17.94 | 4.9403 |
| 18.501 | 4.7917 |
| 19.46 | 4.5577 |
| 19.98 | 4.4402 |
| 20.739 | 4.2795 |
| 21.359 | 4.1566 |
| 22.36 | 3.9727 |
| 22.7 | 3.9139 |
| 23.559 | 3.7732 |
| 23.96 | 3.7109 |
| 24.7 | 3.6014 |
| 25.92 | 3.4346 |
| 26.441 | 3.3681 |
| 27.581 | 3.2315 |
| 28.959 | 3.0807 |
| 29.42 | 3.0335 |
| 31.56 | 2.8325 |
| 33.542 | 2.6695 |
| 34.44 | 2.602 |
| 35.419 | 2.5322 |
| 36.177 | 2.4809 |
| 37.716 | 2.3831 |
| 39.498 | 2.2796 |
| 40.182 | 2.2424 |

### Example 7

### Preparation of the Crystalline Form IV of the Compound of Formula I and Glutaric Acid

Crystalline Form IV of the compound of Formula I and glutaric acid was obtained according to the procedure as follows:

| Steps | Operation |
|---|---|
| 1 | Added 119.9 mg of the compound of Formula I in free base |
| | (0.25 mmol, 1.0 eq.) |
| 2 | Added 4.0 mL of EtOAc and stirred for 2 minutes. |
| 3 | Added 36.3 mg (0.275 mmol, 1.1 eq.) of glutaric acid. |
| 4 | Stirred continuously overnight |
| 5 | Filtered the slurry to give a solid |
| 6 | Dried the cake (40° C. under vacuum overnight) to get the |
| | crystalline Form IV (120.7 mg, yielding 88.4%) |

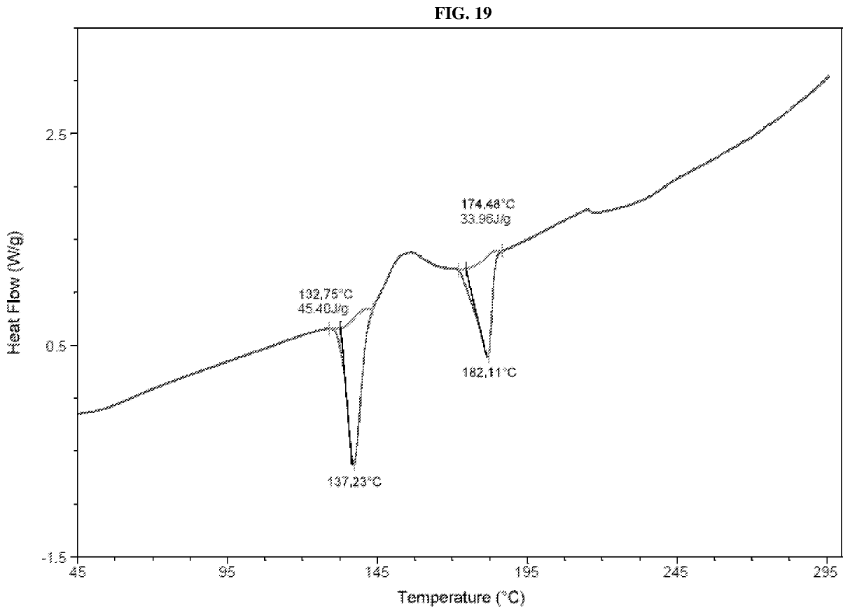

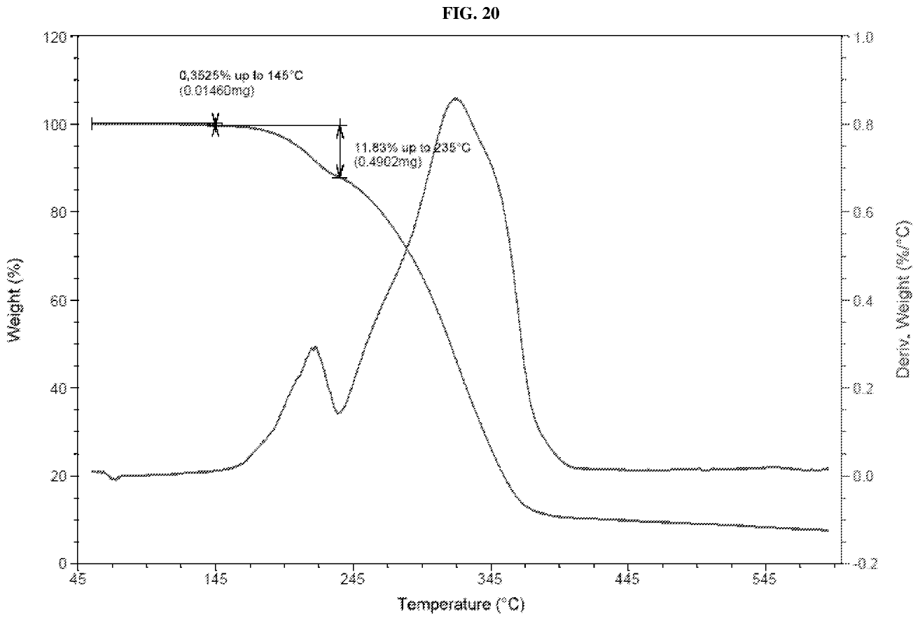

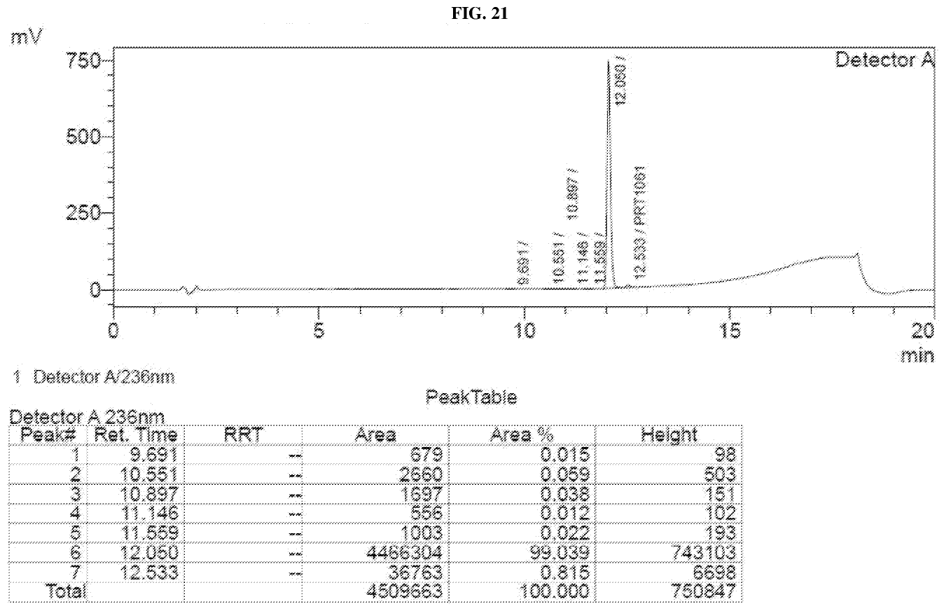

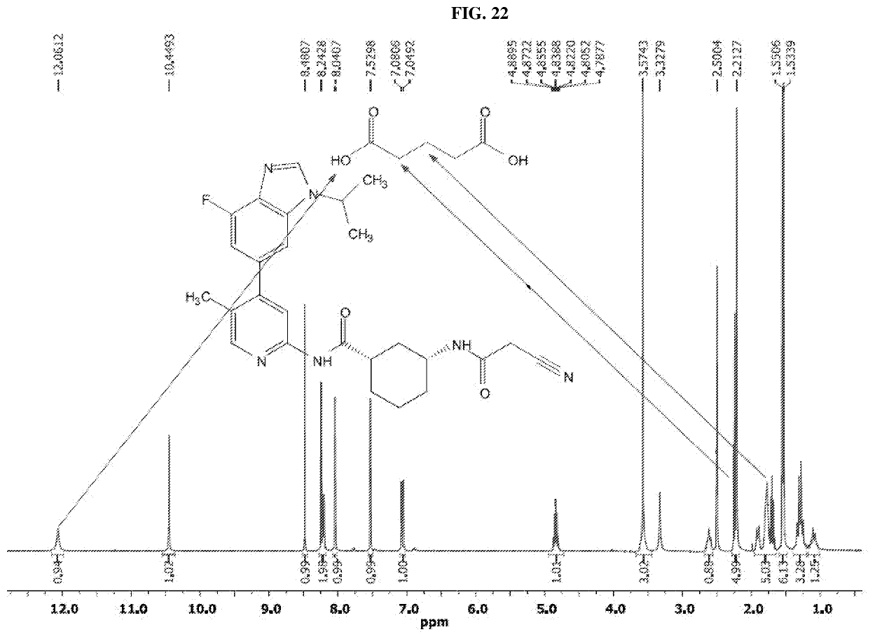

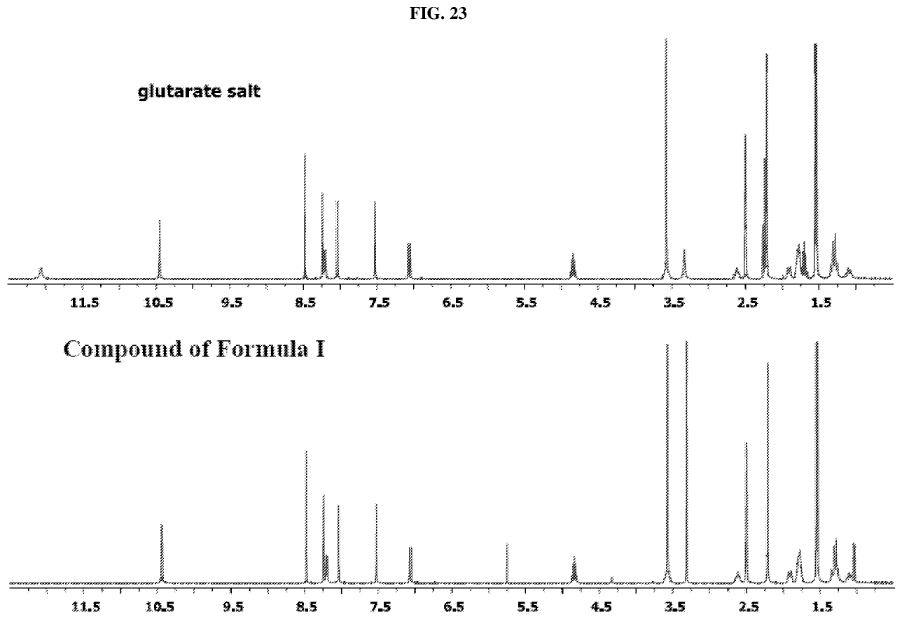

The crystallinity of the crystalline Form IV was confirmed by XPRD as shown in FIG. 18. Its DSC and TGA were shown in FIGS. 19 and 20, respectively. The HPLC purity of the salt was 99.0% (FIG. 21), supported by its ¹H NMR spectrum (FIG. 22) and ¹H NMR comparison to that of the compound of Formula I (FIG. 23). The NMR of the compound of Formula I (FIG. 23) shows a distinct peak of NH connected to pyridine ring at about 10.5 ppm. To determine the stoichiometric ratio of glutaric acid to the compound of Formula I, the NMR signals at about 12.1 ppm (COOH of glutaric acid) and at about 10.5 ppm (NH connected to pyridine ring of the compound of Formula I) are used. The quantitative ¹H NMR spectrum (FIG. 22) of the salt exhibited the stoichiometric ratio of the compound of Formula I and glutaric acid was 2:1. FIG. 18 shows the X-ray powder diffraction pattern for the crystalline Form IV. Peak positions of the crystalline Form IV are provided in Table 6.

TABLE 6

| Angle | d value |
|---|---|
| (2θ) | (Angstrom) |
| 4.54 | 19.4471 |
| 6 | 14.7189 |
| 7.14 | 12.3697 |
| 8.86 | 9.9721 |
| 11.139 | 7.9367 |
| 11.739 | 7.5321 |
| 13.22 | 6.6917 |
| 14.321 | 6.1796 |
| 15.423 | 5.7403 |
| 16.281 | 5.4399 |
| 17.119 | 5.1755 |
| 17.58 | 5.0408 |
| 18.381 | 4.8228 |
| 19.66 | 4.5118 |
| 20.479 | 4.3331 |
| 21.02 | 4.2229 |
| 21.88 | 4.0587 |
| 24.04 | 3.6987 |
| 24.68 | 3.6043 |
| 24.96 | 3.5645 |
| 25.461 | 3.4955 |
| 26.16 | 3.4036 |
| 26.981 | 3.3019 |
| 27.72 | 3.2155 |
| 29.16 | 3.0599 |
| 36.459 | 2.4623 |

### Example 8

### Preparation of the Crystalline Form V of the Compound of Formula I and Adipic Acid

Crystalline Form V of the compound of Formula I and adipic acid was obtained according to the procedure as follows:

| Steps | Operation |
|---|---|
| 1 | Added 120.0 mg of the compound of Formula I in free base |
| | (0.25 mmol, 1.0 eq.) |
| 2 | Added 4.0 mL of EtOH and stirred for 2 minutes. |
| 3 | Added 40.2 mg (0.275 mmol, 1.1 eq.) of adipic acid. |
| 4 | Stirred continuously overnight |
| 5 | Filtered the slurry to give a solid |
| 6 | Dried the cake (40° C. under vacuum overnight) to get the |
| | crystalline Form V (116.3 mg, yielding 74.2%) |

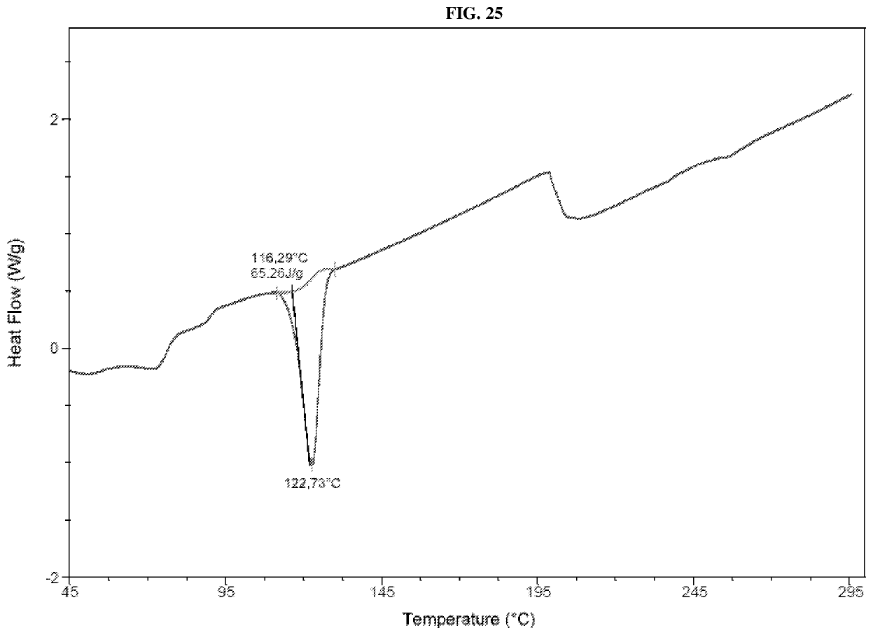

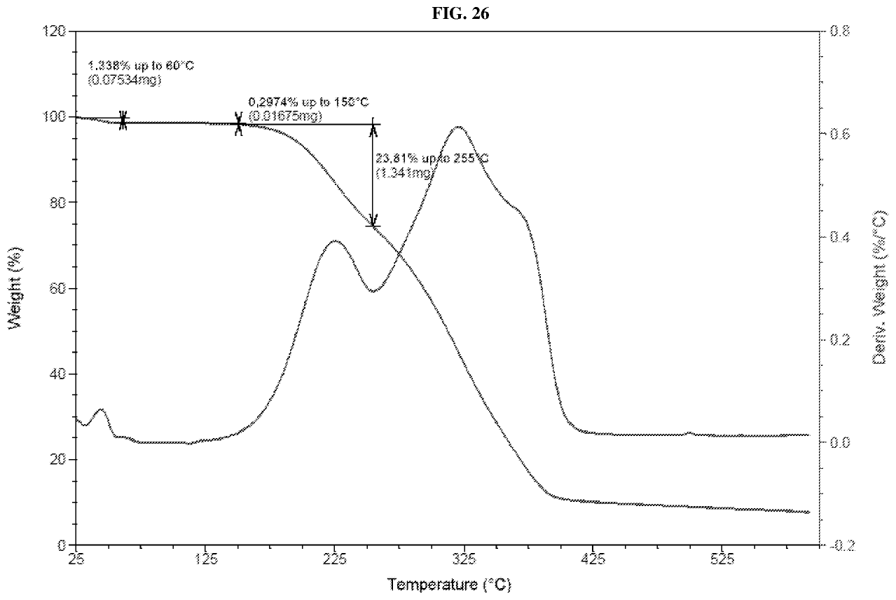

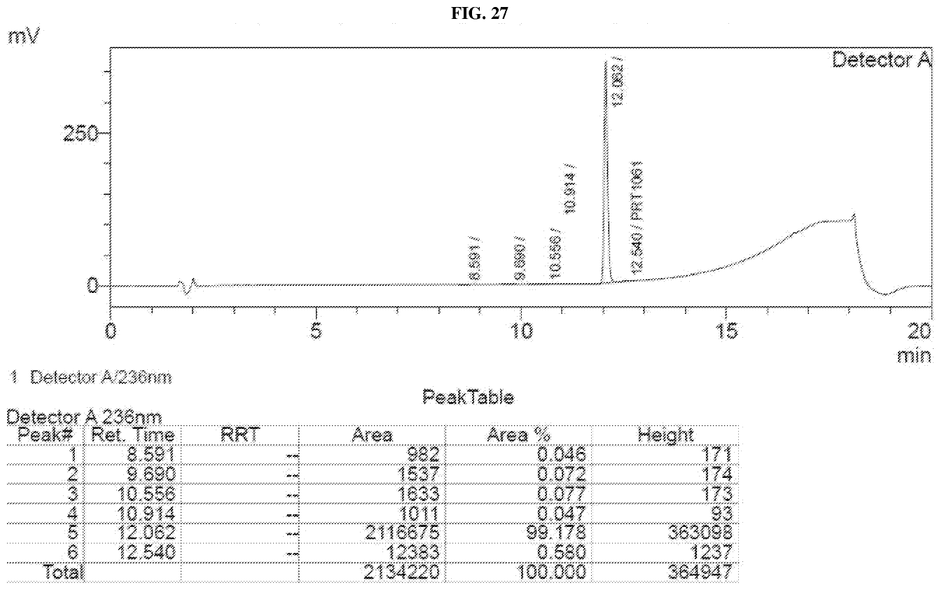

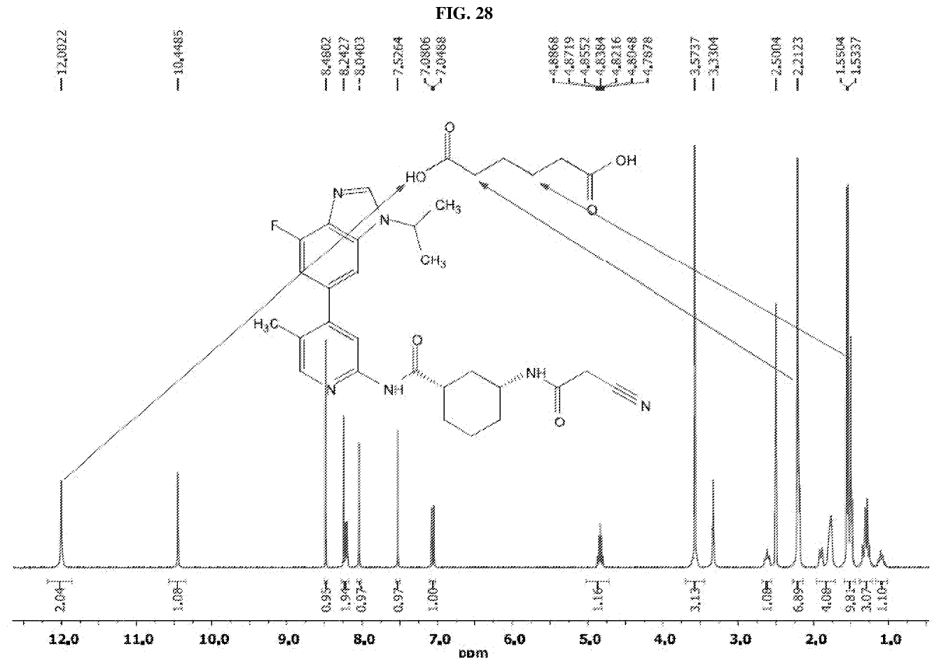

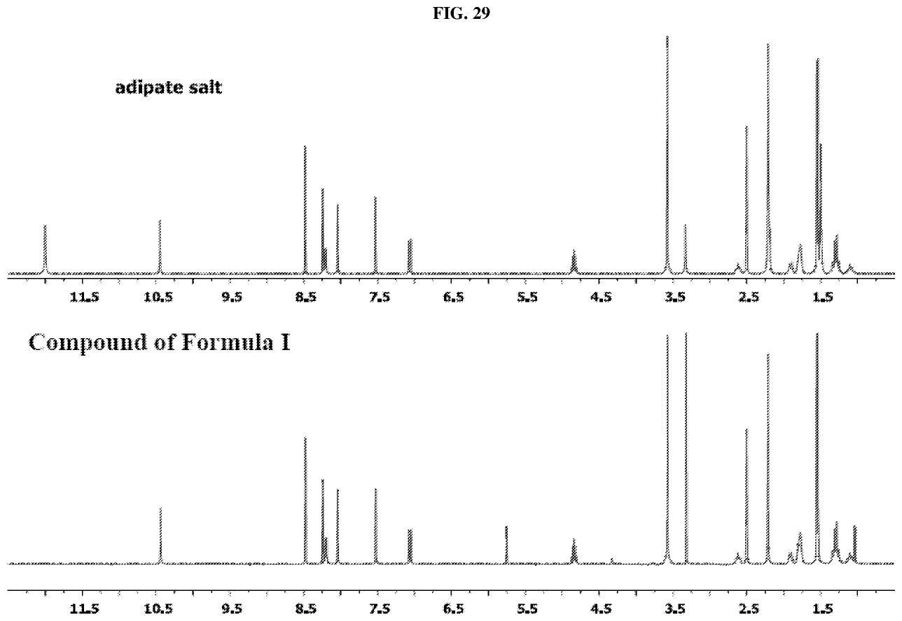

The crystallinity of the crystalline Form V was confirmed by XPRD as shown in FIG. 24. Its DSC and TGA were shown in FIGS. 25 and 26, respectively. The HPLC purity of the salt was 99.2% (FIG. 27), supported by its ¹H NMR spectrum (FIG. 28) and ¹H NMR comparison to that of the compound of Formula I (FIG. 29). The NMR of the compound of Formula I (FIG. 29) shows a distinct peak of NH connected to the pyridine ring at about 10.5 ppm. To determine the stoichiometric ratio of adipic acid to the compound of Formula I, the NMR signals at about 12.0 ppm (COOH of adipic acid) and at about 10.5 ppm (NH connected to pyridine ring of the compound of Formula I) are used. The quantitative ¹H NMR spectrum (FIG. 28) of the salt exhibited the stoichiometric ratio of the compound of Formula I and adipic acid was 1:1. FIG. 24 shows the X-ray powder diffraction pattern for the crystalline Form V. Peak positions of the crystalline Form V are provided in Table 7.

TABLE 7

| Angle | d value |
|---|---|
| (2θ) | (Angstrom) |
| 4.70 | 18.80 |
| 7.40 | 11.93 |
| 9.16 | 9.65 |
| 11.16 | 7.92 |
| 13.80 | 6.41 |
| 17.22 | 5.15 |
| 18.10 | 4.90 |
| 18.90 | 4.69 |
| 25.06 | 3.55 |
| 25.92 | 3.43 |

### Example 9

### Preparation of the Crystalline Form VI of the Compound of Formula I and Gentisic Acid

Crystalline Form VI of the compound of Formula I and gentisic acid was obtained according to the procedure as follows:

| Steps | Operation |
|---|---|
| 1 | Added 119.7 mg of the compound of Formula I in free base |
| | (0.25 mmol, 1.0 eq.) |
| 2 | Added 4.0 mL of acetonitrile and stirred for 2 minutes. |
| 3 | Added 42.4 mg (0.275 mmol, 1.1 eq.) of gentisic acid. |
| 4 | Stirred continuously overnight |
| 5 | Filtered the slurry to give a solid |
| 6 | Dried the cake (40° C. under vacuum overnight) to get the |
| | crystalline Form VI (125.8 mg, yielding 79.2%) |

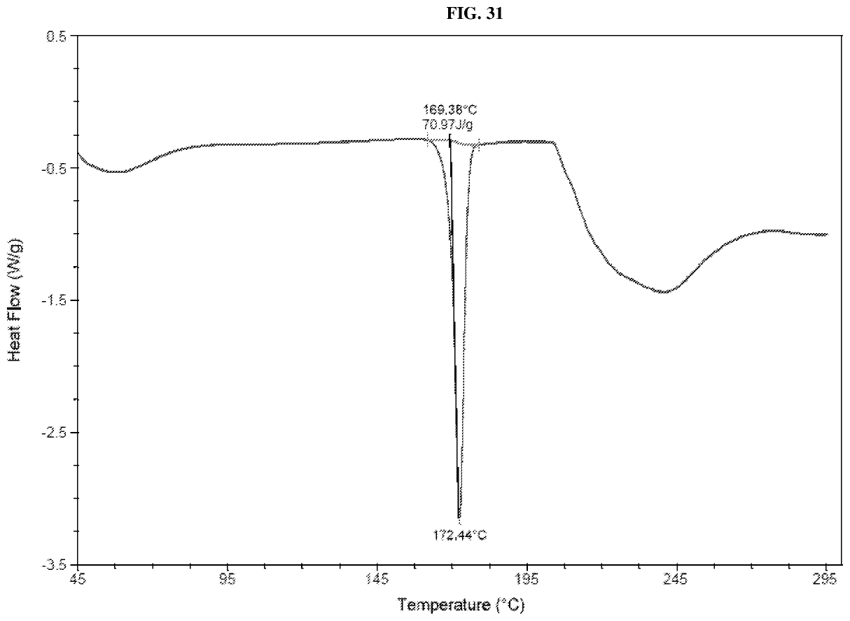

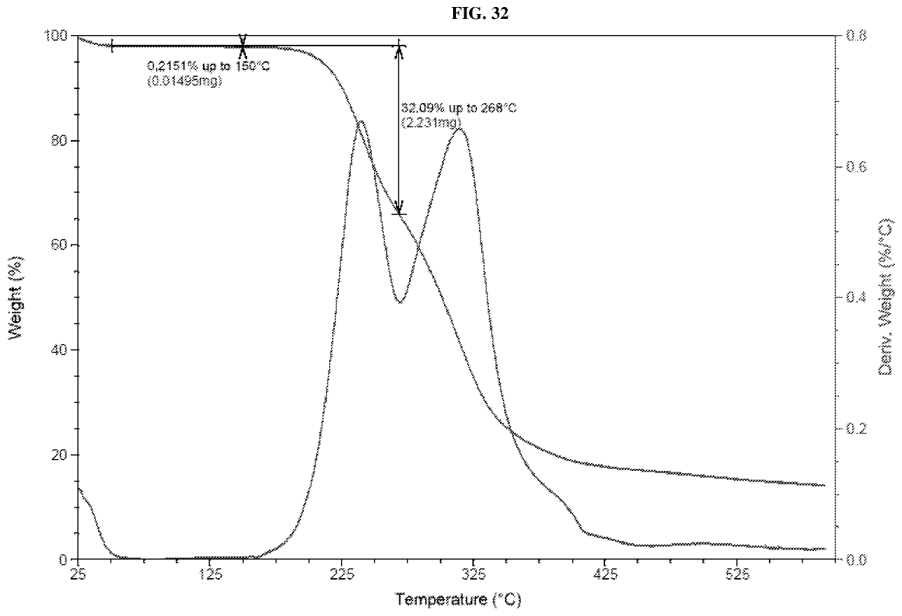

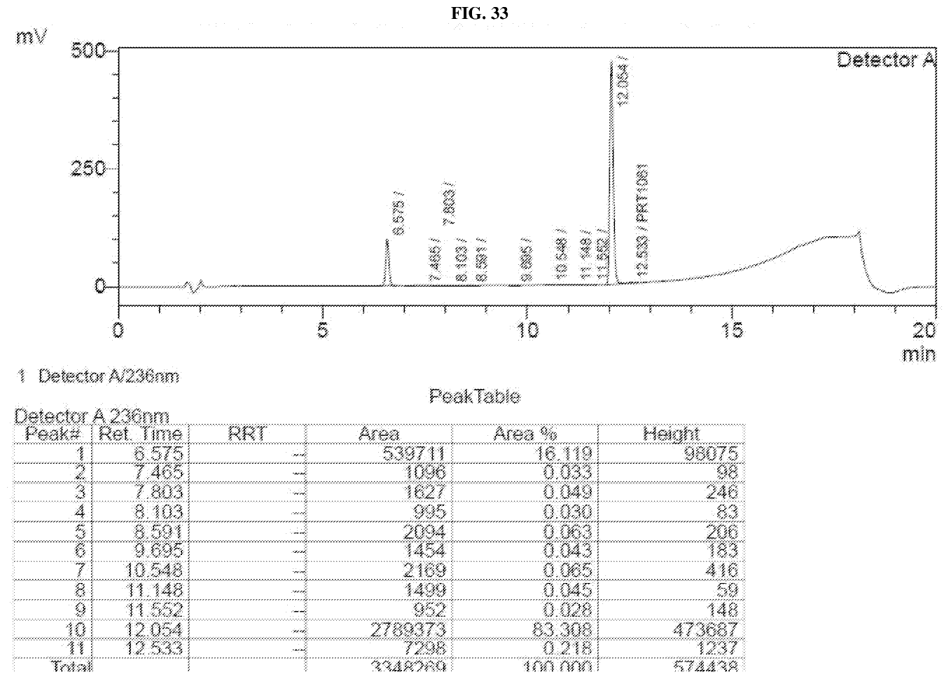

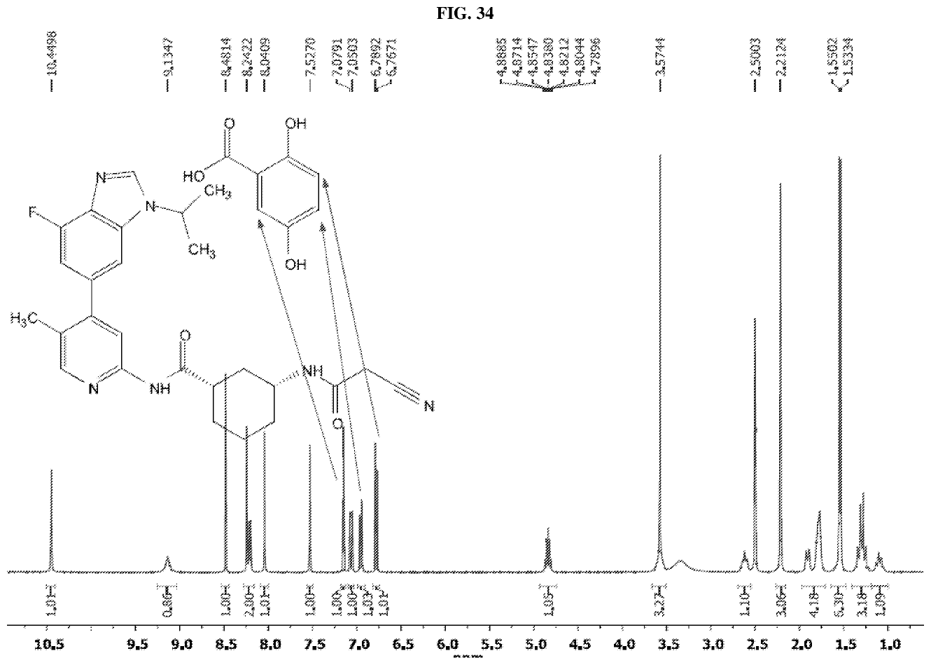

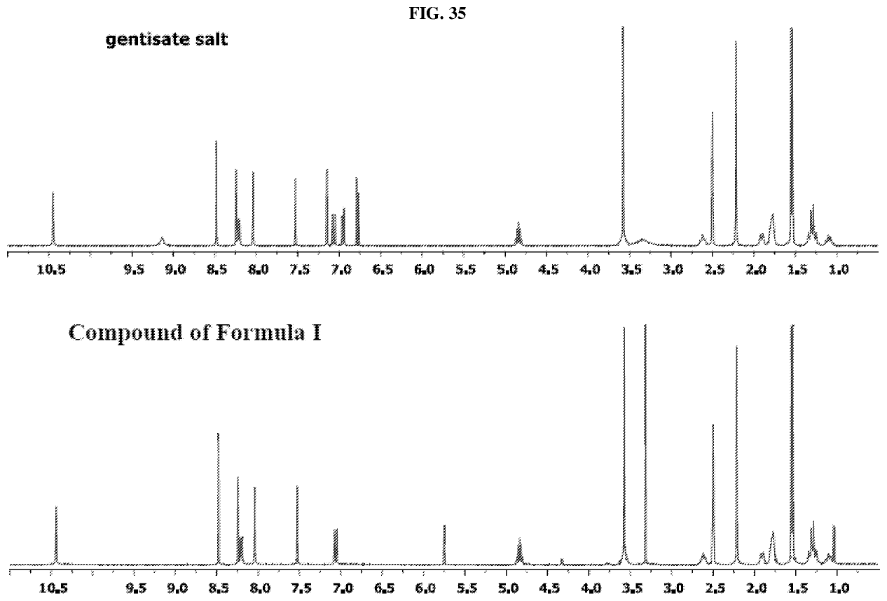

The crystallinity of the crystalline Form VI was confirmed by XPRD as shown in FIG. 30. Its DSC and TGA were shown in FIGS. 31 and 32, respectively. The HPLC purity of the salt was 99.4% (FIG. 33), supported by its ¹H NMR spectrum (FIG. 34) and ¹H NMR comparison to that of the compound of Formula I (FIG. 35). The NMR of the compound of Formula I (FIG. 34) shows a distinct peak of NH connected to pyridine ring at about 10.5 ppm. To determine the stoichiometric ratio of fumaric acid to the compound of Formula I, the NMR signals at about 6.8, 6.9, and 7.2 ppm (three hydrogens on the phenyl ring of fumaric acid) and at about 10.5 ppm (NH connected to pyridine ring of the compound of Formula I) are used. The quantitative ¹H NMR spectrum (FIG. 34) of the salt exhibited the stoichiometric ratio of the compound of Formula I and gentisic acid was 1:1. FIG. 30 shows the X-ray powder diffraction pattern for the crystalline Form VI. Peak positions of the crystalline Form VI are provided in Table 8.

TABLE 8

| Angle | d value |
|---|---|
| (2θ) | (Angstrom) |
| 3.98 | 22.17 |
| 7.36 | 12.00 |
| 9.52 | 9.28 |
| 10.96 | 8.07 |
| 13.56 | 6.52 |
| 14.66 | 6.04 |
| 15.52 | 5.70 |
| 16.36 | 5.41 |
| 17.20 | 5.15 |
| 18.18 | 4.88 |
| 18.76 | 4.73 |
| 19.40 | 4.57 |
| 20.48 | 4.33 |
| 21.54 | 4.12 |
| 23.54 | 3.78 |
| 24.82 | 3.58 |
| 25.70 | 3.46 |
| 26.88 | 3.31 |
| 28.12 | 3.17 |
| 29.36 | 3.04 |
| 30.72 | 2.91 |
| 31.32 | 2.85 |
| 32.82 | 2.73 |
| 35.68 | 2.51 |
| 37.46 | 2.40 |
| 38.86 | 2.32 |
| 40.00 | 2.25 |
| 43.24 | 2.09 |

### Example 10

### Preparation of the Crystalline Form VII of the Compound of Formula I and Fumaric Acid in a Molar Ratio of 2:1

Crystalline Form VII of the compound of Formula I and fumaric acid was obtained according to the procedure as follows:

| Steps | Operation |
|---|---|
| 1 | Added 119.5 mg of the compound of Formula I in free base |
| | (0.25 mmol, 1.0 eq.) |
| 2 | Added 4.0 mL of IPA and stirred for 2 minutes. |
| 3 | Added 32.0 mg (0.275 mmol, 1.1 eq.) of fumaric acid. |
| 4 | Stirred continuously overnight |
| 5 | Filtered the slurry to give a solid |
| 6 | Dried the cake (40° C. under vacuum overnight) to get the |
| | crystalline Form VII (96.3 mg, yielding 71.7%) |

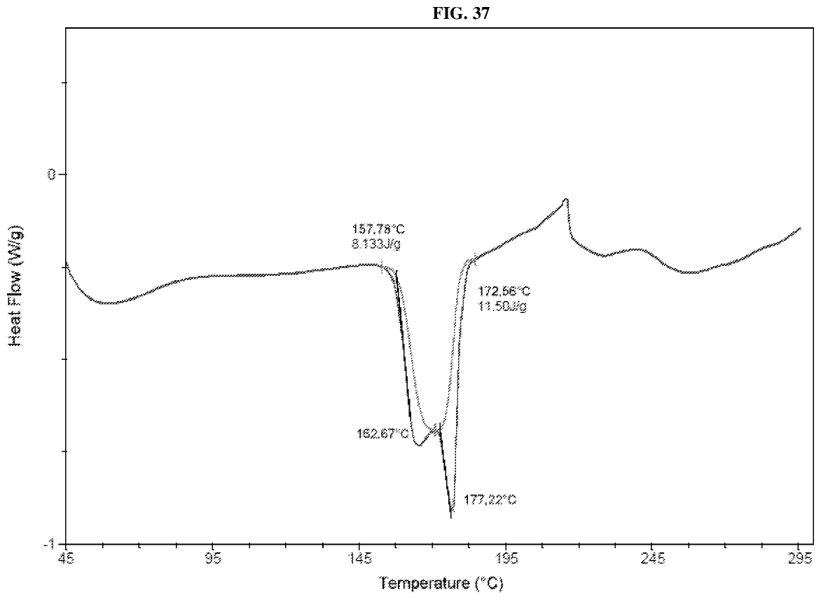

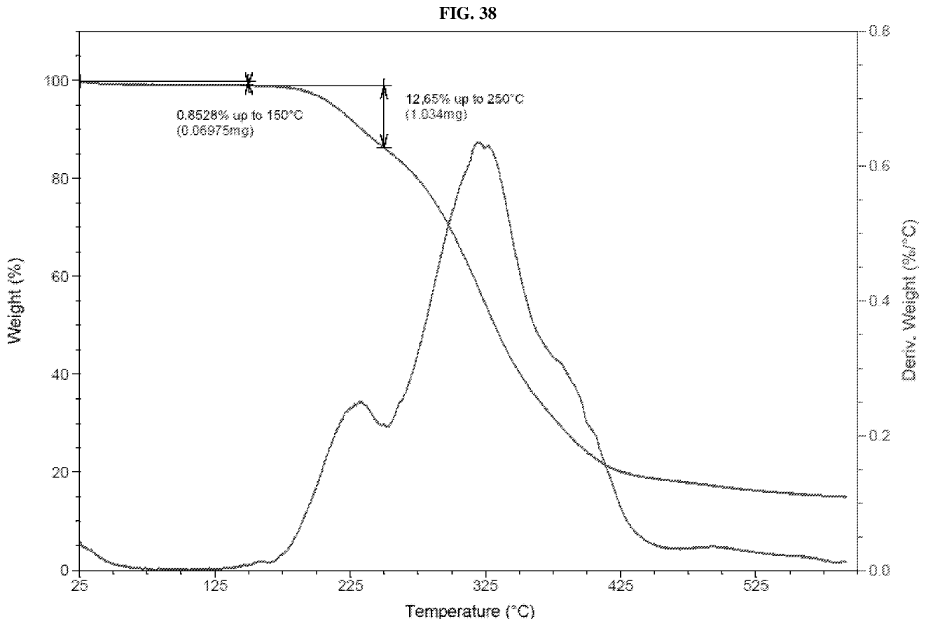

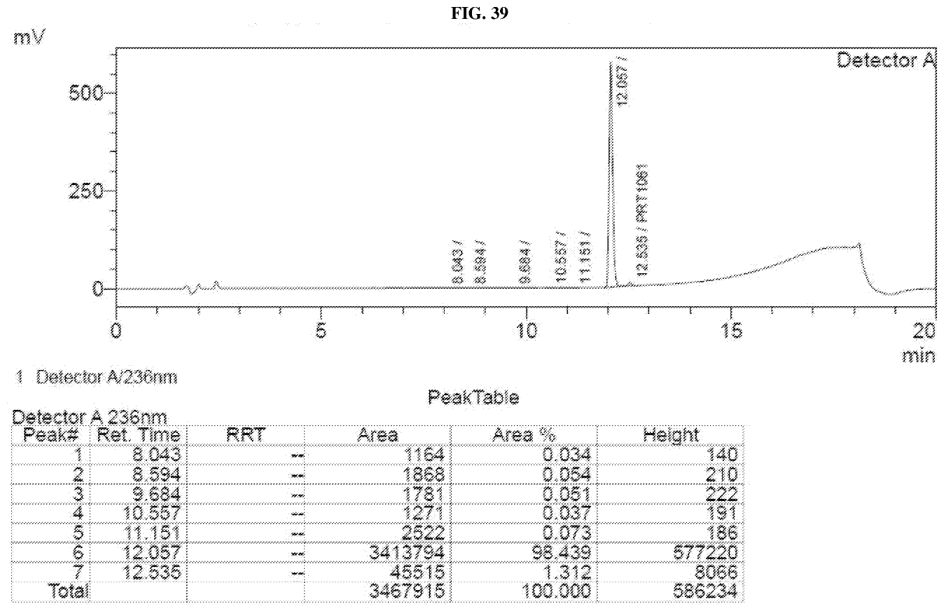

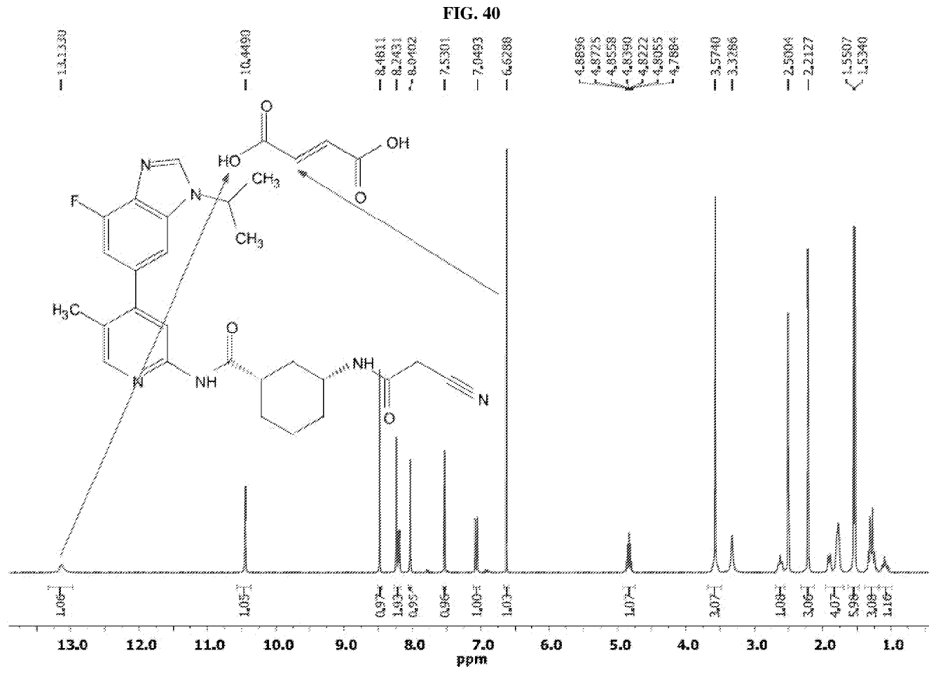

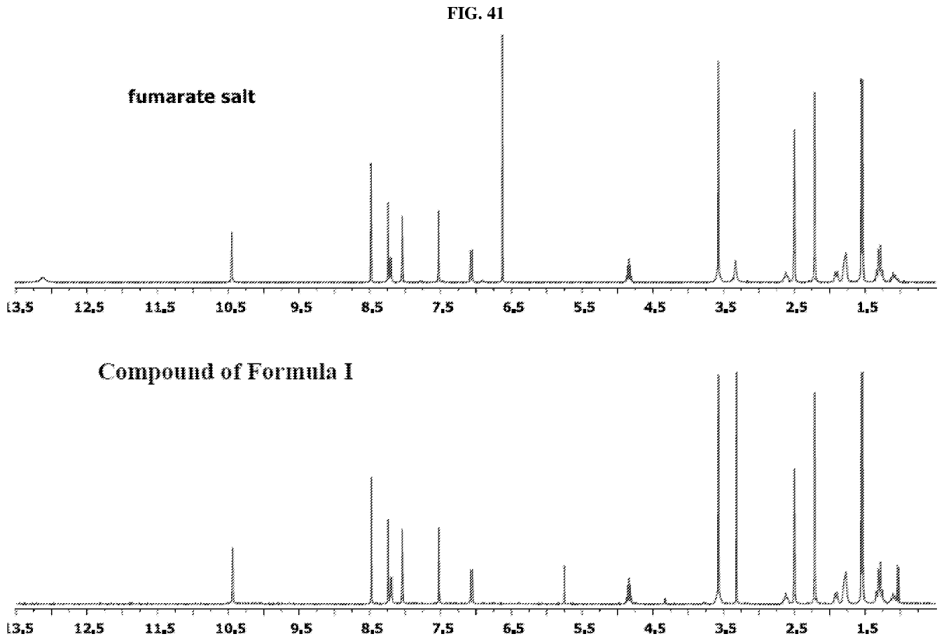

The crystallinity of the crystalline Form VII was confirmed by XPRD as shown in FIG. 36. Its DSC and TGA were shown in FIGS. 37 and 38, respectively. The HPLC purity of the salt was 98.6% (FIG. 39), supported by its ¹H NMR spectrum (FIG. 40) and ¹H NMR comparison to that of the compound of Formula I (FIG. 41). The NMR of the compound of Formula I (FIG. 40) shows a distinct peak of NH connected to pyridine ring at about 10.5 ppm. To determine the stoichiometric ratio of fumaric acid to the compound of Formula I, the NMR signals at about 13.1 ppm (COOH of fumaric acid) and at about 10.5 ppm (NH connected to pyridine ring of the compound of Formula I) are used. The quantitative ¹H NMR spectrum (FIG. 40) of the salt exhibited the stoichiometric/molar ratio of the compound of Formula I and fumaric acid was 2:1. FIG. 36 shows the X-ray powder diffraction pattern for the crystalline Form VII. Peak positions of the crystalline Form VII are provided in Table 9.

TABLE 9

| Angle | d value |
|---|---|
| (2θ) | (Angstrom) |
| 4.68 | 18.87 |
| 5.82 | 15.17 |
| 10.58 | 8.36 |
| 11.30 | 7.82 |
| 11.78 | 7.51 |
| 12.58 | 7.03 |
| 13.06 | 6.77 |
| 14.02 | 6.31 |
| 16.00 | 5.53 |
| 16.96 | 5.22 |
| 17.50 | 5.06 |
| 18.66 | 4.75 |
| 19.34 | 4.59 |
| 21.16 | 4.20 |
| 22.06 | 4.03 |
| 24.18 | 3.68 |
| 24.66 | 3.61 |
| 26.20 | 3.40 |
| 27.44 | 3.25 |

### Example 11

### Preparation of the Crystalline Form VIII of the Compound of Formula I and Fumaric Acid in a Molar Ratio of 1:1

Crystalline Form VIII of the compound of Formula I and fumaric acid was obtained according to the procedure as follows:

| Steps | Operation | Note |
|---|---|---|
| 1 | Added 118.40 mg of the compound of Formula I | |
| | in free base (0.248 mmol) | |
| 2 | Added 1.2 mL of IPA and stirred to give a clear solution | |
| 3 | Added fumaric acid (38.20 mg, 99.0%, 1.31 eq.) and stirred | slurry |
| 4 | Stirred at 65° C. for 3 min. | slurry |
| 5 | Stirred at 65° C. for 5.0 h. | |
| 6 | Cooled to room temperature and stirred overnight | |
| 7 | Added 2.0 mL MTBE and stirred for 2 min | |
| 8 | Filtered | |
| 9 | Dried the cake at 48-50° C. under vacuum | |
| | 24 h to get the crystalline Form VIII | |
| | (95.2 mg, 64.66%) | |

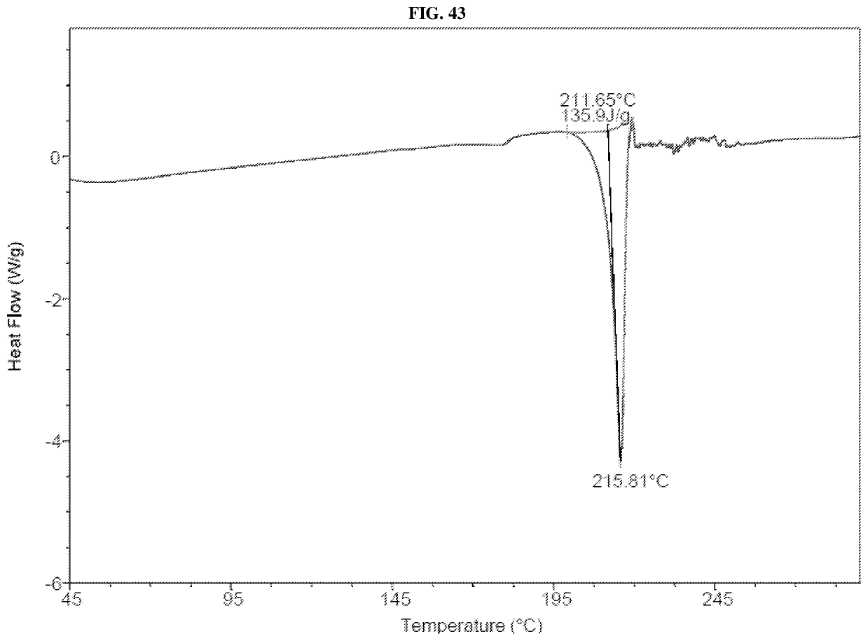

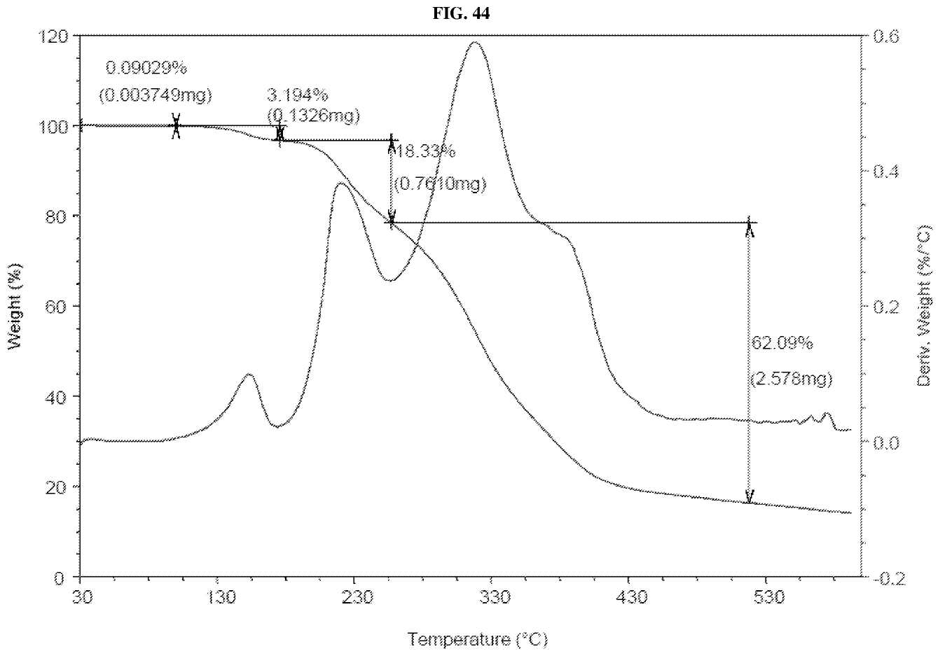

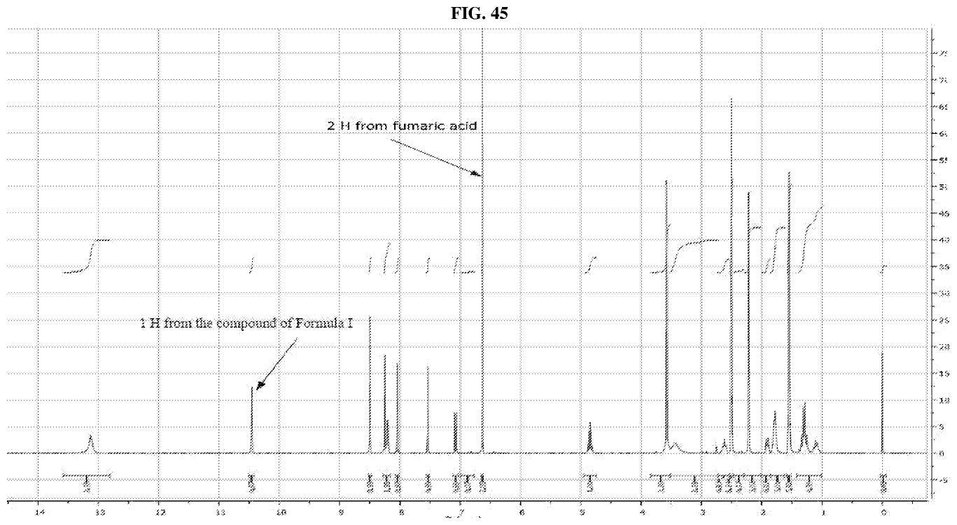

The crystallinity of the crystalline Form VIII was confirmed by XPRD as shown in FIG. 42, and further supported by DSC (FIG. 43), which indicated the salt with an onset temperature at 211.7° C. and a peak at 215.8° C., and TGA (FIG. 44). The stoichiometric/molar ratio of the crystalline Form VIII between the compound of Formula I in free base and fumaric acid was determined by ¹H NMR as 1:1 (FIG. 45). The HPLC purity of the salt was 98.2% as

Signal 1: VMDI A, Wavelength = 254 nm

| Peak | RetTime | | Width | Area | Height | Area |
|---|---|---|---|---|---|---|
| # | [min] | Type | [min] | mAU *s | [mAU] | % |
| 1 | 6.525 | MM | 0.0505 | 1.68496 | 5.56085e−1 | 0.0340 |
| 2 | 7.569 | MM | 0.0476 | 1.07964 | 3.78251e−1 | 0.0218 |
| 3 | 8.169 | MM | 0.0518 | 4.30856 | 1.38676 | 0.086 |
| 4 | 8.338 | VV | 0.0467 | 11.07680 | 3.60742 | 0.2234 |
| 5 | 8.539 | VV | 0.0565 | 4866.46826 | 1332.11731 | 98.1614 |
| 6 | 8.748 | VV | 0.0622 | 63.36488 | 14.63613 | 1.2781 |
| 7 | 9.144 | MM | 0.0820 | 1.94441 | 3.950720e−1 | 0.0092 |
| 8 | 9.712 | MM | 0.0730 | 6.35691 | 1.45100 | 0.1282 |
| 9 | 9.901 | MM | 0.0615 | 1.33512 | 3.621084e−1 | 0.0269 |
| Totals: | | | | 4957.61954 | 1354.89013 | |

indicated below supported by its ¹H NMR spectrum (FIG. 45). To determine the stoichiometric ratio of fumaric acid to the compound of Formula I, the NMR signals at about 6.63 ppm (CH of fumaric acid) and at about 10.5 ppm (NA connected to pyridine ring of the compound of Formula I) are used. The quantitative ¹H NMR spectrum (FIG. 45) of the salt exhibited the stoichiometric ratio of the compound of Formula I and fumaric acid was 1:1. FIG. 42 shows the X-ray powder diffraction pattern for the crystalline Form VIII. Peak positions of the crystalline Form VIII are provided in Table 10.

TABLE 10

| Angle | d value |
|---|---|
| (2θ) | (Angstrom) |
| 3.92 | 22.52 |
| 5.70 | 15.49 |
| 7.08 | 12.48 |
| 8.58 | 10.30 |
| 10.26 | 8.61 |
| 12.14 | 7.28 |
| 14.08 | 6.29 |
| 17.14 | 5.17 |
| 19.06 | 4.65 |
| 20.58 | 4.31 |
| 22.20 | 4.00 |
| 23.00 | 3.86 |
| 24.30 | 3.66 |
| 26.02 | 3.42 |
| 26.52 | 3.36 |
| 28.50 | 3.13 |
| 34.62 | 2.59 |
| 35.38 | 2.53 |
| 36.76 | 2.44 |
| 39.52 | 2.28 |

### Example 12

### Preparation of the Crystalline Form IX of the Compound of Formula I and D-(−)-Tartaric Acid

Crystalline Form IX of the compound of Formula I and D-(−)-tartaric-acid was obtained according to the procedure as follows:

| Steps | Operation | Note |
|---|---|---|
| 1 | Added 110.14 mg of the compound of Formula I in | |
| | free base (0.231 mmol, 1.0 eq) | |
| 2 | Added 1.2 mL of IPA and stirred at 50° C. | |
| | for 5 min. to give a clear solution | |
| 3 | Added D-(−)-tartaric acid at 50° C. | |
| | (41.63 mg, 99.0%, 1.20 eq, Sigma-Aldrich) | |
| 4 | Stirred at 55° C. for 10 min. to give a | |
| | thin slurry, 12 min to give a cream | |
| 5 | Stirred at 60° C. for 20 min. to give a slurry | slurry |
| 6 | Stirred at 75° C. for 6 h. | slurry |
| 7 | Stirred at 40° C. overnight | slurry |
| 8 | Cooled to room temperature | |
| 9 | Filtered and washed with MTBE (8.0 mL) | |
| 10 | Dried at 48-50° C. under vacuum 3 days the cake | |
| | to get the crystalline Form IX (138 mg, 95.29%) | |

The crystallinity of the crystalline Form IX was confirmed by XPRD as shown in

Signal 1: VWDI A, Wavelength = 254 nm

| Peak | RetTime | | Width | Area | Height | Area |
|---|---|---|---|---|---|---|
| # | [min] | Type | [min] | mAU *s | [mAU] | % |
| 1 | 6.473 | MM | 0.0483 | 2.58089 | 8.90366e−1 | 0.0682 |
| 2 | 7.560 | MM | 0.0522 | 1.13896 | 3.63844e−1 | 0.0301 |
| 3 | 8.197 | MM | 0.0556 | 1.76595 | 5.29404e−1 | 0.0467 |
| 4 | 8.383 | BV | 0.0482 | 7.02981 | 2.24502 | 0.1858 |
| 5 | 8.596 | MF | 0.0613 | 3737.65210 | 1015.62982 | 98.7960 |
| 6 | 8.817 | MF | 0.0693 | 27.99952 | 6.73454 | 0.7401 |
| 7 | 9.036 | FM | 0.0736 | 2.51551 | 5.69869e−1 | 0.0665 |
| 8 | 9.752 | MM | 0.0761 | 2.51770 | 5.51062e−1 | 0.0665 |
| Totals: | | | | 3783.20044 | 1027.51393 | |

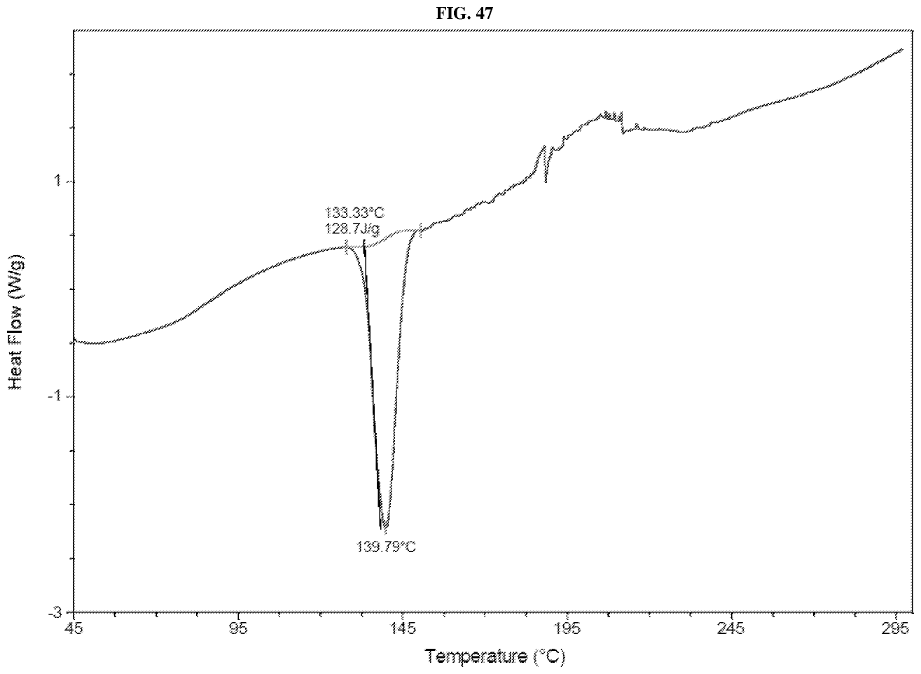

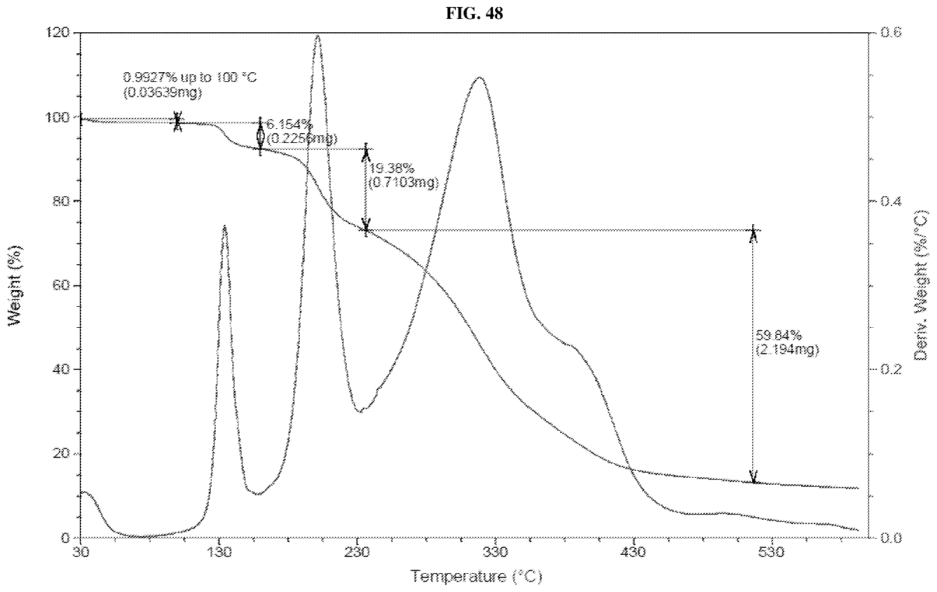

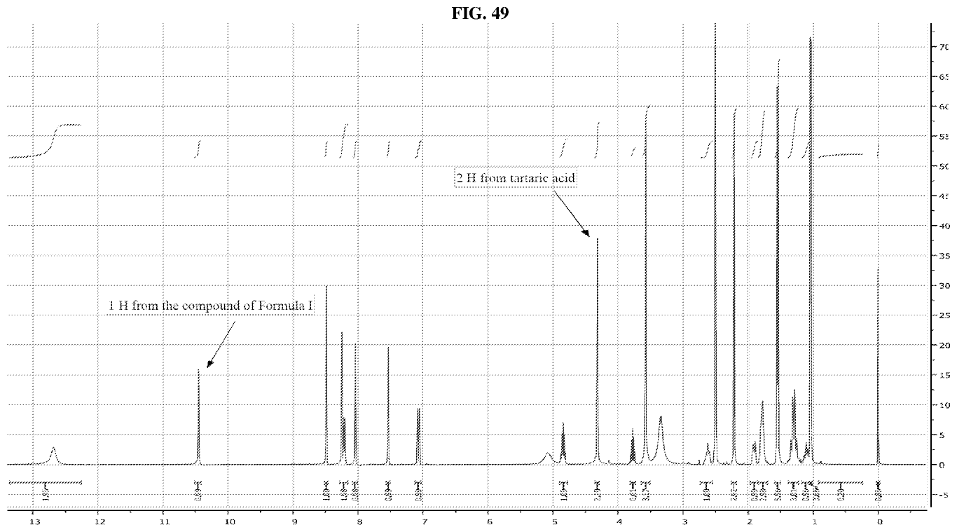

FIG. 46, and further supported by DSC (FIG. 47), which indicated the salt with an onset temperature at 133.3° C. and a peak at 139.8° C., and TGA (FIG. 48). The stoichiometric ratio of the crystalline Form IX between the compound of Formula I in free base and D-(−)-tartaric acid was determined by ¹H NMR as 1:1 (FIG. 49). The HPLC purity of the salt was 98.8% as indicated below supported by its ¹H NMR spectrum (FIG. 49). To determine the stoichiometric ratio of fumaric acid to the compound of Formula I, the NMR signals at about 6.63 ppm (CH of fumaric acid) and at about 10.5 ppm (NH connected to pyridine ring of the compound of Formula I) are used. The quantitative ¹H NMR spectrum (FIG. 49) of the salt exhibited the stoichiometric ratio of the compound of Formula I and fumaric acid was 1:1. FIG. 46 shows the X-ray powder diffraction pattern for the crystalline Form IX. Peak positions of the crystalline Form IX are provided in Table 11.

TABLE 11

| Angle | d value |
|---|---|
| (2θ) | (Angstrom) |
| 5.30 | 16.66 |
| 6.82 | 12.95 |
| 9.00 | 9.82 |
| 10.04 | 8.80 |
| 15.46 | 5.73 |
| 16.40 | 5.40 |
| 17.26 | 5.13 |
| 18.18 | 4.88 |
| 18.82 | 4.71 |
| 19.92 | 4.45 |
| 20.86 | 4.26 |
| 21.32 | 4.16 |
| 22.72 | 3.91 |
| 23.56 | 3.77 |
| 24.31 | 3.66 |
| 25.50 | 3.49 |
| 25.98 | 3.43 |
| 27.06 | 3.29 |
| 28.04 | 3.18 |
| 28.78 | 3.10 |
| 29.84 | 2.99 |
| 30.94 | 2.89 |
| 33.44 | 2.68 |
| 34.16 | 2.62 |
| 34.82 | 2.57 |
| 36.30 | 2.47 |
| 37.80 | 2.38 |
| 38.56 | 2.33 |
| 39.12 | 2.30 |
| 41.04 | 2.20 |
| 41.70 | 2.16 |

### Example 13

### Preparation of the Crystalline Form X of the Compound of Formula I and Hydrochloride Acid

Crystalline Form X of the compound of Formula I and hydrochloride acid was obtained according to the procedure as described herein. The compound of Formula I was mixed with hydrochloric acid in n-propanol, 2-butanone (MEK, methyl ethyl ketone), and n-butanol, respectively, to give the crystalline Form X with a yield of about 30%. The crystallinity of the crystalline Form X was confirmed by XPRD as shown in FIG. 50, and further supported by DSC

Signal 1: VWDI A, Wavelength = 254 nm

| Peak | RetTime | | Width | Area | Height | Area |
|---|---|---|---|---|---|---|
| # | [min] | Type | [min] | mAU *s | [mAU] | % |
| 1 | 6.520 | BB | 0.0505 | 17.14051 | 5.14774 | 0.4499 |
| 2 | 8.223 | MM | 0.0530 | 2.80121 | 8.81093e−1 | 0.0735 |
| 3 | 8.404 | VV | 0.0505 | 11.47131 | 3.44867 | 0.3011 |
| 4 | 8.617 | VV | 0.0566 | 3742.54614 | 1021.93909 | 98.2343 |
| 5 | 8.835 | VV | 0.0622 | 35.85658 | 8.27807 | 0.9412 |
| Totals: | | | | 3809.81575 | 1039.69466 | |

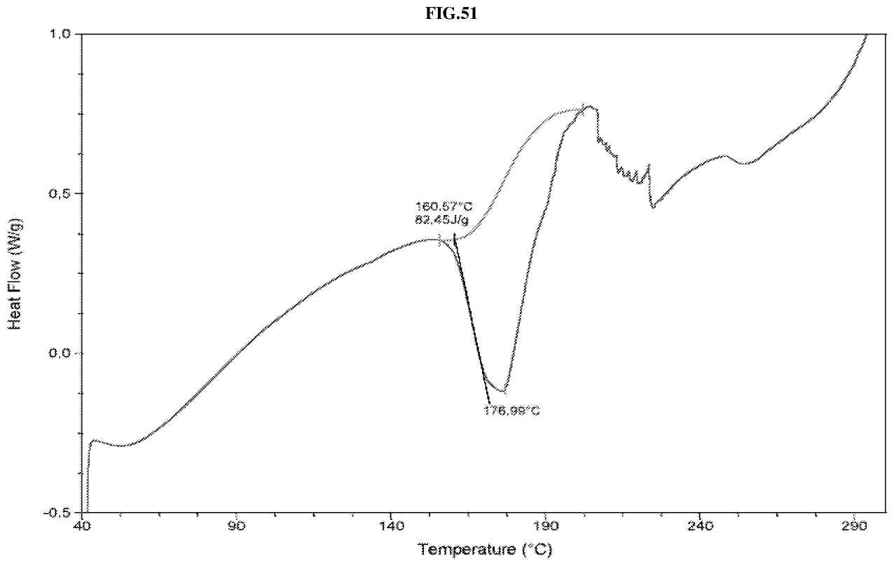

(FIG. 51), which indicated the salt with an onset temperature at 160.57° C. and a peak at 176.99° C. The HPLC purity of the salt was 98.2% as indicated below.

### Example 14

### Preparation of the Crystalline Form XI of the Compound of Formula I and Salicylic Acid

Signal 1: VWDI A, Wavelength = 254 nm

| Peak | RetTime | | Width | Area | Height | Area |
|---|---|---|---|---|---|---|
| # | [min] | Type | [min] | mAU *s | [mAU] | % |
| 1 | 6.473 | MM | 0.0483 | 2.58089 | 8.90366e−1 | 0.0682 |
| 2 | 7.560 | MM | 0.0522 | 1.13896 | 3.63844e−1 | 0.0301 |
| 3 | 8.197 | MM | 0.0556 | 1.76595 | 5.29404e−1 | 0.0467 |
| 4 | 8.383 | BV | 0.0482 | 7.02981 | 2.24502 | 0.1858 |
| 5 | 8.596 | MF | 0.0613 | 3737.65210 | 1015.62982 | 98.7960 |
| 6 | 8.817 | MF | 0.0693 | 27.99952 | 6.73454 | 0.7401 |
| 7 | 9.036 | FM | 0.0736 | 2.51551 | 5.69869e−1 | 0.0665 |
| 8 | 9.752 | MM | 0.0761 | 2.51770 | 5.51062e−1 | 0.0665 |
| Totals: | | | | 3783.20044 | 1027.51393 | |

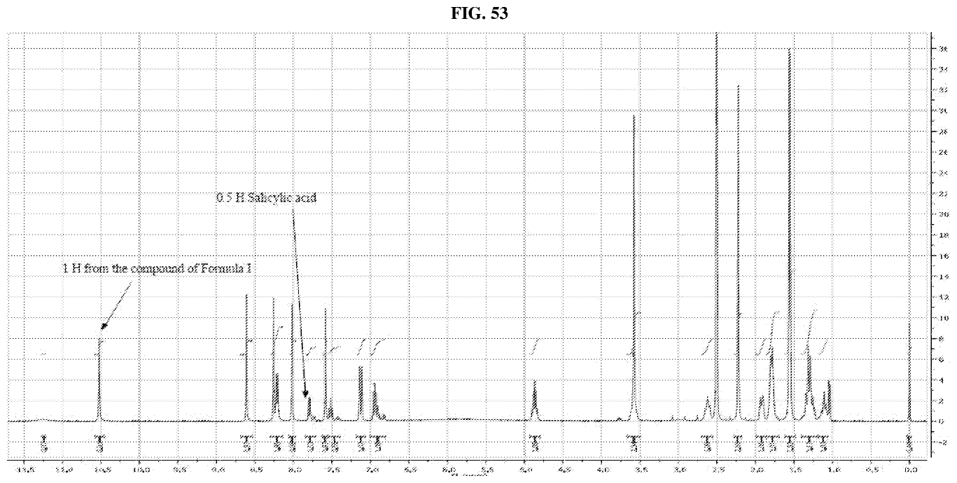

Crystalline Form XI of the compound of Formula I and salicylic acid was obtained according to the procedure as described herein. The compound of Formula I was reacted with salicylic acid in 2-propanol to give the crystalline Form XI. The crystallinity of the crystalline Form XI was confirmed by XPRD as shown in FIG. 52. To determine the stoichiometric ratio of fumaric acid to the compound of Formula I, the NMR signals at about 7.8 ppm (CH on the phenyl ring of salicylic acid) are used as shown in FIG. 53. The quantitative ¹H NMR spectrum (FIG. 53) of the salt exhibited the stoichiometric ratio of the compound of Formula I to salicylic acid was 2:1.

### Example 15

### Preparation of the Crystalline Form XII of the Compound of Formula I and Oxalic Acid

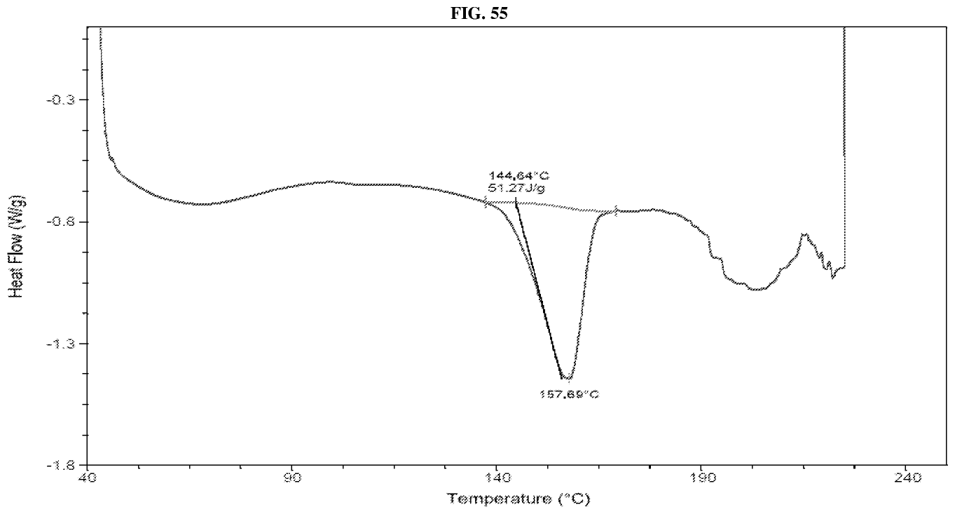

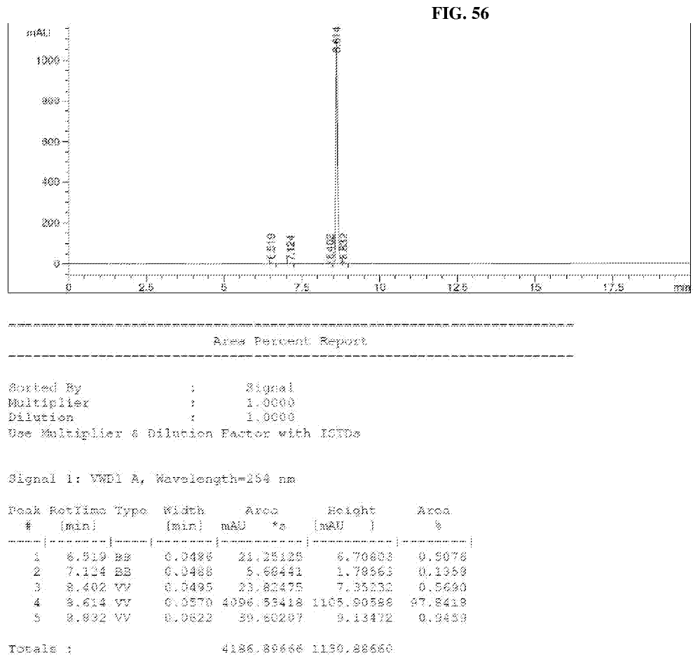

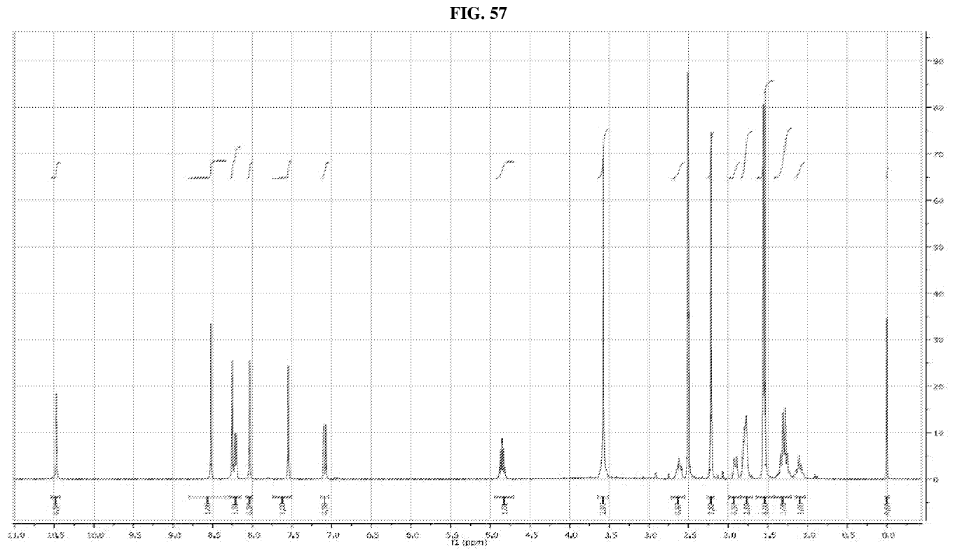

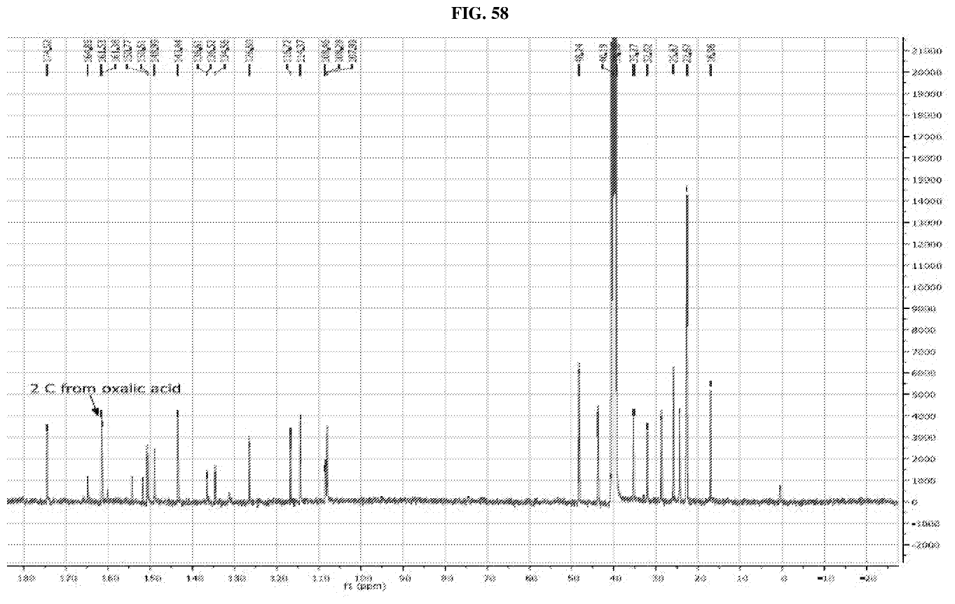

Crystalline Form XII of the compound of Formula I and oxalic acid was obtained according to the procedure as described herein. The compound of Formula I was reacted with oxalic acid in 2-butanone (MEK) to give the crystalline Form XII with a yield of about 70%. The crystallinity of the crystalline Form XII was confirmed by XPRD as shown in FIG. 54 and further supported by DSC (FIG. 55), which indicated the salt with an onset temperature at 144.6° C. and a peak at 157.7° C. The structure was characterized by ¹H NMR (FIG. 57) and ¹³C NMR (FIG. 58), and the purity was analyzed by HPLC (FIG. 56). To determine the stoichiometric ratio of the compound of Formula I to oxalic acid, elemental analysis was used. The result of elemental analysis indicated the crystalline Form XII is a mono-oxalate hydrate: elemental analysis: Calculated for C₂₈H₃₁FN₆O₆·H₂O.: C, 57.53; H, 5.69; N, 14.38. Found: C, 57.32; H, 5.41; N, 13.77. This result showed the stoichiometric ratio of the compound of Formula I to oxalic acid in the crystalline Form XII is 1:1.

### Example 16

### Preparation of the Crystalline Form XIII of the Compound of Formula I and 1-Hydroxy-2-Naphthoic Acid

Crystalline Form XIII of the compound of Formula I and 1-hydroxy-2-naphthoic acid was obtained according to the procedure as described herein. The compound of Formula I was reacted with 1-hydroxy-2-naphthoic acid in methanol to give the crystalline Form XIII. The crystallinity of the crystalline Form XIII was confirmed by XPRD as shown in FIG. 59.

### Example 17

### XRPD Analysis

X-ray powder diffraction (XRPD) measurements were performed with a Rigaku MiniFlex X-ray Powder Diffractometer (XRPD) instrument. The general experimental procedures for XRPD were: (1) X-ray radiation from copper at 1.54056 Å with K filter; (2) X-ray power at 30 KV, 15 mA; and (3) the sample powder was dispersed on a zero-background sample holder. The general measurement conditions for XRPD were: Start Angle 3 degrees; Stop Angle 45 degrees; Sampling 0.02 degrees; and Scan speed 2 degree/min.

### Example 18

### Differential Scanning Calorimetric Analysis (DSC)

Crystalline forms of the compound of Formula I were analyzed using Differential Scanning Calorimetry. DSC measurements Differential Scanning Calorimetry (DSC) was carried out on a TA Instruments Differential Scanning Calorimetry, Model Q200 with autosampler. The DSC instrument conditions were as follows: 20-300° C. at 10° C./min; Tzero aluminum sample pan and lid; and nitrogen gas flow at 50 mL/min.

### Example 19

### Thermogravimetric Analysis (TGA) Thermogram

Crystalline forms of the compound of Formula I were analyzed using a TA Instruments Thermogravimetric Analyzer, TGA Q500 with autosampler. The general experimental conditions for TGA were ramp from 25° C. 600° C. at 20° C./min; nitrogen purge, gas flow at 25 mL/min; platinum sample pan.

### Example 20

### High-Performance Liquid Chromatography (HPLC)

Crystalline forms of the compound of Formula I were analyzed with High-performance liquid chromatography for their purities. The general experimental conditions for HPLC are as follows:

| Instrument | Agilent 1100 | |
|---|---|---|
| Column | Zorbax SB-C18, 3.5 μm, 4.6 × 150 mm | |
| Column Temperature | 40° | C. |
| Mobile Phase A | 0.05% TFA in water | |
| Mobile Phase B | 0.05% TFA In acetonitrile | |
| Flow Rate | 1 | mL/min |
| Injection Volume | 5 | μL |
| Total Run Time | 20 | min |
| UV Detector Wavelength | 254 | nm |

Gradient Table

| Time (min) | Mobile Phase A | Mobile Phase B |
|---|---|---|
| 0 | 95 | 5 |
| 20 | 5 | 95 |

What is claimed is:

1. A crystalline form of a compound having a formula of

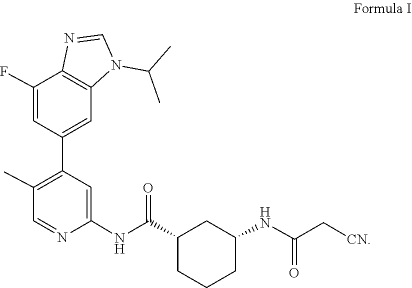 [SMILES: Cc1cnc(NC(=O)[C@H]2CCC[C@@H](NC(=O)CC#N)C2)cc1-c1cc(F)c2ncn(C(C)C)c2c1]

2. A crystalline form of a compound having a formula of 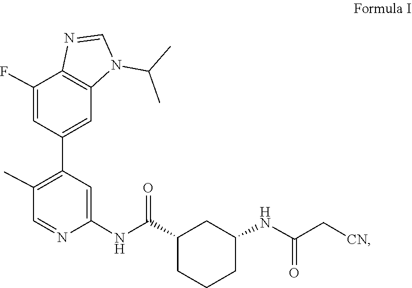 [SMILES: Cc1cnc(NC(=O)[C@H]2CCC[C@@H](NC(=O)CC#N)C2)cc1-c1cc(F)c2ncn(C(C)C)c2c1]
wherein the crystalline form is Form I.

3. The crystalline form of claim 2, wherein the crystalline Form I is characterized by an X-ray powder diffraction pattern comprising one or more peaks at about 7.2±0.5 degrees 2θ, at about 8.0±0.5 degrees 2θ, at about 10.1±0.5 degrees 2θ, at about 11.3±0.5 degrees 2θ, at about 13.0±0.5 degrees 2θ, at about 14.4±0.5 degrees 2θ, at about 15.3±0.5 degrees 2θ, at about 16.8±0.5 degrees 2θ, at about 18.2±0.5 degrees 2θ, at about 20.9±0.5 degrees 2θ, at about 21.6±0.5 degrees 2θ, at about 22.2±0.5 degrees 2θ, and at about 23.1±0.5 degrees 2θ.

4. The crystalline form of claim 2, wherein the crystalline Form I is characterized by an X-ray powder diffraction pattern comprising one or more peaks as shown in FIG. 2.

5. The crystalline form of claim 2, wherein the crystalline Form I is characterized by an X-ray powder diffraction pattern comprising one or more d-spacing values at about 12.3±0.5 degrees angstroms, at about 11.0±0.5 degrees angstroms, at about 8.7±0.5 degrees angstroms, at about 7.8±0.5 degrees angstroms, at about 6.8±0.5 degrees angstroms, at about 6.2±0.5 degrees angstroms, at about 5.8±0.5 degrees angstroms, at about 5.3±0.5 degrees angstroms, at about 4.9±0.5 degrees angstroms, at about 4.3±0.5 degrees angstroms, at about 4.1±0.5 degrees angstroms, at about 4.0±0.5 degrees angstroms, and at about 3.8±0.5 degrees angstroms.

6. A crystalline form of a compound having a formula of 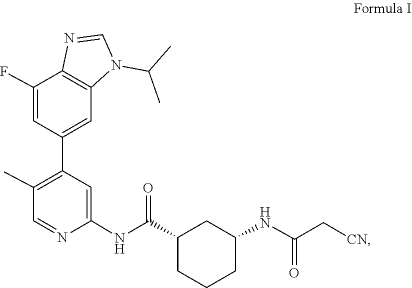 [SMILES: Cc1cnc(NC(=O)[C@H]2CCC[C@@H](NC(=O)CC#N)C2)cc1-c1cc(F)c2ncn(C(C)C)c2c1]
wherein the crystalline form is Form II.

7. The crystalline form of claim 6, wherein the crystalline Form II is characterized by an X-ray powder diffraction pattern comprising one or more peaks at about 7.3±0.5 degrees 2θ, at about 8.1±0.5 degrees 2θ, at about 10.3±0.5 degrees 2θ, at about 11.5±0.5 degrees 2θ, at about 13.1±0.5 degrees 2θ, at about 15.4±0.5 degrees 2θ, at about 16.1±0.5 degrees 2θ, at about 17.0±0.5 degrees 2θ, at about 18.3±0.5 degrees 2θ, at about 19.2±0.5 degrees 2θ, at about 21.0±0.5 degrees 2θ, at about 21.7±0.5 degrees 2θ, at about 22.3±0.5 degrees 2θ, and at about 23.1±0.5 degrees 2θ.

8. The crystalline form of claim 6, wherein the crystalline Form II is characterized by an X-ray powder diffraction pattern comprising one or more peaks as shown in FIG. 6.

9. The crystalline form of claim 6, wherein the crystalline Form II is characterized by an X-ray powder diffraction pattern comprising one or more d-spacing values at about 12.1±0.5 degrees angstroms, at about 10.9±0.5 degrees angstroms, at about 8.6±0.5 degrees angstroms, at about 7.7±0.5 degrees angstroms, at about 6.8±0.5 degrees angstroms, at about 5.7±0.5 degrees angstroms, at about 5.5±0.5 degrees angstroms, at about 5.2±0.5 degrees angstroms, at about 4.8±0.5 degrees angstroms, at about 4.6±0.5 degrees angstroms, at about 4.2±0.5 degrees angstroms, at about 4.1±0.5 degrees angstroms, at about 4.0±0.5 degrees angstroms, and at about 3.8±0.5 degrees angstroms.

10. A crystalline form comprising a compound having a formula of 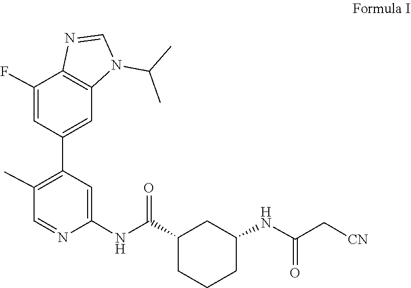 [SMILES: Cc1cnc(NC(=O)[C@H]2CCC[C@@H](NC(=O)CC#N)C2)cc1-c1cc(F)c2ncn(C(C)C)c2c1]
and a coformer.

11. The crystalline form of claim 10, wherein the coformer is an acid.

12. The crystalline form of claim 11, wherein the acid is a pharmaceutically acceptable acid.

13. The crystalline form of claim 12, wherein the pharmaceutically acceptable acid is selected from succinic acid, adipic acid, fumaric acid, glutaric acid, gentisic acid, hydrochloric acid, 1-hydroxy-2-naphthoic acid, salicylic acid, oxalic acid, and D-(−)-tartaric acid.

14. A crystalline form comprising a compound having a formula of 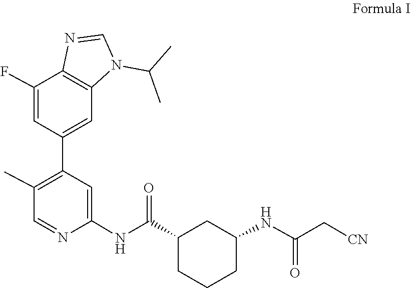 [SMILES: Cc1cnc(NC(=O)[C@H]2CCC[C@@H](NC(=O)CC#N)C2)cc1-c1cc(F)c2ncn(C(C)C)c2c1]
and succinic acid.

15. The crystalline form of claim 14, wherein the molar ratio of the compound of Formula I to succinic acid is about 1:1.

16. The crystalline form of claim 15, wherein the crystalline form is Form III.

17. The crystalline form of claim 16, wherein the crystalline Form III is characterized by an X-ray powder diffraction pattern comprising one or more peaks at about 5.8±0.5 degrees 2θ, at about 8.8±0.5 degrees 2θ, at about 10.5±0.5 degrees 2θ, at about 12.4±0.5 degrees 2θ, at about 14.4±0.5 degrees 2θ, at about 17.5±0.5 degrees 2θ, at about 17.9±0.5 degrees 2θ, at about 18.5±0.5 degrees 2θ, at about 19.5±0.5 degrees 2θ, at about 20.0±0.5 degrees 2θ, at about 20.7±0.5 degrees 2θ, at about 21.4±0.5 degrees 2θ, at about 22.4±0.5 degrees 2θ, at about 22.7±0.5 degrees 2θ, at about 23.6±0.5 degrees 2θ, at about 24.0±0.5 degrees 2θ, at about 24.7±0.5 degrees 2θ, at about 25.9±0.5 degrees 2θ, at about 26.4±0.5 degrees 2θ, at about 27.6±0.5 degrees 2θ, at about 29.0±0.5 degrees 2θ, at about 31.6±0.5 degrees 2θ, and at about 39.5±0.5 degrees 2θ.

18. The crystalline form of claim 16, wherein the crystalline Form III is characterized by an X-ray powder diffraction pattern comprising one or more peaks as shown in FIG. 12.

19. The crystalline form of claim 16, wherein the crystalline Form III is characterized by an X-ray powder diffraction pattern comprising one or more d-spacing values at about 15.1±0.5 degrees angstroms, at about 10.0±0.5 degrees angstroms, at about 8.4±0.5 degrees angstroms, at about 7.1±0.5 degrees angstroms, at about 6.2±0.5 degrees angstroms, at about 5.1±0.5 degrees angstroms, at about 4.9±0.5 degrees angstroms, at about 4.8±0.5 degrees angstroms, at about 4.6±0.5 degrees angstroms, at about 4.4±0.5 degrees angstroms, at about 4.3±0.5 degrees angstroms, at about 4.2±0.5 degrees angstroms, at about 4.0±0.5 degrees angstroms, at about 3.9±0.5 degrees angstroms, at about 3.8±0.5 degrees angstroms, at about 3.7±0.5 degrees angstroms, at about 3.6±0.5 degrees angstroms, at about 3.4±0.5 degrees angstroms, at about 3.4±0.5 degrees angstroms, at about 3.2±0.5 degrees angstroms, at about 3.1±0.5 degrees angstroms, at about 2.8±0.5 degrees angstroms, and at about 2.3±0.5 degrees angstroms.

20. A crystalline form comprising a compound having a formula of 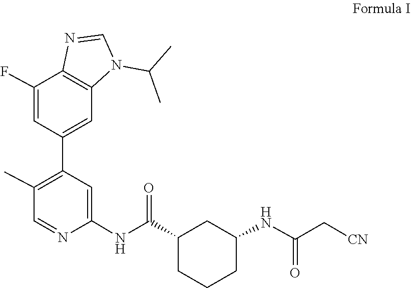 [SMILES: Cc1cnc(NC(=O)[C@H]2CCC[C@@H](NC(=O)CC#N)C2)cc1-c1cc(F)c2ncn(C(C)C)c2c1]
and glutaric acid.

21. The crystalline form of claim 20, wherein the molar ratio of the compound of Formula I to glutaric acid is about 2:1.

22. The crystalline form of claim 21, wherein the crystalline form is Form IV.

23. The crystalline form of claim 22, wherein the crystalline Form IV is characterized by an X-ray powder diffraction pattern comprising one or more peaks at about 4.5±0.5 degrees 2θ, at about 6.0±0.5 degrees 2θ, at about 8.9±0.5 degrees 2θ, at about 11.1±0.5 degrees 2θ, at about 11.7±0.5 degrees 2θ, at about 13.2±0.5 degrees 2θ, at about 16.3±0.5 degrees 2θ, at about 17.1±0.5 degrees 2θ, at about 17.6±0.5 degrees 2θ, at about 18.4±0.5 degrees 2θ, at about 19.7±0.5 degrees 2θ, at about 20.5±0.5 degrees 2θ, at about 21.0±0.5 degrees 2θ, at about 21.9±0.5 degrees 2θ, at about 24.0±0.5 degrees 2θ, at about 24.7±0.5 degrees 2θ, at about 25.0±0.5 degrees 2θ, at about 26.2±0.5 degrees 2θ, and at about 29.2±0.5 degrees 2θ.

24. The crystalline form of claim 22, wherein the crystalline Form IV is characterized by an X-ray powder diffraction pattern comprising one or more peaks as shown in FIG. 18.

25. The crystalline form of claim 22, wherein the crystalline Form IV is characterized by an X-ray powder diffraction pattern comprising one or more d-spacing values at about 19.4±0.5 degrees angstroms, at about 14.7±0.5 degrees angstroms, at about 10.0±0.5 degrees angstroms, at about 7.9±0.5 degrees angstroms, at about 7.5±0.5 degrees angstroms, at about 6.7±0.5 degrees angstroms, at about 5.4±0.5 degrees angstroms, at about 5.2±0.5 degrees angstroms, at about 5.0±0.5 degrees angstroms, at about 4.8±0.5 degrees angstroms, at about 4.5±0.5 degrees angstroms, at about 4.3±0.5 degrees angstroms, at about 4.2±0.5 degrees angstroms, at about 4.1±0.5 degrees angstroms, at about 3.7±0.5 degrees angstroms, at about 3.6±0.5 degrees angstroms, at about 3.6±0.5 degrees angstroms, at about 3.4±0.5 degrees angstroms, and at about 3.1±0.5 degrees angstroms.

26. A crystalline form comprising a compound having a formula of 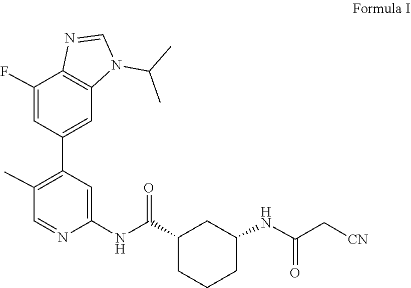 [SMILES: Cc1cnc(NC(=O)[C@H]2CCC[C@@H](NC(=O)CC#N)C2)cc1-c1cc(F)c2ncn(C(C)C)c2c1]
and adipic acid.

27. The crystalline form of claim 26, wherein the molar ratio of the compound of Formula I to adipic acid is about 1:1.

28. The crystalline form of claim 27, wherein the crystalline form is Form V.

29. The crystalline form of claim 28, wherein the crystalline Form V is characterized by an X-ray powder diffraction pattern comprising one or more peaks at about 4.7±0.5 degrees 2θ, at about 7.4±0.5 degrees 2θ, at about 9.2±0.5 degrees 2θ, at about 11.2±0.5 degrees 2θ, at about 13.8±0.5 degrees 2θ, at about 17.2±0.5 degrees 2θ, at about 18.1±0.5 degrees 2θ, at about 18.9±0.5 degrees 2θ, at about 25.1±0.5 degrees 2θ, and at about 25.9±0.5 degrees 2θ.

30. The crystalline form of claim 28, wherein the crystalline Form V is characterized by an X-ray powder diffraction pattern comprising one or more peaks as shown in FIG. 24.

31. The crystalline form of claim 28, wherein the crystalline Form V is characterized by an X-ray powder diffraction pattern comprising one or more d-spacing values at about 18.8±0.5 degrees angstroms, at about 11.9±0.5 degrees angstroms, at about 9.6±0.5 degrees angstroms, at about 7.9±0.5 degrees angstroms, at about 6.4±0.5 degrees angstroms, at about 5.1±0.5 degrees angstroms, at about 4.9±0.5 degrees angstroms, at about 4.7±0.5 degrees angstroms, at about 3.6±0.5 degrees angstroms, and at about 3.4±0.5 degrees angstroms.

32. A crystalline form comprising a compound having a formula of 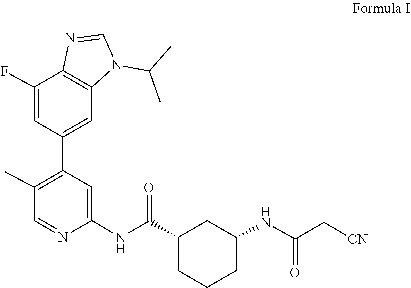 [SMILES: Cc1cnc(NC(=O)[C@H]2CCC[C@@H](NC(=O)CC#N)C2)cc1-c1cc(F)c2ncn(C(C)C)c2c1]
and gentisic acid.

33. The crystalline form of claim 32, wherein the molar ratio of the compound of Formula I to gentisic acid is about 2:1.

34. The crystalline form of claim 33, wherein the crystalline form is Form VI.

35. The crystalline form of claim 34, wherein the crystalline Form VI is characterized by an X-ray powder diffraction pattern comprising one or more peaks at about 7.4±0.5 degrees 2θ, at about 9.5±0.5 degrees 2θ, at about 13.6±0.5 degrees 2θ, at about 14.7±0.5 degrees 2θ, at about 15.5±0.5 degrees 2θ, at about 16.4±0.5 degrees 2θ, at about 17.2±0.5 degrees 2θ, at about 18.2±0.5 degrees 2θ, at about 19.4±0.5 degrees 2θ, at about 20.5±0.5 degrees 2θ, at about 21.5±0.5 degrees 2θ, at about 23.5±0.5 degrees 2θ, at about 24.8±0.5 degrees 2θ, at about 25.7±0.5 degrees 2θ, at about 26.9±0.5 degrees 2θ, at about 29.4±0.5 degrees 2θ, and at about 30.7±0.5 degrees 2θ.

36. The crystalline form of claim 34, wherein the crystalline Form VI is characterized by an X-ray powder diffraction pattern comprising one or more peaks as shown in FIG. 36.

37. The crystalline form of claim 34, wherein the crystalline Form VI is characterized by an X-ray powder diffraction pattern comprising one or more d-spacing values at about 12.0±0.5 degrees angstroms, at about 9.3±0.5 degrees angstroms, at about 6.5±0.5 degrees angstroms, at about 6.0±0.5 degrees angstroms, at about 5.7±0.5 degrees angstroms, at about 5.4±0.5 degrees angstroms, at about 5.2±0.5 degrees angstroms, at about 4.9±0.5 degrees angstroms, at about 4.6±0.5 degrees angstroms, at about 4.3±0.5 degrees angstroms, at about 4.1±0.5 degrees angstroms, at about 3.8±0.5 degrees angstroms, at about 3.6±0.5 degrees angstroms, at about 3.5±0.5 degrees angstroms, at about 3.3±0.5 degrees angstroms, at about 3.0±0.5 degrees angstroms, and at about 2.9±0.5 degrees angstroms.

38. A crystalline form comprising a compound having a formula of 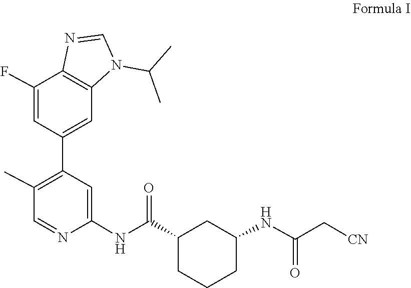 [SMILES: Cc1cnc(NC(=O)[C@H]2CCC[C@@H](NC(=O)CC#N)C2)cc1-c1cc(F)c2ncn(C(C)C)c2c1]
and fumaric acid.

39. The crystalline form of claim 38, wherein the molar ratio of the compound of Formula I to fumaric acid is about 2:1.

40. The crystalline form of claim 39, wherein the crystalline form is Form VII.

41. The crystalline form of claim 40, wherein the crystalline Form VII is characterized by an X-ray powder diffraction pattern comprising one or more peaks at about 4.7±0.5 degrees 2θ, at about 5.8±0.5 degrees 2θ, at about 10.6±0.5 degrees 2θ, at about 11.3±0.5 degrees 2θ, at about 11.8±0.5 degrees 2θ, at about 12.6±0.5 degrees 2θ, at about 13.1±0.5 degrees 2θ, at about 14.0±0.5 degrees 2θ, at about 16.0±0.5 degrees 2θ, at about 17.0±0.5 degrees 2θ, at about 17.5±0.5 degrees 2θ, at about 18.7±0.5 degrees 2θ, at about 19.3±0.5 degrees 2θ, at about 21.2±0.5 degrees 2θ, at about 22.1±0.5 degrees 2θ, at about 24.2±0.5 degrees 2θ, at about 24.7±0.5 degrees 2θ, at about 26.2±0.5 degrees 2θ, and at about 27.4±0.5 degrees 2θ.

42. The crystalline form of claim 40, wherein the crystalline Form VII is characterized by an X-ray powder diffraction pattern comprising one or more peaks as shown in FIG. 30.

43. The crystalline form of claim 40, wherein the crystalline Form VII is characterized by an X-ray powder diffraction pattern comprising one or more d-spacing values at about 18.9±0.5 degrees angstroms, at about 15.2±0.5 degrees angstroms, at about 8.4±0.5 degrees angstroms, at about 7.8±0.5 degrees angstroms, at about 7.5±0.5 degrees angstroms, at about 7.0±0.5 degrees angstroms, at about 6.8±0.5 degrees angstroms, at about 6.3±0.5 degrees angstroms, at about 5.5±0.5 degrees angstroms, at about 5.2±0.5 degrees angstroms, at about 5.1±0.5 degrees angstroms, at about 4.8±0.5 degrees angstroms, at about 4.6±0.5 degrees angstroms, at about 4.2±0.5 degrees angstroms, at about 4.0±0.5 degrees angstroms, at about 3.7±0.5 degrees angstroms, at about 3.6±0.5 degrees angstroms, at about 3.4±0.5 degrees angstroms, and at about 3.2±0.5 degrees angstroms.

44. The crystalline form of claim 38, wherein the molar ratio of the compound of Formula I to fumaric acid is about 1:1.

45. The crystalline form of claim 39, wherein the crystalline form is Form VIII.

46. The crystalline form of claim 45, wherein the crystalline Form VIII is characterized by an X-ray powder diffraction pattern comprising one or more peaks at about 3.9±0.5 degrees 2θ, at about 5.7±0.5 degrees 2θ, at about 7.1±0.5 degrees 2θ, at about 8.6±0.5 degrees 2θ, at about 10.3±0.5 degrees 2θ, at about 12.1±0.5 degrees 2θ, at about 14.1±0.5 degrees 2θ, at about 17.1±0.5 degrees 2θ, at about 19.1±0.5 degrees 2θ, at about 20.6±0.5 degrees 2θ, at about 22.2±0.5 degrees 2θ, at about 23.0±0.5 degrees 2θ, at about 24.3±0.5 degrees 2θ, at about 26.0±0.5 degrees 2θ, at about 26.5±0.5 degrees 2θ, at about 28.5±0.5 degrees 2θ, at about 34.6±0.5 degrees 2θ, at about 35.4±0.5 degrees 2θ, at about 36.8±0.5 degrees 2θ, and at about 39.5±0.5 degrees 2θ.

47. The crystalline form of claim 45, wherein the crystalline Form VIII is characterized by an X-ray powder diffraction pattern comprising one or more peaks as shown in FIG. 42.

48. The crystalline form of claim 45, wherein the crystalline Form VIII is characterized by an X-ray powder diffraction pattern comprising one or more d-spacing values at about 22.5±0.5 degrees angstroms, at about 15.5±0.5 degrees angstroms, at about 12.5±0.5 degrees angstroms, at about 10.3±0.5 degrees angstroms, at about 8.6±0.5 degrees angstroms, at about 7.3±0.5 degrees angstroms, at about 6.3±0.5 degrees angstroms, at about 5.2±0.5 degrees angstroms, at about 4.7±0.5 degrees angstroms, at about 4.3±0.5 degrees angstroms, at about 4.0±0.5 degrees angstroms, at about 3.9±0.5 degrees angstroms, at about 3.7±0.5 degrees angstroms, at about 3.4±0.5 degrees angstroms, at about 3.4±0.5 degrees angstroms, at about 3.1±0.5 degrees angstroms, at about 2.6±0.5 degrees angstroms, at about 2.5±0.5 degrees angstroms, at about 2.4±0.5 degrees angstroms, and at about 2.3±0.5 degrees angstroms.

49. A crystalline form comprising a compound having a formula of 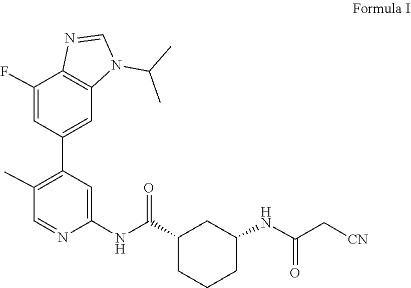 [SMILES: Cc1cnc(NC(=O)[C@H]2CCC[C@@H](NC(=O)CC#N)C2)cc1-c1cc(F)c2ncn(C(C)C)c2c1]
and D-(−)-tartaric acid.

50. The crystalline form of claim 49, wherein the molar ratio of the compound of Formula I to D-(−)-tartaric acid is about 1:1.

51. The crystalline form of claim 50, wherein the crystalline form is Form IX.

52. The crystalline form of claim 51, wherein the crystalline Form IX is characterized by an X-ray powder diffraction pattern comprising one or more peaks at about 5.3±0.5 degrees 2θ, at about 6.8±0.5 degrees 2θ, at about 9.0±0.5 degrees 2θ, at about 10.0±0.5 degrees 2θ, at about 15.5±0.5 degrees 2θ, at about 17.3±0.5 degrees 2θ, at about 18.2±0.5 degrees 2θ, at about 18.8±0.5 degrees 2θ, at about 19.9±0.5 degrees 2θ, at about 20.9±0.5 degrees 2θ, at about 21.3±0.5 degrees 2θ, at about 22.7±0.5 degrees 2θ, at about 23.6±0.5 degrees 2θ, at about 24.3±0.5 degrees 2θ, at about 25.5±0.5 degrees 2θ, at about 26.0±0.5 degrees 2θ, at about 27.1±0.5 degrees 2θ, at about 28.0±0.5 degrees 2θ, at about 28.8±0.5 degrees 2θ, at about 29.8±0.5 degrees 2θ, at about 33.4±0.5 degrees 2θ, at about 34.2±0.5 degrees 2θ, at about 36.3±0.5 degrees 2θ, at about 38.6±0.5 degrees 2θ, and at about 39.1±0.5 degrees 2θ.

53. The crystalline form of claim 51, wherein the crystalline Form IX is characterized by an X-ray powder diffraction pattern comprising one or more peaks as shown in FIG. 46.

54. The crystalline form of claim 51, wherein the crystalline Form IX is characterized by an X-ray powder diffraction pattern comprising one or more d-spacing values at about 16.7±0.5 degrees angstroms, at about 12.9±0.5 degrees angstroms, at about 9.8±0.5 degrees angstroms, at about 8.8±0.5 degrees angstroms, at about 5.7±0.5 degrees angstroms, at about 5.1±0.5 degrees angstroms, at about 4.9±0.5 degrees angstroms, at about 4.7±0.5 degrees angstroms, at about 4.5±0.5 degrees angstroms, at about 4.3±0.5 degrees angstroms, at about 4.2±0.5 degrees angstroms, at about 3.9±0.5 degrees angstroms, at about 3.8±0.5 degrees angstroms, at about 3.7±0.5 degrees angstroms, at about 3.5±0.5 degrees angstroms, at about 3.4±0.5 degrees angstroms, at about 3.3±0.5 degrees angstroms, at about 3.2±0.5 degrees angstroms, at about 3.1±0.5 degrees angstroms, at about 3.0±0.5 degrees angstroms, at about 2.7±0.5 degrees angstroms, at about 2.6±0.5 degrees angstroms, at about 2.5±0.5 degrees angstroms, at about 2.3±0.5 degrees angstroms, and at about 2.3±0.5 degrees angstroms.

55. A crystalline form comprising a compound having a formula of 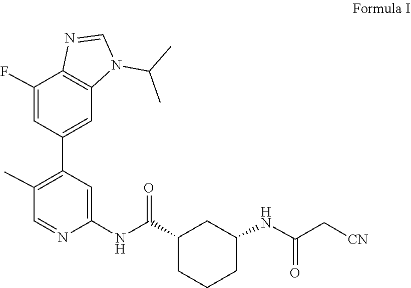 [SMILES: Cc1cnc(NC(=O)[C@H]2CCC[C@@H](NC(=O)CC#N)C2)cc1-c1cc(F)c2ncn(C(C)C)c2c1]
and hydrochloric acid.

56. The crystalline form of claim 55, wherein the crystalline form is Form X.

57. The crystalline form of claim 56, wherein the crystalline Form X is characterized by an X-ray powder diffraction pattern comprising one or more peaks as shown in FIG. 50.

58. A crystalline form comprising a compound having a formula of 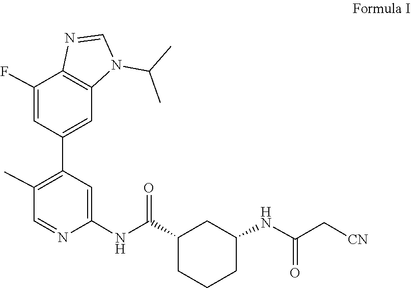 [SMILES: Cc1cnc(NC(=O)[C@H]2CCC[C@@H](NC(=O)CC#N)C2)cc1-c1cc(F)c2ncn(C(C)C)c2c1]
and salicylic acid.

59. The crystalline form of claim 58, wherein the molar ratio of the compound of Formula I to salicylic acid is about 2:1.

60. The crystalline form of claim 59, wherein the crystalline form is Form XI.

61. The crystalline form of claim 60, wherein the crystalline Form XI is characterized by an X-ray powder diffraction pattern comprising one or more peaks as shown in FIG. 52.

62. A crystalline form comprising a compound having a formula of 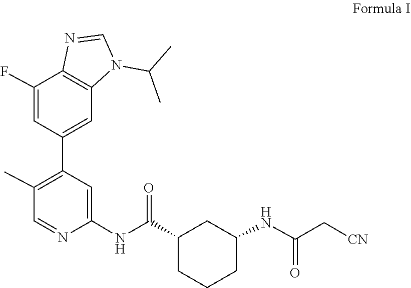 [SMILES: Cc1cnc(NC(=O)[C@H]2CCC[C@@H](NC(=O)CC#N)C2)cc1-c1cc(F)c2ncn(C(C)C)c2c1]
and oxalic acid.

63. The crystalline form of claim 62, wherein the molar ratio of the compound of Formula I to oxalic acid is about 1:1.

64. The crystalline form of claim 63, wherein the crystalline form is Form XII.

65. The crystalline form of claim 64, wherein the crystalline Form XII is characterized by an X-ray powder diffraction pattern comprising one or more peaks as shown in FIG. 54.

66. A crystalline form comprising a compound having a formula of 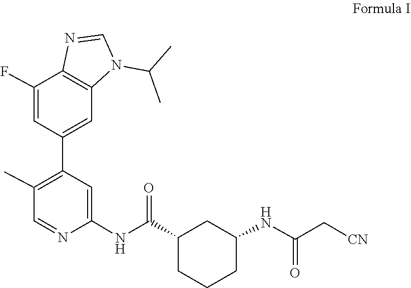 [SMILES: Cc1cnc(NC(=O)[C@H]2CCC[C@@H](NC(=O)CC#N)C2)cc1-c1cc(F)c2ncn(C(C)C)c2c1]
and 1-hydroxy-2-naphthoic acid.

67. The crystalline form of claim 66, wherein the crystalline form is Form XIII.

68. The crystalline form of claim 67, wherein the crystalline Form XIII is characterized by an X-ray powder diffraction pattern comprising one or more peaks as shown in FIG. 59.

69. A pharmaceutical composition comprising a crystalline form of any one of claims 1-68.

70. A pharmaceutical composition comprising the crystalline Form I of any one of claims 2-5.

71. A pharmaceutical composition comprising the crystalline Form II of any one of claims 6-9.

72. A pharmaceutical composition comprising the crystalline Form III of any one of claims 16-19.

73. A pharmaceutical composition comprising the crystalline Form IV of any one of claims 22-25.

74. A pharmaceutical composition comprising the crystalline Form V of any one of claims 28-31.

75. A pharmaceutical composition comprising the crystalline Form VI of any one of claims 34-37.

76. A pharmaceutical composition comprising the crystalline Form VII of any one of claims 40-43.

77. A pharmaceutical composition comprising the crystalline Form VIII of any one of claims 45-48.

78. A pharmaceutical composition comprising the crystalline Form IX of any one of claims 51-54.

79. A pharmaceutical composition comprising the crystalline Form X of any one of claims 56-57.

80. A pharmaceutical composition comprising the crystalline Form XI of any one of claims 60-61.

81. A pharmaceutical composition comprising the crystalline Form XII of any one of claims 64-65.

82. A pharmaceutical composition comprising the crystalline Form XIII of any one of claims 67-68.

83. The pharmaceutical composition of claim 69, further comprising a pharmaceutically acceptable excipient.

84. A process for preparing a crystalline form of any one of claims 1-68, comprising crystallizing the compound of 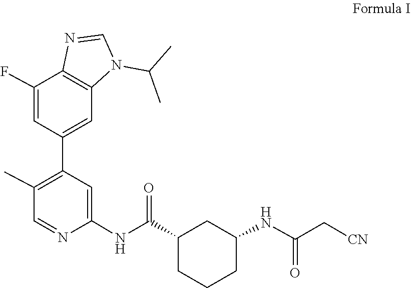 [SMILES: Cc1cnc(NC(=O)[C@H]2CCC[C@@H](NC(=O)CC#N)C2)cc1-c1cc(F)c2ncn(C(C)C)c2c1]
to form the crystalline form and optionally isolating the crystalline form.

85. The process of claim 84, wherein the crystallizing comprises dissolving the compound of Formula I in an organic solvent and crystallizing the compound of Formula I to form the crystalline Form I or Form II therefrom.

86. The process of claim 84, wherein the crystallizing comprises dissolving the compound of Formula I with a pharmaceutically acceptable acid in an organic solvent and crystallizing the compound of Formula I to form any one of the crystalline Form III to Form XIII therefrom.

87. The process of claim 86, wherein the pharmaceutically acceptable acid is selected from succinic acid, adipic acid, fumaric acid, glutaric acid, gentisic acid, hydrochloric acid, 1-hydroxy-2-naphthoic acid, salicylic acid, oxalic acid, and D-(−)-tartaric acid.

88. The process of claim 84, wherein the crystallizing comprises dissolving the compound of Formula I with succinic acid in an organic solvent and crystallizing the compound of Formula I to form the crystalline Form III therefrom.

89. The process of any one of claims 85-88, wherein the organic solvent is selected from the group consisting of acetonitrile, n-butanol, methyl ethyl ketone, methanol, ethyl acetate, acetone, tetrahydrofuran, 2-propanol, ethanol, isopropyl acetate, toluene, cyclohexane, dichloromethane, chloroform, H₂O, nitromethane, n-pentane, n-hexane, 1-propanol, methyl acetate, ethyl ether, octane, and any combination thereof.

90. The process of claim 85, wherein the solvent is acetonitrile.

91. The process of claim 88, wherein the solvent is ethyl acetate.

92. A method of inhibiting a CDK enzyme comprising: contacting the CDK enzyme with an effective amount of a crystalline form of any one of claims 1-68.

93. The method of claim 92, wherein the CDK enzyme is CDK9.

94. A method of treating a disease or disorder associated with aberrant CDK activity in a subject or a subject in need thereof comprising administering to the subject a crystalline form of any one of claims 1-68.

95. The method of claim 94, wherein the disease or disorder associated with aberrant CDK activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

96. A method of treating cancer in a subject or a subject in need thereof comprising administering to the subject a crystalline form of any one of claims 1-68.

97. The method of claim 96, wherein the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

98. A method of inducing apoptosis in a cancer or tumor cell in a subject or a subject in need thereof comprising contacting the cancer or tumor cell with, or administering to the subject an effective amount of a crystalline form of any one of claims 1-68.

99. A method of inhibiting phosphorylation of Ser2RNAP2 in a cancer or tumor cell in a subject or a subject in need thereof comprising contacting the cancer or tumor cell with, or administering to the subject an effective amount of a crystalline form of any one of claims 1-68.

100. A method of reducing the level of induced myeloid leukemia cell differentiation protein Mcl-1 (MCL1) in a cancer or tumor cell in a subject or a subject in need thereof comprising contacting the cancer or tumor cell with, or administering to the subject an effective amount of a crystalline form of any one of claims 1-68.

101. A method of reducing the level of MYC protein in a cancer or tumor cell in a subject or a subject in need thereof comprising contacting the cancer or tumor cell with, or administering to the subject an effective amount of a crystalline form of any one of claims 1-68.

102. A method of inhibiting proliferation of a cancer or tumor cell in a subject or a subject in need thereof comprising contacting the cancer or tumor cell with, or administering to the subject an effective amount of a crystalline form of any one of claims 1-68.

103. The method of any one of claims 98-102, wherein the cancer or tumor has high levels of MYC amplification and overexpression.

104. The method of any one of claims 98-103, wherein the cancer cell is malignant.

105. The method of any one of claims 98-103, wherein the cancer cell is a hematological cancer cell.

106. The method of claim 105, wherein the hematological cancer is a B-Cell Acute Lymphoblastic Leukemia (B-ALL), T-Cell Acute Lymphoblastic Leukemia (T-ALL), Acute Myeloid Leukemia (AML), non-Hodgkin's lymphoma, sarcoma, prostate, adenoid cystic carcinoma (ACC), or non-small cell lung cancer (NSCLC).

107. The method of any one of claims 98-102, wherein the tumor cell is from a solid tumor.

108. The method of claim 106, wherein the solid tumor is pancreatic carcinoma, gastric and gastroesophageal carcinomas, NSCLC, or sarcoma.

109. The method according to claims 107 or 108, further comprising contacting the tumor cell with a targeted therapy.

110. The method of claim 109, wherein the targeted therapy is a BCL2 inhibitor.

111. The method of claim 110, wherein the BCL2 inhibitor is venetoclax.

112. The method of any one of claims 109-111, wherein a complete tumor regression is achieved.
